(19) United States
(12) Patent Application Publication
(10) Pub. No.: US 2017/0033293 A1
(43) Pub. Date: Feb. 2, 2017

(54) ELECTRON TRANSPORT MATERIAL AND ORGANIC ELECTROLUMINESCENT DEVICE USING THE SAME

(71) Applicant: JNC CORPORATION, Tokyo (JP)

(72) Inventors: Daisuke BABA, Chiba (JP); Yohei ONO, Chiba (JP)

(73) Assignee: JNC CORPORATION, Tokyo (JP)

(21) Appl. No.: 15/124,671

(22) PCT Filed: Mar. 16, 2015
(86) PCT No.: PCT/JP2015/057617

§ 371 Date: Sep. 8, 2016

(30) Foreign Application Priority Data
Mar. 17, 2014 (JP) 2014-053838

(51) Int. Cl. H01L 51/00; C07D 213/85

(52) U.S. Cl. CPC H01L 51/0067; C07D 213/85; H01L 51/0058; H01L 51/0052; H01L 51/0056; H01L 51/0054; H01L 51/0077; H01L 51/5072

(57) ABSTRACT

The present invention is a compound represented by formula (1).

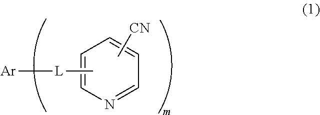

Ar is an m-valent group derived from an aromatic hydrocarbon having 6 to 40 carbons or an aromatic heterocycle having 2 to 40 carbons; m is an integer from 1 to 4; L is a single bond or one selected from a group of divalent groups represented by formulae (L-1) and (L-2) below; X¹ to X⁶ and X⁷ to X¹⁴ are independently ═CR¹— or ═N—, wherein at least two are ═CR¹—, R¹ in two ═CR¹— is a bond, and R¹ in the other ═CR¹— is hydrogen; and at least one hydrogen of each ring and alkyl in formula (1) may be replaced by deuterium.

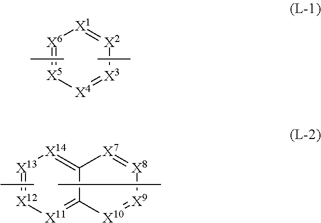

This application is the U.S. National Phase under 35 U.S.C. §371 of International Application No. PCT/JP2015/057617, filed Mar. 16, 2015, designated the U.S., and published in Japanese as WO 2015/141608 on Sep. 24, 2015, which claims priority to Japanese Patent Application No. 2014-053838, filed Mar. 17, 2014, the entire contents of which are incorporated herein by reference.

### TECHNICAL FIELD

The present invention relates to a new electron transport material having a cyanopyridyl group, an organic electroluminescent device (hereinafter, may be abbreviated as organic EL device, or merely device) using this electron transport material, and the like.

### BACKGROUND ART

In recent years, organic EL devices have attracted attention as a next-generation full color flat panel display, and actively researched. In order to promote practical use of organic EL devices, reduction of power consumption of the device (reduction of voltage and improvement in external quantum yield) and extended lifetime are essential elements, and a new electron transport material has been developed to achieve such. In particular, achievement of low power consumption or extended lifetime of blue devices is a challenge, and various electron transport materials have been investigated. As described in Patent Documents 1 to 4 and Non-Patent Document 1, it is known that an organic EL device can be driven at low voltage by using a pyridine derivative or a bipyridine derivative as an electron transport material. A part thereof has already been practically applied, but such organic EL device has insufficient characteristics in order to be used for a larger number of displays, and further improvement is required.

### DOCUMENTS OF RELATED ART

### Patent Documents

Patent Document 1: Japanese Unexamined Patent Application, First Publication No. 2003-123983
Patent Document 2: Japanese Unexamined Patent Application, First Publication No. 2002-158093
Patent Document 3: Japanese Unexamined Patent Application, First Publication No. 2009-173642
Patent Document 4: International Publication No. WO 2007/086552

### Non-Patent Document

Non-Patent Document 1: Proceedings of the 10th International Workshop on Inorganic and Organic Electroluminescence (2000)

### SUMMARY OF THE INVENTION

### Technical Problem

The present invention has been made in view of such problems of the prior art. An object of the present invention is to provide an electron transport material that can achieve a good balance in improvement of characteristics required for organic EL devices, such as reduction of driving voltage, high efficiency, and extended lifetime. Furthermore, an objective of the present invention is to provide an organic EL device using this electron transport material.

### Solution to Problem

The inventors of the present invention, as a result of carrying out earnest research, found that a good balance in improvement of characteristics, such as reduction of driving voltage, high efficiency, and extended lifetime, can be achieved by, in an electron transport layer of an organic EL device, using an aromatic hydrocarbon or aromatic heterocycle in which there has been substitution with a cyanopyridyl group via a linker, and thus have completed the present invention based on this finding.

The aforementioned problems can be solved by each item shown below.

[1] A compound represented by formula (1) below:

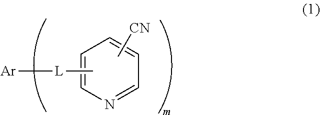

wherein, in formula (1),
Ar is an m-valent group derived from an aromatic hydrocarbon having 6 to 40 carbons or an m-valent group derived from an aromatic heterocycle having 2 to 40 carbons, and at least one hydrogen in these groups may be replaced by an alkyl having 1 to 6 carbons;
m is an integer from 1 to 4, and when m is 2, 3, or 4, the group formed by the pyridine ring and L may be identical or different;
L is a single bond or one selected from the group of divalent groups represented by formulae (L-1) and (L-2) below:

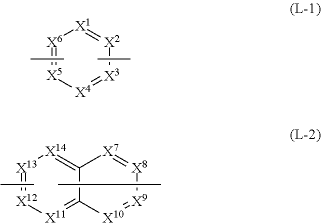

wherein,
in formula (L-1), X¹ to X⁶ are independently ═CR¹— or ═N—, at least two of X¹ to X⁶ are R¹ in two ═CR¹— of X¹ to X⁶ is a bond for bonding with Ar or the pyridine ring, and R¹ in the other ═CR¹— is hydrogen,
in formula (L-2), X⁷ to X¹⁴ are independently ═CR¹— or ═N—, at least two of X⁷ to X¹⁴ are ═CR¹—, R¹ in two ═CR¹— of X⁷ to X¹⁴ is a bond for bonding with Ar or the pyridine ring, and R¹ in the other ═CR¹— is a hydrogen, and
at least one hydrogen of L may be replaced by an alkyl having 1 to 4 carbons or an aryl having 6 to 18 carbons;
at least one hydrogen of the pyridine ring may be replaced by an alkyl having 1 to 4 carbons, phenyl, or naphthyl; and
at least one hydrogen of each ring and alkyl in formula (1) may be replaced by deuterium.

[2] The compound according to the aforementioned item [1], wherein, in formula (1), Ar is one selected from the group of groups represented by formulae (Ar1-1) to (Ar1-12), (Ar2-1) to (Ar2-21), (Ar3-1), (Ar3-2), and (Ar4-1) below:

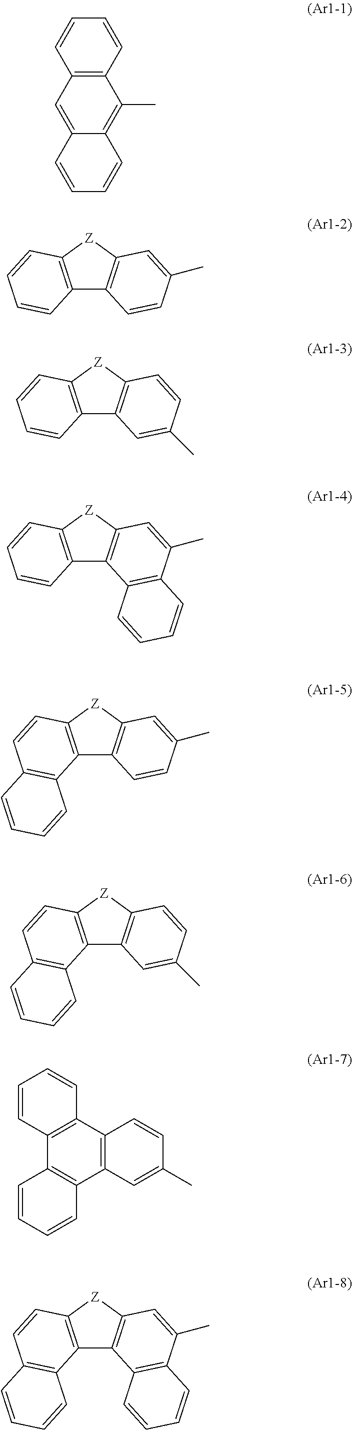

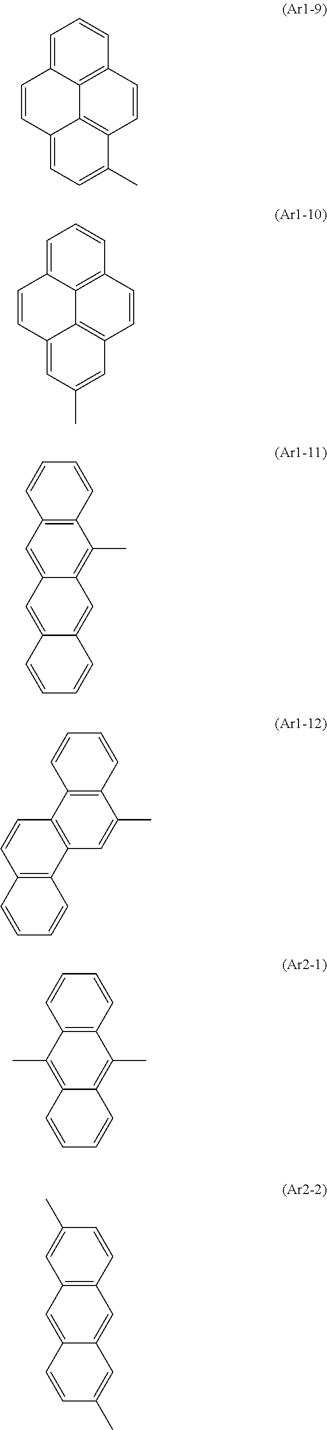

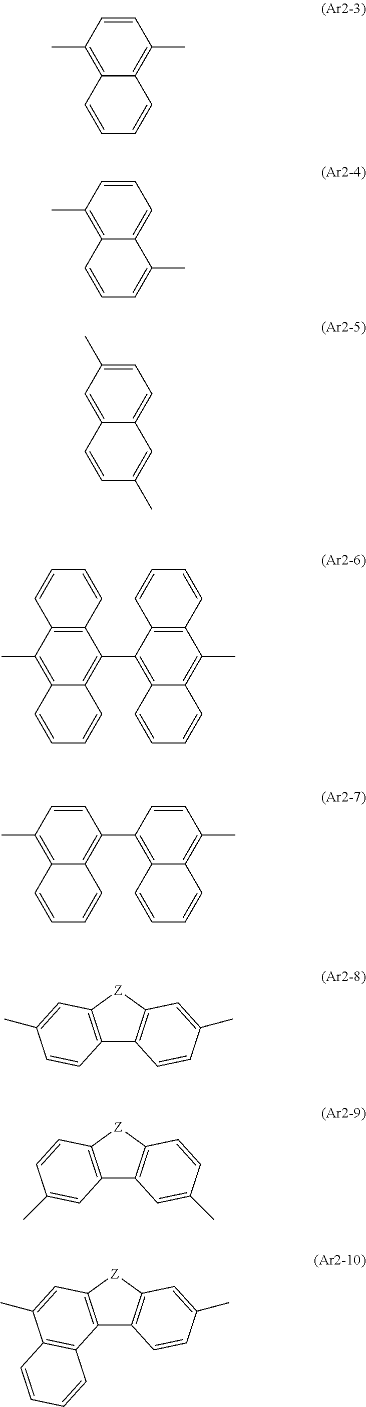

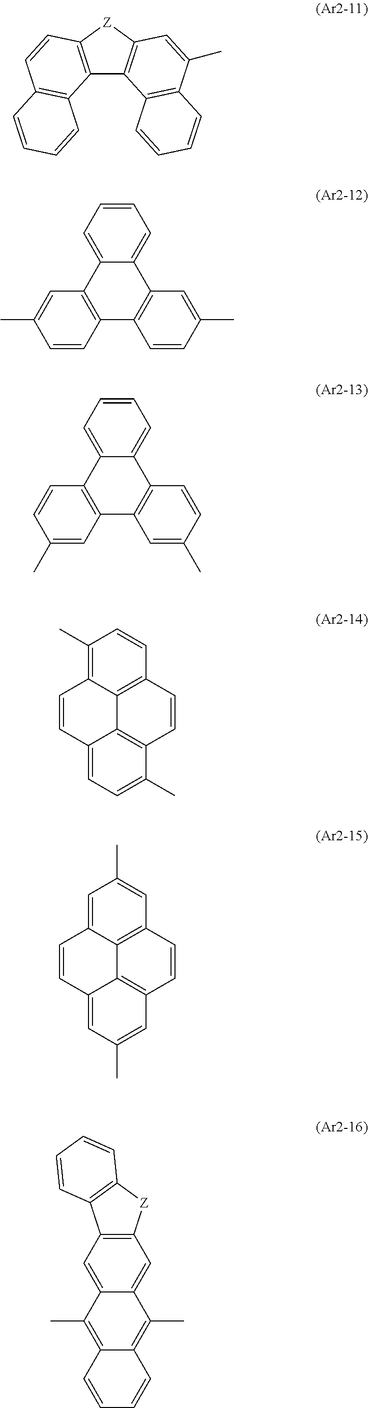

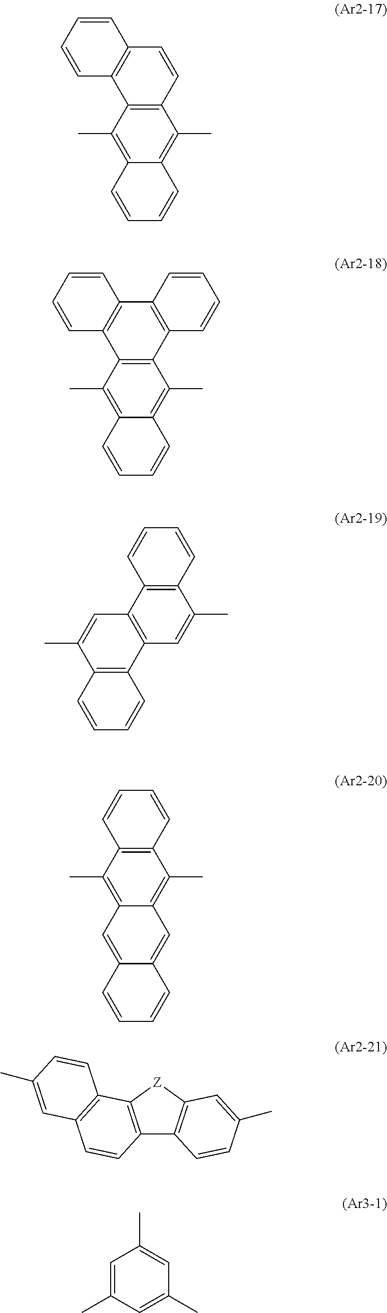

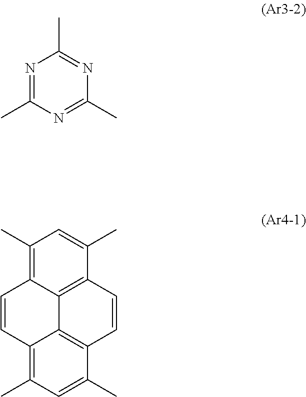

wherein, in formulae (Ar1-1) to (Ar1-12), (Ar2-1) to (Ar2-21), (Ar3-1), (Ar3-2), and (Ar4-1), Z is independently —O—, —S—, or one selected from the group of divalent groups represented by formulae (2) and (3) below, and at least one hydrogen in each group may be replaced by an alkyl having 1 to 4 carbons or an aryl having 6 to 18 carbons:

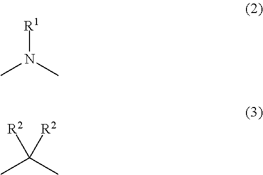

wherein, in formula (2), R¹ is phenyl, naphthyl, biphenylyl, or terphenylyl, and, in formula (3), R² is independently methyl or phenyl, and R² may be linked with each other to form a ring.

[3] The compound according to the aforementioned item [1], wherein, in formula (1), Ar is one selected from the group of groups represented by formulae (Ar1-1) to (Ar1-7), (Ar2-1), (Ar2-3), (Ar2-6) to (Ar2-10), (Ar2-12), (Ar2-21), (Ar3-1), and (Ar3-2) below:

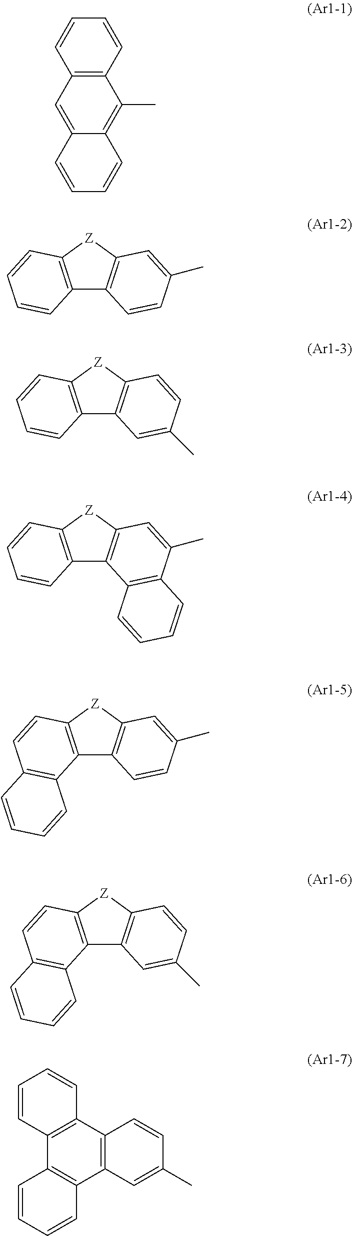

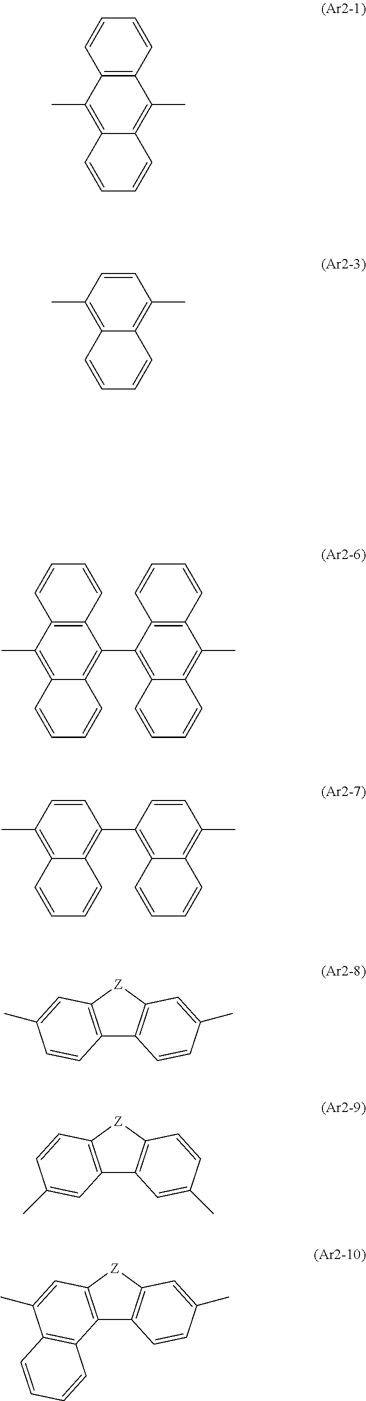

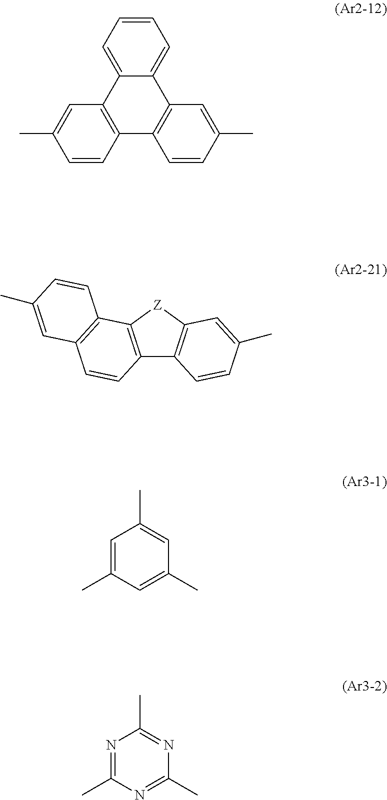

wherein, in formulae (Ar1-1) to (Ar1-7), (Ar2-1), (Ar2-3), (Ar2-6) to (Ar2-10), (Ar2-12), (Ar2-21), (Ar3-1), and (Ar3-2), Z is independently one selected from the group of divalent groups represented by formulae (2) and (3) below, and at least one hydrogen in each group may be replaced by an alkyl having 1 to 4 carbons or an aryl having 6 to 18 carbons:

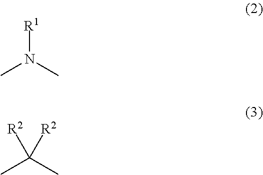

wherein, in formula (2), R¹ is phenyl, naphthyl, biphenylyl, or terphenylyl, and, in formula (3), R² is independently methyl or phenyl, and R² may be linked with each other to form a ring.

[4] The compound according to the aforementioned item [1], wherein, in formula (1), Ar is one selected from the group of groups represented by formulae (Ar1-1), (Ar2-1), (Ar2-8), (Ar2-12), and (Ar2-21) below:

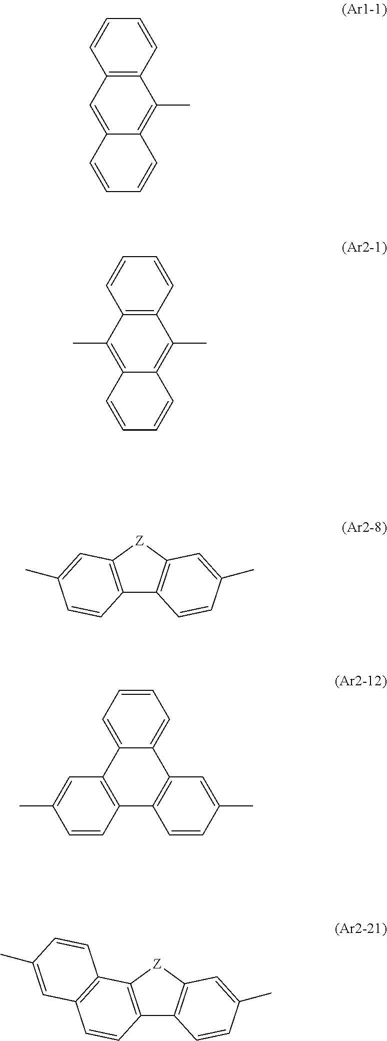

wherein, in formulae (Ar1-1), (Ar2-1), (Ar2-8), (Ar2-12), and (Ar2-21), Z is independently a divalent group represented by formula (4) below, and at least one hydrogen in each group may be replaced by an alkyl having 1 to 4 carbons or an aryl having 6 to 18 carbons:

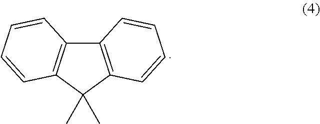

[5] The compound according to the aforementioned item [1], wherein, in formula (1), Ar is one selected from the group of groups represented by formulae (Ar1-1) and (Ar2-1) below:

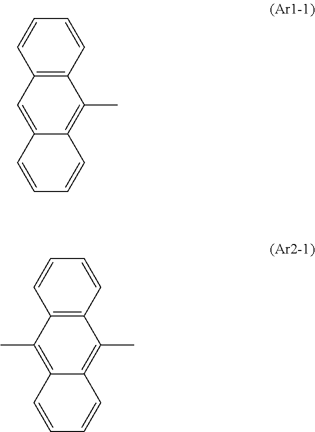

wherein at least one hydrogen in formulae (Ar1-1) and (Ar2-1) may be replaced by an alkyl having 1 to 4 carbons or an aryl having 6 to 18 carbons.

[6] The compound according to the aforementioned item [1] represented by formula (1-1-2) below:

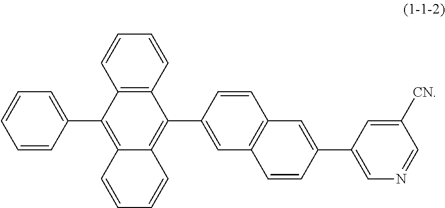 [SMILES: N#Cc1cncc(-c2ccc3cc(-c4c5ccccc5c(-c5ccccc5)c5ccccc45)ccc3c2)c1]

[7] The compound according to the aforementioned item [1] represented by formula (1-2-27) below:

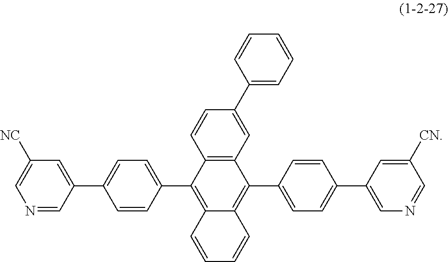 [SMILES: N#Cc1cncc(-c2ccc(-c3c4ccccc4c(-c4ccc(-c5cncc(C#N)c5)cc4)c4cc(-c5ccccc5)ccc34)cc2)c1]

[8] The compound according to the aforementioned item [1] represented by formulae (1-2-48), (1-2-173), (1-2-179), (1-2-365), (1-2-506), or (1-2-507) below:

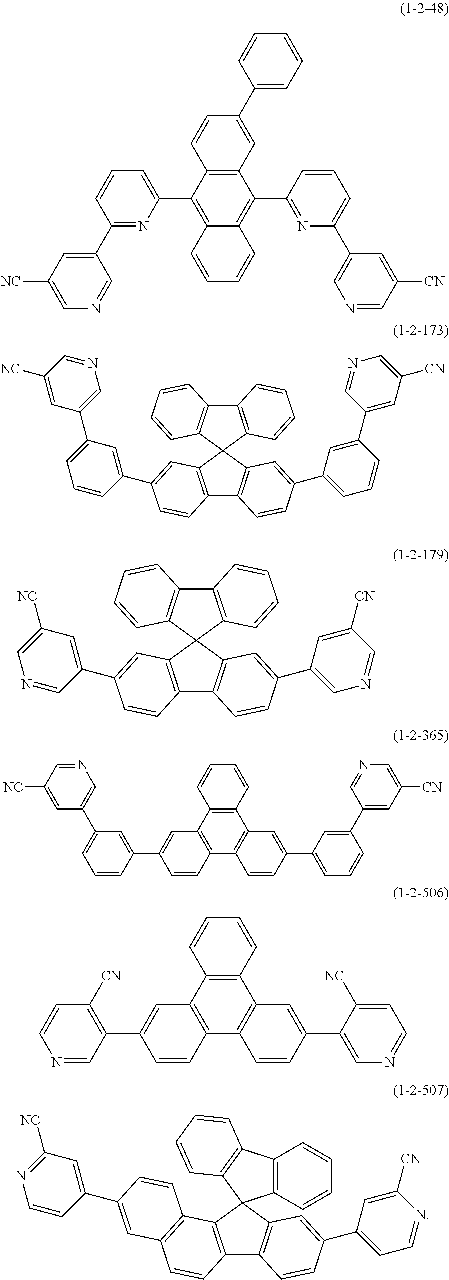

[9] An electron transport material containing the compound according to any one of the aforementioned items [1] to [8].

[10] An organic electroluminescent device having: a pair of electrodes formed of an anode and a cathode; an emission layer arranged between the pair of electrodes; and an electron transport layer and/or an electron injection layer which contain(s) the electron transport material according to the aforementioned item [9] and which are/is arranged between the cathode and the emission layer.

[11] An organic electroluminescent device having: a pair of electrodes formed of an anode and a cathode; an emission layer arranged between the pair of electrodes; and an electron transport layer and an electron injection layer which contain the electron transport material according to the aforementioned item [9] and which are arranged between the cathode and the emission layer.

[12] The organic electroluminescent device according to the aforementioned item [10] or [11], wherein at least one of the electron transport layer and the electron injection layer further contains at least one selected from the group consisting of quinolinol metal complexes, bipyridine derivatives, phenanthroline derivatives, and borane derivatives.

[13] The organic electroluminescent device according to any one of the aforementioned items [10] to [12], wherein at least one of the electron transport layer and the electron injection layer further contains at least one selected from the group consisting of alkali metals, alkali earth metals, rare earth metals, alkali metal oxides, alkali metal halides, alkali earth metal oxides, alkali earth metal halides, rare earth metal oxides, rare earth metal halides, organic complexes of an alkali metal, organic complexes of an alkali earth metal, and organic complexes of a rare earth metal.

### Effects of the Invention

The compound of the present invention has the features of being stable even if voltage is applied in a thin film state and having a high electric charge transport capacity. The compound of the present invention is suitable as an electron transport material in an organic EL device. By using the compound of the present invention in the electron transport layer of an organic EL device, a good balance in improvement of characteristics such as reduction of driving voltage, high efficiency, and extended lifetime, can be achieved. A high performance display device for full color display and the like can be produced by using the organic EL device of the present invention.

### DESCRIPTION OF EMBODIMENTS

The present invention is described in more detail below. Note that, in the present specification, “compound represented by formula (1-1-2)” for example may be referred to as “compound (1-1-2)”. “Compound represented by formula (1-2-27)” may be referred to as “compound (1-2-27)”. The other formula symbols and formula numbers are also handled in the same way.

### Explanation of Compound

The first invention of the present invention is a compound which is represented by formula (1) below and which has cyanopyridyl.

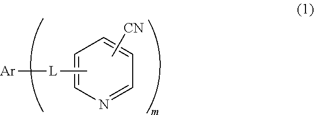

In formula (1), Ar is an m-valent group derived from an aromatic hydrocarbon having 6 to 40 carbons or an m-valent group derived from an aromatic heterocycle having 2 to 40 carbons. At least one hydrogen in these groups may be replaced by an alkyl having 1 to 6 carbons. m is an integer from 1 to 4, and when m is 2, 3, or 4, the group formed by the pyridine ring and L may be identical or different. L is a single bond or one selected from the group of divalent groups represented by formulae (L-1) and (L-2) below.

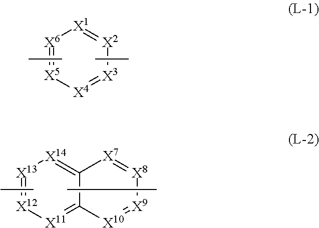

In formula (L-1), X¹ to X⁶ are independently ═CR¹— or ═N—, at least two of X¹ to X⁶ are ═CR¹—, R¹ in two ═CR¹— of X¹ to X⁶ is a bond for bonding with Ar or the pyridine ring, and R¹ in the other ═CR¹— is hydrogen. In formula (L-2), X⁷ to X¹⁴ are independently ═CR¹— or ═N—, at least two of X⁷ to X¹⁴ are ═CR¹—, R¹ in two ═CR¹— of X⁷ to X¹⁴ is a bond for bonding with Ar or the pyridine ring, and R¹ in the other ═CR¹— is hydrogen. At least one hydrogen of L may be replaced by an alkyl having 1 to 4 carbons or an aryl having 6 to 18 carbons.

In formula (1), at least one hydrogen of the pyridine group may be replaced by an alkyl having 1 to 4 carbons, phenyl, or naphthyl. The alkyl having 1 to 4 carbons may be either linear or branched. That is, it may be a linear alkyl having 1 to 4 carbons, or a branched alkyl having 3 or 4 carbons. As specific examples, methyl, ethyl, n-propyl, isopropyl, n-butyl, isobutyl, s-butyl, t-butyl, and the like can be mentioned, and methyl, ethyl, or t-butyl is more preferable.

When m=1, a preferable Ar in formula (1) is specifically one selected from the group of groups represented by formulae (Ar1-1) to (Ar1-12) below. Among these, one selected from the group of groups represented by formulae (Ar1-1) to (Ar1-7) is more preferable, and formula (Ar1-1) is even more preferable.

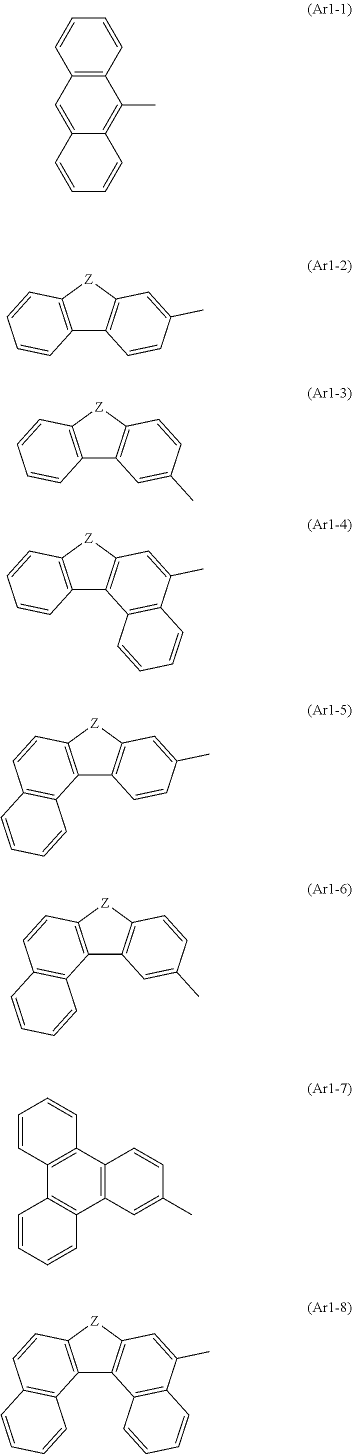

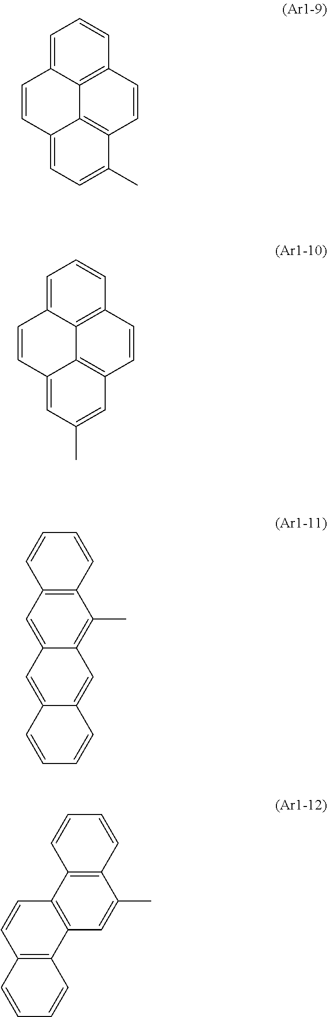

In (Ar1-1) to (Ar1-12), Z is independently —O—, —S—, or one selected from the group of divalent groups represented by formulae (2) and (3) below, and is preferably one selected from the group of divalent groups represented by formulae (2) and (3). At least one hydrogen in each group may be replaced by an alkyl having 1 to 4 carbons or an aryl having 6 to 18 carbons.

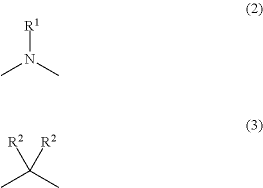

In formula (2), R¹ is phenyl, naphthyl, biphenylyl, or terphenylyl, and, in formula (3), R² is independently methyl or phenyl, and R² may be linked with each other to form a ring. Specifically, a structure in which ortho positions of two phenyls are connected by a single bond so as to form a spiro ring can be mentioned.

When m=2, Ar in formula (1) is preferably one group selected from the group of groups represented by formulae (Ar2-1) to (Ar2-21). Among these, it is more preferably one selected from the group of groups represented by formulae (Ar2-1) to (Ar2-12) and (Ar2-21), and is even more preferably one selected from the group of groups represented by formulae (Ar2-1), (Ar2-8), (Ar2-12), and (Ar2-21).

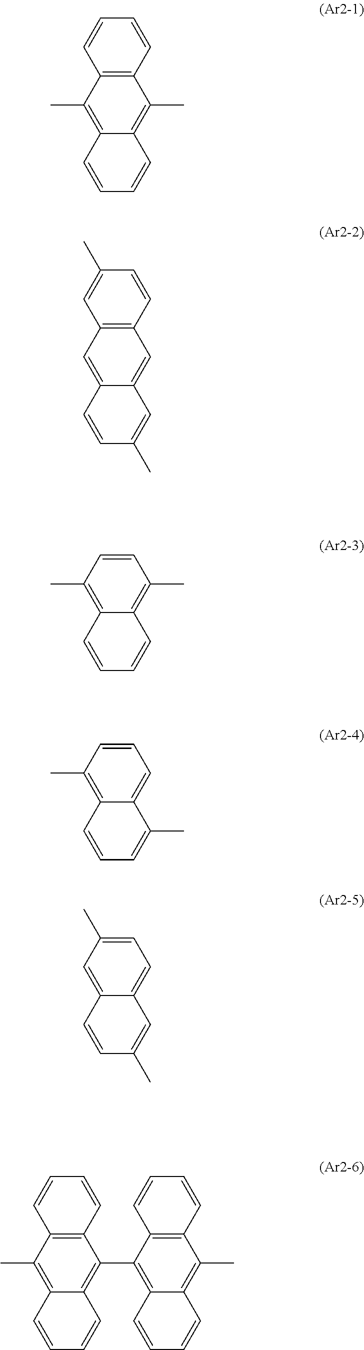

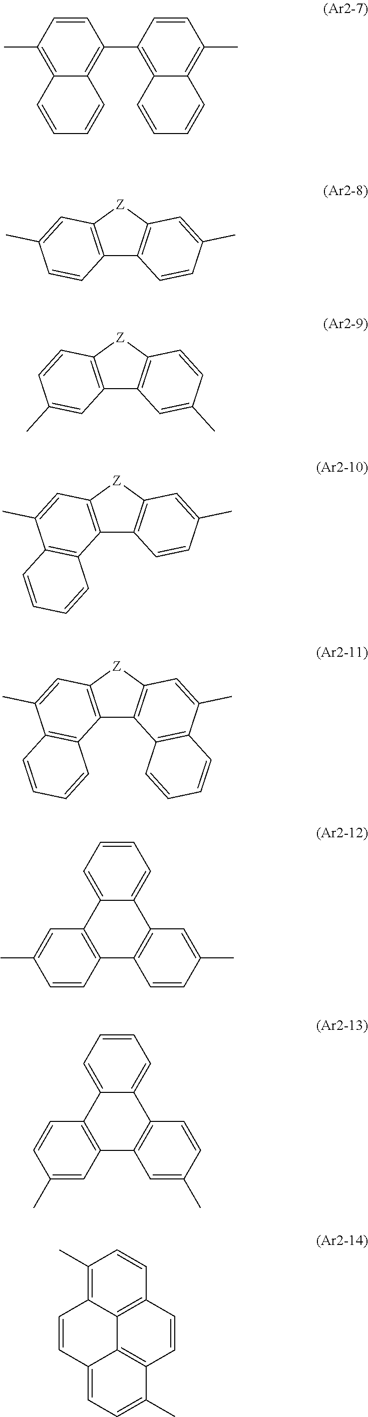

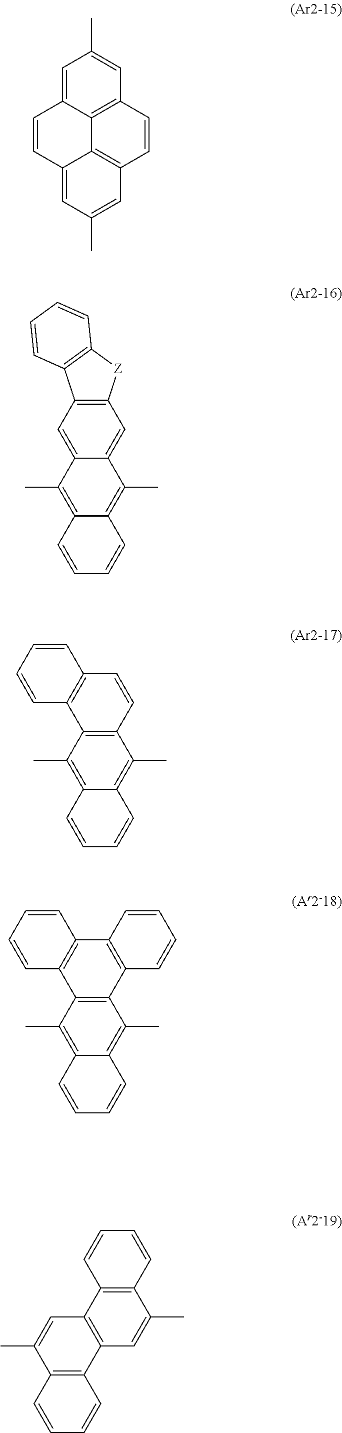

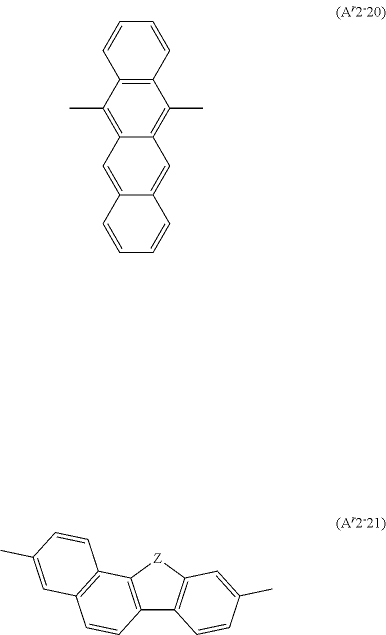

In formulae (Ar2-1) to (Ar2-20), Z is independently —O—, —S—, or one selected from the group of divalent groups represented by formulae (2) and (3) below, and is preferably one selected from the group of divalent groups represented by formulae (2) and (3). At least one hydrogen in each group may be replaced by an alkyl having 1 to 4 carbons or an aryl having 6 to 12 carbons.

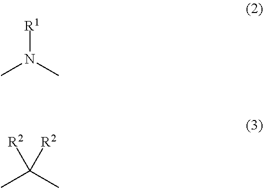

In formula (2), R¹ is phenyl, naphthyl, biphenylyl, or terphenylyl, and, in formula (3), R² is independently methyl or phenyl, and R² may be linked with each other to form a ring. Specifically, a structure in which ortho positions of two phenyls are connected by a single bond so as to form a spiro ring can be mentioned.

In formulae (Ar2-8) to (Ar2-21), Z is even more preferably formula (4) below.

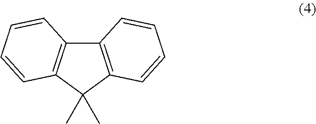

When m=3, Ar in formula (1) is preferably one selected from the group of groups represented by formulae (Ar3-1) and (Ar3-2) below. When m=4, Ar in formula (1) is preferably the group represented by formula (Ar4-1) below.

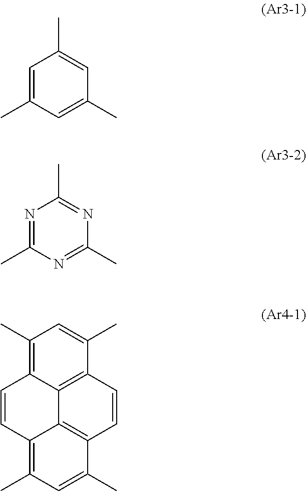

At least one hydrogen of the groups represented by formulae (Ar1-1) to (Ar1-12), (Ar2-1) to (Ar2-20), (Ar3-1), (Ar3-2), and (Ar4-1) may be replaced by an alkyl having 1 to 4 carbons or an aryl having 6 to 18 carbons.

The alkyl having 1 to 4 carbons which may replace at least one hydrogen of the groups represented by formulae (Ar1-1) to (Ar1-12), (Ar2-1) to (Ar2-20), (Ar3-1), (Ar3-2), and (Ar4-1) may be linear or branched. That is, it is a linear alkyl having 1 to 4 carbons, or a branched alkyl having 3 or 4 carbons. As specific examples, methyl, ethyl, n-propyl, isopropyl, n-butyl, isobutyl, s-butyl, t-butyl, and the like can be mentioned, and is preferably methyl, ethyl or t-butyl.

As specific examples of the aryl having 6 to 18 carbons which may replace at least one hydrogen of the groups represented by formulae (Ar1-1) to (Ar1-12), (Ar2-1) to (Ar2-20), (Ar3-1), (Ar3-2), and (Ar4-1), phenyl, (o-, m-, p-)tolyl, (2,3-, 2,4-, 2,5-, 2,6-, 3,4-, 3,5-)xylyl, mesityl(2,4,6-trimethylphenyl), and (o-, m-, p-)cumenyl, which are monocyclic aryls; (2-, 3-, 4-)biphenylyl, which are bicyclic aryls; (1-, 2-)naphthyl, which are fused bicyclic aryls; terphenylyl (m-terphenyl-2′-yl, m-terphenyl-4′-yl, m-terphenyl-5′-yl, o-terphenyl-3′-yl, o-terphenyl-4′-yl, p-terphenyl-2′-yl, m-terphenyl-2-yl, m-terphenyl-3-yl, m-terphenyl-4-yl, o-terphenyl-2-yl, o-terphenyl-3-yl, o-terphenyl-4-yl, p-terphenyl-2-yl, p-terphenyl-3-yl, p-terphenyl-4-yl), which are tricyclic aryls, can be mentioned.

Preferred examples of a preferable “aryl having 6 to 18 carbons” are phenyl, 2-biphenylyl, 3-biphenylyl, 4-biphenylyl, 1-naphthyl, 2-naphthyl, and m-terphenyl-5′-yl.

In formula (1), the linker represented by formula (L-1) specifically is preferably a benzene ring, a pyridine ring, a pyrimidine ring, a pyrazine ring or a pyridazine ring, and is more preferably a benzene ring or a pyridine ring.

In formula (1), the linker represented by formula (L-2) specifically is desirably a naphthalene ring, a quinoline ring, an isoquinoline ring or a quinoxaline ring, and more preferably a naphthalene ring.

In formula (1), specific examples of the group formed by the pyridine ring and L are 4-(6-cyanopyridin-2-yl)phenyl, 4-(5-cyanopyridin-2-yl)phenyl, 4-(4-cyanopyridin-2-yl)phenyl, 4-(3-cyanopyridin-2-yl)phenyl, 4-(2-cyanopyridin-3-yl)phenyl, 4-(6-cyanopyridin-3-yl)phenyl, 4-(5-cyanopyridin-3-yl)phenyl, 4-(4-cyanopyridin-3-yl)phenyl, 4-(3-cyanopyridin-4-yl)phenyl, 4-(2-cyanopyridin-4-yl)phenyl, 3-(6-cyanopyridin-2-yl)phenyl, 3-(5-cyanopyridin-2-yl)phenyl, 3-(4-cyanopyridin-2-yl)phenyl, 3-(3-cyanopyridin-2-yl)phenyl, 3-(2-cyanopyridin-3-yl)phenyl, 3-(6-cyanopyridin-3-yl)phenyl, 3-(5-cyanopyridin-3-yl)phenyl, 3-(4-cyanopyridin-3-yl)phenyl, 3-(3-cyanopyridin-4-yl)phenyl, 3-(2-cyanopyridin-4-yl)phenyl, 2-(6-cyanopyridin-2-yl)phenyl, 2-(5-cyanopyridin-2-yl)phenyl, 2-(4-cyanopyridin-2-yl)phenyl, 2-(3-cyanopyridin-2-yl)phenyl, 2-(2-cyanopyridin-3-yl)phenyl, 2-(6-cyanopyridin-3-yl)phenyl, 2-(5-cyanopyridin-3-yl)phenyl, 2-(4-cyanopyridin-3-yl)phenyl, 2-(3-cyanopyridin-4-yl)phenyl, 2-(2-cyanopyridin-4-yl)phenyl,

6′-cyano-2,2′-bipyridin-5-yl, 5′-cyano-2,2′-bipyridin-5-yl, 4′-cyano-2,2′-bipyridin-5-yl, 3′-cyano-2,2′-bipyridin-5-yl, 6′-cyano-2,3′-bipyridin-5-yl, 5′-cyano-2,3′-bipyridin-5-yl, 4′-cyano-2,3′-bipyridin-5-yl, 2′-cyano-2,3′-bipyridin-5-yl, 2′-cyano-2,4′-bipyridin-5-yl, 3′-cyano-2,4′-bipyridin-5-yl, 6′-cyano-2,2′-bipyridin-6-yl, 5′-cyano-2,2′-bipyridin-6-yl, 4′-cyano-2,2′-bipyridin-6-yl, 3′-cyano-2,2′-bipyridin-6-yl, 6′-cyano-2,3′-bipyridin-6-yl, 5′-cyano-2,3′ bipyridin-6-yl, 4′-cyano-2,3′-bipyridin-6-yl, 2′-cyano-2,3′-bipyridin-6-yl, 2′-cyano-2,4′-bipyridin-6-yl, 3′-cyano-2,4′-bipyridin-6-yl,

6-(6-cyanopyridin-2-yl)naphthalen-2-yl, 6-(5-cyanopyridin-2-yl)naphthalen-2-yl, 6-(4-cyanopyridin-2-yl)naphthalen-2-yl, 6-(3-cyanopyridin-2-yl)naphthalen-2-yl, 6-(6-cyanopyridin-3-yl)naphthalen-2-yl, 2-(5-cyanopyridin-3-yl)naphthalen-6-yl, 6-(4-cyanopyridin-3-yl)naphthalen-2-yl, 6-(2-cyanopyridin-3-yl)naphthalen-2-yl, 6-(3-cyanopyridin-4-yl)naphthalen-2-yl, 6-(2-cyanopyridin-4-yl)naphthalen-2-yl,

7-(6-cyanopyridin-2-yl)naphthalen-2-yl, 7-(5-cyanopyridin-2-yl)naphthalen-2-yl, 7-(4-cyanopyridin-2-yl)naphthalen-2-yl, 7-(3-cyanopyridin-2-yl)naphthalen-2-yl, 7-(6-cyanopyridin-3-yl)naphthalen-2-yl, 7-(5-cyanopyridin-3-yl)naphthalen-2-yl, 7-(4-cyanopyridin-3-yl)naphthalen-2-yl, 7-(2-cyanopyridin-3-yl)naphthalen-2-yl, 7-(3-cyanopyridin-4-yl)naphthalen-2-yl, 7-(2-cyanopyridin-4-yl)naphthalen-2-yl,

4-(6-cyanopyridin-2-yl)naphthalen-1-yl, 4-(5-cyanopyridin-2-yl)naphthalen-1-yl, 4-(4-cyanopyridin-2-yl)naphthalen-1-yl, 4-(3-cyanopyridin-2-yl)naphthalen-1-yl, 4-(6-cyanopyridin-3-yl)naphthalen-1-yl, 4-(5-cyanopyridin-3-yl)naphthalen-1-yl, 4-(4-cyanopyridin-3-yl)naphthalen-1-yl, 4-(2-cyanopyridin-3-yl)naphthalen-1-yl, 4-(3-cyanopyridin-4-yl)naphthalen-1-yl, 4-(2-cyanopyridin-4-yl)naphthalen-1-yl,

6-(6-cyanopyridin-2-yl)pyrazin-2-yl, 6-(5-cyanopyridin-2-yl)pyrazin-2-yl, 6-(4-cyanopyridin-2-yl)pyrazin-2-yl, 6-(3-cyanopyridin-2-yl)pyrazin-2-yl, 6-(6-cyanopyridin-3-yl)pyrazin-2-yl, 6-(5-cyanopyridin-3-yl)pyrazin-2-yl, 6-(4-cyanopyridin-3-yl)pyrazin-2-yl, 6-(2-cyanopyridin-3-yl)pyrazin-2-yl, 6-(3-cyanopyridin-4-yl)pyrazin-2-yl, 6-(2-cyanopyridin-4-yl)pyrazin-2-yl, 2-(6-cyanopyridin-2-yl)quinolin-6-yl, 2-(5-cyanopyridin-2-yl)quinolin-6-yl, 2-(4-cyanopyridin-2-yl)quinolin-6-yl, 2-(3-cyanopyridin-2-yl)quinolin-6-yl, 2-(6-cyanopyridin-3-yl)quinolin-6-yl, 2-(5-cyanopyridin-3-yl)quinolin-6-yl, 2-(4-cyanopyridin-3-yl)quinolin-6-yl, 2-(2-cyanopyridin-3-yl)quinolin-6-yl, 2-(3-cyanopyridin-4-yl)quinolin-6-yl, 2-(2-cyanopyridin-4-yl)quinolin-6-yl,

6-cyanopyridin-2-yl, 5-cyanopyridin-2-yl, 4-cyanopyridin-2-yl, 3-cyanopyridin-2-yl, 6-cyanopyridin-3-yl, 5-cyanopyridin-3-yl, 4-cyanopyridin-3-yl, 2-cyanopyridin-3-yl, 3-cyanopyridin-4-yl, and 2-cyanopyridin-4-yl.

Among these, preferable groups are 4-(6-cyanopyridin-2-yl)phenyl, 4-(5-cyanopyridin-2-yl)phenyl, 4-(4-cyanopyridin-2-yl)phenyl, 4-(3-cyanopyridin-2-yl)phenyl, 4-(2-cyanopyridin-3-yl)phenyl, 4-(6-cyanopyridin-3-yl)phenyl, 4-(5-cyanopyridin-3-yl)phenyl, 4-(4-cyanopyridin-3-yl)phenyl, 4-(3-cyanopyridin-4-yl)phenyl, 4-(2-cyanopyridin-4-yl)phenyl, 3-(6-cyanopyridin-2-yl)phenyl, 3-(5-cyanopyridin-2-yl)phenyl, 3-(4-cyanopyridin-2-yl)phenyl, 3-(3-cyanopyridin-2-yl)phenyl, 3-(2-cyanopyridin-3-yl)phenyl, 3-(6-cyanopyridin-3-yl)phenyl, 3-(5-cyanopyridin-3-yl)phenyl, 3-(4-cyanopyridin-3-yl)phenyl, 3-(3-cyanopyridin-4-yl)phenyl, 3-(2-cyanopyridin-4-yl)phenyl, 6′-cyano-2,2′-bipyridin-5-yl, 5′-cyano-2,2′-bipyridin-5-yl, 4′-cyano-2,2′-bipyridin-5-yl, 3′-cyano-2,2′-bipyridin-5-yl, 6′-cyano-2,3′-bipyridin-5-yl, 5′-cyano-2,3′-bipyridin-5-yl, 4′-cyano-2,3′-bipyridin-5-yl, 2′-cyano-2,3′-bipyridin-5-yl, 2′-cyano-2,4′-bipyridin-5-yl, 3′-cyano-2,4′-bipyridin-5-yl, 6′-cyano-2,3′-bipyridin-6-yl, 5′-cyano-2,3′-bipyridin-6-yl, 4′-cyano-2,3′-bipyridin-6-yl, 2′-cyano-2,3′-bipyridin-6-yl, 2′-cyano-2,4′-bipyridin-6-yl, 3′-cyano-2,4′-bipyridin-6-yl, 2-((6-cyanopyridin)-2-yl)naphthalen-6-yl, 2-((5-cyanopyridin)-2-yl)naphthalen-6-yl, 2-((4-cyanopyridin)-2-yl)naphthalen-6-yl, 2-((3-cyanopyridin)-2-yl)naphthalen-6-yl, 2-((6-cyanopyridin)-3-yl)naphthalen-6-yl, 2-((5-cyanopyridin)-3-yl)naphthalen-6-yl, 2-((4-cyanopyridin)-3-yl)naphthalen-6-yl, 2-((2-cyanopyridin)-3-yl)naphthalen-6-yl, 2-((3-cyanopyridin)-4-yl)naphthalen-6-yl, and 2-((2-cyanopyridin)-4-yl)naphthalen-6-yl.

### Specific Examples of Compound Represented by Formula (1)

Although specific examples of the compound of the present invention are shown by the formulae listed below, the present invention is not limited by the disclosure of these specific structures.

Specific examples of the compound represented by formula (1) when m=1 are shown in the following formulae (1-1-1) to (1-1-668).

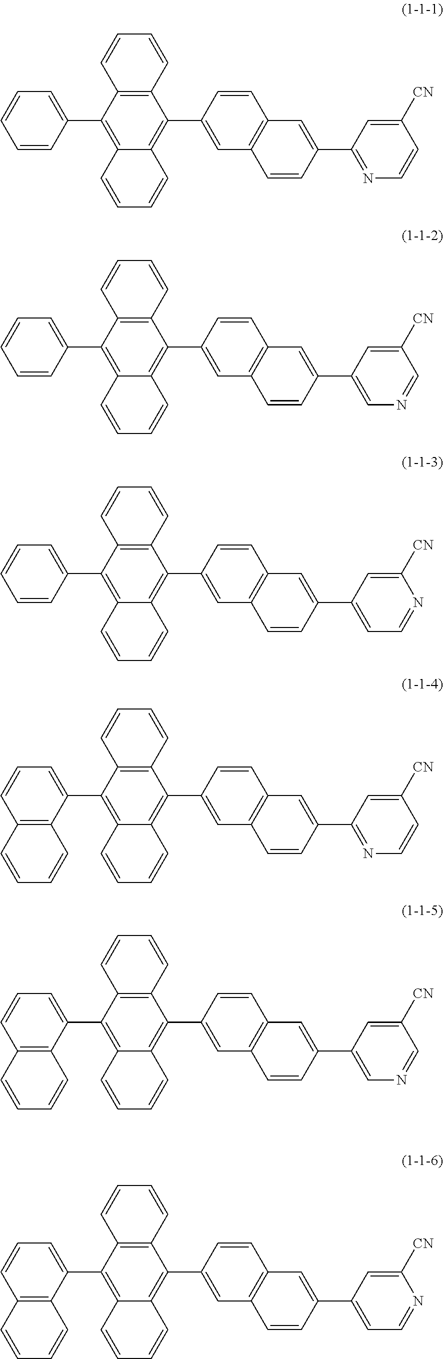 [SMILES: N#Cc1ccnc(-c2ccc3cc(-c4c5ccccc5c(-c5ccccc5)c5ccccc45)ccc3c2)c1 ; N#Cc1cncc(-c2ccc3cc(-c4c5ccccc5c(-c5ccccc5)c5ccccc45)ccc3c2)c1 ; N#Cc1cc(-c2ccc3cc(-c4c5ccccc5c(-c5ccccc5)c5ccccc45)ccc3c2)ccn1 ; N#Cc1ccnc(-c2ccc3cc(-c4c5ccccc5c(-c5cccc6ccccc56)c5ccccc45)ccc3c2)c1 ; N#Cc1cncc(-c2ccc3cc(-c4c5ccccc5c(-c5cccc6ccccc56)c5ccccc45)ccc3c2)c1 ; N#Cc1cc(-c2ccc3cc(-c4c5ccccc5c(-c5cccc6ccccc56)c5ccccc45)ccc3c2)ccn1]

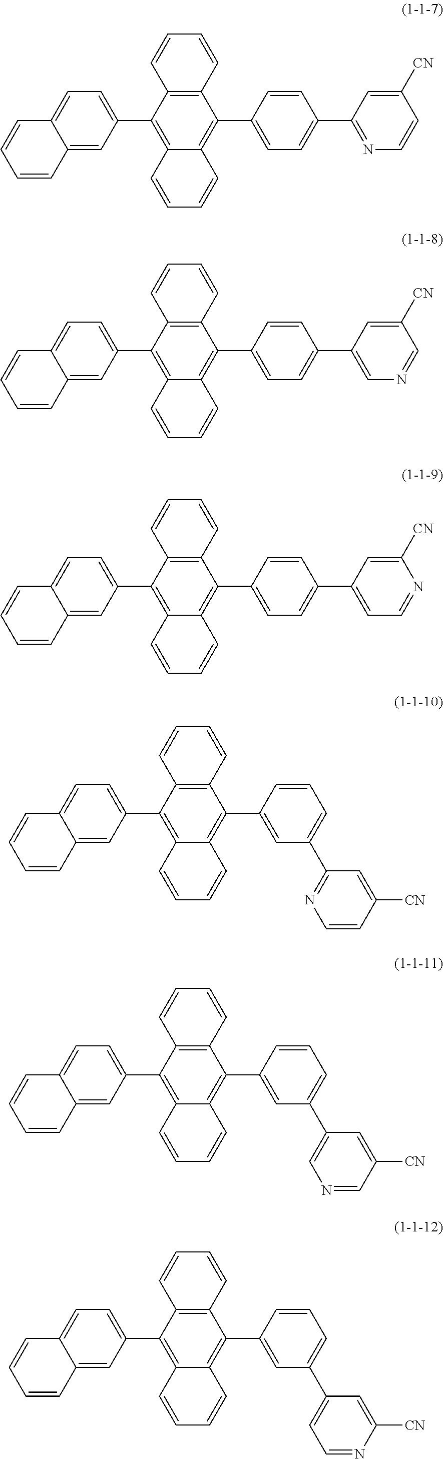 [SMILES: N#Cc1ccnc(-c2ccc(-c3c4ccccc4c(-c4ccc5ccccc5c4)c4ccccc34)cc2)c1 ; N#Cc1cncc(-c2ccc(-c3c4ccccc4c(-c4ccc5ccccc5c4)c4ccccc34)cc2)c1 ; N#Cc1cc(-c2ccc(-c3c4ccccc4c(-c4ccc5ccccc5c4)c4ccccc34)cc2)ccn1 ; N#Cc1ccnc(-c2cccc(-c3c4ccccc4c(-c4ccc5ccccc5c4)c4ccccc34)c2)c1 ; N#Cc1cncc(-c2cccc(-c3c4ccccc4c(-c4ccc5ccccc5c4)c4ccccc34)c2)c1 ; N#Cc1cc(-c2cccc(-c3c4ccccc4c(-c4ccc5ccccc5c4)c4ccccc34)c2)ccn1]

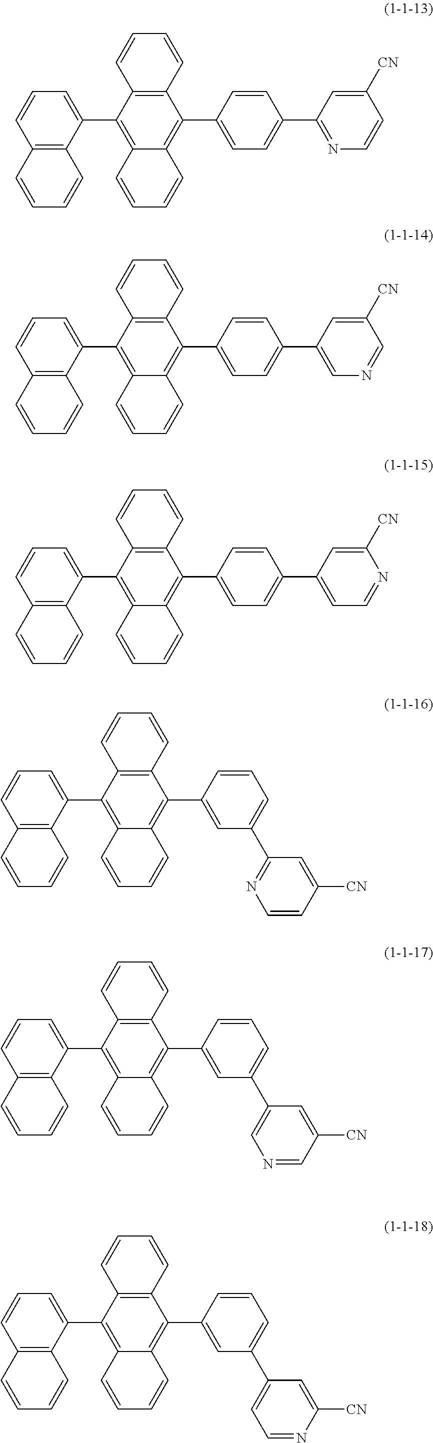 [SMILES: N#Cc1ccnc(-c2ccc(-c3c4ccccc4c(-c4cccc5ccccc45)c4ccccc34)cc2)c1 ; N#Cc1cncc(-c2ccc(-c3c4ccccc4c(-c4cccc5ccccc45)c4ccccc34)cc2)c1 ; N#Cc1cc(-c2ccc(-c3c4ccccc4c(-c4cccc5ccccc45)c4ccccc34)cc2)ccn1 ; N#Cc1ccnc(-c2cccc(-c3c4ccccc4c(-c4cccc5ccccc45)c4ccccc34)c2)c1 ; N#Cc1cncc(-c2cccc(-c3c4ccccc4c(-c4cccc5ccccc45)c4ccccc34)c2)c1 ; N#Cc1cc(-c2cccc(-c3c4ccccc4c(-c4cccc5ccccc45)c4ccccc34)c2)ccn1]

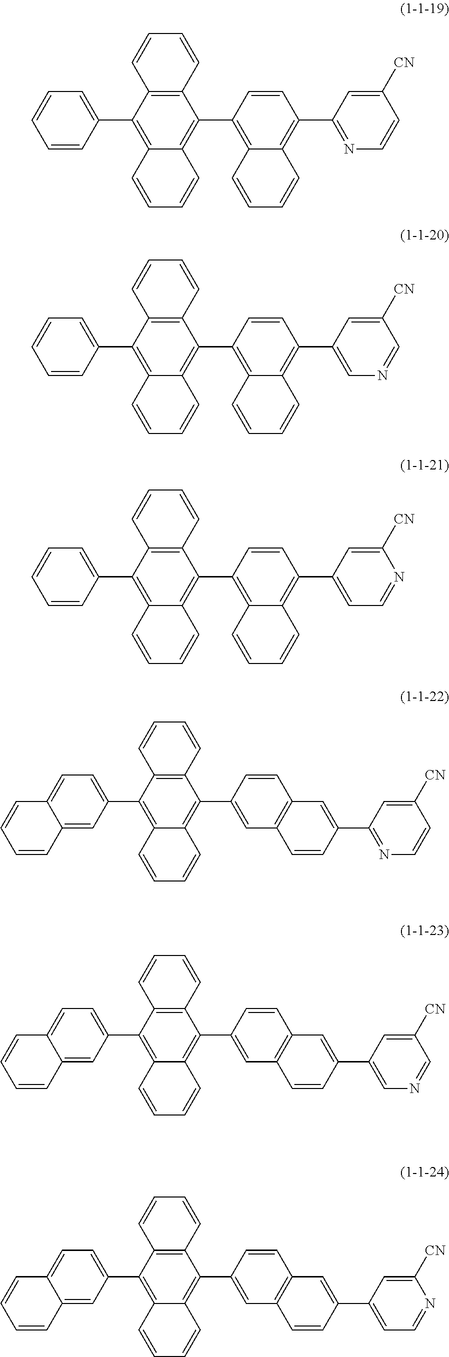 [SMILES: N#Cc1ccnc(-c2ccc(-c3c4ccccc4c(-c4ccccc4)c4ccccc34)c3ccccc23)c1 ; N#Cc1cncc(-c2ccc(-c3c4ccccc4c(-c4ccccc4)c4ccccc34)c3ccccc23)c1 ; N#Cc1cc(-c2ccc(-c3c4ccccc4c(-c4ccccc4)c4ccccc34)c3ccccc23)ccn1 ; N#Cc1ccnc(-c2ccc3cc(-c4c5ccccc5c(-c5ccc6ccccc6c5)c5ccccc45)ccc3c2)c1 ; N#Cc1cncc(-c2ccc3cc(-c4c5ccccc5c(-c5ccc6ccccc6c5)c5ccccc45)ccc3c2)c1 ; N#Cc1cc(-c2ccc3cc(-c4c5ccccc5c(-c5ccc6ccccc6c5)c5ccccc45)ccc3c2)ccn1]

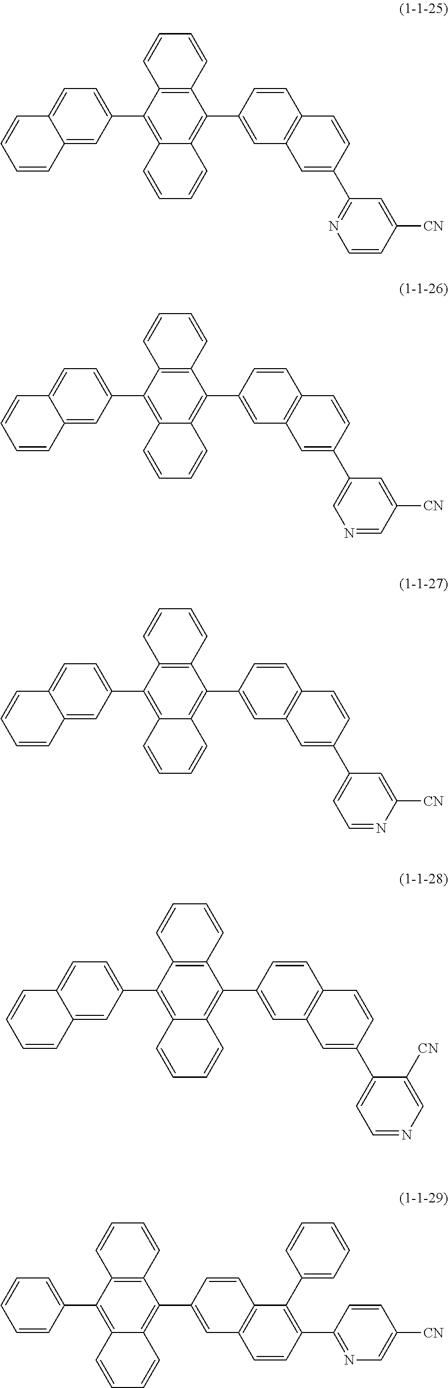 [SMILES: N#Cc1ccnc(-c2ccc3ccc(-c4c5ccccc5c(-c5ccc6ccccc6c5)c5ccccc45)cc3c2)c1 ; N#Cc1cncc(-c2ccc3ccc(-c4c5ccccc5c(-c5ccc6ccccc6c5)c5ccccc45)cc3c2)c1 ; N#Cc1cc(-c2ccc3ccc(-c4c5ccccc5c(-c5ccc6ccccc6c5)c5ccccc45)cc3c2)ccn1 ; N#Cc1cnccc1-c1ccc2ccc(-c3c4ccccc4c(-c4ccc5ccccc5c4)c4ccccc34)cc2c1 ; N#Cc1ccc(-c2ccc3cc(-c4c5ccccc5c(-c5ccccc5)c5ccccc45)ccc3c2-c2ccccc2)nc1]

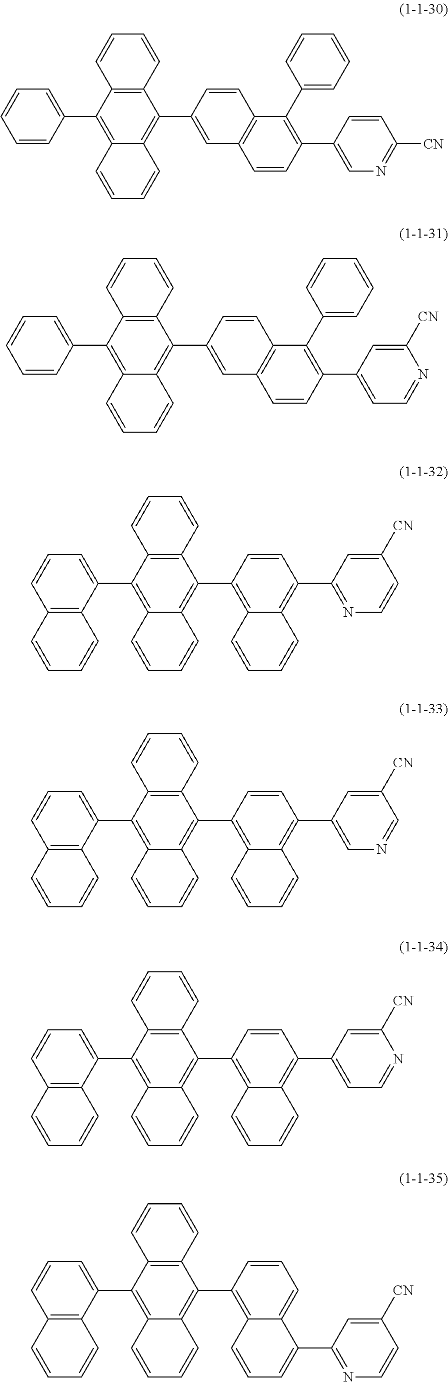 [SMILES: N#Cc1ccc(-c2ccc3cc(-c4c5ccccc5c(-c5ccccc5)c5ccccc45)ccc3c2-c2ccccc2)cn1 ; N#Cc1cc(-c2ccc3cc(-c4c5ccccc5c(-c5ccccc5)c5ccccc45)ccc3c2-c2ccccc2)ccn1 ; N#Cc1ccnc(-c2ccc(-c3c4ccccc4c(-c4cccc5ccccc45)c4ccccc34)c3ccccc23)c1 ; N#Cc1cncc(-c2ccc(-c3c4ccccc4c(-c4cccc5ccccc45)c4ccccc34)c3ccccc23)c1 ; N#Cc1cc(-c2ccc(-c3c4ccccc4c(-c4cccc5ccccc45)c4ccccc34)c3ccccc23)ccn1 ; N#Cc1ccnc(-c2cccc3c(-c4c5ccccc5c(-c5cccc6ccccc56)c5ccccc45)cccc23)c1]

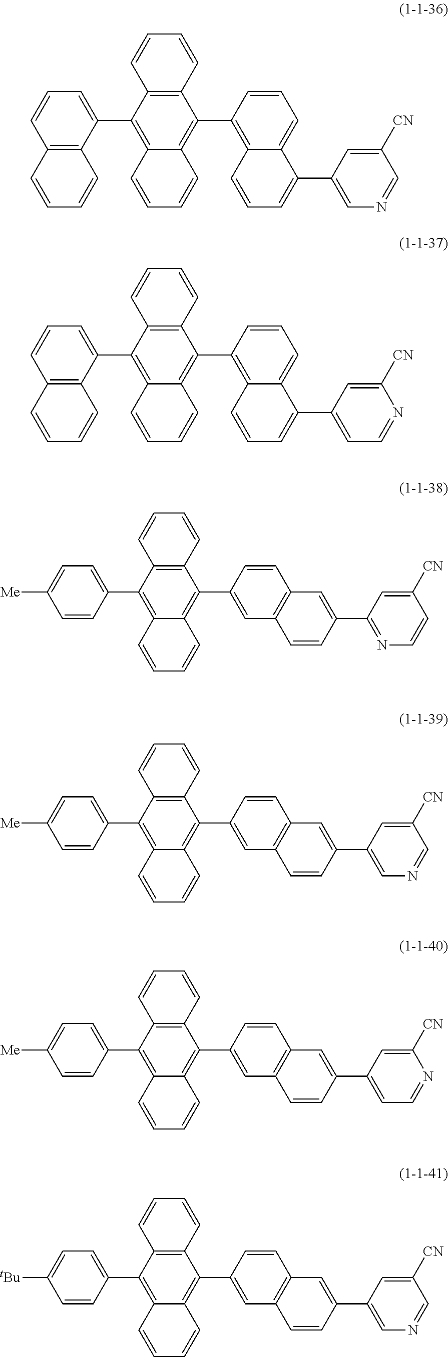 [SMILES: N#Cc1cncc(-c2cccc3c(-c4c5ccccc5c(-c5cccc6ccccc56)c5ccccc45)cccc23)c1 ; N#Cc1cc(-c2cccc3c(-c4c5ccccc5c(-c5cccc6ccccc56)c5ccccc45)cccc23)ccn1 ; Cc1ccc(-c2c3ccccc3c(-c3ccc4cc(-c5cc(C#N)ccn5)ccc4c3)c3ccccc23)cc1 ; Cc1ccc(-c2c3ccccc3c(-c3ccc4cc(-c5cncc(C#N)c5)ccc4c3)c3ccccc23)cc1 ; Cc1ccc(-c2c3ccccc3c(-c3ccc4cc(-c5ccnc(C#N)c5)ccc4c3)c3ccccc23)cc1 ; CC(C)(C)c1ccc(-c2c3ccccc3c(-c3ccc4cc(-c5cncc(C#N)c5)ccc4c3)c3ccccc23)cc1]

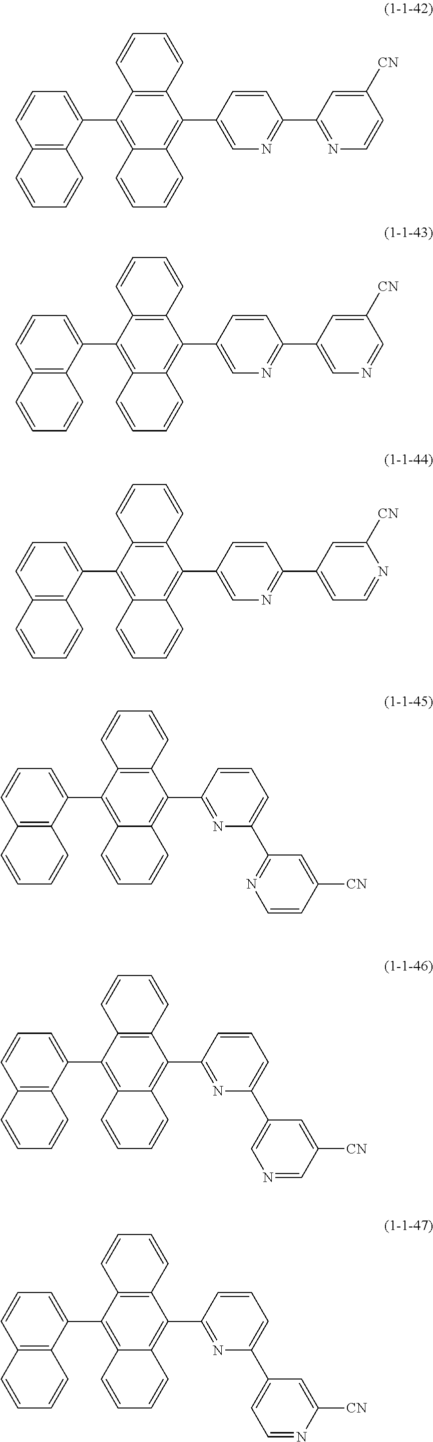 [SMILES: N#Cc1ccnc(-c2ccc(-c3c4ccccc4c(-c4cccc5ccccc45)c4ccccc34)cn2)c1 ; N#Cc1cncc(-c2ccc(-c3c4ccccc4c(-c4cccc5ccccc45)c4ccccc34)cn2)c1 ; N#Cc1cc(-c2ccc(-c3c4ccccc4c(-c4cccc5ccccc45)c4ccccc34)cn2)ccn1 ; N#Cc1ccnc(-c2cccc(-c3c4ccccc4c(-c4cccc5ccccc45)c4ccccc34)n2)c1 ; N#Cc1cncc(-c2cccc(-c3c4ccccc4c(-c4cccc5ccccc45)c4ccccc34)n2)c1 ; N#Cc1cc(-c2cccc(-c3c4ccccc4c(-c4cccc5ccccc45)c4ccccc34)n2)ccn1]

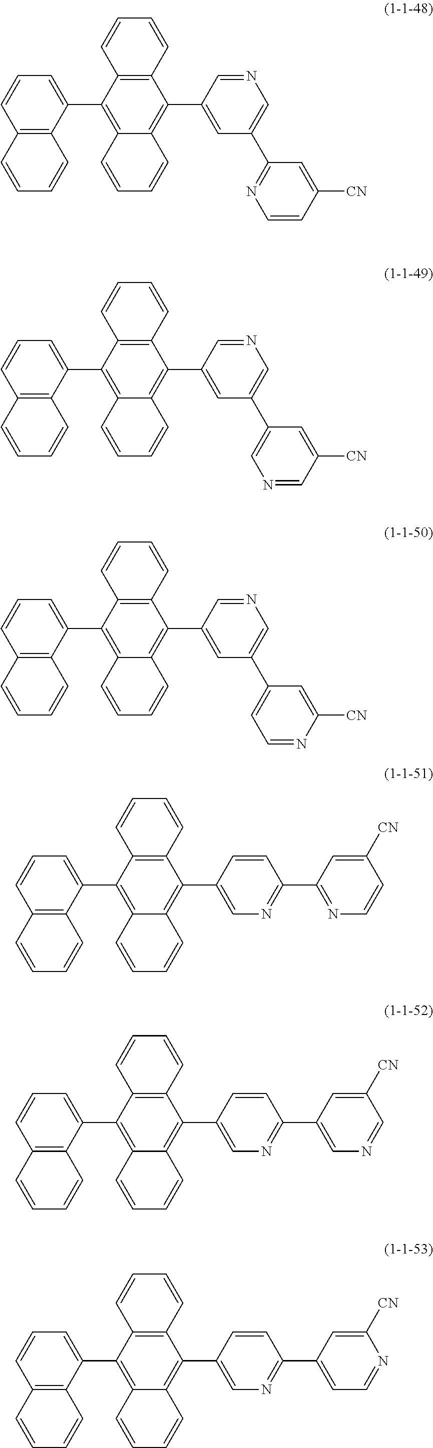 [SMILES: N#Cc1ccnc(-c2cncc(-c3c4ccccc4c(-c4cccc5ccccc45)c4ccccc34)c2)c1 ; N#Cc1cncc(-c2cncc(-c3c4ccccc4c(-c4cccc5ccccc45)c4ccccc34)c2)c1 ; N#Cc1cc(-c2cncc(-c3c4ccccc4c(-c4cccc5ccccc45)c4ccccc34)c2)ccn1 ; N#Cc1ccnc(-c2ccc(-c3c4ccccc4c(-c4cccc5ccccc45)c4ccccc34)cn2)c1 ; N#Cc1cncc(-c2ccc(-c3c4ccccc4c(-c4cccc5ccccc45)c4ccccc34)cn2)c1 ; N#Cc1cc(-c2ccc(-c3c4ccccc4c(-c4cccc5ccccc45)c4ccccc34)cn2)ccn1]

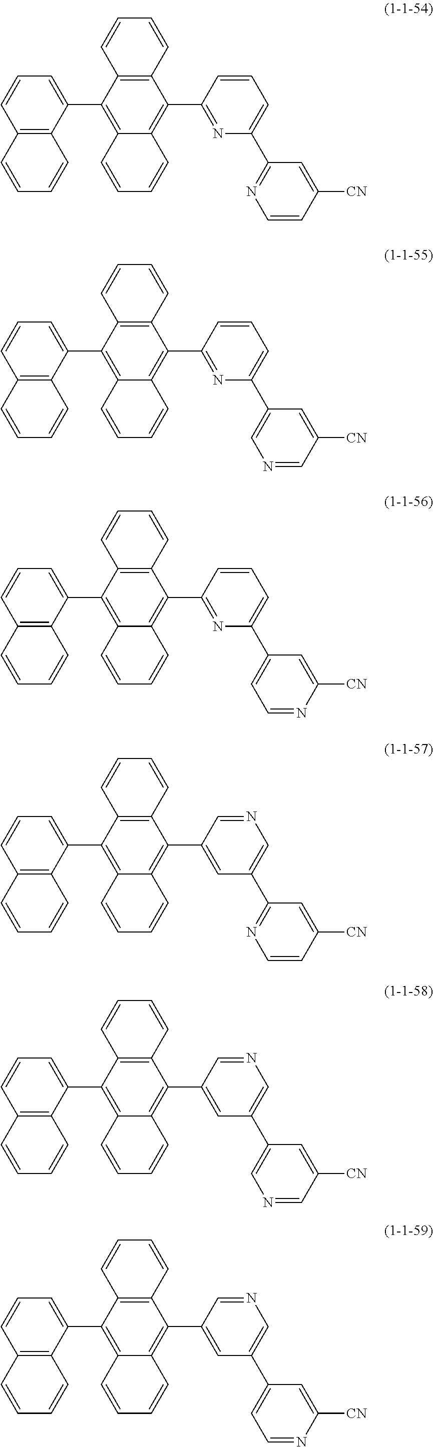 [SMILES: N#Cc1ccnc(-c2cccc(-c3c4ccccc4c(-c4cccc5ccccc45)c4ccccc34)n2)c1 ; N#Cc1cncc(-c2cccc(-c3c4ccccc4c(-c4cccc5ccccc45)c4ccccc34)n2)c1 ; N#Cc1cc(-c2cccc(-c3c4ccccc4c(-c4cccc5ccccc45)c4ccccc34)n2)ccn1 ; N#Cc1ccnc(-c2cncc(-c3c4ccccc4c(-c4cccc5ccccc45)c4ccccc34)c2)c1 ; N#Cc1cncc(-c2cncc(-c3c4ccccc4c(-c4cccc5ccccc45)c4ccccc34)c2)c1 ; N#Cc1cc(-c2cncc(-c3c4ccccc4c(-c4cccc5ccccc45)c4ccccc34)c2)ccn1]

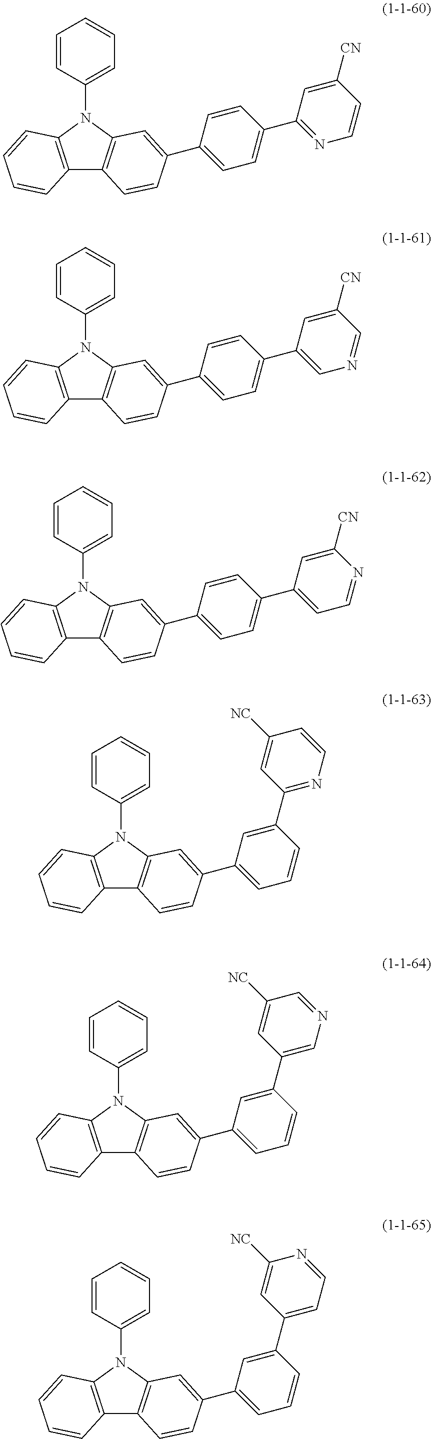 [SMILES: N#Cc1ccnc(-c2ccc(-c3ccc4c5ccccc5n(-c5ccccc5)c4c3)cc2)c1 ; N#Cc1cncc(-c2ccc(-c3ccc4c5ccccc5n(-c5ccccc5)c4c3)cc2)c1 ; N#Cc1cc(-c2ccc(-c3ccc4c5ccccc5n(-c5ccccc5)c4c3)cc2)ccn1 ; N#Cc1ccnc(-c2cccc(-c3ccc4c5ccccc5n(-c5ccccc5)c4c3)c2)c1 ; N#Cc1cncc(-c2cccc(-c3ccc4c5ccccc5n(-c5ccccc5)c4c3)c2)c1 ; N#Cc1cc(-c2cccc(-c3ccc4c5ccccc5n(-c5ccccc5)c4c3)c2)ccn1]

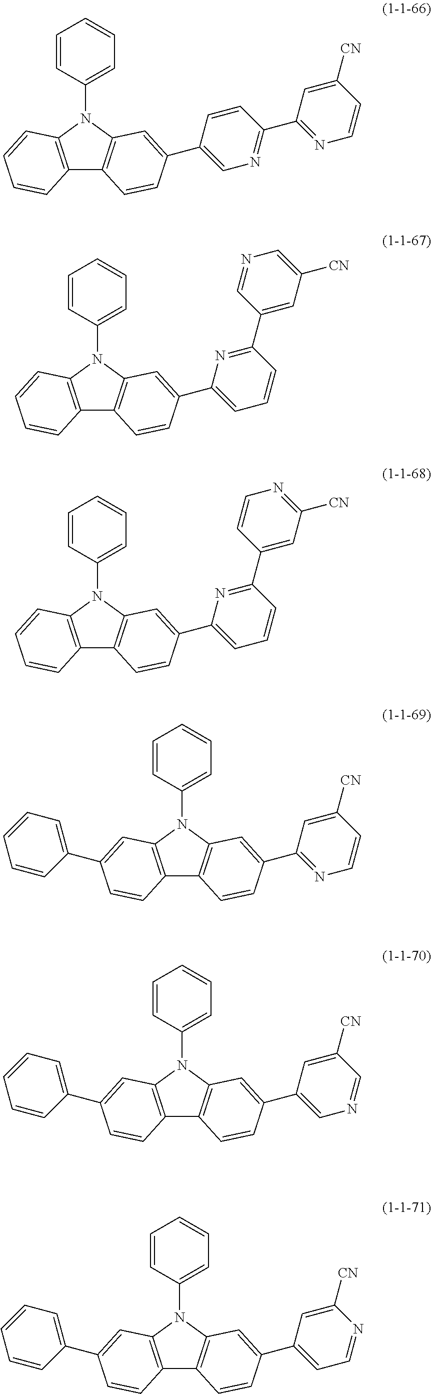 [SMILES: N#Cc1ccnc(-c2ccc(-c3ccc4c5ccccc5n(-c5ccccc5)c4c3)cn2)c1 ; N#Cc1cncc(-c2cccc(-c3ccc4c5ccccc5n(-c5ccccc5)c4c3)n2)c1 ; N#Cc1cc(-c2cccc(-c3ccc4c5ccccc5n(-c5ccccc5)c4c3)n2)ccn1 ; N#Cc1ccnc(-c2ccc3c4ccc(-c5ccccc5)cc4n(-c4ccccc4)c3c2)c1 ; N#Cc1cncc(-c2ccc3c4ccc(-c5ccccc5)cc4n(-c4ccccc4)c3c2)c1 ; N#Cc1cc(-c2ccc3c4ccc(-c5ccccc5)cc4n(-c4ccccc4)c3c2)ccn1]

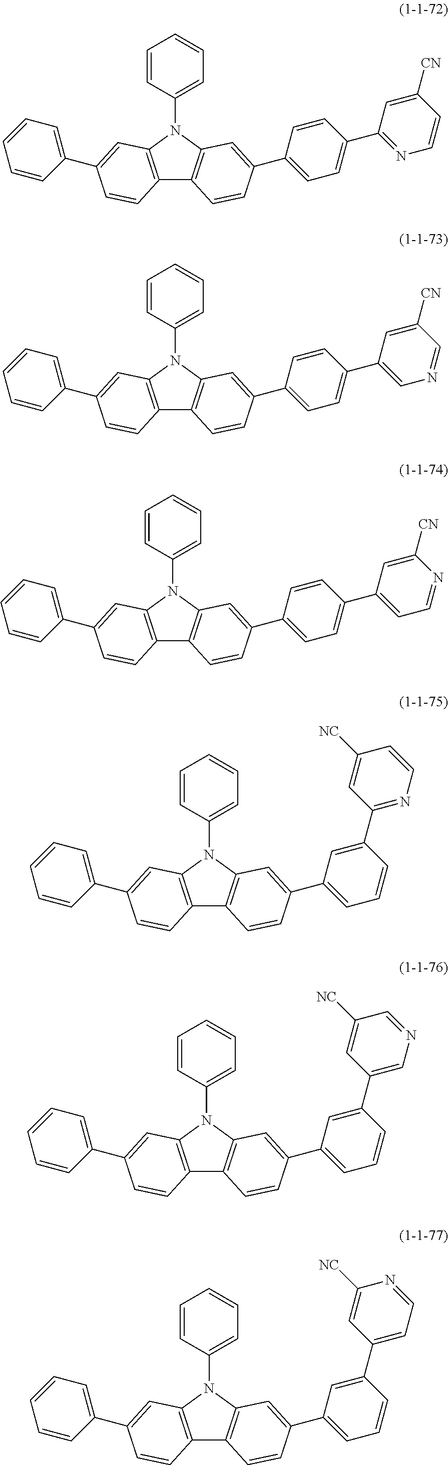 [SMILES: N#Cc1ccnc(-c2ccc(-c3ccc4c5ccc(-c6ccccc6)cc5n(-c5ccccc5)c4c3)cc2)c1 ; N#Cc1cncc(-c2ccc(-c3ccc4c5ccc(-c6ccccc6)cc5n(-c5ccccc5)c4c3)cc2)c1 ; N#Cc1cc(-c2ccc(-c3ccc4c5ccc(-c6ccccc6)cc5n(-c5ccccc5)c4c3)cc2)ccn1 ; N#Cc1ccnc(-c2cccc(-c3ccc4c5ccc(-c6ccccc6)cc5n(-c5ccccc5)c4c3)c2)c1 ; N#Cc1cncc(-c2cccc(-c3ccc4c5ccc(-c6ccccc6)cc5n(-c5ccccc5)c4c3)c2)c1 ; N#Cc1cc(-c2cccc(-c3ccc4c5ccc(-c6ccccc6)cc5n(-c5ccccc5)c4c3)c2)ccn1]

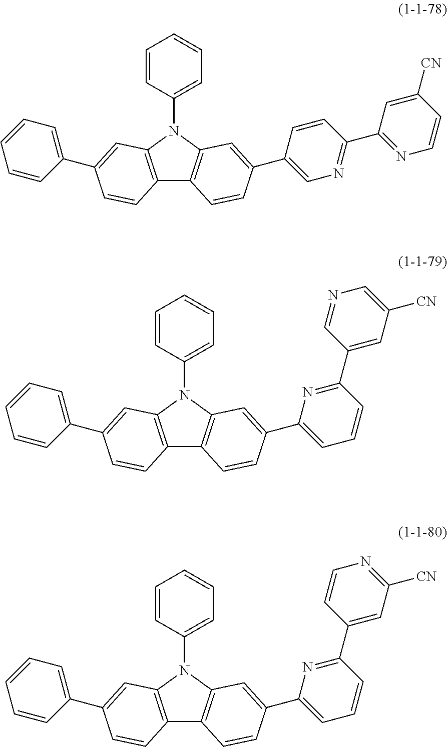 [SMILES: N#Cc1ccnc(-c2ccc(-c3ccc4c5ccc(-c6ccccc6)cc5n(-c5ccccc5)c4c3)cn2)c1 ; N#Cc1cncc(-c2cccc(-c3ccc4c5ccc(-c6ccccc6)cc5n(-c5ccccc5)c4c3)n2)c1 ; N#Cc1cc(-c2cccc(-c3ccc4c5ccc(-c6ccccc6)cc5n(-c5ccccc5)c4c3)n2)ccn1]

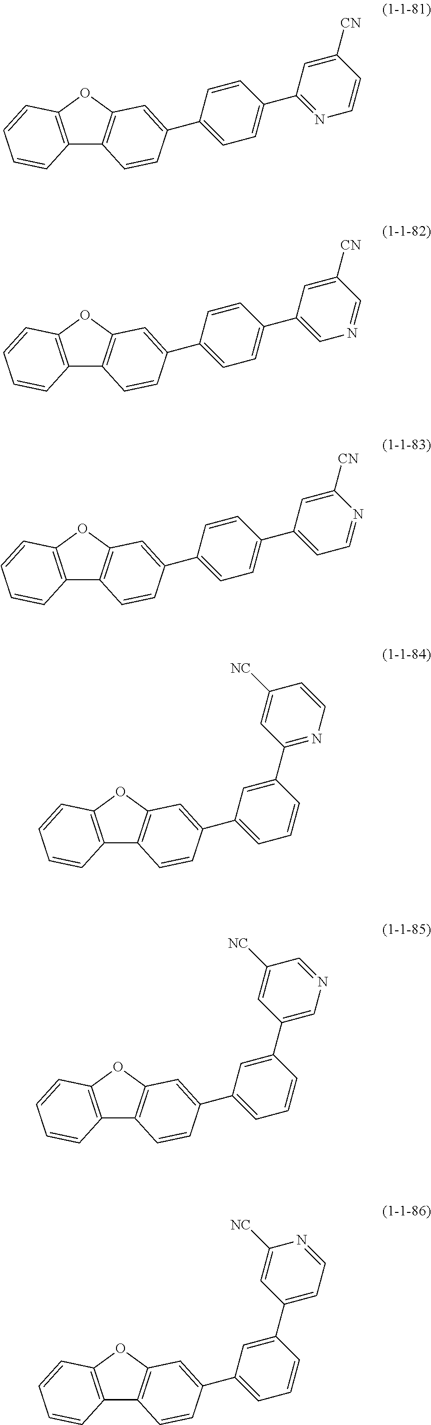 [SMILES: N#Cc1ccnc(-c2ccc(-c3ccc4c(c3)oc3ccccc34)cc2)c1 ; N#Cc1cncc(-c2ccc(-c3ccc4c(c3)oc3ccccc34)cc2)c1 ; N#Cc1cc(-c2ccc(-c3ccc4c(c3)oc3ccccc34)cc2)ccn1 ; N#Cc1ccnc(-c2cccc(-c3ccc4c(c3)oc3ccccc34)c2)c1 ; N#Cc1cncc(-c2cccc(-c3ccc4c(c3)oc3ccccc34)c2)c1 ; N#Cc1cc(-c2cccc(-c3ccc4c(c3)oc3ccccc34)c2)ccn1]

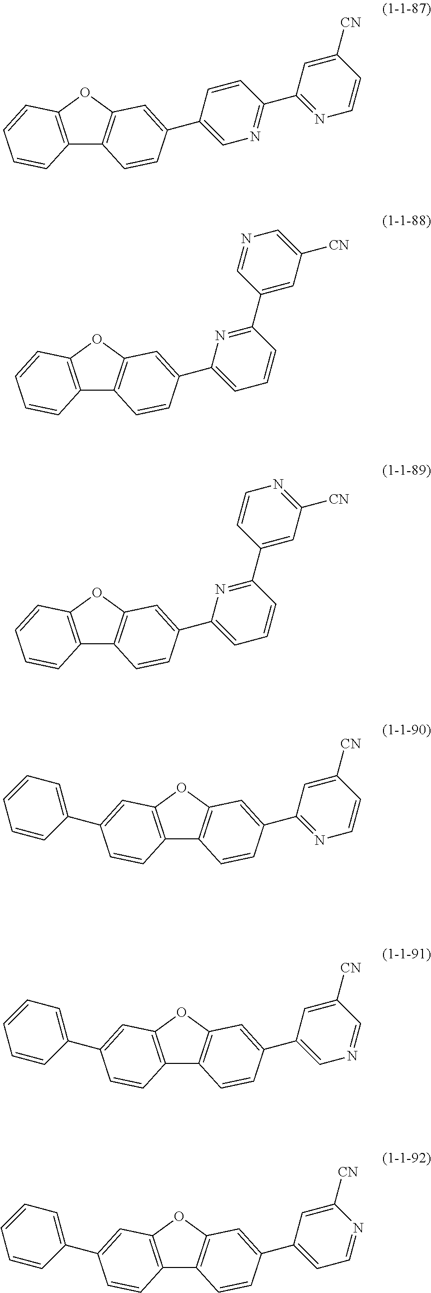 [SMILES: N#Cc1ccnc(-c2ccc(-c3ccc4c(c3)oc3ccccc34)cn2)c1 ; N#Cc1cncc(-c2cccc(-c3ccc4c(c3)oc3ccccc34)n2)c1 ; N#Cc1cc(-c2cccc(-c3ccc4c(c3)oc3ccccc34)n2)ccn1 ; N#Cc1ccnc(-c2ccc3c(c2)oc2cc(-c4ccccc4)ccc23)c1 ; N#Cc1cncc(-c2ccc3c(c2)oc2cc(-c4ccccc4)ccc23)c1 ; N#Cc1cc(-c2ccc3c(c2)oc2cc(-c4ccccc4)ccc23)ccn1]

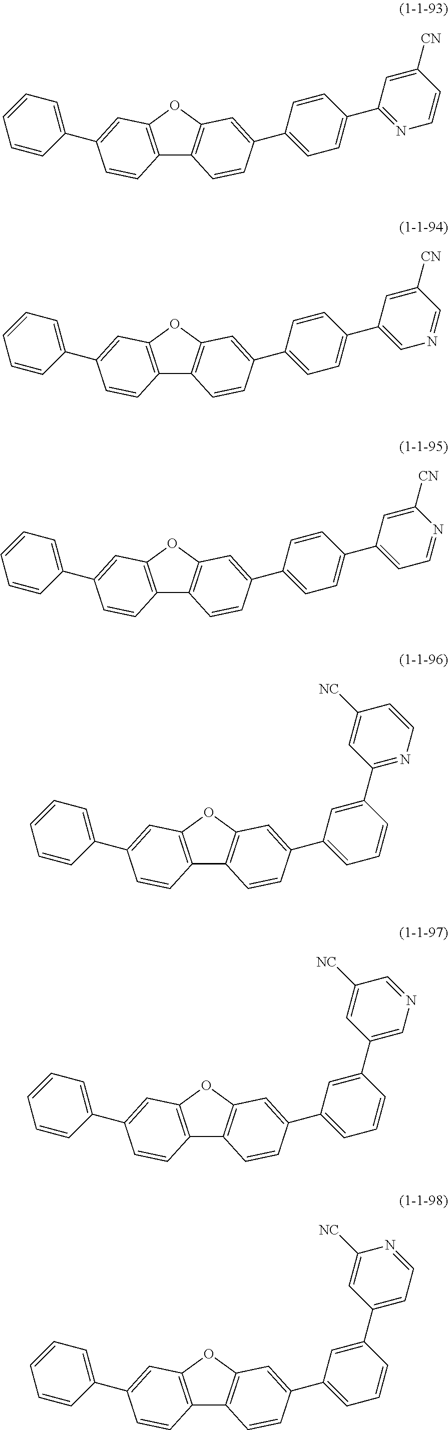 [SMILES: N#Cc1ccnc(-c2ccc(-c3ccc4c(c3)oc3cc(-c5ccccc5)ccc34)cc2)c1 ; N#Cc1cncc(-c2ccc(-c3ccc4c(c3)oc3cc(-c5ccccc5)ccc34)cc2)c1 ; N#Cc1cc(-c2ccc(-c3ccc4c(c3)oc3cc(-c5ccccc5)ccc34)cc2)ccn1 ; N#Cc1ccnc(-c2cccc(-c3ccc4c(c3)oc3cc(-c5ccccc5)ccc34)c2)c1 ; N#Cc1cncc(-c2cccc(-c3ccc4c(c3)oc3cc(-c5ccccc5)ccc34)c2)c1 ; N#Cc1cc(-c2cccc(-c3ccc4c(c3)oc3cc(-c5ccccc5)ccc34)c2)ccn1]

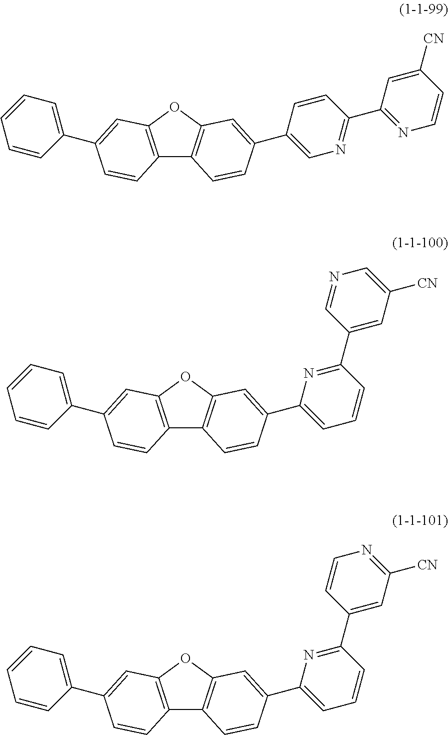 [SMILES: N#Cc1ccnc(-c2ccc(-c3ccc4c(c3)oc3cc(-c5ccccc5)ccc34)cn2)c1 ; N#Cc1cncc(-c2cccc(-c3ccc4c(c3)oc3cc(-c5ccccc5)ccc34)n2)c1 ; N#Cc1cc(-c2cccc(-c3ccc4c(c3)oc3cc(-c5ccccc5)ccc34)n2)ccn1]

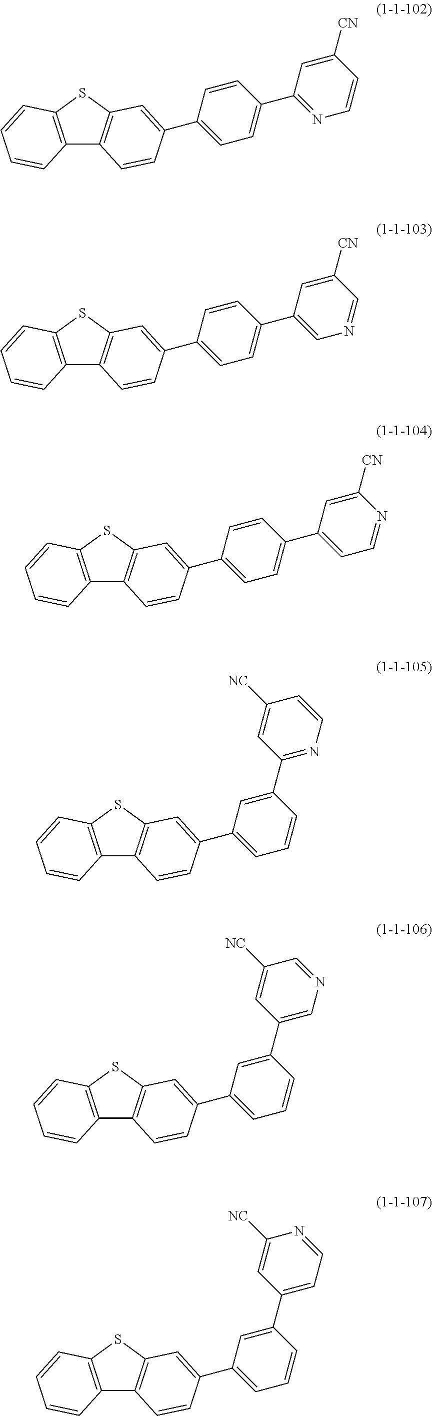 [SMILES: N#Cc1ccnc(-c2ccc(-c3ccc4c(c3)sc3ccccc34)cc2)c1 ; N#Cc1cncc(-c2ccc(-c3ccc4c(c3)sc3ccccc34)cc2)c1 ; N#Cc1cc(-c2ccc(-c3ccc4c(c3)sc3ccccc34)cc2)ccn1 ; N#Cc1ccnc(-c2cccc(-c3ccc4c(c3)sc3ccccc34)c2)c1 ; N#Cc1cncc(-c2cccc(-c3ccc4c(c3)sc3ccccc34)c2)c1 ; N#Cc1cc(-c2cccc(-c3ccc4c(c3)sc3ccccc34)c2)ccn1]

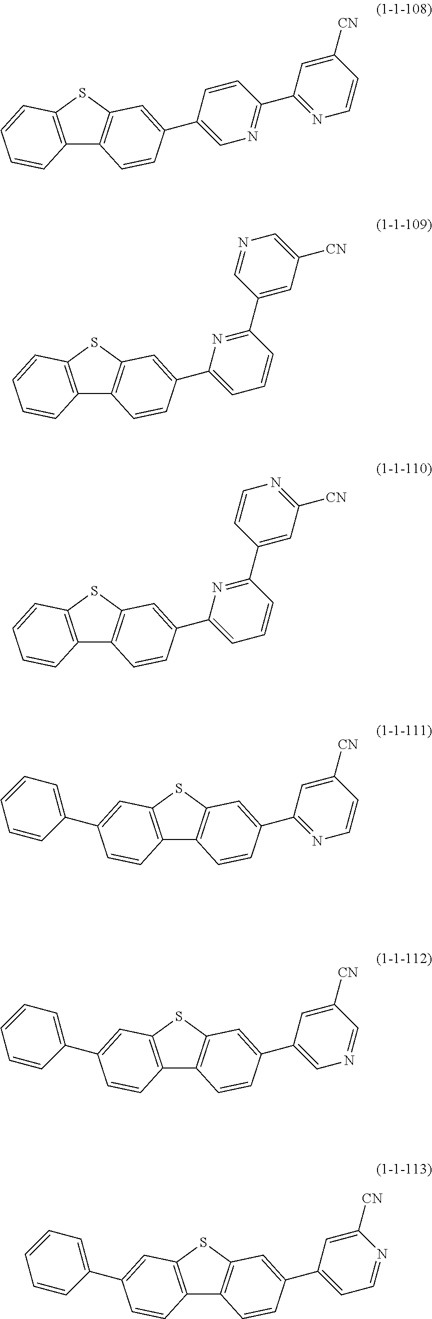 [SMILES: N#Cc1ccnc(-c2ccc(-c3ccc4c(c3)sc3ccccc34)cn2)c1 ; N#Cc1cncc(-c2cccc(-c3ccc4c(c3)sc3ccccc34)n2)c1 ; N#Cc1cc(-c2cccc(-c3ccc4c(c3)sc3ccccc34)n2)ccn1 ; N#Cc1ccnc(-c2ccc3c(c2)sc2cc(-c4ccccc4)ccc23)c1 ; N#Cc1cncc(-c2ccc3c(c2)sc2cc(-c4ccccc4)ccc23)c1 ; N#Cc1cc(-c2ccc3c(c2)sc2cc(-c4ccccc4)ccc23)ccn1]

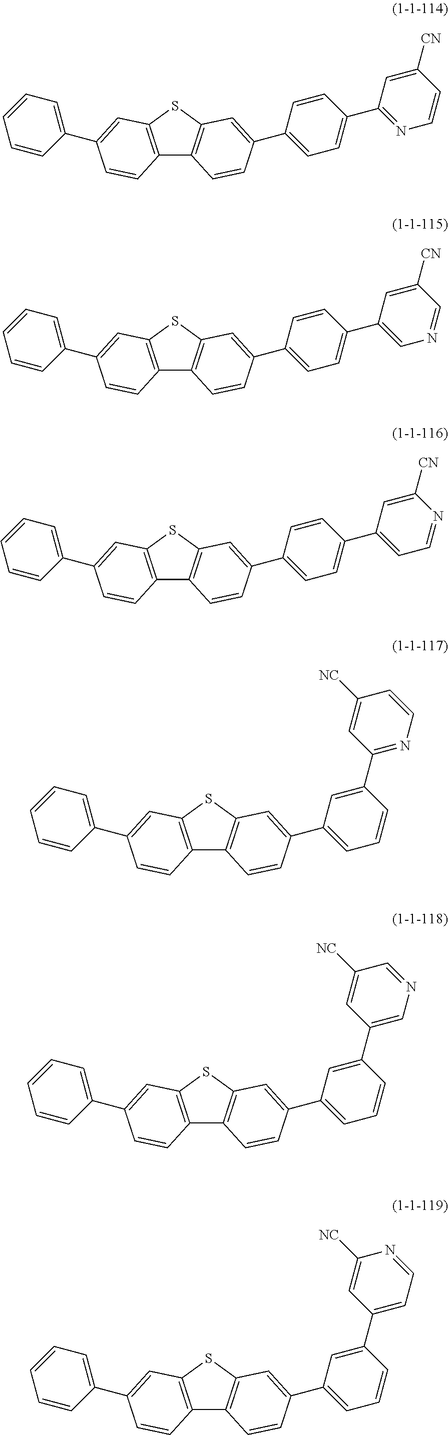 [SMILES: N#Cc1ccnc(-c2ccc(-c3ccc4c(c3)sc3cc(-c5ccccc5)ccc34)cc2)c1 ; N#Cc1cncc(-c2ccc(-c3ccc4c(c3)sc3cc(-c5ccccc5)ccc34)cc2)c1 ; N#Cc1cc(-c2ccc(-c3ccc4c(c3)sc3cc(-c5ccccc5)ccc34)cc2)ccn1 ; N#Cc1ccnc(-c2cccc(-c3ccc4c(c3)sc3cc(-c5ccccc5)ccc34)c2)c1 ; N#Cc1cncc(-c2cccc(-c3ccc4c(c3)sc3cc(-c5ccccc5)ccc34)c2)c1 ; N#Cc1cc(-c2cccc(-c3ccc4c(c3)sc3cc(-c5ccccc5)ccc34)c2)ccn1]

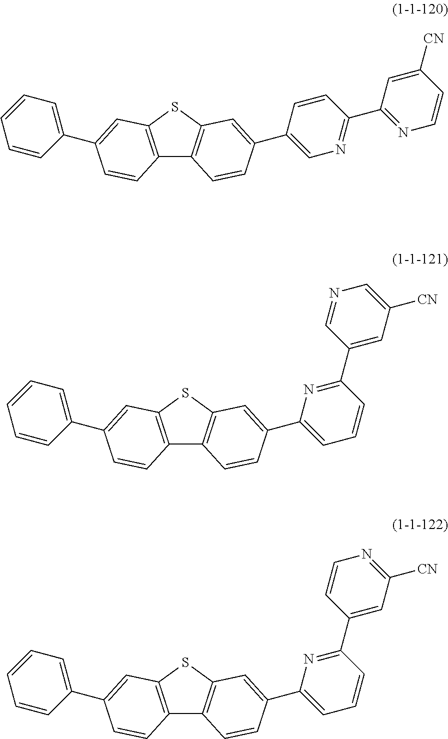 [SMILES: N#Cc1ccnc(-c2ccc(-c3ccc4c(c3)sc3cc(-c5ccccc5)ccc34)cn2)c1 ; N#Cc1cncc(-c2cccc(-c3ccc4c(c3)sc3cc(-c5ccccc5)ccc34)n2)c1 ; N#Cc1cc(-c2cccc(-c3ccc4c(c3)sc3cc(-c5ccccc5)ccc34)n2)ccn1]

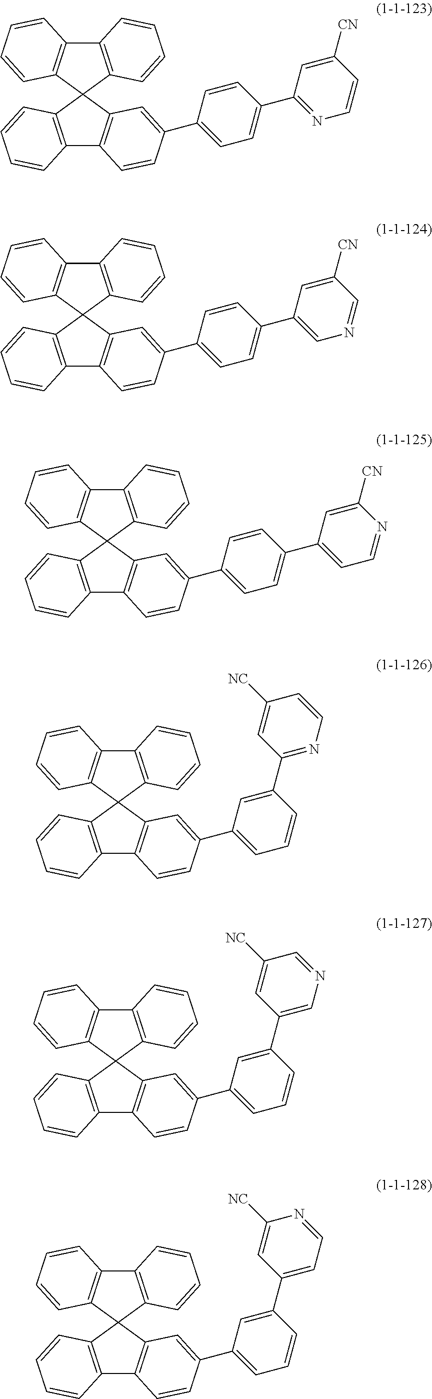 [SMILES: N#Cc1ccnc(-c2ccc(-c3ccc4c(c3)C3(c5ccccc5-c5ccccc53)c3ccccc3-4)cc2)c1 ; N#Cc1cncc(-c2ccc(-c3ccc4c(c3)C3(c5ccccc5-c5ccccc53)c3ccccc3-4)cc2)c1 ; N#Cc1cc(-c2ccc(-c3ccc4c(c3)C3(c5ccccc5-c5ccccc53)c3ccccc3-4)cc2)ccn1 ; N#Cc1ccnc(-c2cccc(-c3ccc4c(c3)C3(c5ccccc5-c5ccccc53)c3ccccc3-4)c2)c1 ; N#Cc1cncc(-c2cccc(-c3ccc4c(c3)C3(c5ccccc5-c5ccccc53)c3ccccc3-4)c2)c1 ; N#Cc1cc(-c2cccc(-c3ccc4c(c3)C3(c5ccccc5-c5ccccc53)c3ccccc3-4)c2)ccn1]

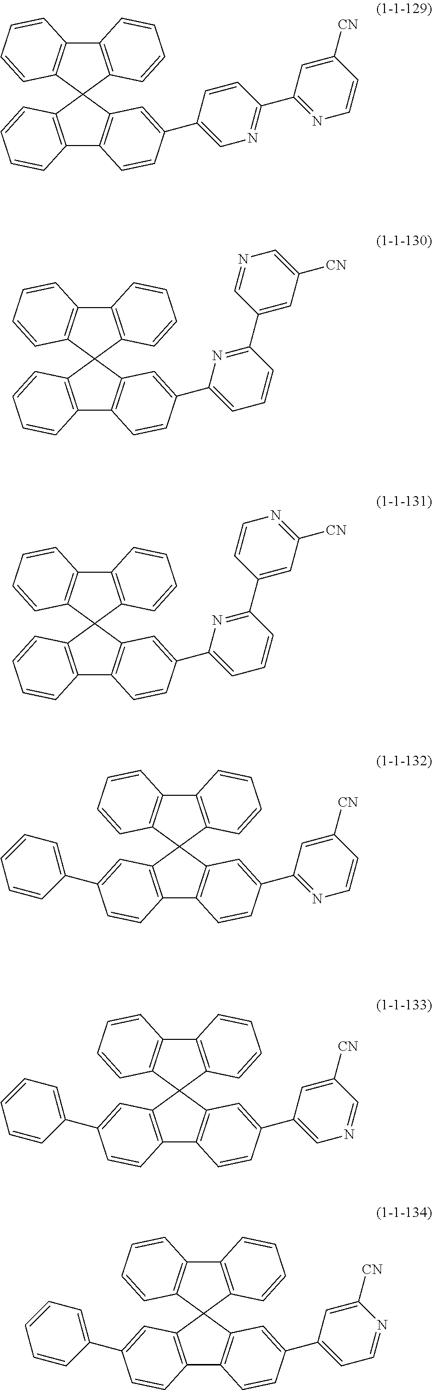 [SMILES: N#Cc1ccnc(-c2ccc(-c3ccc4c(c3)C3(c5ccccc5-c5ccccc53)c3ccccc3-4)cn2)c1 ; N#Cc1cncc(-c2cccc(-c3ccc4c(c3)C3(c5ccccc5-c5ccccc53)c3ccccc3-4)n2)c1 ; N#Cc1cc(-c2cccc(-c3ccc4c(c3)C3(c5ccccc5-c5ccccc53)c3ccccc3-4)n2)ccn1 ; N#Cc1ccnc(-c2ccc3c(c2)C2(c4ccccc4-c4ccccc42)c2cc(-c4ccccc4)ccc2-3)c1 ; N#Cc1cncc(-c2ccc3c(c2)C2(c4ccccc4-c4ccccc42)c2cc(-c4ccccc4)ccc2-3)c1 ; N#Cc1cc(-c2ccc3c(c2)C2(c4ccccc4-c4ccccc42)c2cc(-c4ccccc4)ccc2-3)ccn1]

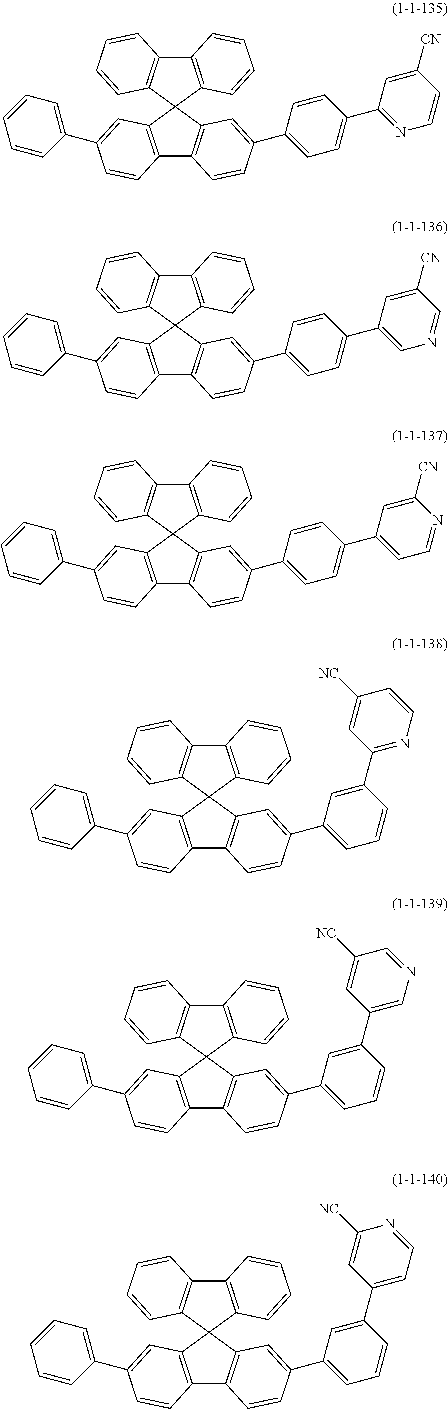 [SMILES: N#Cc1ccnc(-c2ccc(-c3ccc4c(c3)C3(c5ccccc5-c5ccccc53)c3cc(-c5ccccc5)ccc3-4)cc2)c1 ; N#Cc1cncc(-c2ccc(-c3ccc4c(c3)C3(c5ccccc5-c5ccccc53)c3cc(-c5ccccc5)ccc3-4)cc2)c1 ; N#Cc1cc(-c2ccc(-c3ccc4c(c3)C3(c5ccccc5-c5ccccc53)c3cc(-c5ccccc5)ccc3-4)cc2)ccn1 ; N#Cc1ccnc(-c2cccc(-c3ccc4c(c3)C3(c5ccccc5-c5ccccc53)c3cc(-c5ccccc5)ccc3-4)c2)c1 ; N#Cc1cncc(-c2cccc(-c3ccc4c(c3)C3(c5ccccc5-c5ccccc53)c3cc(-c5ccccc5)ccc3-4)c2)c1 ; N#Cc1cc(-c2cccc(-c3ccc4c(c3)C3(c5ccccc5-c5ccccc53)c3cc(-c5ccccc5)ccc3-4)c2)ccn1]

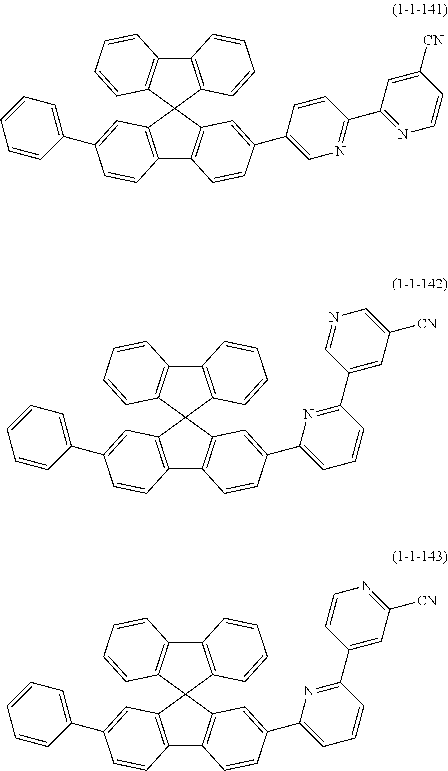 [SMILES: N#Cc1ccnc(-c2ccc(-c3ccc4c(c3)C3(c5ccccc5-c5ccccc53)c3cc(-c5ccccc5)ccc3-4)cn2)c1 ; N#Cc1cncc(-c2cccc(-c3ccc4c(c3)C3(c5ccccc5-c5ccccc53)c3cc(-c5ccccc5)ccc3-4)n2)c1 ; N#Cc1cc(-c2cccc(-c3ccc4c(c3)C3(c5ccccc5-c5ccccc53)c3cc(-c5ccccc5)ccc3-4)n2)ccn1]

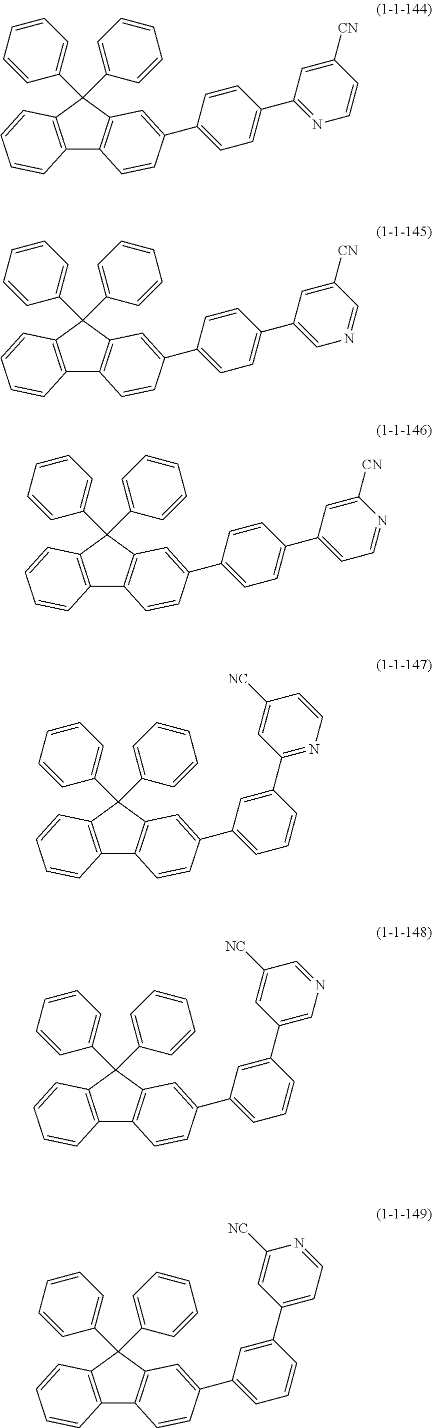 [SMILES: N#Cc1ccnc(-c2ccc(-c3ccc4c(c3)C(c3ccccc3)(c3ccccc3)c3ccccc3-4)cc2)c1 ; N#Cc1cncc(-c2ccc(-c3ccc4c(c3)C(c3ccccc3)(c3ccccc3)c3ccccc3-4)cc2)c1 ; N#Cc1cc(-c2ccc(-c3ccc4c(c3)C(c3ccccc3)(c3ccccc3)c3ccccc3-4)cc2)ccn1 ; N#Cc1ccnc(-c2cccc(-c3ccc4c(c3)C(c3ccccc3)(c3ccccc3)c3ccccc3-4)c2)c1 ; N#Cc1cncc(-c2cccc(-c3ccc4c(c3)C(c3ccccc3)(c3ccccc3)c3ccccc3-4)c2)c1 ; N#Cc1cc(-c2cccc(-c3ccc4c(c3)C(c3ccccc3)(c3ccccc3)c3ccccc3-4)c2)ccn1]

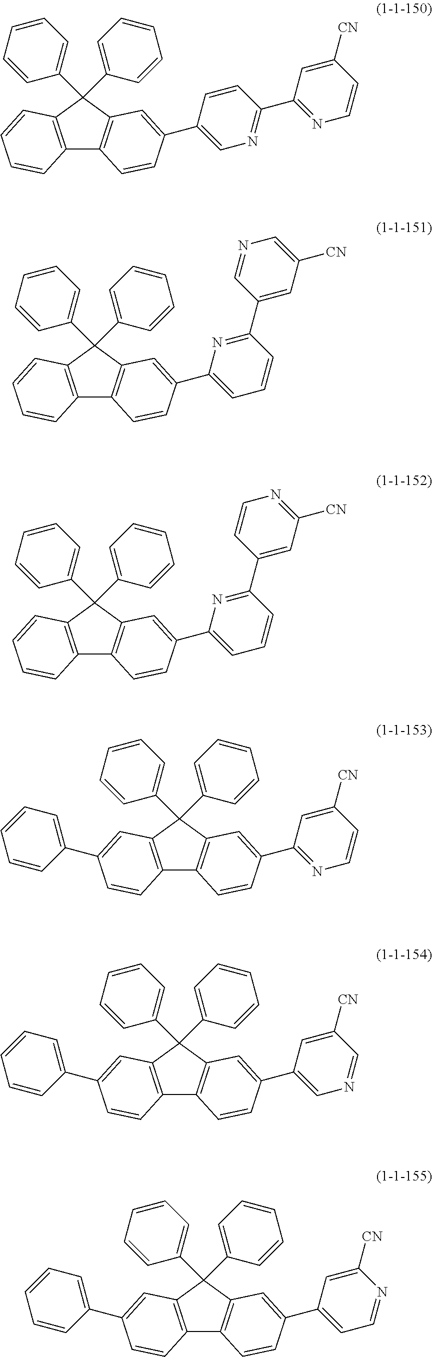 [SMILES: N#Cc1ccnc(-c2ccc(-c3ccc4c(c3)C(c3ccccc3)(c3ccccc3)c3ccccc3-4)cn2)c1 ; N#Cc1cncc(-c2cccc(-c3ccc4c(c3)C(c3ccccc3)(c3ccccc3)c3ccccc3-4)n2)c1 ; N#Cc1cc(-c2cccc(-c3ccc4c(c3)C(c3ccccc3)(c3ccccc3)c3ccccc3-4)n2)ccn1 ; N#Cc1ccnc(-c2ccc3c(c2)C(c2ccccc2)(c2ccccc2)c2cc(-c4ccccc4)ccc2-3)c1 ; N#Cc1cncc(-c2ccc3c(c2)C(c2ccccc2)(c2ccccc2)c2cc(-c4ccccc4)ccc2-3)c1 ; N#Cc1cc(-c2ccc3c(c2)C(c2ccccc2)(c2ccccc2)c2cc(-c4ccccc4)ccc2-3)ccn1]

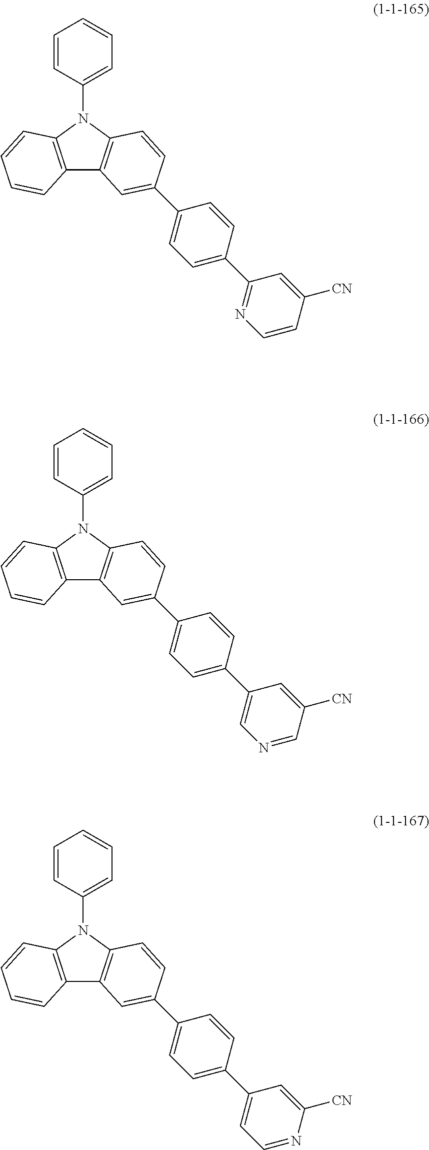 [SMILES: N#Cc1ccnc(-c2ccc(-c3ccc4c(c3)c3ccccc3n4-c3ccccc3)cc2)c1 ; N#Cc1cncc(-c2ccc(-c3ccc4c(c3)c3ccccc3n4-c3ccccc3)cc2)c1 ; N#Cc1cc(-c2ccc(-c3ccc4c(c3)c3ccccc3n4-c3ccccc3)cc2)ccn1]

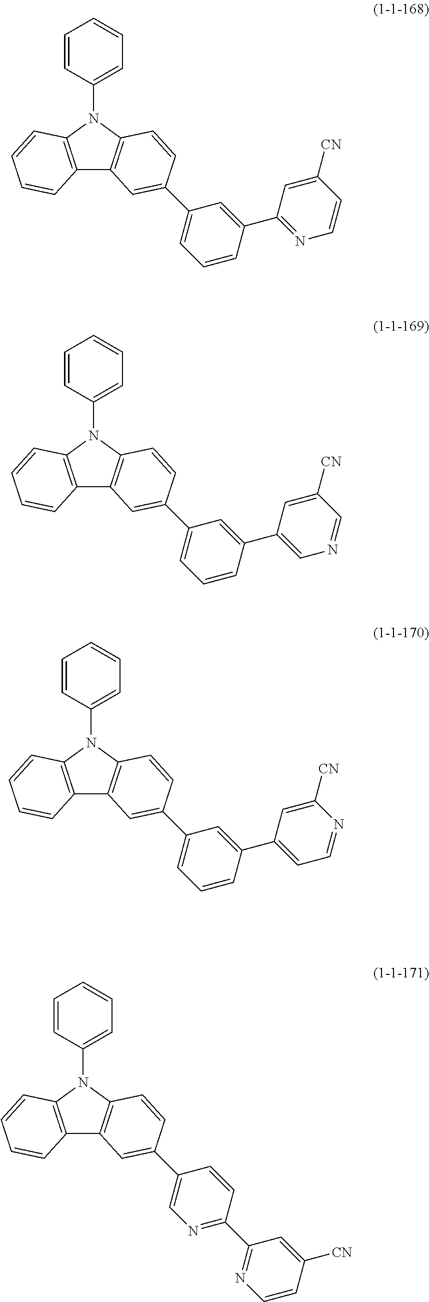 [SMILES: N#Cc1ccnc(-c2cccc(-c3ccc4c(c3)c3ccccc3n4-c3ccccc3)c2)c1 ; N#Cc1cncc(-c2cccc(-c3ccc4c(c3)c3ccccc3n4-c3ccccc3)c2)c1 ; N#Cc1cc(-c2cccc(-c3ccc4c(c3)c3ccccc3n4-c3ccccc3)c2)ccn1 ; N#Cc1ccnc(-c2ccc(-c3ccc4c(c3)c3ccccc3n4-c3ccccc3)cn2)c1]

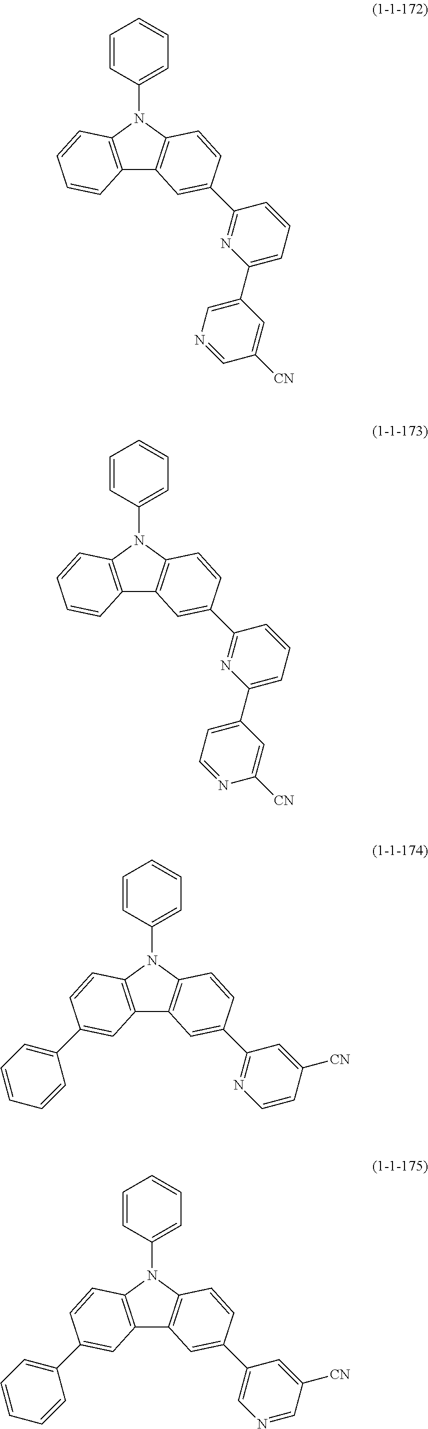 [SMILES: N#Cc1cncc(-c2cccc(-c3ccc4c(c3)c3ccccc3n4-c3ccccc3)n2)c1 ; N#Cc1cc(-c2cccc(-c3ccc4c(c3)c3ccccc3n4-c3ccccc3)n2)ccn1 ; N#Cc1ccnc(-c2ccc3c(c2)c2cc(-c4ccccc4)ccc2n3-c2ccccc2)c1 ; N#Cc1cncc(-c2ccc3c(c2)c2cc(-c4ccccc4)ccc2n3-c2ccccc2)c1]

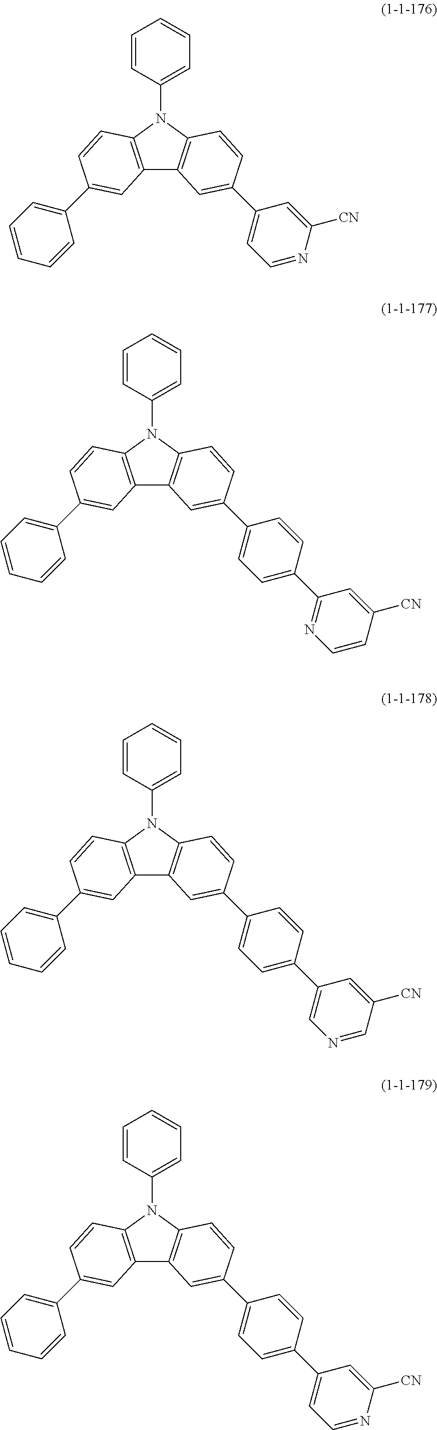 [SMILES: N#Cc1cc(-c2ccc3c(c2)c2cc(-c4ccccc4)ccc2n3-c2ccccc2)ccn1 ; N#Cc1ccnc(-c2ccc(-c3ccc4c(c3)c3cc(-c5ccccc5)ccc3n4-c3ccccc3)cc2)c1 ; N#Cc1cncc(-c2ccc(-c3ccc4c(c3)c3cc(-c5ccccc5)ccc3n4-c3ccccc3)cc2)c1 ; N#Cc1cc(-c2ccc(-c3ccc4c(c3)c3cc(-c5ccccc5)ccc3n4-c3ccccc3)cc2)ccn1]

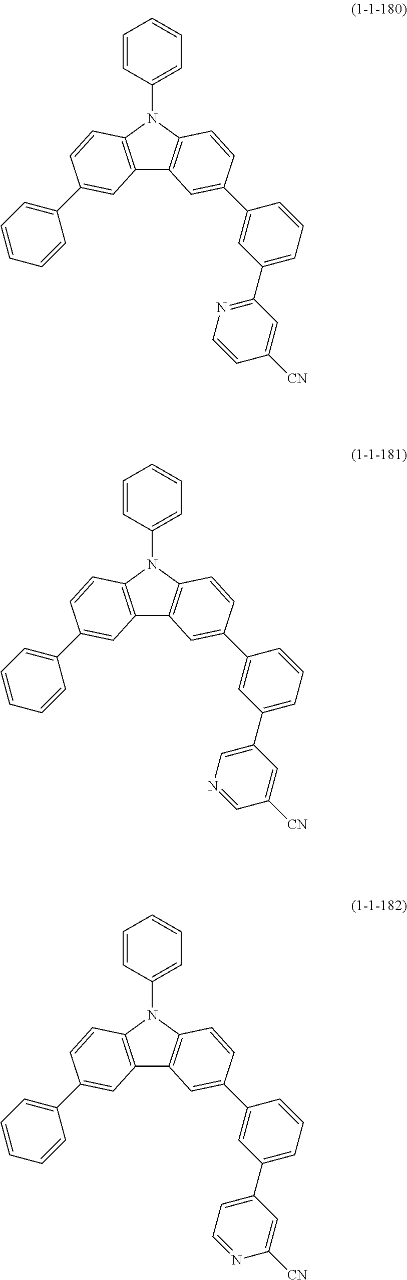 [SMILES: N#Cc1ccnc(-c2cccc(-c3ccc4c(c3)c3cc(-c5ccccc5)ccc3n4-c3ccccc3)c2)c1 ; N#Cc1cncc(-c2cccc(-c3ccc4c(c3)c3cc(-c5ccccc5)ccc3n4-c3ccccc3)c2)c1 ; N#Cc1cc(-c2cccc(-c3ccc4c(c3)c3cc(-c5ccccc5)ccc3n4-c3ccccc3)c2)ccn1]

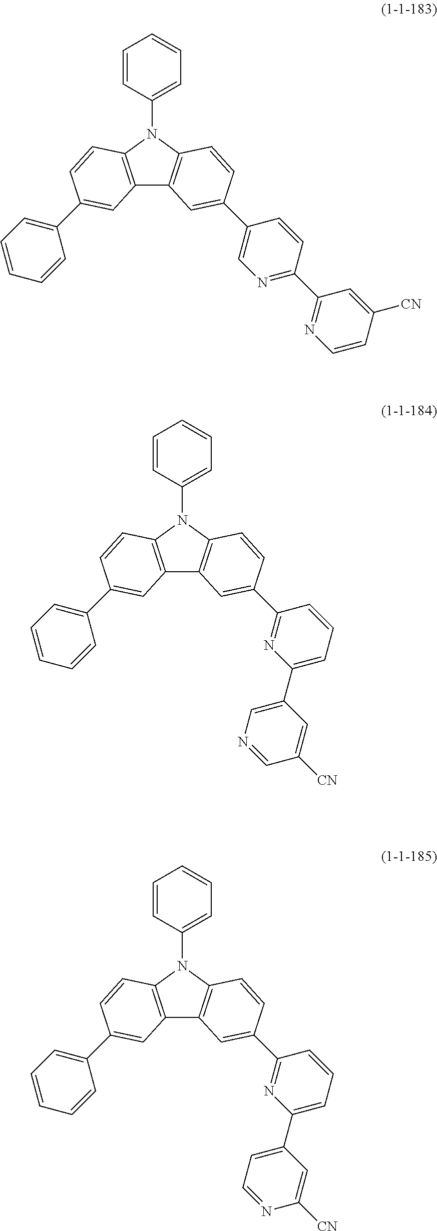 [SMILES: N#Cc1ccnc(-c2ccc(-c3ccc4c(c3)c3cc(-c5ccccc5)ccc3n4-c3ccccc3)cn2)c1 ; N#Cc1cncc(-c2cccc(-c3ccc4c(c3)c3cc(-c5ccccc5)ccc3n4-c3ccccc3)n2)c1 ; N#Cc1cc(-c2cccc(-c3ccc4c(c3)c3cc(-c5ccccc5)ccc3n4-c3ccccc3)n2)ccn1]

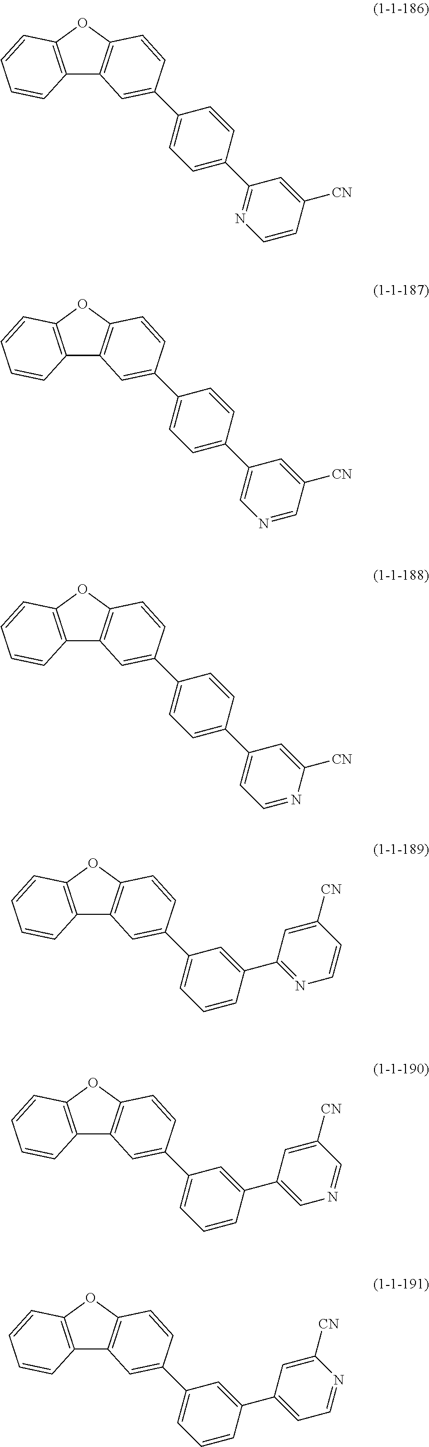 [SMILES: N#Cc1ccnc(-c2ccc(-c3ccc4oc5ccccc5c4c3)cc2)c1 ; N#Cc1cncc(-c2ccc(-c3ccc4oc5ccccc5c4c3)cc2)c1 ; N#Cc1cc(-c2ccc(-c3ccc4oc5ccccc5c4c3)cc2)ccn1 ; N#Cc1ccnc(-c2cccc(-c3ccc4oc5ccccc5c4c3)c2)c1 ; N#Cc1cncc(-c2cccc(-c3ccc4oc5ccccc5c4c3)c2)c1 ; N#Cc1cc(-c2cccc(-c3ccc4oc5ccccc5c4c3)c2)ccn1]

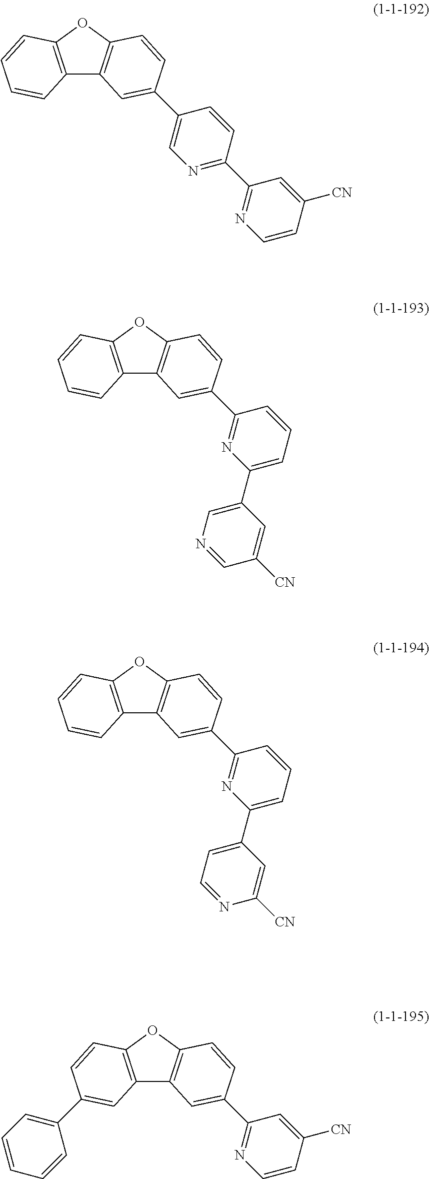 [SMILES: N#Cc1ccnc(-c2ccc(-c3ccc4oc5ccccc5c4c3)cn2)c1 ; N#Cc1cncc(-c2cccc(-c3ccc4oc5ccccc5c4c3)n2)c1 ; N#Cc1cc(-c2cccc(-c3ccc4oc5ccccc5c4c3)n2)ccn1 ; N#Cc1ccnc(-c2ccc3oc4ccc(-c5ccccc5)cc4c3c2)c1]

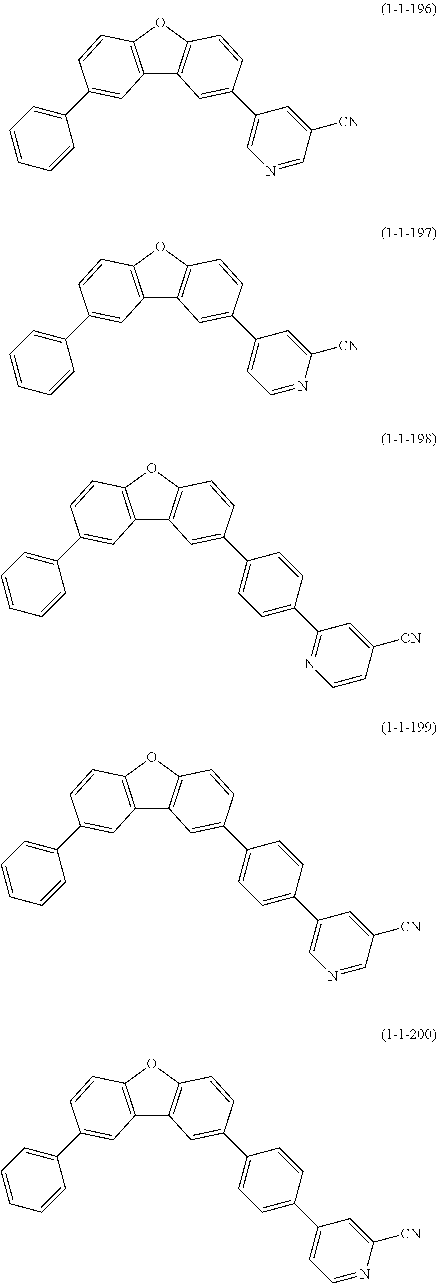 [SMILES: N#Cc1cncc(-c2ccc3oc4ccc(-c5ccccc5)cc4c3c2)c1 ; N#Cc1cc(-c2ccc3oc4ccc(-c5ccccc5)cc4c3c2)ccn1 ; N#Cc1ccnc(-c2ccc(-c3ccc4oc5ccc(-c6ccccc6)cc5c4c3)cc2)c1 ; N#Cc1cncc(-c2ccc(-c3ccc4oc5ccc(-c6ccccc6)cc5c4c3)cc2)c1 ; N#Cc1cc(-c2ccc(-c3ccc4oc5ccc(-c6ccccc6)cc5c4c3)cc2)ccn1]

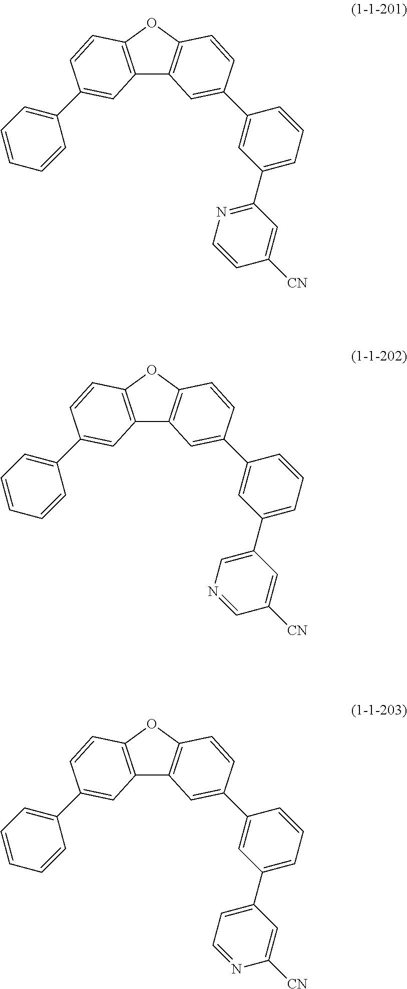 [SMILES: N#Cc1ccnc(-c2cccc(-c3ccc4oc5ccc(-c6ccccc6)cc5c4c3)c2)c1 ; N#Cc1cncc(-c2cccc(-c3ccc4oc5ccc(-c6ccccc6)cc5c4c3)c2)c1 ; N#Cc1cc(-c2cccc(-c3ccc4oc5ccc(-c6ccccc6)cc5c4c3)c2)ccn1]

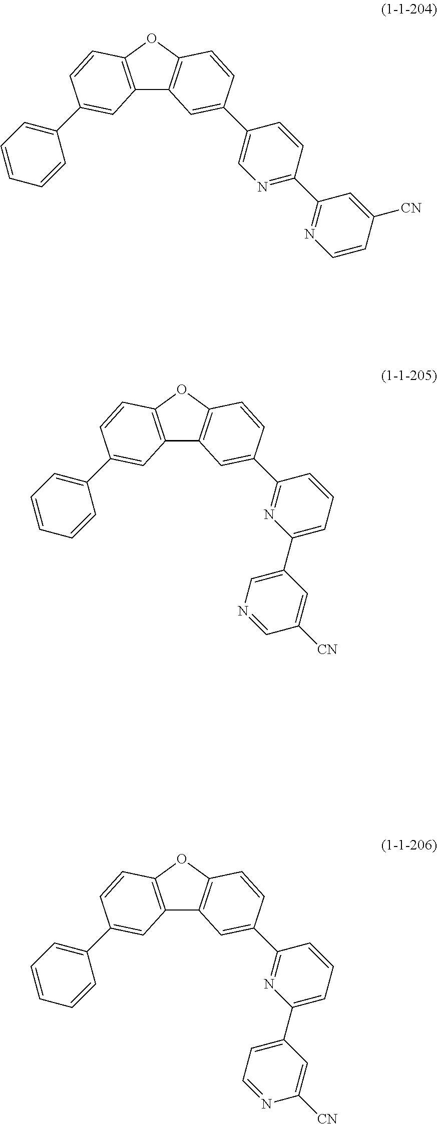 [SMILES: N#Cc1ccnc(-c2ccc(-c3ccc4oc5ccc(-c6ccccc6)cc5c4c3)cn2)c1 ; N#Cc1cncc(-c2cccc(-c3ccc4oc5ccc(-c6ccccc6)cc5c4c3)n2)c1 ; N#Cc1cc(-c2cccc(-c3ccc4oc5ccc(-c6ccccc6)cc5c4c3)n2)ccn1]

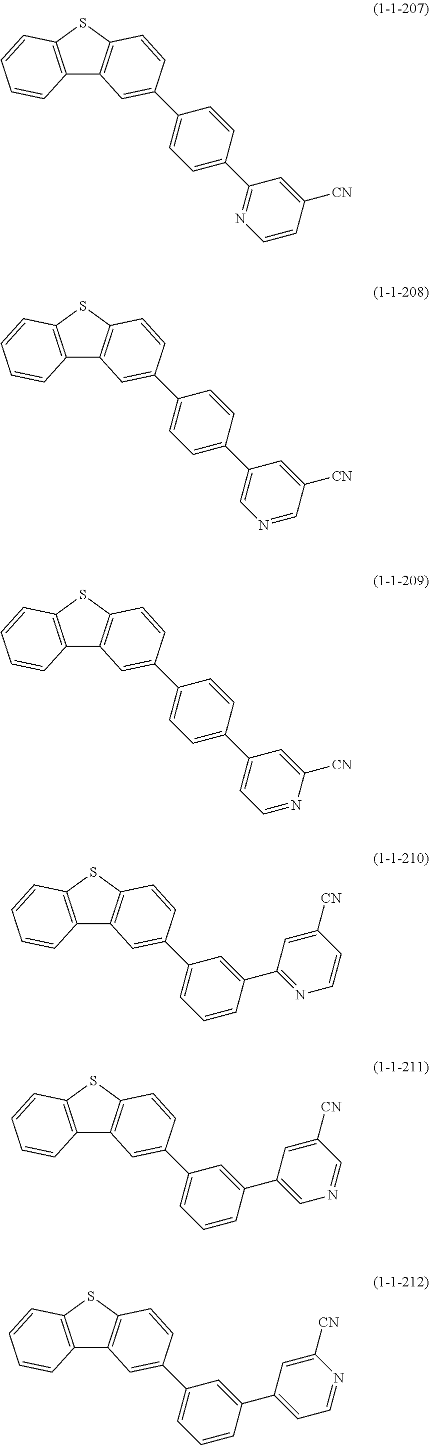 [SMILES: N#Cc1ccnc(-c2ccc(-c3ccc4sc5ccccc5c4c3)cc2)c1 ; N#Cc1cncc(-c2ccc(-c3ccc4sc5ccccc5c4c3)cc2)c1 ; N#Cc1cc(-c2ccc(-c3ccc4sc5ccccc5c4c3)cc2)ccn1 ; N#Cc1ccnc(-c2cccc(-c3ccc4sc5ccccc5c4c3)c2)c1 ; N#Cc1cncc(-c2cccc(-c3ccc4sc5ccccc5c4c3)c2)c1 ; N#Cc1cc(-c2cccc(-c3ccc4sc5ccccc5c4c3)c2)ccn1]

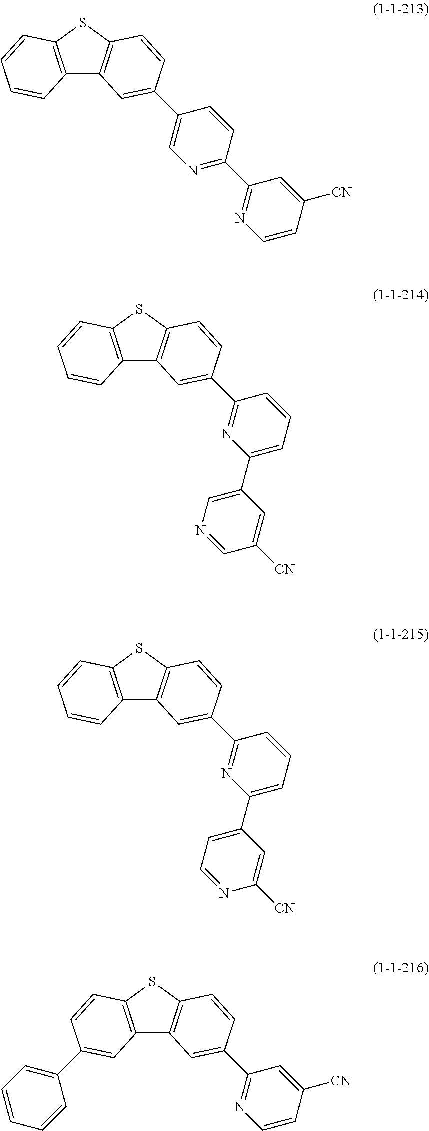 [SMILES: N#Cc1ccnc(-c2ccc(-c3ccc4sc5ccccc5c4c3)cn2)c1 ; N#Cc1cncc(-c2cccc(-c3ccc4sc5ccccc5c4c3)n2)c1 ; N#Cc1cc(-c2cccc(-c3ccc4sc5ccccc5c4c3)n2)ccn1 ; N#Cc1ccnc(-c2ccc3sc4ccc(-c5ccccc5)cc4c3c2)c1]

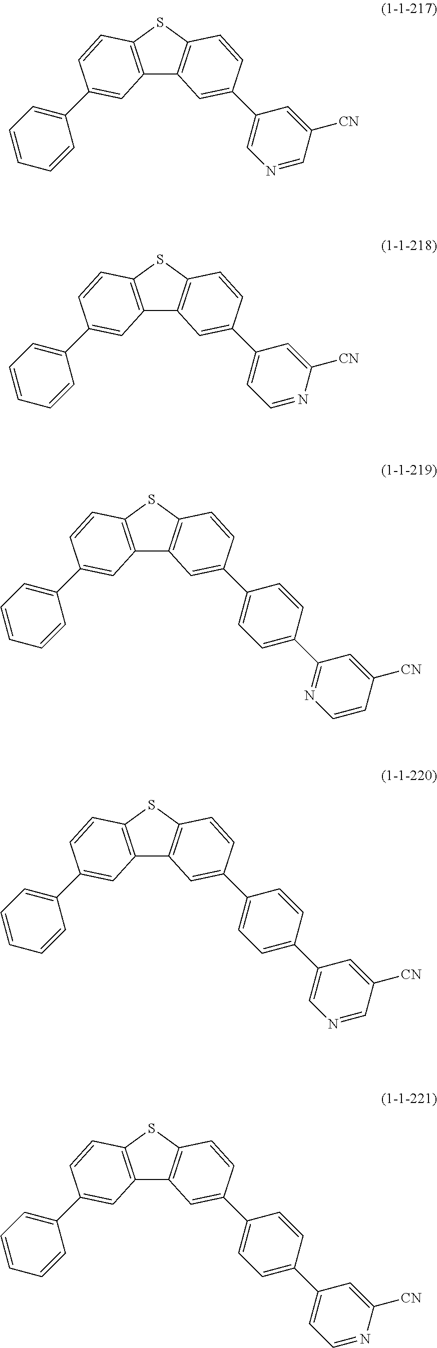 [SMILES: N#Cc1cncc(-c2ccc3sc4ccc(-c5ccccc5)cc4c3c2)c1 ; N#Cc1cc(-c2ccc3sc4ccc(-c5ccccc5)cc4c3c2)ccn1 ; N#Cc1ccnc(-c2ccc(-c3ccc4sc5ccc(-c6ccccc6)cc5c4c3)cc2)c1 ; N#Cc1cncc(-c2ccc(-c3ccc4sc5ccc(-c6ccccc6)cc5c4c3)cc2)c1 ; N#Cc1cc(-c2ccc(-c3ccc4sc5ccc(-c6ccccc6)cc5c4c3)cc2)ccn1]

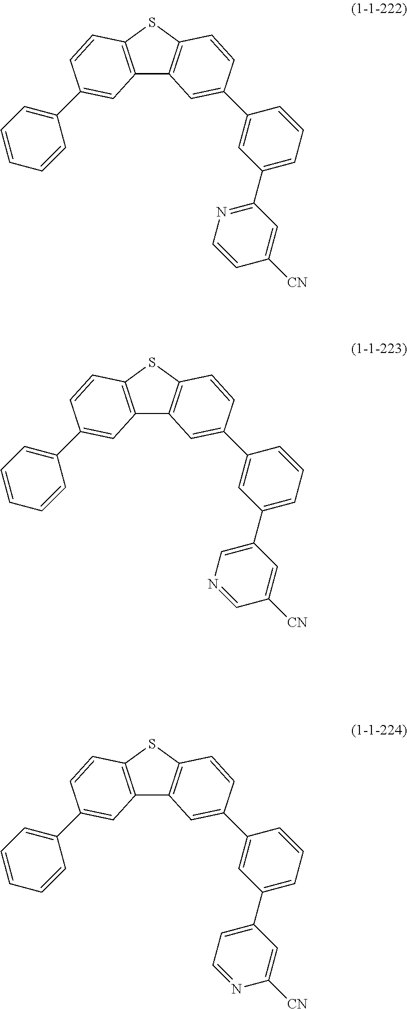 [SMILES: N#Cc1ccnc(-c2cccc(-c3ccc4sc5ccc(-c6ccccc6)cc5c4c3)c2)c1 ; N#Cc1cncc(-c2cccc(-c3ccc4sc5ccc(-c6ccccc6)cc5c4c3)c2)c1 ; N#Cc1cc(-c2cccc(-c3ccc4sc5ccc(-c6ccccc6)cc5c4c3)c2)ccn1]

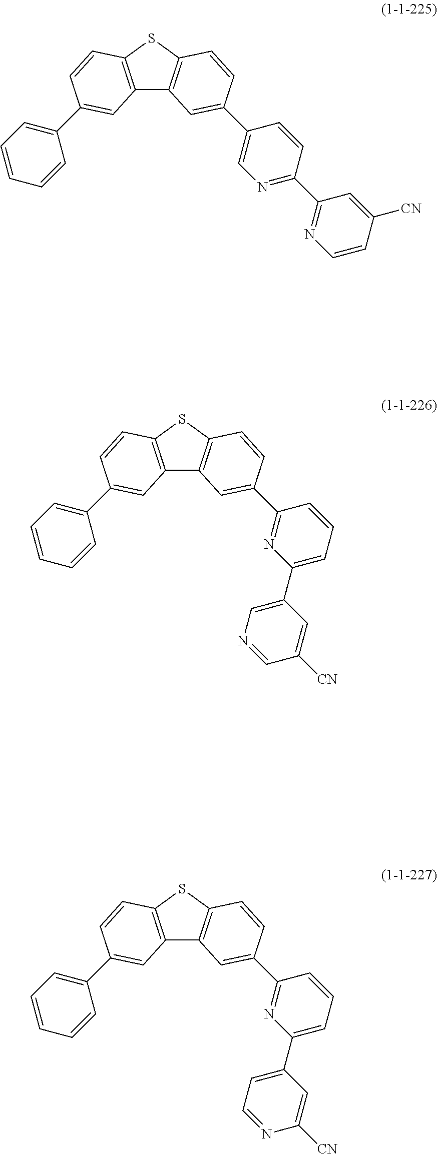 [SMILES: N#Cc1ccnc(-c2ccc(-c3ccc4sc5ccc(-c6ccccc6)cc5c4c3)cn2)c1 ; N#Cc1cncc(-c2cccc(-c3ccc4sc5ccc(-c6ccccc6)cc5c4c3)n2)c1 ; N#Cc1cc(-c2cccc(-c3ccc4sc5ccc(-c6ccccc6)cc5c4c3)n2)ccn1]

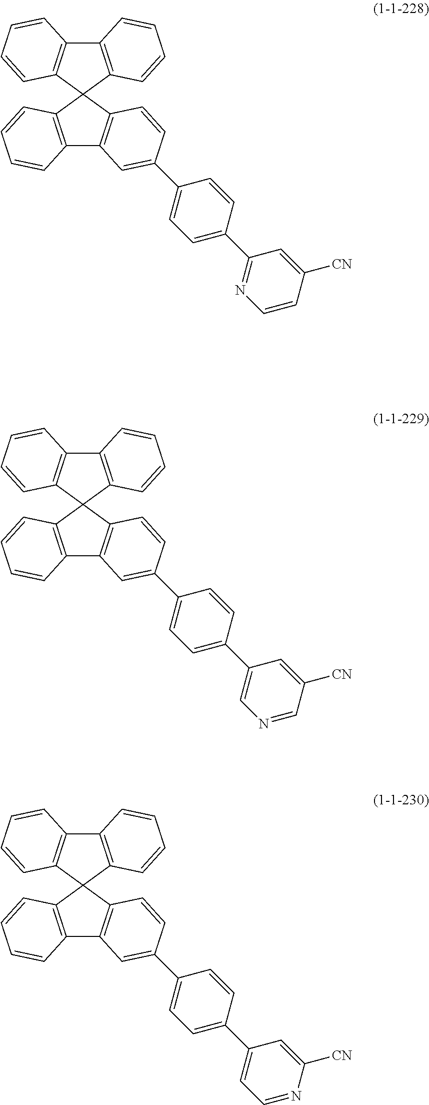 [SMILES: N#Cc1ccnc(-c2ccc(-c3ccc4c(c3)-c3ccccc3C43c4ccccc4-c4ccccc43)cc2)c1 ; N#Cc1cncc(-c2ccc(-c3ccc4c(c3)-c3ccccc3C43c4ccccc4-c4ccccc43)cc2)c1 ; N#Cc1cc(-c2ccc(-c3ccc4c(c3)-c3ccccc3C43c4ccccc4-c4ccccc43)cc2)ccn1]

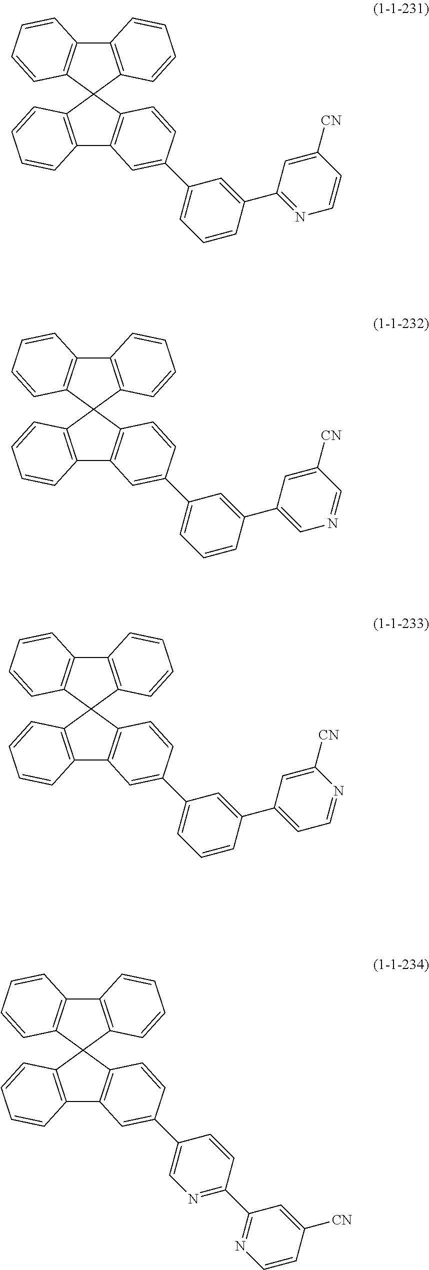 [SMILES: N#Cc1ccnc(-c2cccc(-c3ccc4c(c3)-c3ccccc3C43c4ccccc4-c4ccccc43)c2)c1 ; N#Cc1cncc(-c2cccc(-c3ccc4c(c3)-c3ccccc3C43c4ccccc4-c4ccccc43)c2)c1 ; N#Cc1cc(-c2cccc(-c3ccc4c(c3)-c3ccccc3C43c4ccccc4-c4ccccc43)c2)ccn1 ; N#Cc1ccnc(-c2ccc(-c3ccc4c(c3)-c3ccccc3C43c4ccccc4-c4ccccc43)cn2)c1]

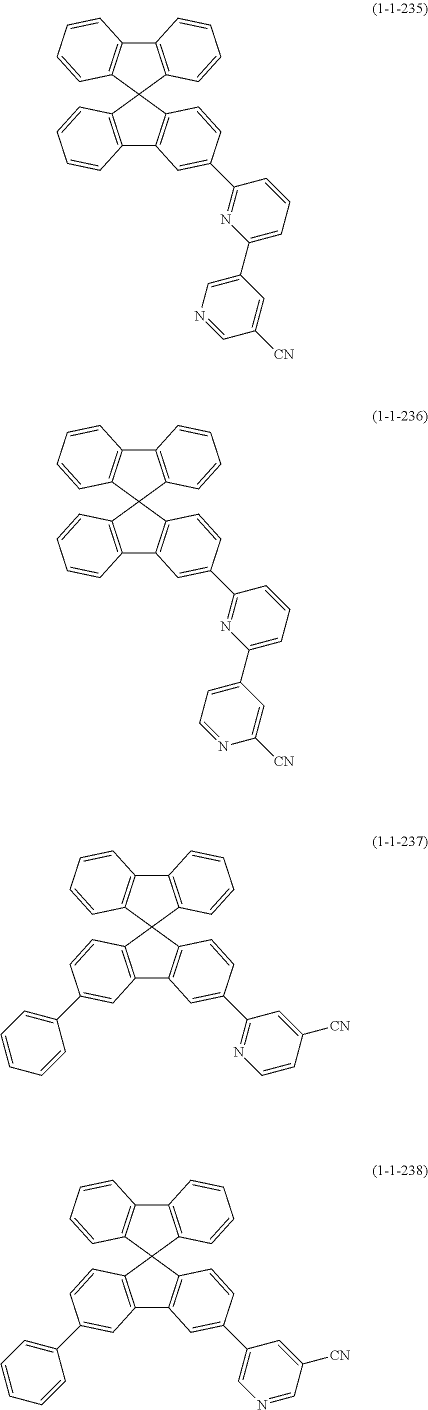 [SMILES: N#Cc1cncc(-c2cccc(-c3ccc4c(c3)-c3ccccc3C43c4ccccc4-c4ccccc43)n2)c1 ; N#Cc1cc(-c2cccc(-c3ccc4c(c3)-c3ccccc3C43c4ccccc4-c4ccccc43)n2)ccn1 ; N#Cc1ccnc(-c2ccc3c(c2)-c2cc(-c4ccccc4)ccc2C32c3ccccc3-c3ccccc32)c1 ; N#Cc1cncc(-c2ccc3c(c2)-c2cc(-c4ccccc4)ccc2C32c3ccccc3-c3ccccc32)c1]

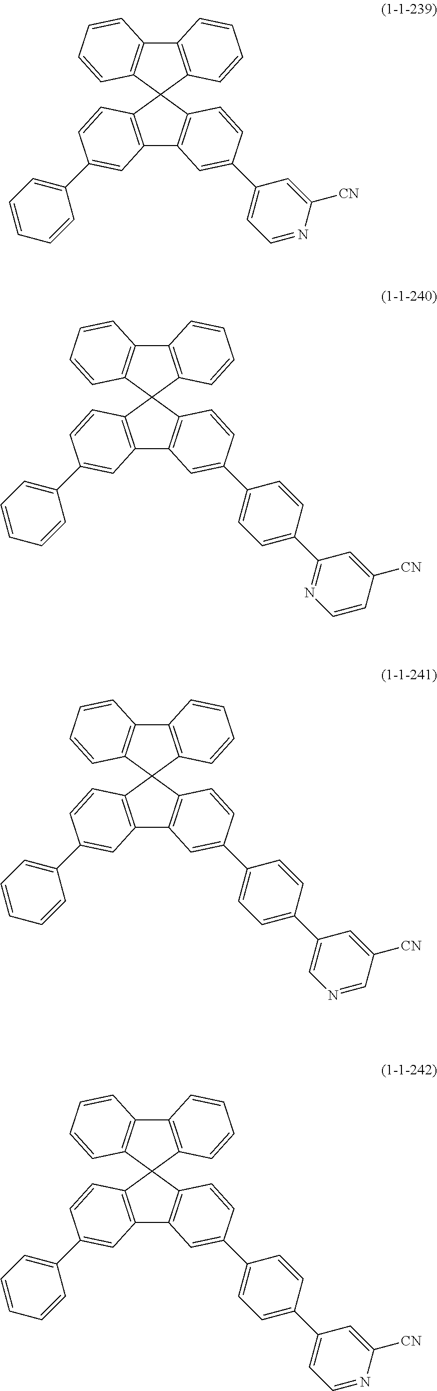 [SMILES: N#Cc1cc(-c2ccc3c(c2)-c2cc(-c4ccccc4)ccc2C32c3ccccc3-c3ccccc32)ccn1 ; N#Cc1ccnc(-c2ccc(-c3ccc4c(c3)-c3cc(-c5ccccc5)ccc3C43c4ccccc4-c4ccccc43)cc2)c1 ; N#Cc1cncc(-c2ccc(-c3ccc4c(c3)-c3cc(-c5ccccc5)ccc3C43c4ccccc4-c4ccccc43)cc2)c1 ; N#Cc1cc(-c2ccc(-c3ccc4c(c3)-c3cc(-c5ccccc5)ccc3C43c4ccccc4-c4ccccc43)cc2)ccn1]

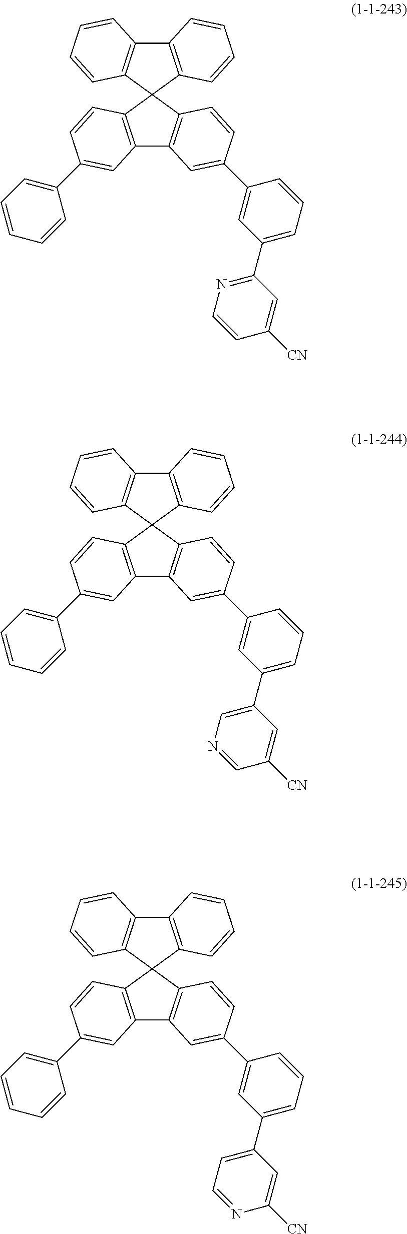 [SMILES: N#Cc1ccnc(-c2cccc(-c3ccc4c(c3)-c3cc(-c5ccccc5)ccc3C43c4ccccc4-c4ccccc43)c2)c1 ; N#Cc1cncc(-c2cccc(-c3ccc4c(c3)-c3cc(-c5ccccc5)ccc3C43c4ccccc4-c4ccccc43)c2)c1 ; N#Cc1cc(-c2cccc(-c3ccc4c(c3)-c3cc(-c5ccccc5)ccc3C43c4ccccc4-c4ccccc43)c2)ccn1]

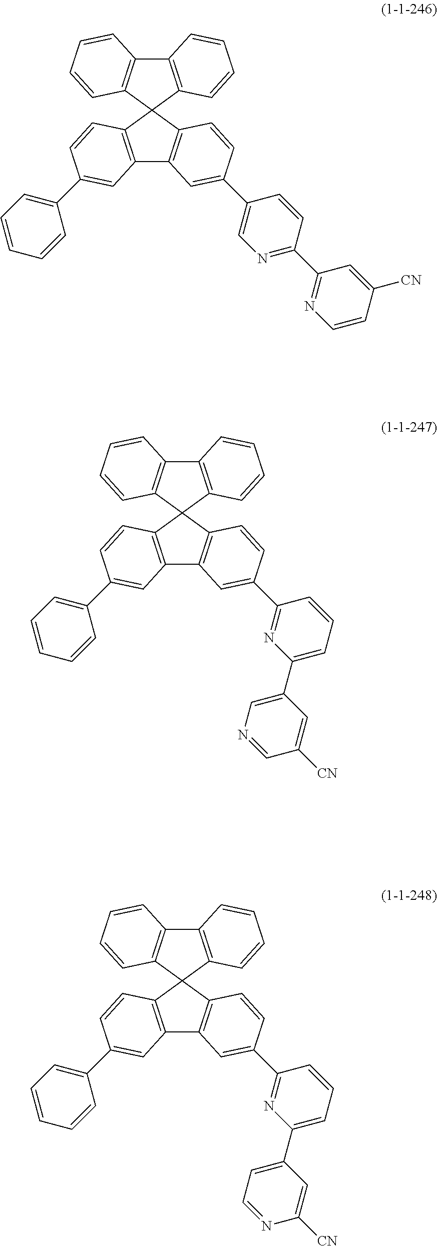 [SMILES: N#Cc1ccnc(-c2ccc(-c3ccc4c(c3)-c3cc(-c5ccccc5)ccc3C43c4ccccc4-c4ccccc43)cn2)c1 ; N#Cc1cncc(-c2cccc(-c3ccc4c(c3)-c3cc(-c5ccccc5)ccc3C43c4ccccc4-c4ccccc43)n2)c1 ; N#Cc1cc(-c2cccc(-c3ccc4c(c3)-c3cc(-c5ccccc5)ccc3C43c4ccccc4-c4ccccc43)n2)ccn1]

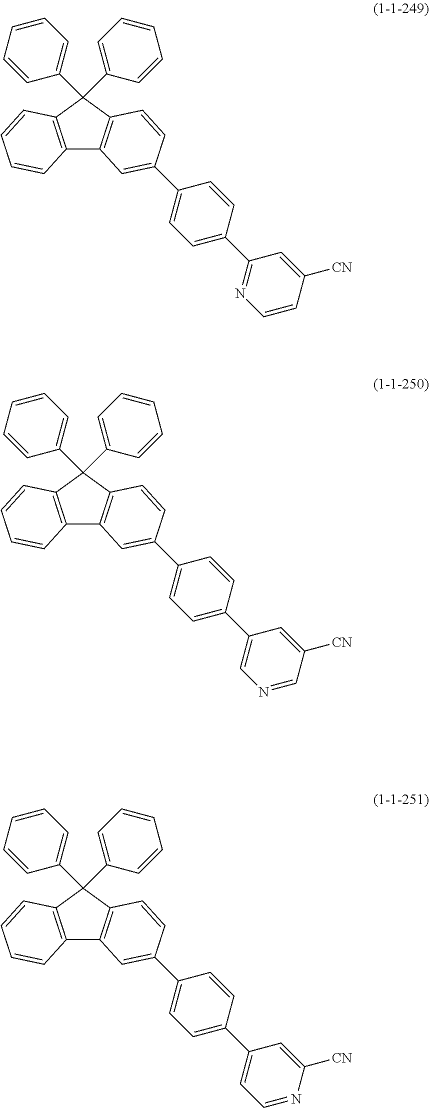 [SMILES: N#Cc1ccnc(-c2ccc(-c3ccc4c(c3)-c3ccccc3C4(c3ccccc3)c3ccccc3)cc2)c1 ; N#Cc1cncc(-c2ccc(-c3ccc4c(c3)-c3ccccc3C4(c3ccccc3)c3ccccc3)cc2)c1 ; N#Cc1cc(-c2ccc(-c3ccc4c(c3)-c3ccccc3C4(c3ccccc3)c3ccccc3)cc2)ccn1]

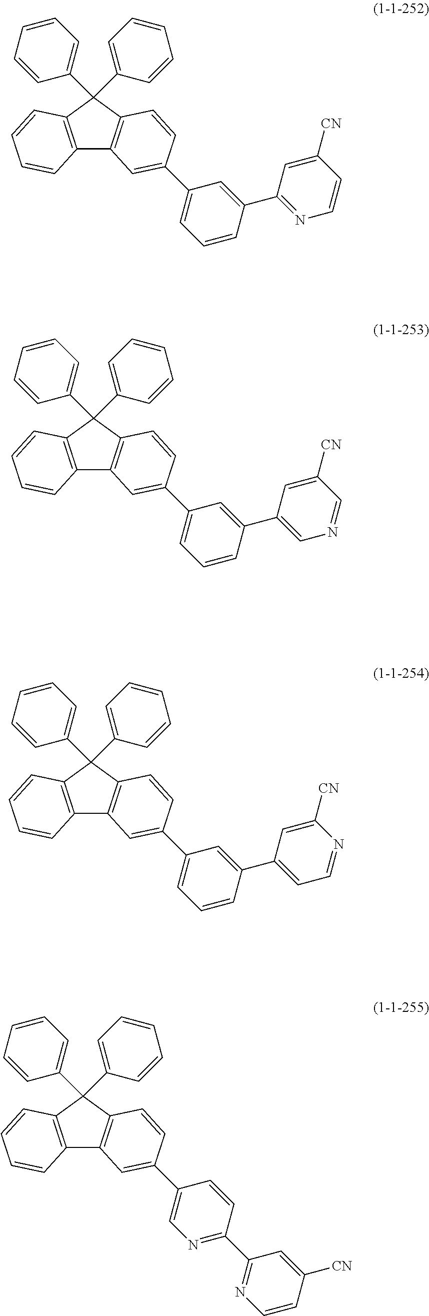 [SMILES: N#Cc1ccnc(-c2cccc(-c3ccc4c(c3)-c3ccccc3C4(c3ccccc3)c3ccccc3)c2)c1 ; N#Cc1cncc(-c2cccc(-c3ccc4c(c3)-c3ccccc3C4(c3ccccc3)c3ccccc3)c2)c1 ; N#Cc1cc(-c2cccc(-c3ccc4c(c3)-c3ccccc3C4(c3ccccc3)c3ccccc3)c2)ccn1 ; N#Cc1ccnc(-c2ccc(-c3ccc4c(c3)-c3ccccc3C4(c3ccccc3)c3ccccc3)cn2)c1]

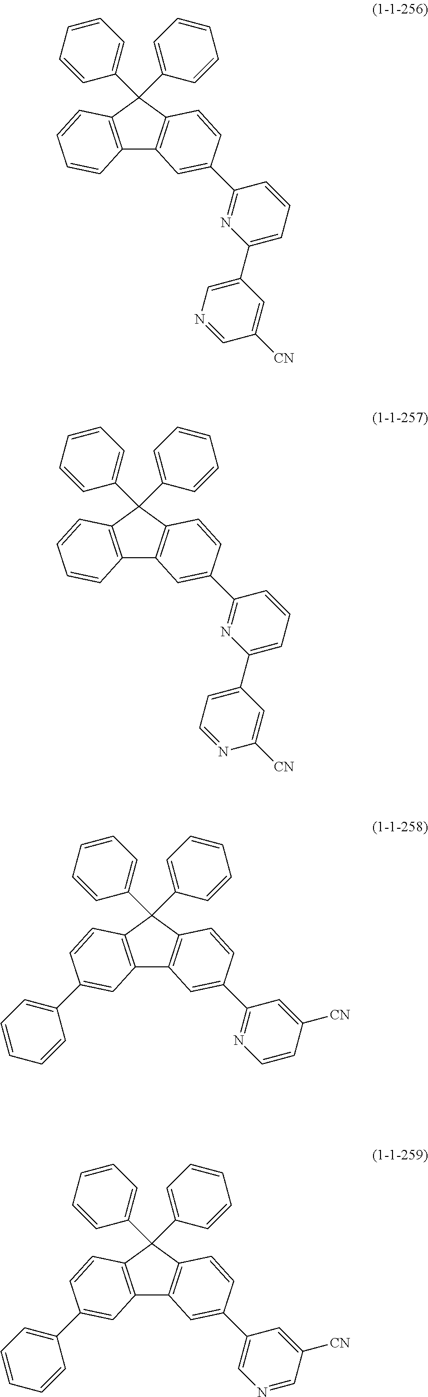 [SMILES: N#Cc1cncc(-c2cccc(-c3ccc4c(c3)-c3ccccc3C4(c3ccccc3)c3ccccc3)n2)c1 ; N#Cc1cc(-c2cccc(-c3ccc4c(c3)-c3ccccc3C4(c3ccccc3)c3ccccc3)n2)ccn1 ; N#Cc1ccnc(-c2ccc3c(c2)-c2cc(-c4ccccc4)ccc2C3(c2ccccc2)c2ccccc2)c1 ; N#Cc1cncc(-c2ccc3c(c2)-c2cc(-c4ccccc4)ccc2C3(c2ccccc2)c2ccccc2)c1]

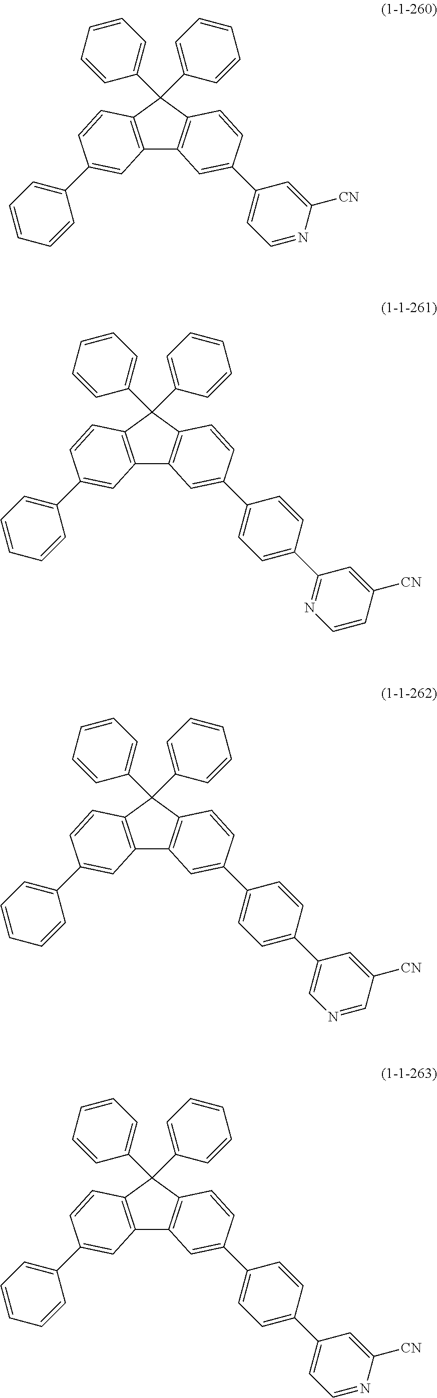 [SMILES: N#Cc1cc(-c2ccc3c(c2)-c2cc(-c4ccccc4)ccc2C3(c2ccccc2)c2ccccc2)ccn1 ; N#Cc1ccnc(-c2ccc(-c3ccc4c(c3)-c3cc(-c5ccccc5)ccc3C4(c3ccccc3)c3ccccc3)cc2)c1 ; N#Cc1cncc(-c2ccc(-c3ccc4c(c3)-c3cc(-c5ccccc5)ccc3C4(c3ccccc3)c3ccccc3)cc2)c1 ; N#Cc1cc(-c2ccc(-c3ccc4c(c3)-c3cc(-c5ccccc5)ccc3C4(c3ccccc3)c3ccccc3)cc2)ccn1]

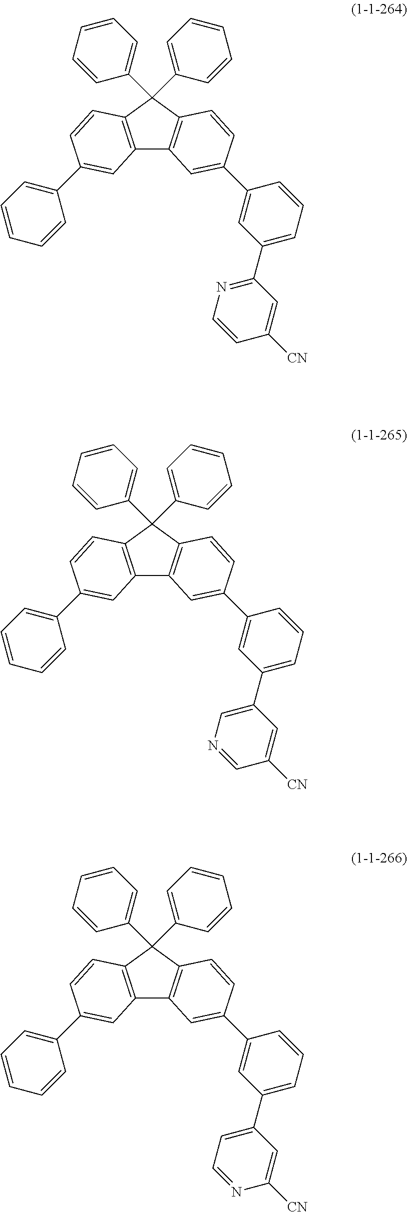 [SMILES: N#Cc1ccnc(-c2cccc(-c3ccc4c(c3)-c3cc(-c5ccccc5)ccc3C4(c3ccccc3)c3ccccc3)c2)c1 ; N#Cc1cncc(-c2cccc(-c3ccc4c(c3)-c3cc(-c5ccccc5)ccc3C4(c3ccccc3)c3ccccc3)c2)c1 ; N#Cc1cc(-c2cccc(-c3ccc4c(c3)-c3cc(-c5ccccc5)ccc3C4(c3ccccc3)c3ccccc3)c2)ccn1]

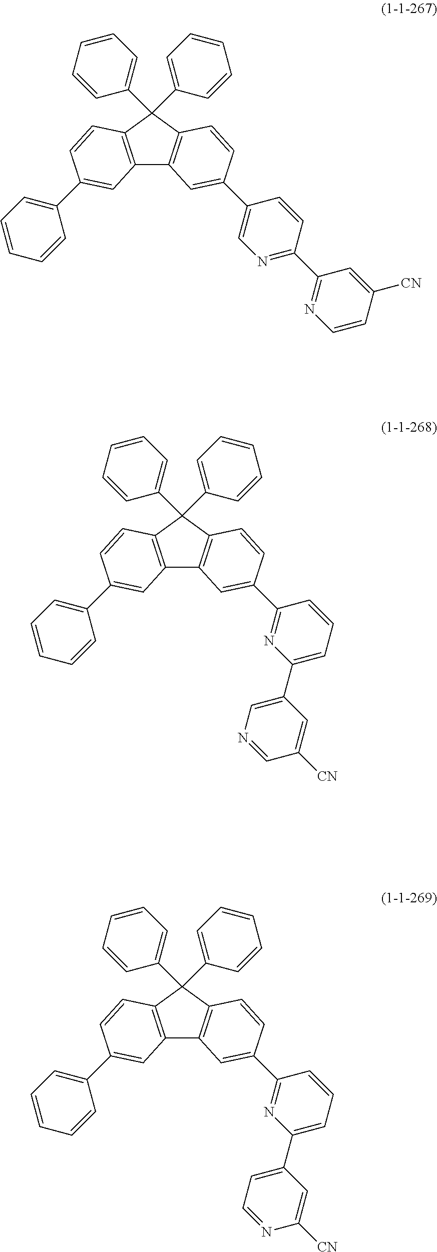 [SMILES: N#Cc1ccnc(-c2ccc(-c3ccc4c(c3)-c3cc(-c5ccccc5)ccc3C4(c3ccccc3)c3ccccc3)cn2)c1 ; N#Cc1cncc(-c2cccc(-c3ccc4c(c3)-c3cc(-c5ccccc5)ccc3C4(c3ccccc3)c3ccccc3)n2)c1 ; N#Cc1cc(-c2cccc(-c3ccc4c(c3)-c3cc(-c5ccccc5)ccc3C4(c3ccccc3)c3ccccc3)n2)ccn1]

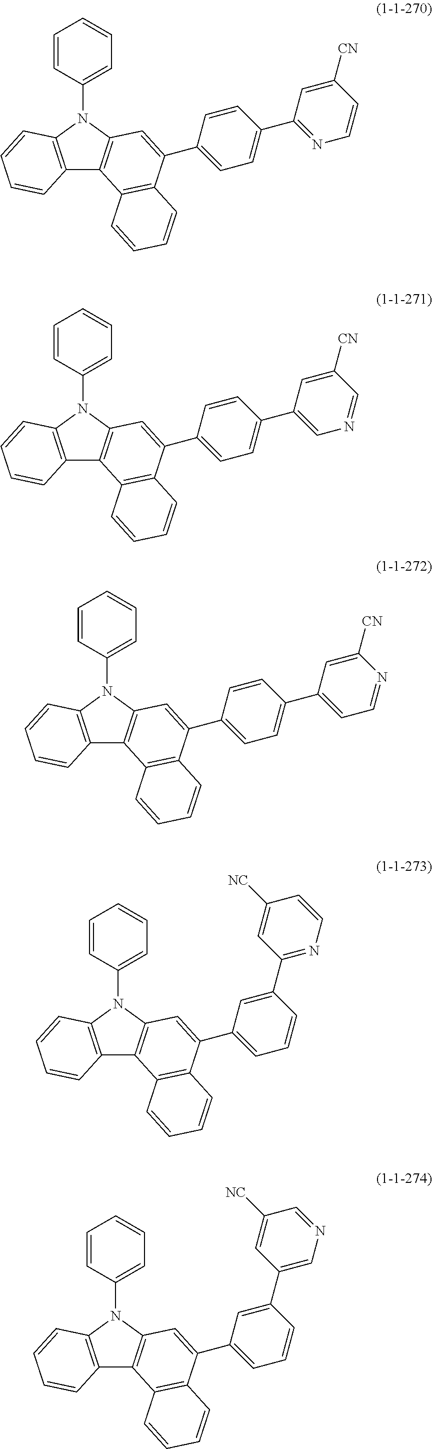 [SMILES: N#Cc1ccnc(-c2ccc(-c3cc4c(c5ccccc35)c3ccccc3n4-c3ccccc3)cc2)c1 ; N#Cc1cncc(-c2ccc(-c3cc4c(c5ccccc35)c3ccccc3n4-c3ccccc3)cc2)c1 ; N#Cc1cc(-c2ccc(-c3cc4c(c5ccccc35)c3ccccc3n4-c3ccccc3)cc2)ccn1 ; N#Cc1ccnc(-c2cccc(-c3cc4c(c5ccccc35)c3ccccc3n4-c3ccccc3)c2)c1 ; N#Cc1cncc(-c2cccc(-c3cc4c(c5ccccc35)c3ccccc3n4-c3ccccc3)c2)c1]

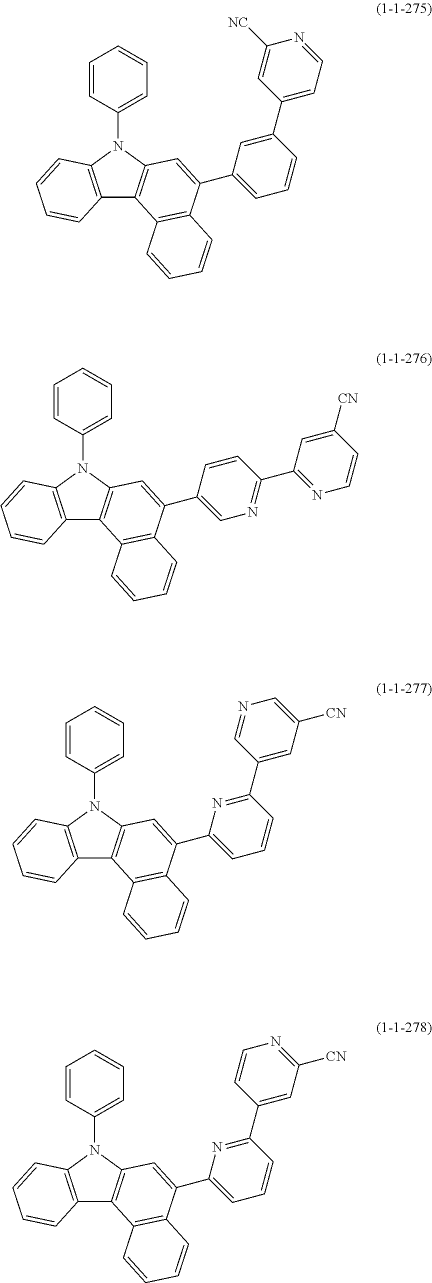 [SMILES: N#Cc1cc(-c2cccc(-c3cc4c(c5ccccc35)c3ccccc3n4-c3ccccc3)c2)ccn1 ; N#Cc1ccnc(-c2ccc(-c3cc4c(c5ccccc35)c3ccccc3n4-c3ccccc3)cn2)c1 ; N#Cc1cncc(-c2cccc(-c3cc4c(c5ccccc35)c3ccccc3n4-c3ccccc3)n2)c1 ; N#Cc1cc(-c2cccc(-c3cc4c(c5ccccc35)c3ccccc3n4-c3ccccc3)n2)ccn1]

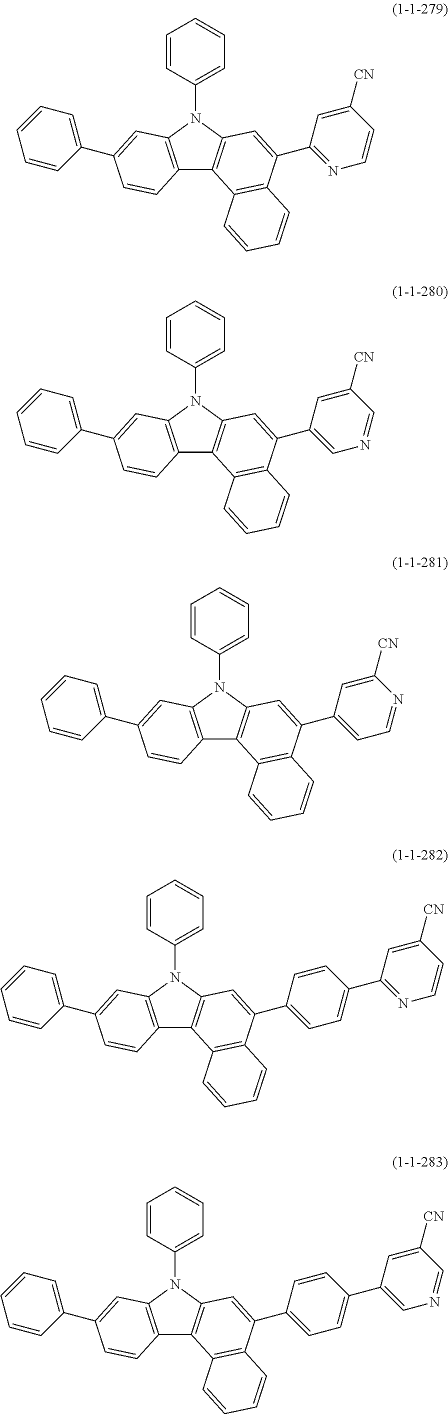 [SMILES: N#Cc1ccnc(-c2cc3c(c4ccccc24)c2ccc(-c4ccccc4)cc2n3-c2ccccc2)c1 ; N#Cc1cncc(-c2cc3c(c4ccccc24)c2ccc(-c4ccccc4)cc2n3-c2ccccc2)c1 ; N#Cc1cc(-c2cc3c(c4ccccc24)c2ccc(-c4ccccc4)cc2n3-c2ccccc2)ccn1 ; N#Cc1ccnc(-c2ccc(-c3cc4c(c5ccccc35)c3ccc(-c5ccccc5)cc3n4-c3ccccc3)cc2)c1 ; N#Cc1cncc(-c2ccc(-c3cc4c(c5ccccc35)c3ccc(-c5ccccc5)cc3n4-c3ccccc3)cc2)c1]

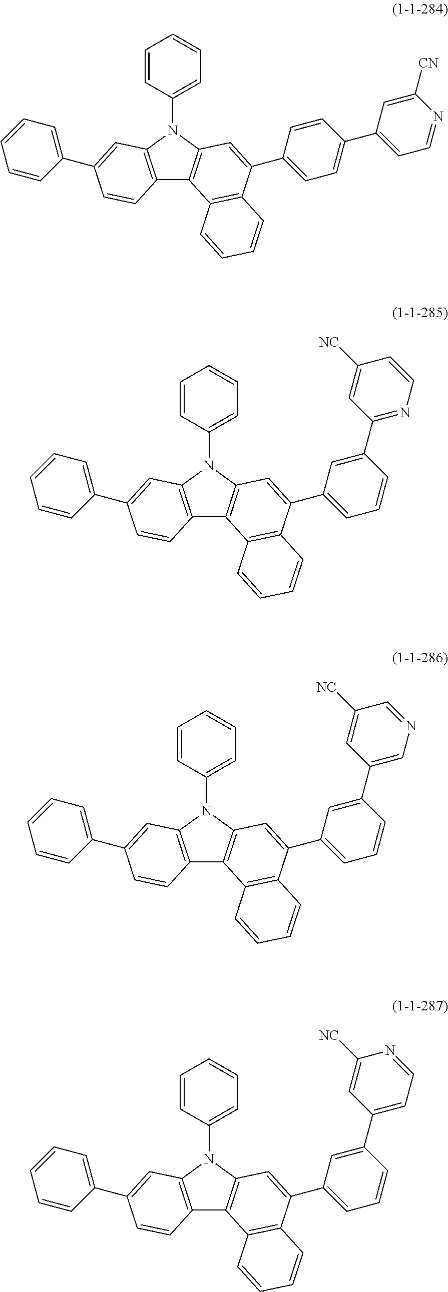 [SMILES: N#Cc1cc(-c2ccc(-c3cc4c(c5ccccc35)c3ccc(-c5ccccc5)cc3n4-c3ccccc3)cc2)ccn1 ; N#Cc1ccnc(-c2cccc(-c3cc4c(c5ccccc35)c3ccc(-c5ccccc5)cc3n4-c3ccccc3)c2)c1 ; N#Cc1cncc(-c2cccc(-c3cc4c(c5ccccc35)c3ccc(-c5ccccc5)cc3n4-c3ccccc3)c2)c1 ; N#Cc1cc(-c2cccc(-c3cc4c(c5ccccc35)c3ccc(-c5ccccc5)cc3n4-c3ccccc3)c2)ccn1]

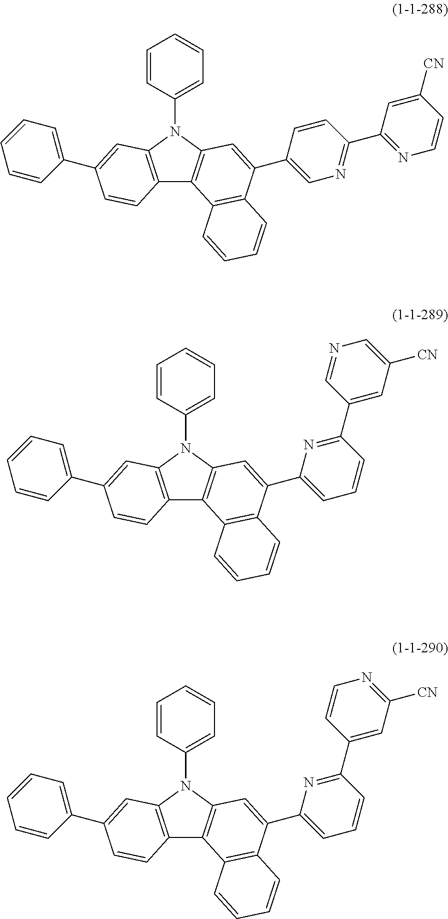 [SMILES: N#Cc1ccnc(-c2ccc(-c3cc4c(c5ccccc35)c3ccc(-c5ccccc5)cc3n4-c3ccccc3)cn2)c1 ; N#Cc1cncc(-c2cccc(-c3cc4c(c5ccccc35)c3ccc(-c5ccccc5)cc3n4-c3ccccc3)n2)c1 ; N#Cc1cc(-c2cccc(-c3cc4c(c5ccccc35)c3ccc(-c5ccccc5)cc3n4-c3ccccc3)n2)ccn1]

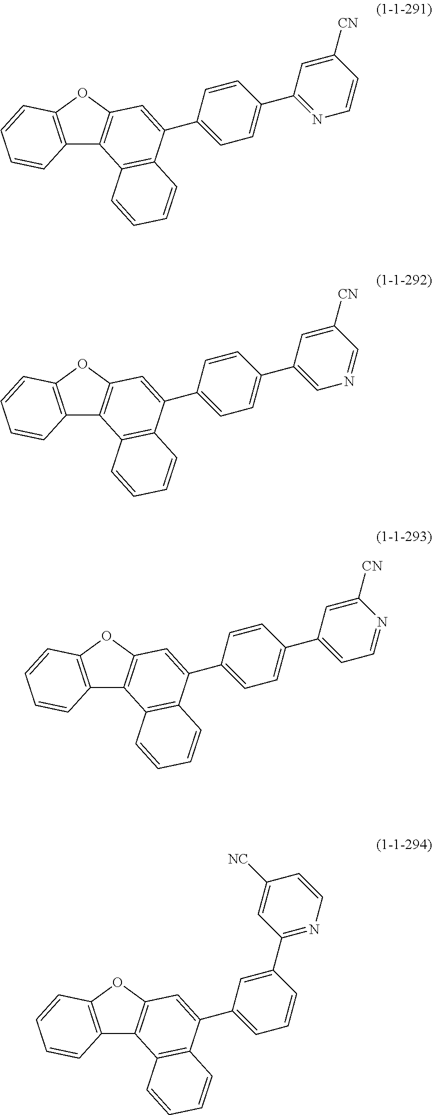 [SMILES: N#Cc1ccnc(-c2ccc(-c3cc4oc5ccccc5c4c4ccccc34)cc2)c1 ; N#Cc1cncc(-c2ccc(-c3cc4oc5ccccc5c4c4ccccc34)cc2)c1 ; N#Cc1cc(-c2ccc(-c3cc4oc5ccccc5c4c4ccccc34)cc2)ccn1 ; N#Cc1ccnc(-c2cccc(-c3cc4oc5ccccc5c4c4ccccc34)c2)c1]

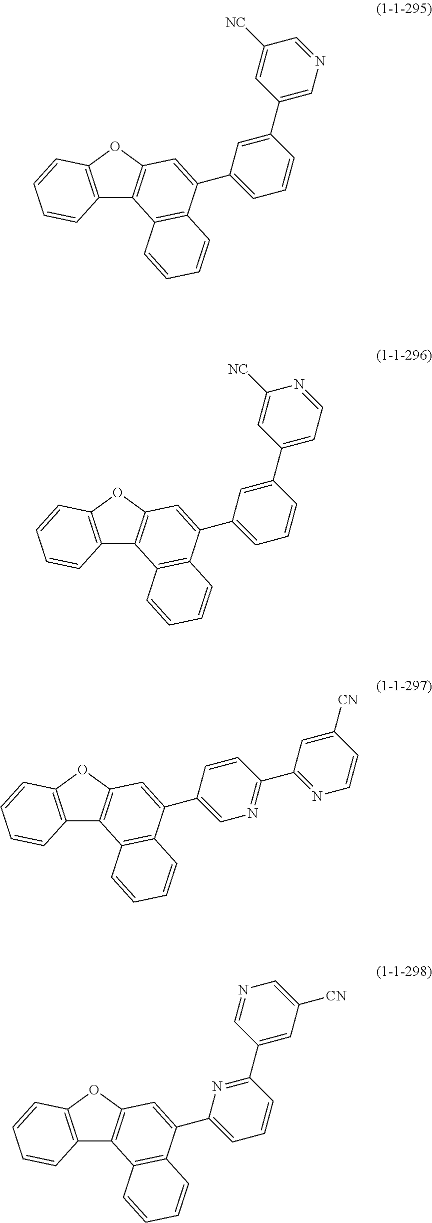 [SMILES: N#Cc1cncc(-c2cccc(-c3cc4oc5ccccc5c4c4ccccc34)c2)c1 ; N#Cc1cc(-c2cccc(-c3cc4oc5ccccc5c4c4ccccc34)c2)ccn1 ; N#Cc1ccnc(-c2ccc(-c3cc4oc5ccccc5c4c4ccccc34)cn2)c1 ; N#Cc1cncc(-c2cccc(-c3cc4oc5ccccc5c4c4ccccc34)n2)c1]

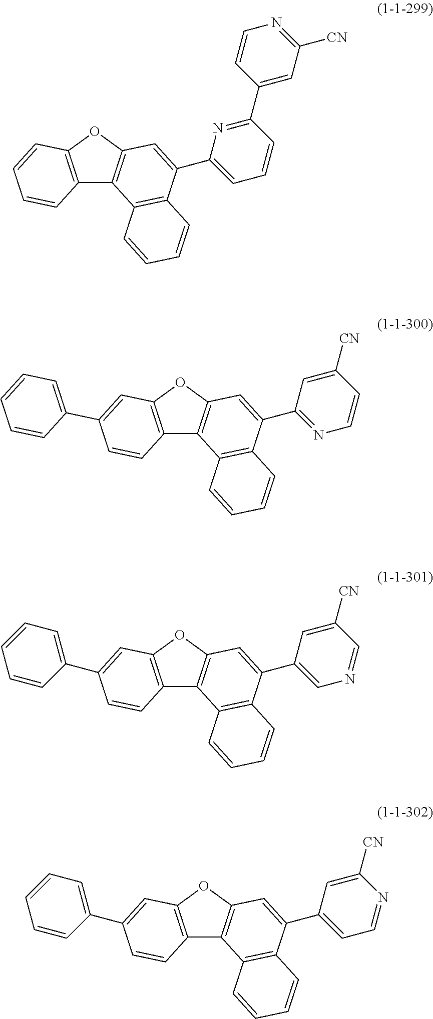 [SMILES: N#Cc1cc(-c2cccc(-c3cc4oc5ccccc5c4c4ccccc34)n2)ccn1 ; N#Cc1ccnc(-c2cc3oc4cc(-c5ccccc5)ccc4c3c3ccccc23)c1 ; N#Cc1cncc(-c2cc3oc4cc(-c5ccccc5)ccc4c3c3ccccc23)c1 ; N#Cc1cc(-c2cc3oc4cc(-c5ccccc5)ccc4c3c3ccccc23)ccn1]

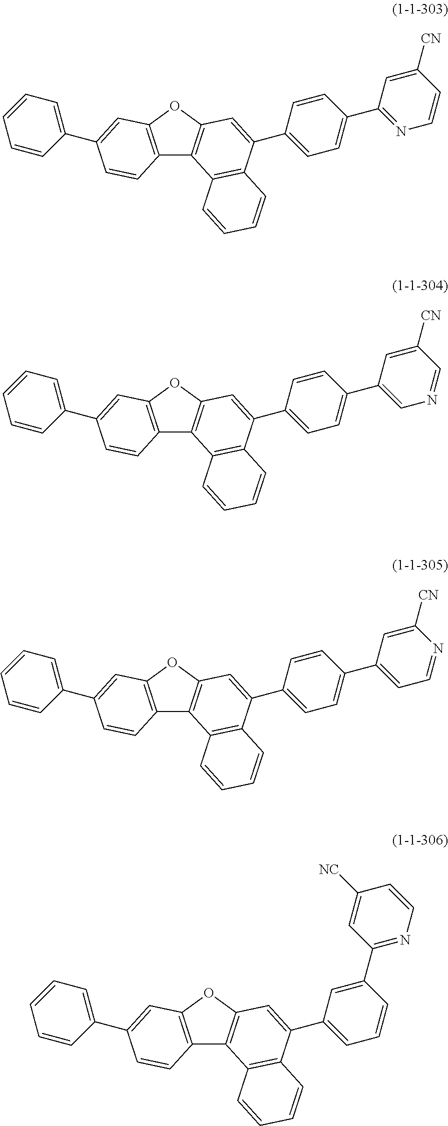 [SMILES: N#Cc1ccnc(-c2ccc(-c3cc4oc5cc(-c6ccccc6)ccc5c4c4ccccc34)cc2)c1 ; N#Cc1cncc(-c2ccc(-c3cc4oc5cc(-c6ccccc6)ccc5c4c4ccccc34)cc2)c1 ; N#Cc1cc(-c2ccc(-c3cc4oc5cc(-c6ccccc6)ccc5c4c4ccccc34)cc2)ccn1 ; N#Cc1ccnc(-c2cccc(-c3cc4oc5cc(-c6ccccc6)ccc5c4c4ccccc34)c2)c1]

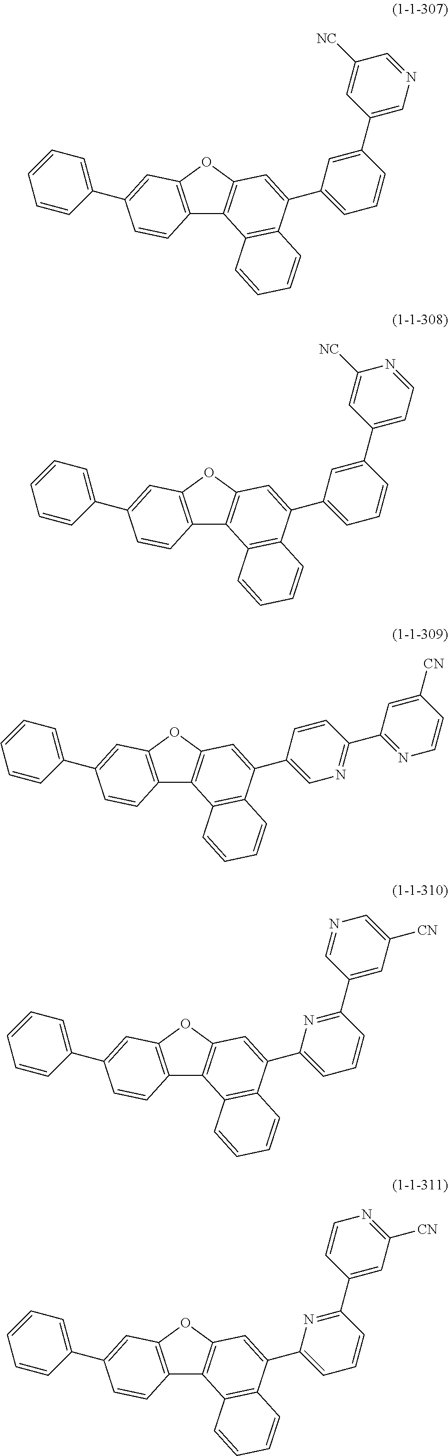 [SMILES: N#Cc1cncc(-c2cccc(-c3cc4oc5cc(-c6ccccc6)ccc5c4c4ccccc34)c2)c1 ; N#Cc1cc(-c2cccc(-c3cc4oc5cc(-c6ccccc6)ccc5c4c4ccccc34)c2)ccn1 ; N#Cc1ccnc(-c2ccc(-c3cc4oc5cc(-c6ccccc6)ccc5c4c4ccccc34)cn2)c1 ; N#Cc1cncc(-c2cccc(-c3cc4oc5cc(-c6ccccc6)ccc5c4c4ccccc34)n2)c1 ; N#Cc1cc(-c2cccc(-c3cc4oc5cc(-c6ccccc6)ccc5c4c4ccccc34)n2)ccn1]

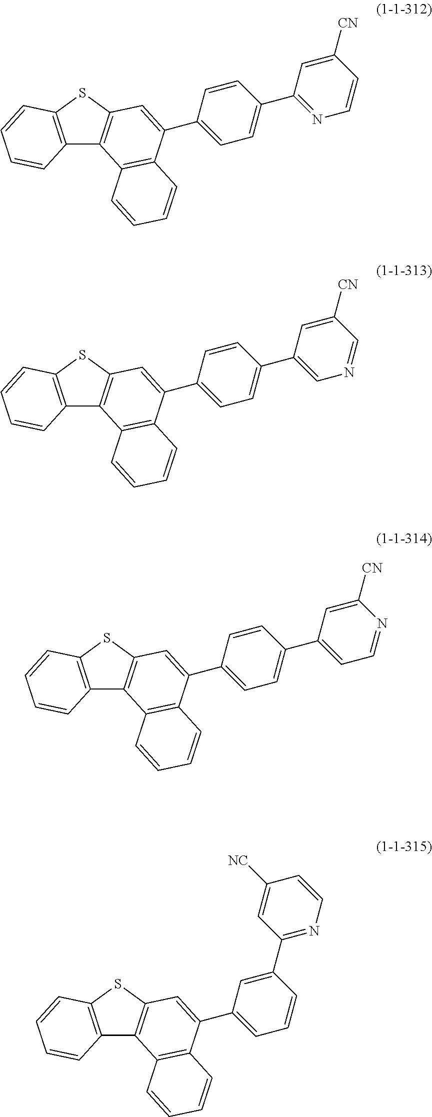 [SMILES: N#Cc1ccnc(-c2ccc(-c3cc4sc5ccccc5c4c4ccccc34)cc2)c1 ; N#Cc1cncc(-c2ccc(-c3cc4sc5ccccc5c4c4ccccc34)cc2)c1 ; N#Cc1cc(-c2ccc(-c3cc4sc5ccccc5c4c4ccccc34)cc2)ccn1 ; N#Cc1ccnc(-c2cccc(-c3cc4sc5ccccc5c4c4ccccc34)c2)c1]

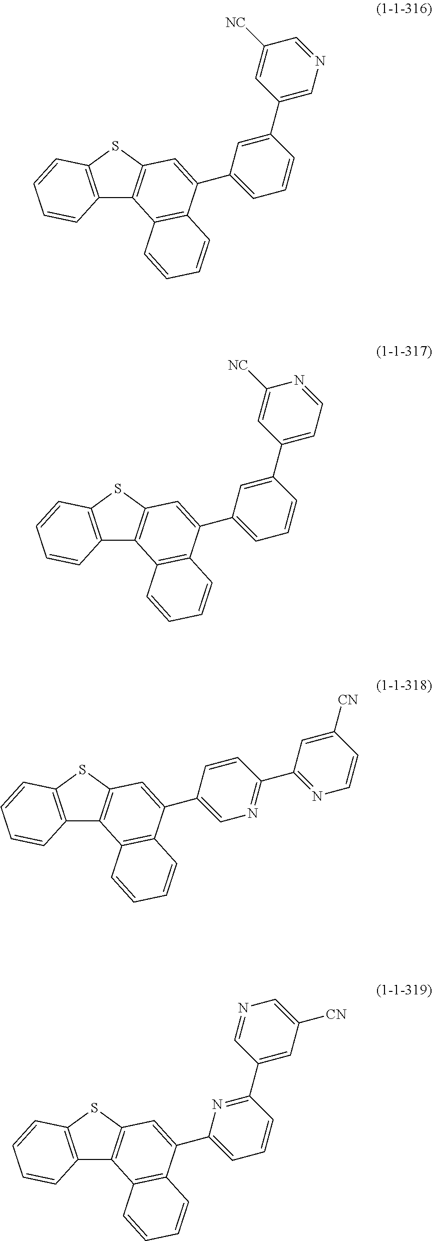 [SMILES: N#Cc1cncc(-c2cccc(-c3cc4sc5ccccc5c4c4ccccc34)c2)c1 ; N#Cc1cc(-c2cccc(-c3cc4sc5ccccc5c4c4ccccc34)c2)ccn1 ; N#Cc1ccnc(-c2ccc(-c3cc4sc5ccccc5c4c4ccccc34)cn2)c1 ; N#Cc1cncc(-c2cccc(-c3cc4sc5ccccc5c4c4ccccc34)n2)c1]

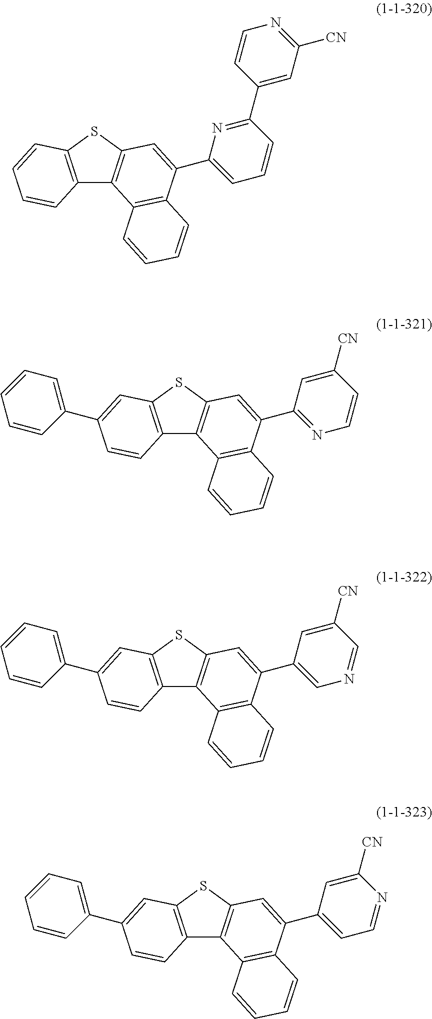 [SMILES: N#Cc1cc(-c2cccc(-c3cc4sc5ccccc5c4c4ccccc34)n2)ccn1 ; N#Cc1ccnc(-c2cc3sc4cc(-c5ccccc5)ccc4c3c3ccccc23)c1 ; N#Cc1cncc(-c2cc3sc4cc(-c5ccccc5)ccc4c3c3ccccc23)c1 ; N#Cc1cc(-c2cc3sc4cc(-c5ccccc5)ccc4c3c3ccccc23)ccn1]

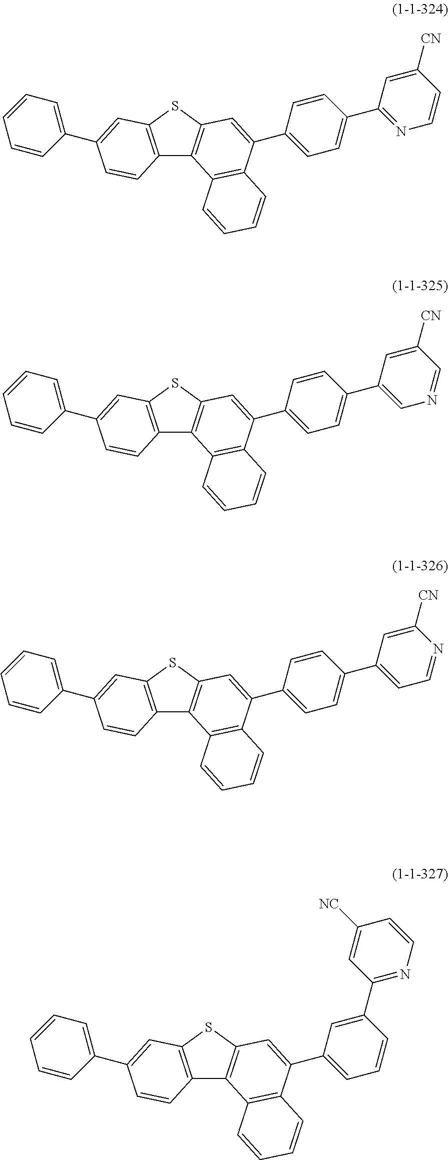 [SMILES: N#Cc1ccnc(-c2ccc(-c3cc4sc5cc(-c6ccccc6)ccc5c4c4ccccc34)cc2)c1 ; N#Cc1cncc(-c2ccc(-c3cc4sc5cc(-c6ccccc6)ccc5c4c4ccccc34)cc2)c1 ; N#Cc1cc(-c2ccc(-c3cc4sc5cc(-c6ccccc6)ccc5c4c4ccccc34)cc2)ccn1 ; N#Cc1ccnc(-c2cccc(-c3cc4sc5cc(-c6ccccc6)ccc5c4c4ccccc34)c2)c1]

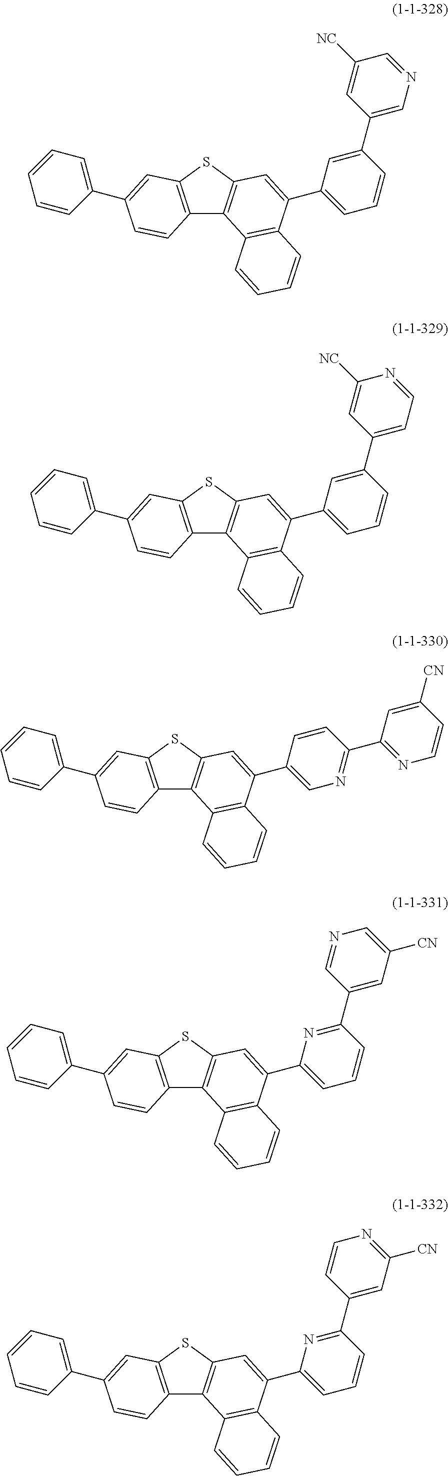 [SMILES: N#Cc1cncc(-c2cccc(-c3cc4sc5cc(-c6ccccc6)ccc5c4c4ccccc34)c2)c1 ; N#Cc1cc(-c2cccc(-c3cc4sc5cc(-c6ccccc6)ccc5c4c4ccccc34)c2)ccn1 ; N#Cc1ccnc(-c2ccc(-c3cc4sc5cc(-c6ccccc6)ccc5c4c4ccccc34)cn2)c1 ; N#Cc1cncc(-c2cccc(-c3cc4sc5cc(-c6ccccc6)ccc5c4c4ccccc34)n2)c1 ; N#Cc1cc(-c2cccc(-c3cc4sc5cc(-c6ccccc6)ccc5c4c4ccccc34)n2)ccn1]

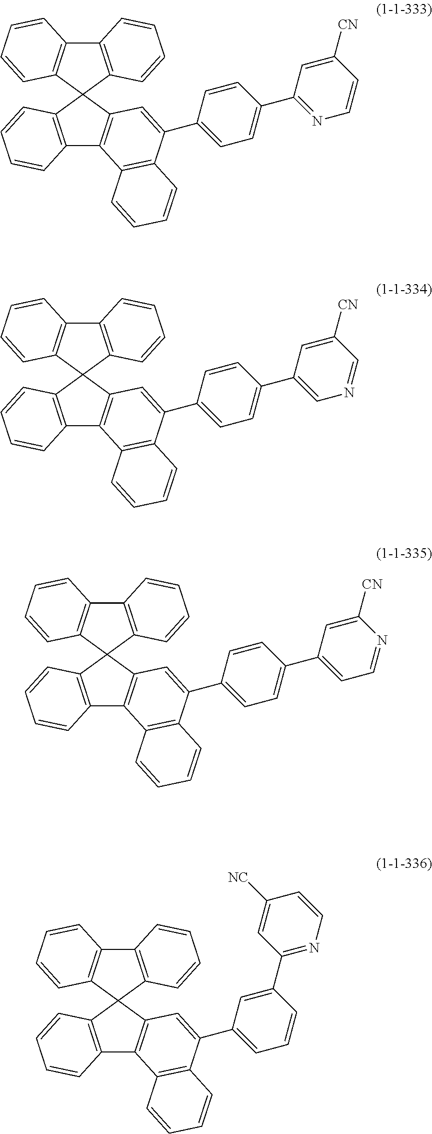 [SMILES: N#Cc1ccnc(-c2ccc(-c3cc4c(c5ccccc35)-c3ccccc3C43c4ccccc4-c4ccccc43)cc2)c1 ; N#Cc1cncc(-c2ccc(-c3cc4c(c5ccccc35)-c3ccccc3C43c4ccccc4-c4ccccc43)cc2)c1 ; N#Cc1cc(-c2ccc(-c3cc4c(c5ccccc35)-c3ccccc3C43c4ccccc4-c4ccccc43)cc2)ccn1 ; N#Cc1ccnc(-c2cccc(-c3cc4c(c5ccccc35)-c3ccccc3C43c4ccccc4-c4ccccc43)c2)c1]

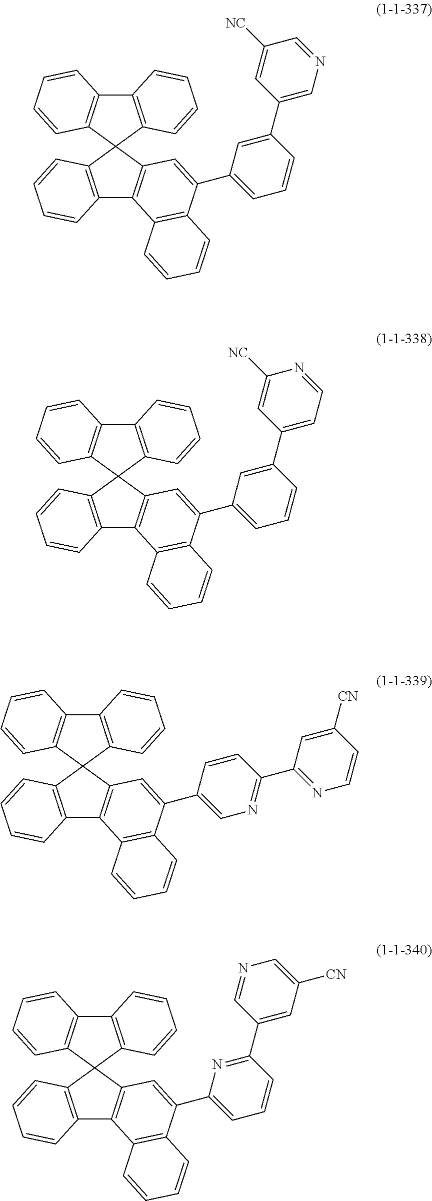 [SMILES: N#Cc1cncc(-c2cccc(-c3cc4c(c5ccccc35)-c3ccccc3C43c4ccccc4-c4ccccc43)c2)c1 ; N#Cc1cc(-c2cccc(-c3cc4c(c5ccccc35)-c3ccccc3C43c4ccccc4-c4ccccc43)c2)ccn1 ; N#Cc1ccnc(-c2ccc(-c3cc4c(c5ccccc35)-c3ccccc3C43c4ccccc4-c4ccccc43)cn2)c1 ; N#Cc1cncc(-c2cccc(-c3cc4c(c5ccccc35)-c3ccccc3C43c4ccccc4-c4ccccc43)n2)c1]

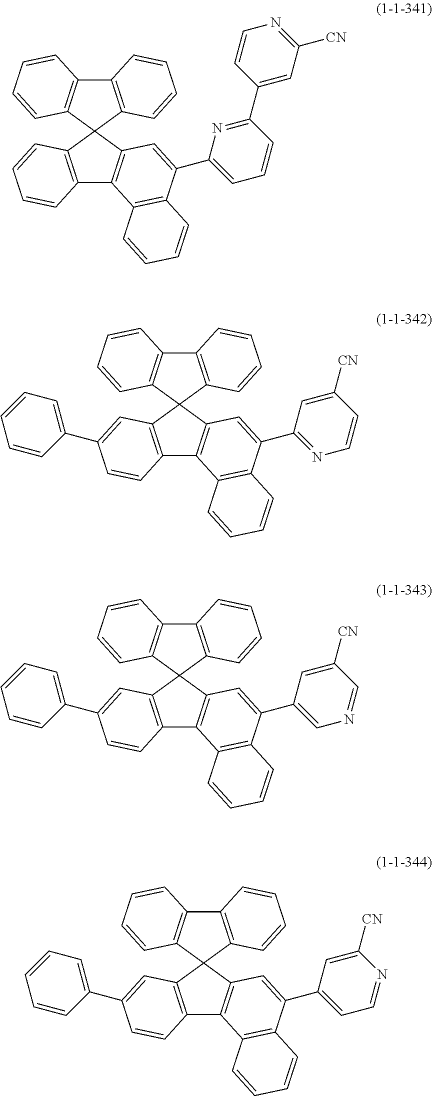 [SMILES: N#Cc1cc(-c2cccc(-c3cc4c(c5ccccc35)-c3ccccc3C43c4ccccc4-c4ccccc43)n2)ccn1 ; N#Cc1ccnc(-c2cc3c(c4ccccc24)-c2ccc(-c4ccccc4)cc2C32c3ccccc3-c3ccccc32)c1 ; N#Cc1cncc(-c2cc3c(c4ccccc24)-c2ccc(-c4ccccc4)cc2C32c3ccccc3-c3ccccc32)c1 ; N#Cc1cc(-c2cc3c(c4ccccc24)-c2ccc(-c4ccccc4)cc2C32c3ccccc3-c3ccccc32)ccn1]

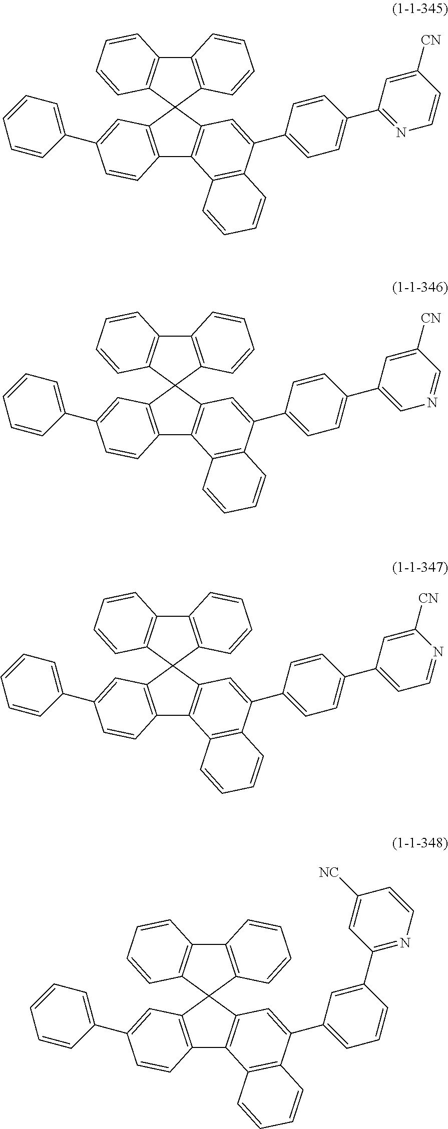 [SMILES: N#Cc1ccnc(-c2ccc(-c3cc4c(c5ccccc35)-c3ccc(-c5ccccc5)cc3C43c4ccccc4-c4ccccc43)cc2)c1 ; N#Cc1cncc(-c2ccc(-c3cc4c(c5ccccc35)-c3ccc(-c5ccccc5)cc3C43c4ccccc4-c4ccccc43)cc2)c1 ; N#Cc1cc(-c2ccc(-c3cc4c(c5ccccc35)-c3ccc(-c5ccccc5)cc3C43c4ccccc4-c4ccccc43)cc2)ccn1 ; N#Cc1ccnc(-c2cccc(-c3cc4c(c5ccccc35)-c3ccc(-c5ccccc5)cc3C43c4ccccc4-c4ccccc43)c2)c1]

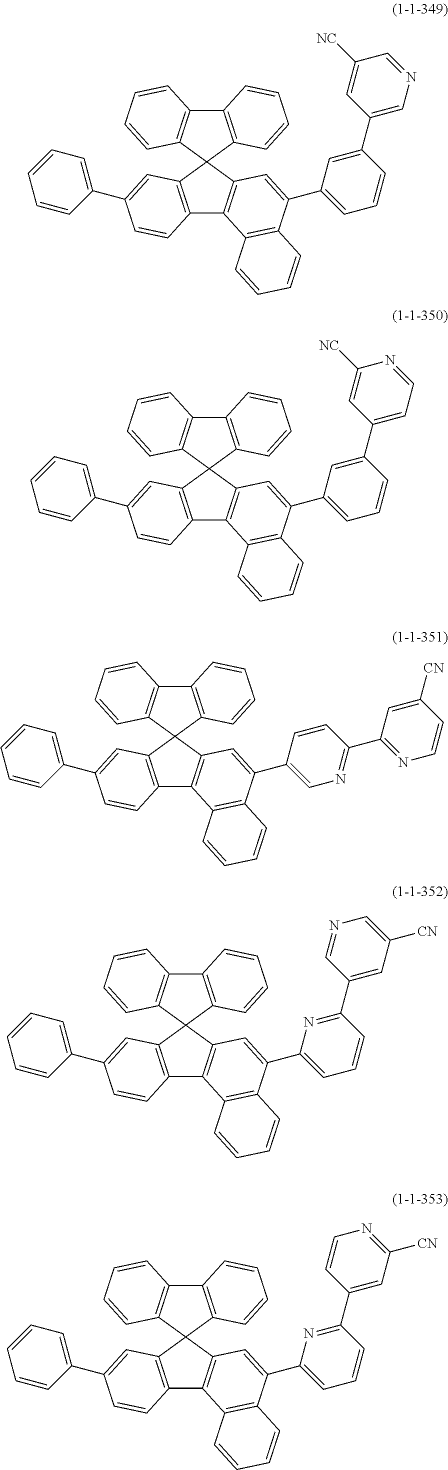 [SMILES: N#Cc1cncc(-c2cccc(-c3cc4c(c5ccccc35)-c3ccc(-c5ccccc5)cc3C43c4ccccc4-c4ccccc43)c2)c1 ; N#Cc1cc(-c2cccc(-c3cc4c(c5ccccc35)-c3ccc(-c5ccccc5)cc3C43c4ccccc4-c4ccccc43)c2)ccn1 ; N#Cc1ccnc(-c2ccc(-c3cc4c(c5ccccc35)-c3ccc(-c5ccccc5)cc3C43c4ccccc4-c4ccccc43)cn2)c1 ; N#Cc1cncc(-c2cccc(-c3cc4c(c5ccccc35)-c3ccc(-c5ccccc5)cc3C43c4ccccc4-c4ccccc43)n2)c1 ; N#Cc1cc(-c2cccc(-c3cc4c(c5ccccc35)-c3ccc(-c5ccccc5)cc3C43c4ccccc4-c4ccccc43)n2)ccn1]

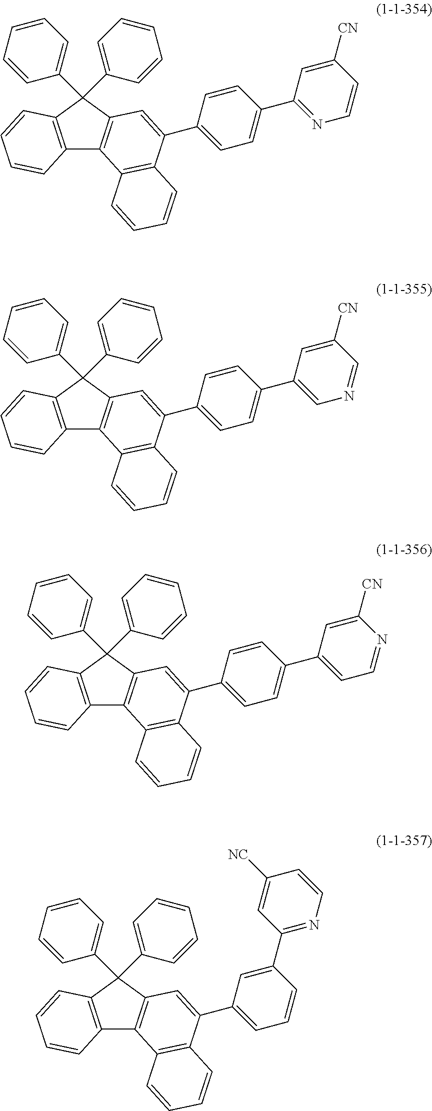 [SMILES: N#Cc1ccnc(-c2ccc(-c3cc4c(c5ccccc35)-c3ccccc3C4(c3ccccc3)c3ccccc3)cc2)c1 ; N#Cc1cncc(-c2ccc(-c3cc4c(c5ccccc35)-c3ccccc3C4(c3ccccc3)c3ccccc3)cc2)c1 ; N#Cc1cc(-c2ccc(-c3cc4c(c5ccccc35)-c3ccccc3C4(c3ccccc3)c3ccccc3)cc2)ccn1 ; N#Cc1ccnc(-c2cccc(-c3cc4c(c5ccccc35)-c3ccccc3C4(c3ccccc3)c3ccccc3)c2)c1]

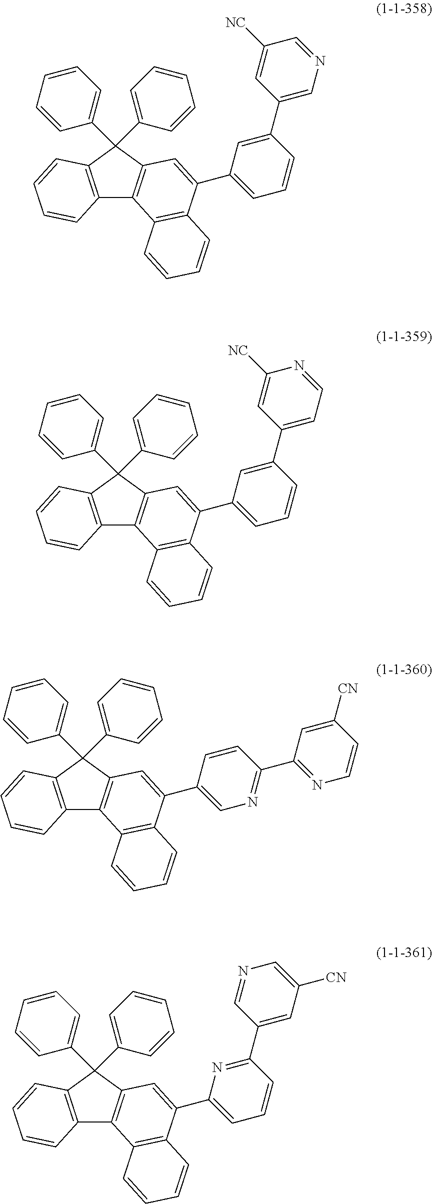 [SMILES: N#Cc1cncc(-c2cccc(-c3cc4c(c5ccccc35)-c3ccccc3C4(c3ccccc3)c3ccccc3)c2)c1 ; N#Cc1cc(-c2cccc(-c3cc4c(c5ccccc35)-c3ccccc3C4(c3ccccc3)c3ccccc3)c2)ccn1 ; N#Cc1ccnc(-c2ccc(-c3cc4c(c5ccccc35)-c3ccccc3C4(c3ccccc3)c3ccccc3)cn2)c1 ; N#Cc1cncc(-c2cccc(-c3cc4c(c5ccccc35)-c3ccccc3C4(c3ccccc3)c3ccccc3)n2)c1]

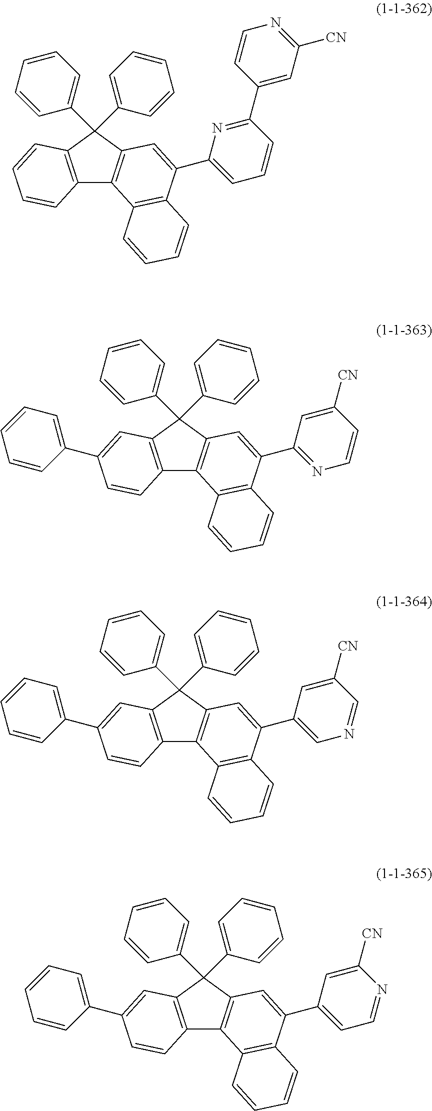 [SMILES: N#Cc1cc(-c2cccc(-c3cc4c(c5ccccc35)-c3ccccc3C4(c3ccccc3)c3ccccc3)n2)ccn1 ; N#Cc1ccnc(-c2cc3c(c4ccccc24)-c2ccc(-c4ccccc4)cc2C3(c2ccccc2)c2ccccc2)c1 ; N#Cc1cncc(-c2cc3c(c4ccccc24)-c2ccc(-c4ccccc4)cc2C3(c2ccccc2)c2ccccc2)c1 ; N#Cc1cc(-c2cc3c(c4ccccc24)-c2ccc(-c4ccccc4)cc2C3(c2ccccc2)c2ccccc2)ccn1]

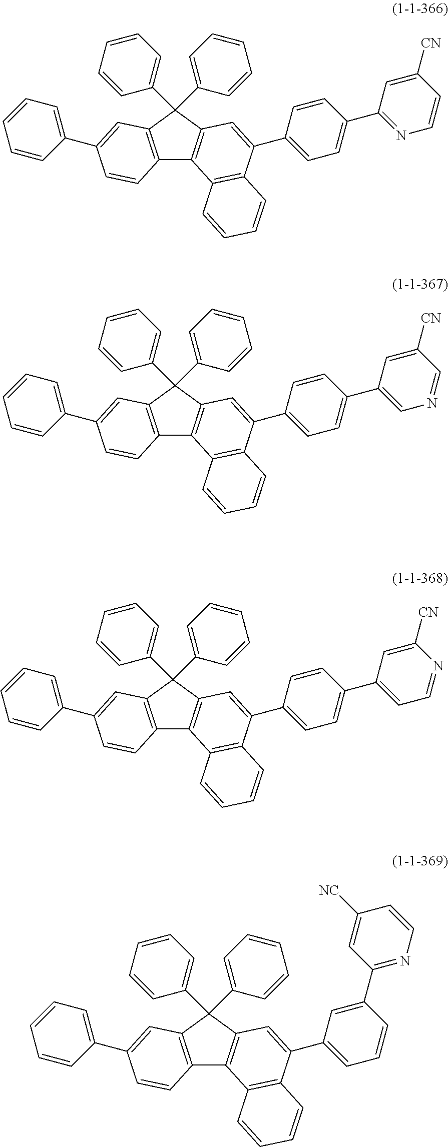 [SMILES: N#Cc1ccnc(-c2ccc(-c3cc4c(c5ccccc35)-c3ccc(-c5ccccc5)cc3C4(c3ccccc3)c3ccccc3)cc2)c1 ; N#Cc1cncc(-c2ccc(-c3cc4c(c5ccccc35)-c3ccc(-c5ccccc5)cc3C4(c3ccccc3)c3ccccc3)cc2)c1 ; N#Cc1cc(-c2ccc(-c3cc4c(c5ccccc35)-c3ccc(-c5ccccc5)cc3C4(c3ccccc3)c3ccccc3)cc2)ccn1 ; N#Cc1ccnc(-c2cccc(-c3cc4c(c5ccccc35)-c3ccc(-c5ccccc5)cc3C4(c3ccccc3)c3ccccc3)c2)c1]

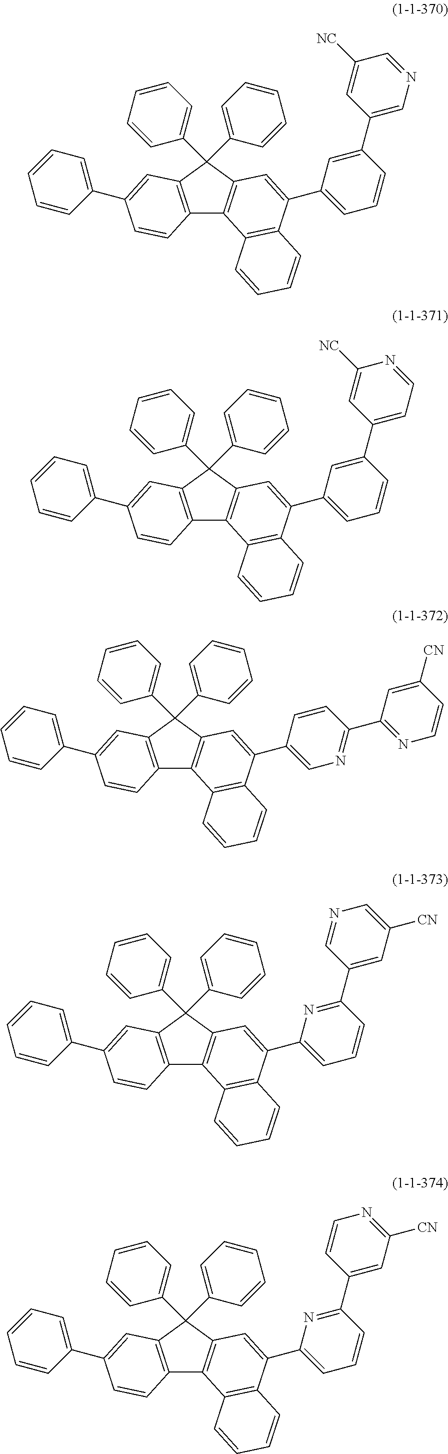 [SMILES: N#Cc1cncc(-c2cccc(-c3cc4c(c5ccccc35)-c3ccc(-c5ccccc5)cc3C4(c3ccccc3)c3ccccc3)c2)c1 ; N#Cc1cc(-c2cccc(-c3cc4c(c5ccccc35)-c3ccc(-c5ccccc5)cc3C4(c3ccccc3)c3ccccc3)c2)ccn1 ; N#Cc1ccnc(-c2ccc(-c3cc4c(c5ccccc35)-c3ccc(-c5ccccc5)cc3C4(c3ccccc3)c3ccccc3)cn2)c1 ; N#Cc1cncc(-c2cccc(-c3cc4c(c5ccccc35)-c3ccc(-c5ccccc5)cc3C4(c3ccccc3)c3ccccc3)n2)c1 ; N#Cc1cc(-c2cccc(-c3cc4c(c5ccccc35)-c3ccc(-c5ccccc5)cc3C4(c3ccccc3)c3ccccc3)n2)ccn1]

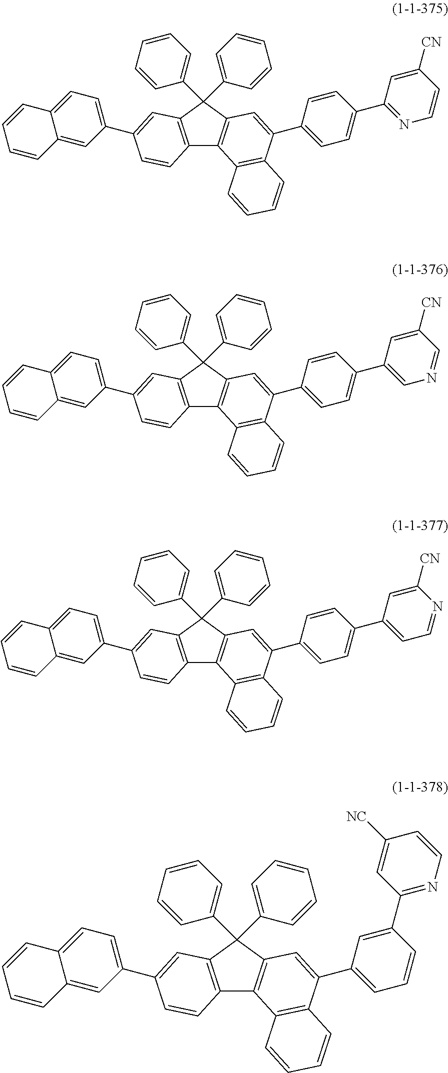 [SMILES: N#Cc1ccnc(-c2ccc(-c3cc4c(c5ccccc35)-c3ccc(-c5ccc6ccccc6c5)cc3C4(c3ccccc3)c3ccccc3)cc2)c1 ; N#Cc1cncc(-c2ccc(-c3cc4c(c5ccccc35)-c3ccc(-c5ccc6ccccc6c5)cc3C4(c3ccccc3)c3ccccc3)cc2)c1 ; N#Cc1cc(-c2ccc(-c3cc4c(c5ccccc35)-c3ccc(-c5ccc6ccccc6c5)cc3C4(c3ccccc3)c3ccccc3)cc2)ccn1 ; N#Cc1ccnc(-c2cccc(-c3cc4c(c5ccccc35)-c3ccc(-c5ccc6ccccc6c5)cc3C4(c3ccccc3)c3ccccc3)c2)c1]

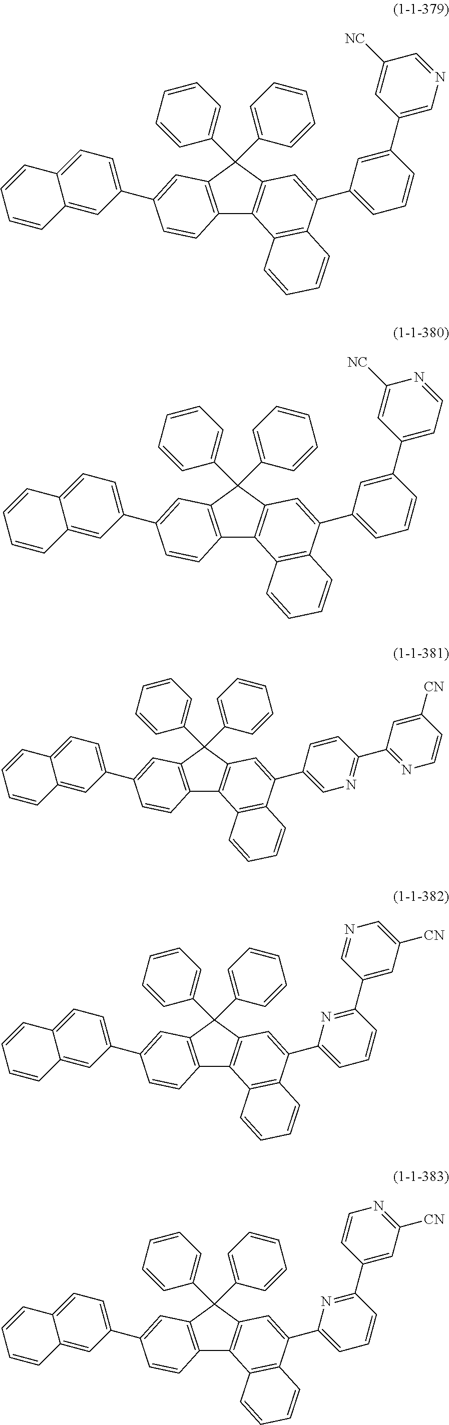 [SMILES: N#Cc1cncc(-c2cccc(-c3cc4c(c5ccccc35)-c3ccc(-c5ccc6ccccc6c5)cc3C4(c3ccccc3)c3ccccc3)c2)c1 ; N#Cc1cc(-c2cccc(-c3cc4c(c5ccccc35)-c3ccc(-c5ccc6ccccc6c5)cc3C4(c3ccccc3)c3ccccc3)c2)ccn1 ; N#Cc1ccnc(-c2ccc(-c3cc4c(c5ccccc35)-c3ccc(-c5ccc6ccccc6c5)cc3C4(c3ccccc3)c3ccccc3)cn2)c1 ; N#Cc1cncc(-c2cccc(-c3cc4c(c5ccccc35)-c3ccc(-c5ccc6ccccc6c5)cc3C4(c3ccccc3)c3ccccc3)n2)c1 ; N#Cc1cc(-c2cccc(-c3cc4c(c5ccccc35)-c3ccc(-c5ccc6ccccc6c5)cc3C4(c3ccccc3)c3ccccc3)n2)ccn1]

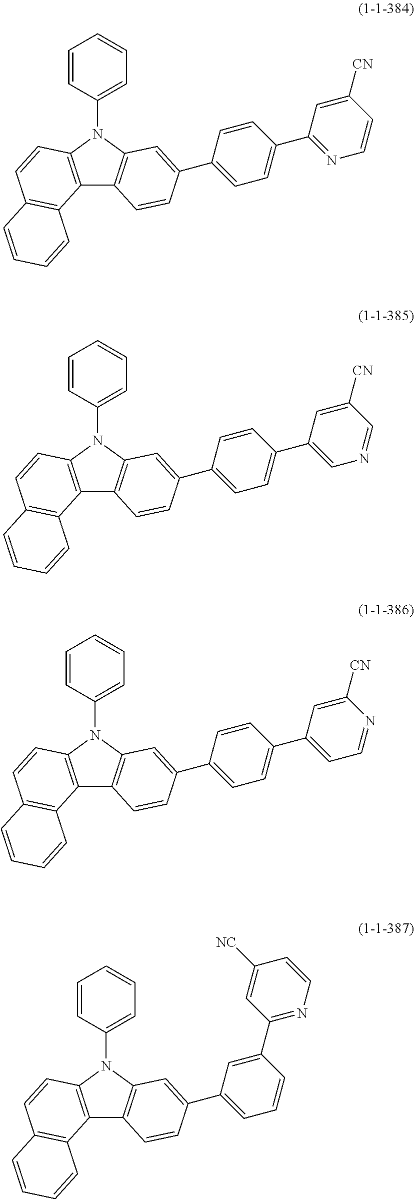 [SMILES: N#Cc1ccnc(-c2ccc(-c3ccc4c5c6ccccc6ccc5n(-c5ccccc5)c4c3)cc2)c1 ; N#Cc1cncc(-c2ccc(-c3ccc4c5c6ccccc6ccc5n(-c5ccccc5)c4c3)cc2)c1 ; N#Cc1cc(-c2ccc(-c3ccc4c5c6ccccc6ccc5n(-c5ccccc5)c4c3)cc2)ccn1 ; N#Cc1ccnc(-c2cccc(-c3ccc4c5c6ccccc6ccc5n(-c5ccccc5)c4c3)c2)c1]

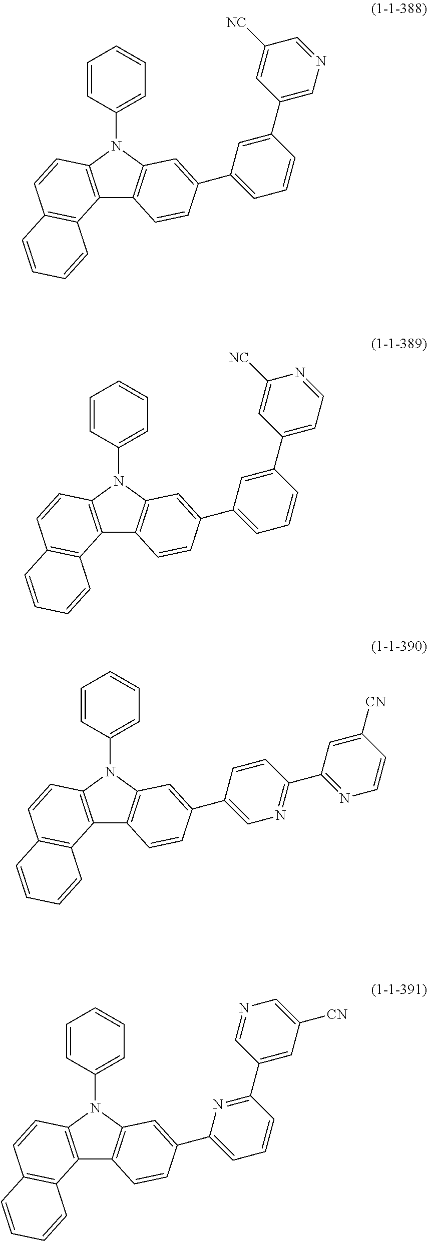 [SMILES: N#Cc1cncc(-c2cccc(-c3ccc4c5c6ccccc6ccc5n(-c5ccccc5)c4c3)c2)c1 ; N#Cc1cc(-c2cccc(-c3ccc4c5c6ccccc6ccc5n(-c5ccccc5)c4c3)c2)ccn1 ; N#Cc1ccnc(-c2ccc(-c3ccc4c5c6ccccc6ccc5n(-c5ccccc5)c4c3)cn2)c1 ; N#Cc1cncc(-c2cccc(-c3ccc4c5c6ccccc6ccc5n(-c5ccccc5)c4c3)n2)c1]

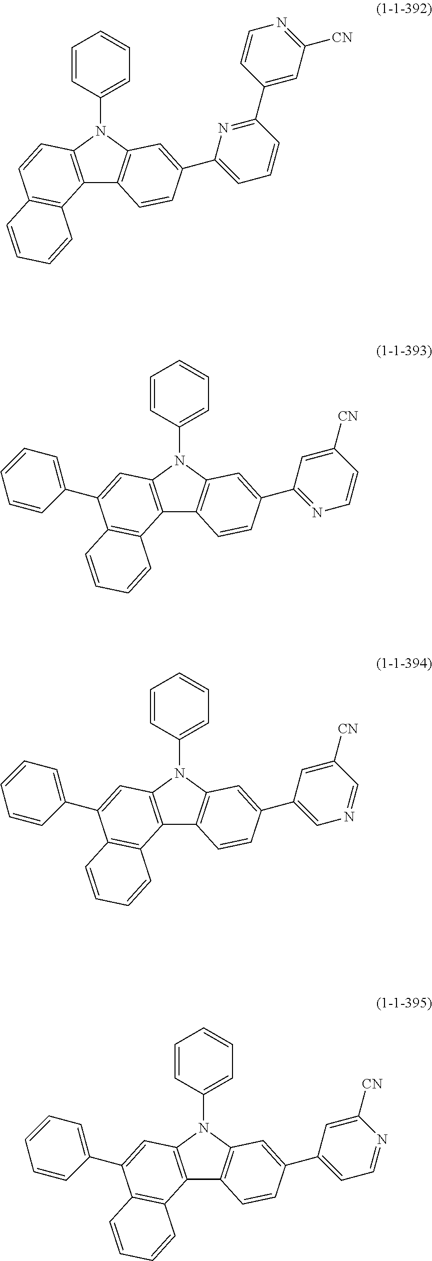 [SMILES: N#Cc1cc(-c2cccc(-c3ccc4c5c6ccccc6ccc5n(-c5ccccc5)c4c3)n2)ccn1 ; N#Cc1ccnc(-c2ccc3c4c5ccccc5c(-c5ccccc5)cc4n(-c4ccccc4)c3c2)c1 ; N#Cc1cncc(-c2ccc3c4c5ccccc5c(-c5ccccc5)cc4n(-c4ccccc4)c3c2)c1 ; N#Cc1cc(-c2ccc3c4c5ccccc5c(-c5ccccc5)cc4n(-c4ccccc4)c3c2)ccn1]

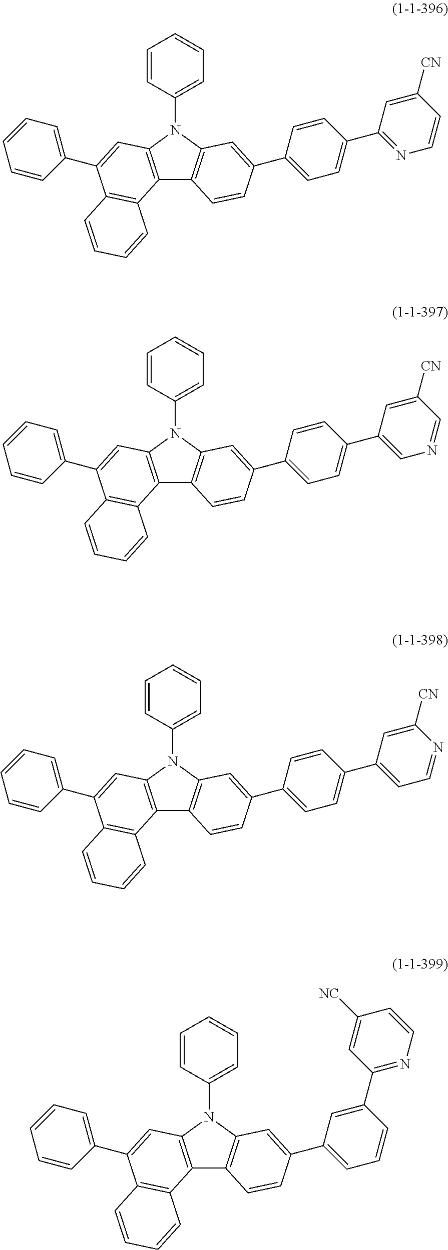 [SMILES: N#Cc1ccnc(-c2ccc(-c3ccc4c5c6ccccc6c(-c6ccccc6)cc5n(-c5ccccc5)c4c3)cc2)c1 ; N#Cc1cncc(-c2ccc(-c3ccc4c5c6ccccc6c(-c6ccccc6)cc5n(-c5ccccc5)c4c3)cc2)c1 ; N#Cc1cc(-c2ccc(-c3ccc4c5c6ccccc6c(-c6ccccc6)cc5n(-c5ccccc5)c4c3)cc2)ccn1 ; N#Cc1ccnc(-c2cccc(-c3ccc4c5c6ccccc6c(-c6ccccc6)cc5n(-c5ccccc5)c4c3)c2)c1]

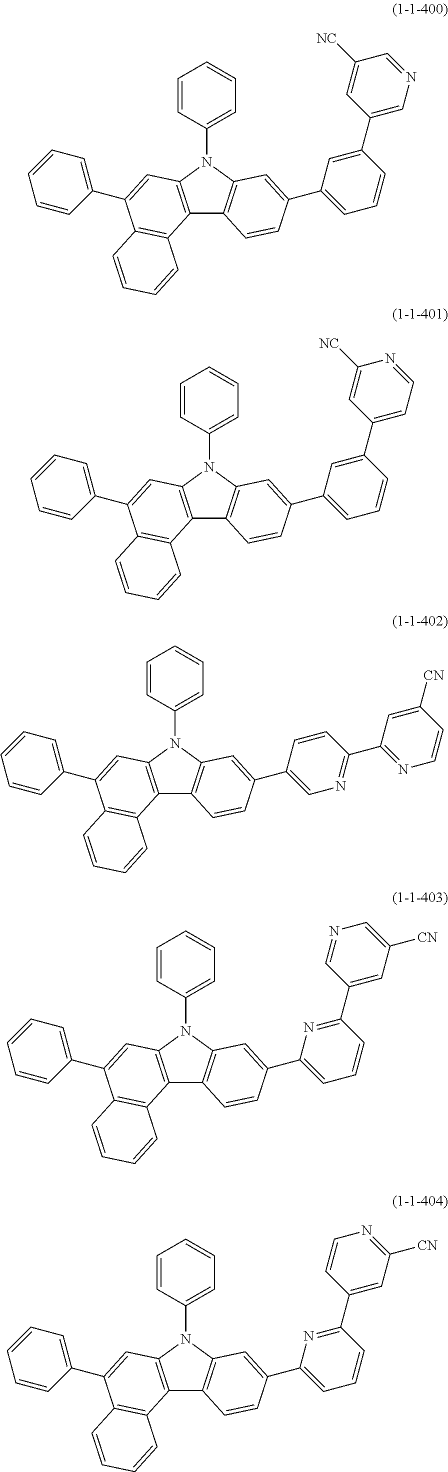 [SMILES: N#Cc1cncc(-c2cccc(-c3ccc4c5c6ccccc6c(-c6ccccc6)cc5n(-c5ccccc5)c4c3)c2)c1 ; N#Cc1cc(-c2cccc(-c3ccc4c5c6ccccc6c(-c6ccccc6)cc5n(-c5ccccc5)c4c3)c2)ccn1 ; N#Cc1ccnc(-c2ccc(-c3ccc4c5c6ccccc6c(-c6ccccc6)cc5n(-c5ccccc5)c4c3)cn2)c1 ; N#Cc1cncc(-c2cccc(-c3ccc4c5c6ccccc6c(-c6ccccc6)cc5n(-c5ccccc5)c4c3)n2)c1 ; N#Cc1cc(-c2cccc(-c3ccc4c5c6ccccc6c(-c6ccccc6)cc5n(-c5ccccc5)c4c3)n2)ccn1]

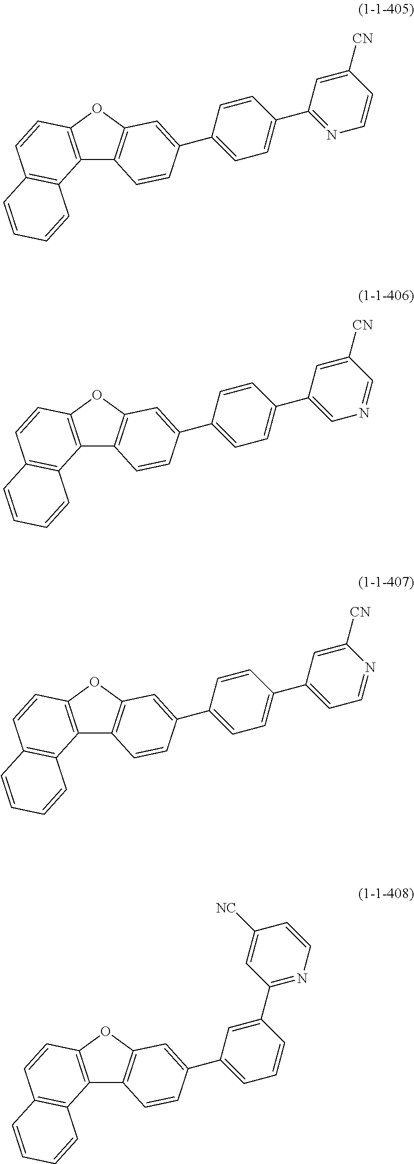 [SMILES: N#Cc1ccnc(-c2ccc(-c3ccc4c(c3)oc3ccc5ccccc5c34)cc2)c1 ; N#Cc1cncc(-c2ccc(-c3ccc4c(c3)oc3ccc5ccccc5c34)cc2)c1 ; N#Cc1cc(-c2ccc(-c3ccc4c(c3)oc3ccc5ccccc5c34)cc2)ccn1 ; N#Cc1ccnc(-c2cccc(-c3ccc4c(c3)oc3ccc5ccccc5c34)c2)c1]

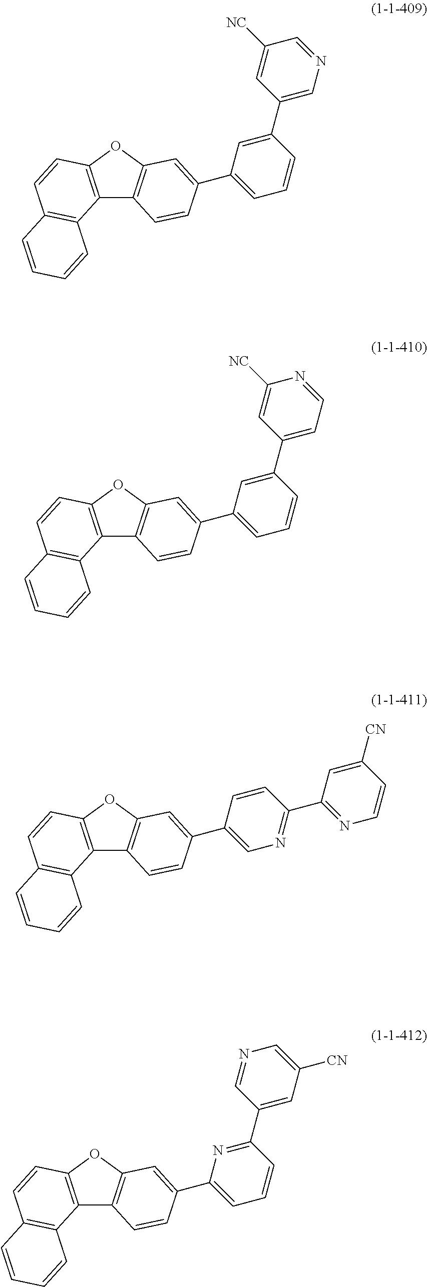 [SMILES: N#Cc1cncc(-c2cccc(-c3ccc4c(c3)oc3ccc5ccccc5c34)c2)c1 ; N#Cc1cc(-c2cccc(-c3ccc4c(c3)oc3ccc5ccccc5c34)c2)ccn1 ; N#Cc1ccnc(-c2ccc(-c3ccc4c(c3)oc3ccc5ccccc5c34)cn2)c1 ; N#Cc1cncc(-c2cccc(-c3ccc4c(c3)oc3ccc5ccccc5c34)n2)c1]

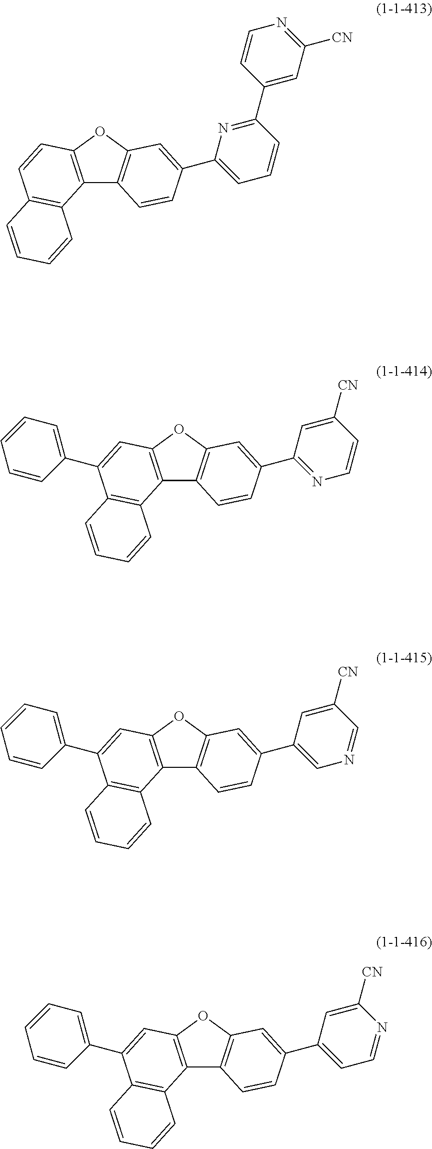 [SMILES: N#Cc1cc(-c2cccc(-c3ccc4c(c3)oc3ccc5ccccc5c34)n2)ccn1 ; N#Cc1ccnc(-c2ccc3c(c2)oc2cc(-c4ccccc4)c4ccccc4c23)c1 ; N#Cc1cncc(-c2ccc3c(c2)oc2cc(-c4ccccc4)c4ccccc4c23)c1 ; N#Cc1cc(-c2ccc3c(c2)oc2cc(-c4ccccc4)c4ccccc4c23)ccn1]

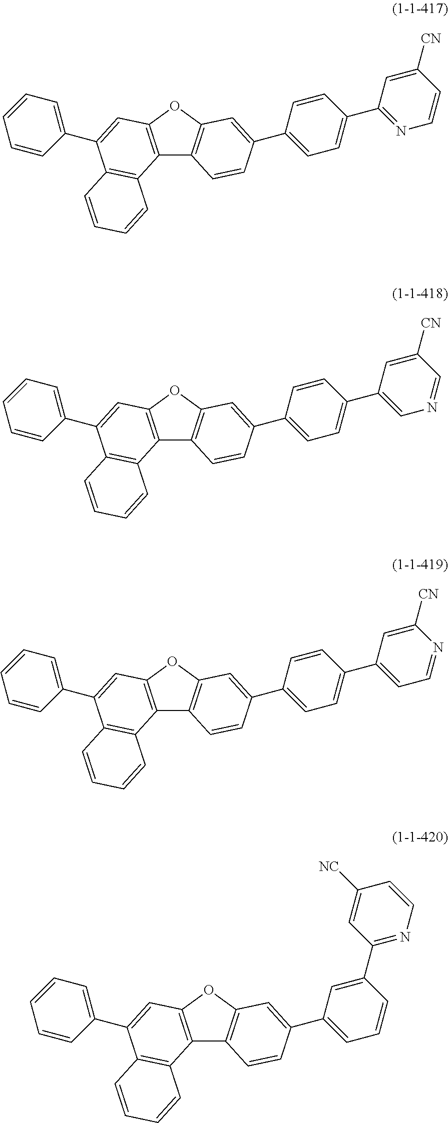 [SMILES: N#Cc1ccnc(-c2ccc(-c3ccc4c(c3)oc3cc(-c5ccccc5)c5ccccc5c34)cc2)c1 ; N#Cc1cncc(-c2ccc(-c3ccc4c(c3)oc3cc(-c5ccccc5)c5ccccc5c34)cc2)c1 ; N#Cc1cc(-c2ccc(-c3ccc4c(c3)oc3cc(-c5ccccc5)c5ccccc5c34)cc2)ccn1 ; N#Cc1ccnc(-c2cccc(-c3ccc4c(c3)oc3cc(-c5ccccc5)c5ccccc5c34)c2)c1]

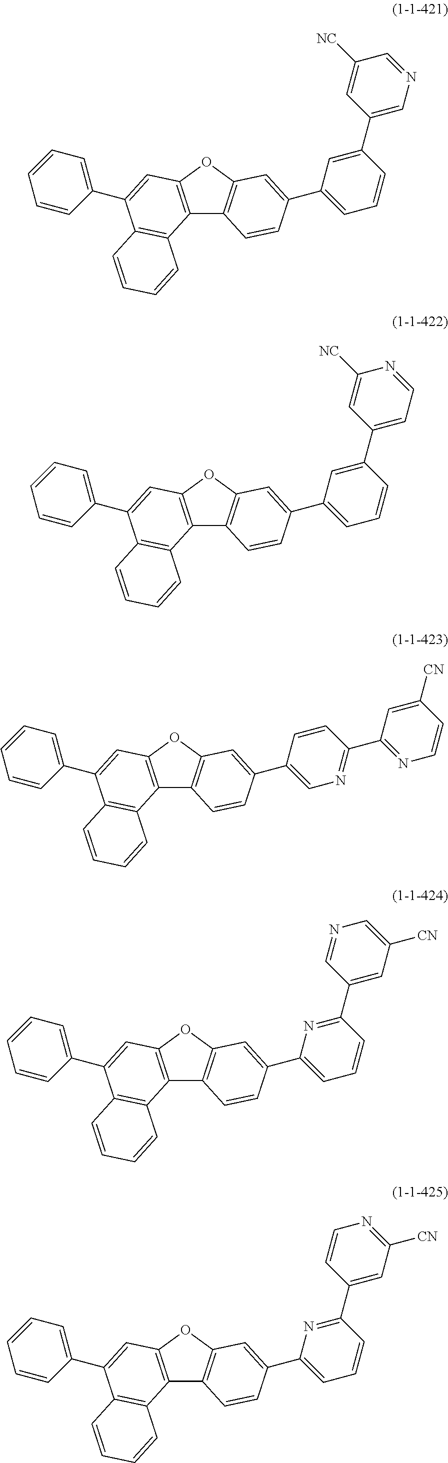 [SMILES: N#Cc1cncc(-c2cccc(-c3ccc4c(c3)oc3cc(-c5ccccc5)c5ccccc5c34)c2)c1 ; N#Cc1cc(-c2cccc(-c3ccc4c(c3)oc3cc(-c5ccccc5)c5ccccc5c34)c2)ccn1 ; N#Cc1ccnc(-c2ccc(-c3ccc4c(c3)oc3cc(-c5ccccc5)c5ccccc5c34)cn2)c1 ; N#Cc1cncc(-c2cccc(-c3ccc4c(c3)oc3cc(-c5ccccc5)c5ccccc5c34)n2)c1 ; N#Cc1cc(-c2cccc(-c3ccc4c(c3)oc3cc(-c5ccccc5)c5ccccc5c34)n2)ccn1]

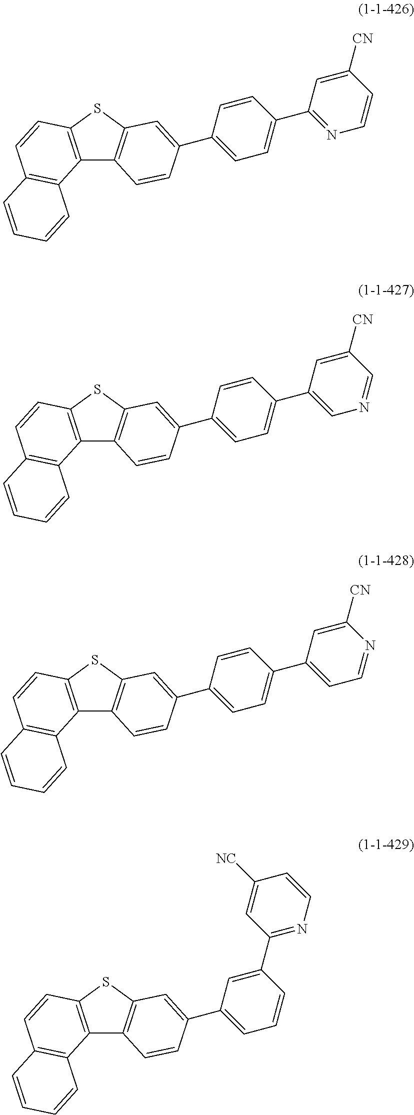 [SMILES: N#Cc1ccnc(-c2ccc(-c3ccc4c(c3)sc3ccc5ccccc5c34)cc2)c1 ; N#Cc1cncc(-c2ccc(-c3ccc4c(c3)sc3ccc5ccccc5c34)cc2)c1 ; N#Cc1cc(-c2ccc(-c3ccc4c(c3)sc3ccc5ccccc5c34)cc2)ccn1 ; N#Cc1ccnc(-c2cccc(-c3ccc4c(c3)sc3ccc5ccccc5c34)c2)c1]

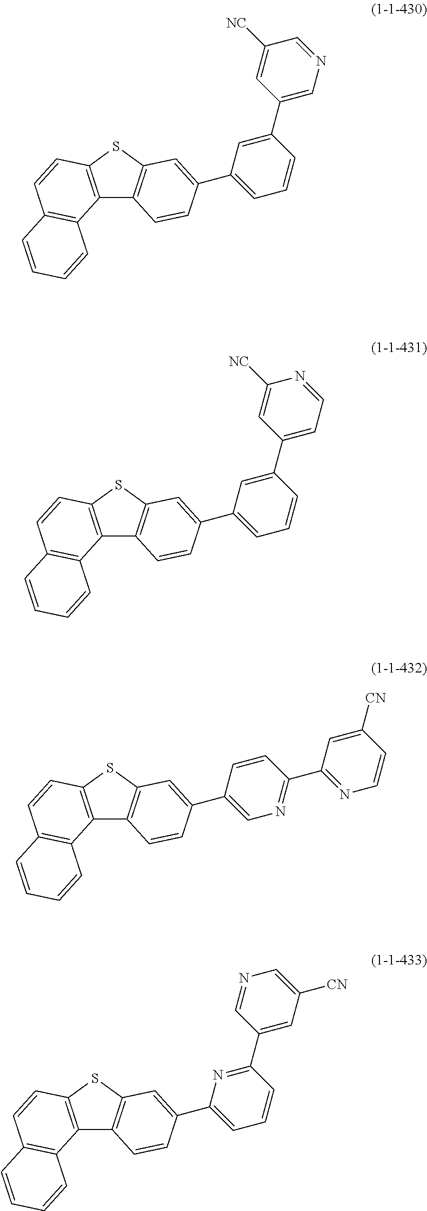 [SMILES: N#Cc1cncc(-c2cccc(-c3ccc4c(c3)sc3ccc5ccccc5c34)c2)c1 ; N#Cc1cc(-c2cccc(-c3ccc4c(c3)sc3ccc5ccccc5c34)c2)ccn1 ; N#Cc1ccnc(-c2ccc(-c3ccc4c(c3)sc3ccc5ccccc5c34)cn2)c1 ; N#Cc1cncc(-c2cccc(-c3ccc4c(c3)sc3ccc5ccccc5c34)n2)c1]

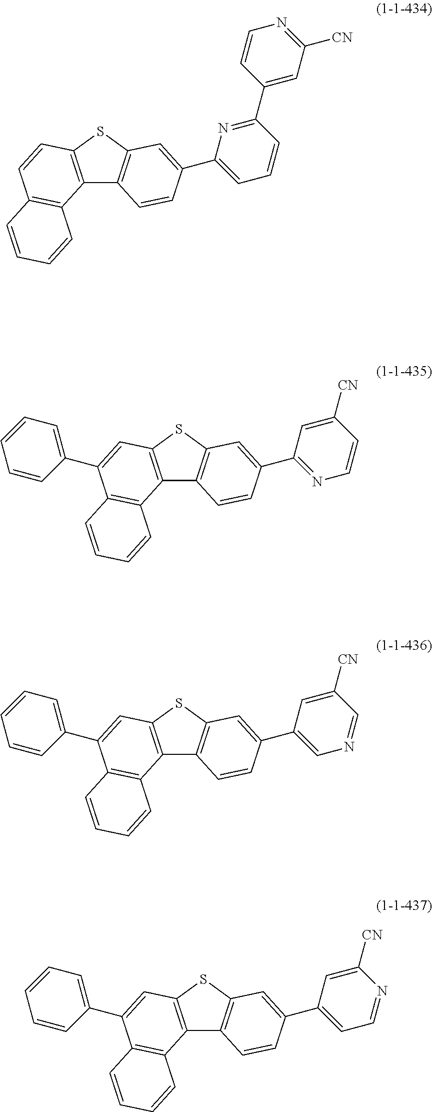 [SMILES: N#Cc1cc(-c2cccc(-c3ccc4c(c3)sc3ccc5ccccc5c34)n2)ccn1 ; N#Cc1ccnc(-c2ccc3c(c2)sc2cc(-c4ccccc4)c4ccccc4c23)c1 ; N#Cc1cncc(-c2ccc3c(c2)sc2cc(-c4ccccc4)c4ccccc4c23)c1 ; N#Cc1cc(-c2ccc3c(c2)sc2cc(-c4ccccc4)c4ccccc4c23)ccn1]

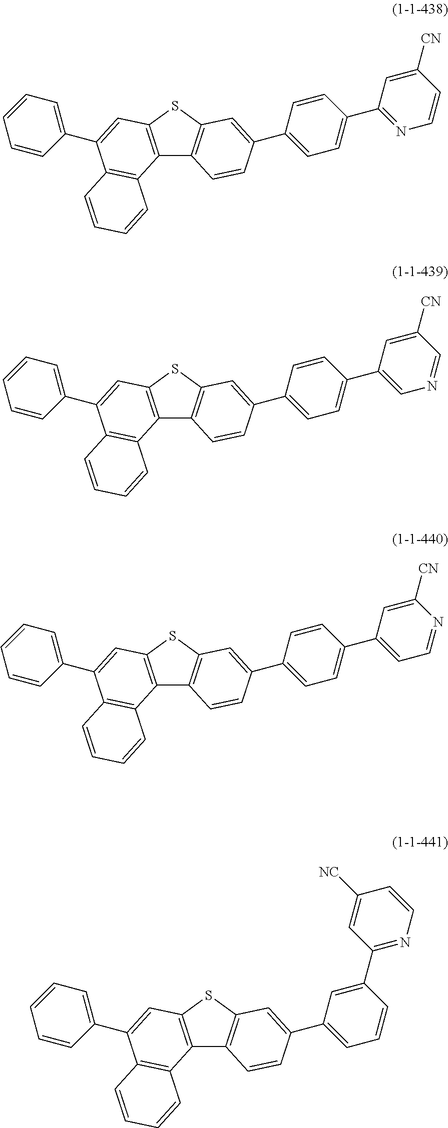 [SMILES: N#Cc1ccnc(-c2ccc(-c3ccc4c(c3)sc3cc(-c5ccccc5)c5ccccc5c34)cc2)c1 ; N#Cc1cncc(-c2ccc(-c3ccc4c(c3)sc3cc(-c5ccccc5)c5ccccc5c34)cc2)c1 ; N#Cc1cc(-c2ccc(-c3ccc4c(c3)sc3cc(-c5ccccc5)c5ccccc5c34)cc2)ccn1 ; N#Cc1ccnc(-c2cccc(-c3ccc4c(c3)sc3cc(-c5ccccc5)c5ccccc5c34)c2)c1]

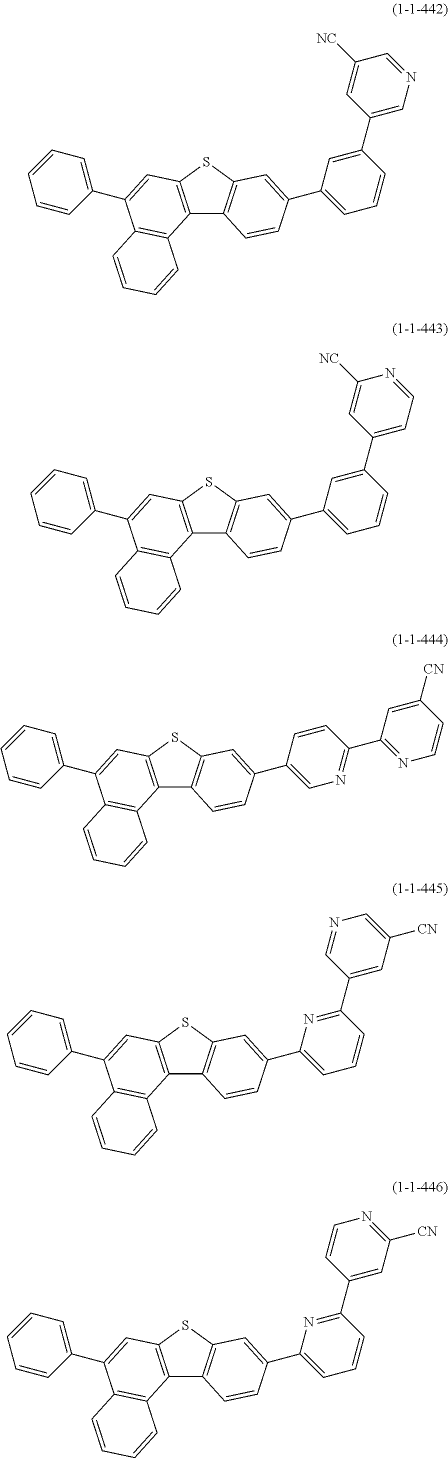 [SMILES: N#Cc1cncc(-c2cccc(-c3ccc4c(c3)sc3cc(-c5ccccc5)c5ccccc5c34)c2)c1 ; N#Cc1cc(-c2cccc(-c3ccc4c(c3)sc3cc(-c5ccccc5)c5ccccc5c34)c2)ccn1 ; N#Cc1ccnc(-c2ccc(-c3ccc4c(c3)sc3cc(-c5ccccc5)c5ccccc5c34)cn2)c1 ; N#Cc1cncc(-c2cccc(-c3ccc4c(c3)sc3cc(-c5ccccc5)c5ccccc5c34)n2)c1 ; N#Cc1cc(-c2cccc(-c3ccc4c(c3)sc3cc(-c5ccccc5)c5ccccc5c34)n2)ccn1]

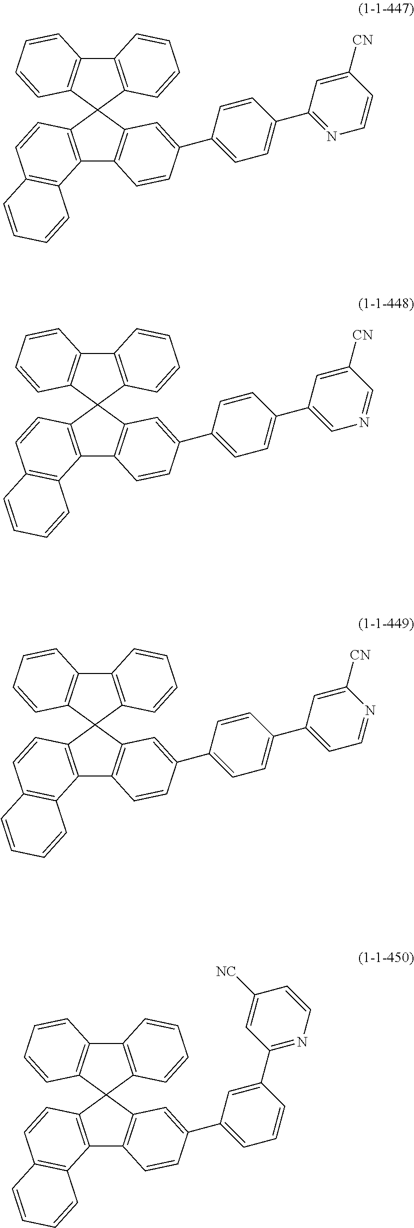 [SMILES: N#Cc1ccnc(-c2ccc(-c3ccc4c(c3)C3(c5ccccc5-c5ccccc53)c3ccc5ccccc5c3-4)cc2)c1 ; N#Cc1cncc(-c2ccc(-c3ccc4c(c3)C3(c5ccccc5-c5ccccc53)c3ccc5ccccc5c3-4)cc2)c1 ; N#Cc1cc(-c2ccc(-c3ccc4c(c3)C3(c5ccccc5-c5ccccc53)c3ccc5ccccc5c3-4)cc2)ccn1 ; N#Cc1ccnc(-c2cccc(-c3ccc4c(c3)C3(c5ccccc5-c5ccccc53)c3ccc5ccccc5c3-4)c2)c1]

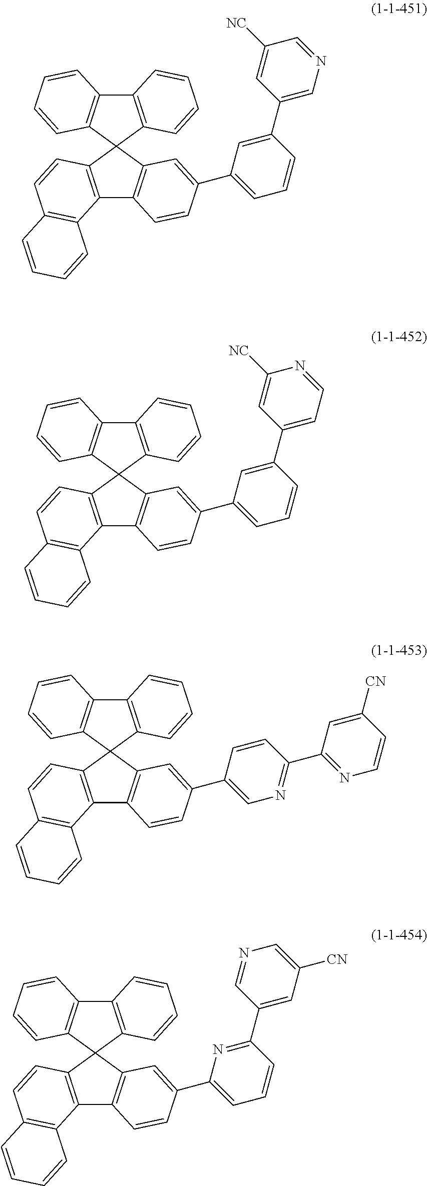 [SMILES: N#Cc1cncc(-c2cccc(-c3ccc4c(c3)C3(c5ccccc5-c5ccccc53)c3ccc5ccccc5c3-4)c2)c1 ; N#Cc1cc(-c2cccc(-c3ccc4c(c3)C3(c5ccccc5-c5ccccc53)c3ccc5ccccc5c3-4)c2)ccn1 ; N#Cc1ccnc(-c2ccc(-c3ccc4c(c3)C3(c5ccccc5-c5ccccc53)c3ccc5ccccc5c3-4)cn2)c1 ; N#Cc1cncc(-c2cccc(-c3ccc4c(c3)C3(c5ccccc5-c5ccccc53)c3ccc5ccccc5c3-4)n2)c1]

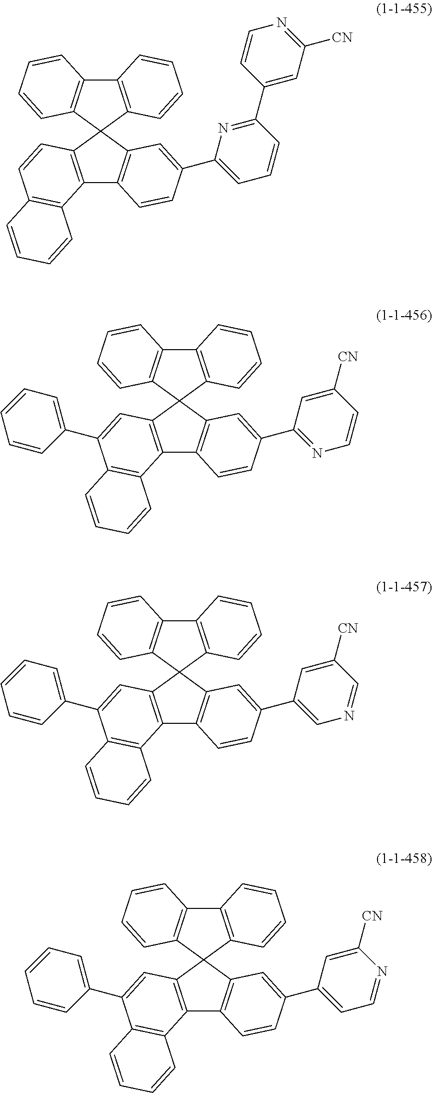 [SMILES: N#Cc1cc(-c2cccc(-c3ccc4c(c3)C3(c5ccccc5-c5ccccc53)c3ccc5ccccc5c3-4)n2)ccn1 ; N#Cc1ccnc(-c2ccc3c(c2)C2(c4ccccc4-c4ccccc42)c2cc(-c4ccccc4)c4ccccc4c2-3)c1 ; N#Cc1cncc(-c2ccc3c(c2)C2(c4ccccc4-c4ccccc42)c2cc(-c4ccccc4)c4ccccc4c2-3)c1 ; N#Cc1cc(-c2ccc3c(c2)C2(c4ccccc4-c4ccccc42)c2cc(-c4ccccc4)c4ccccc4c2-3)ccn1]

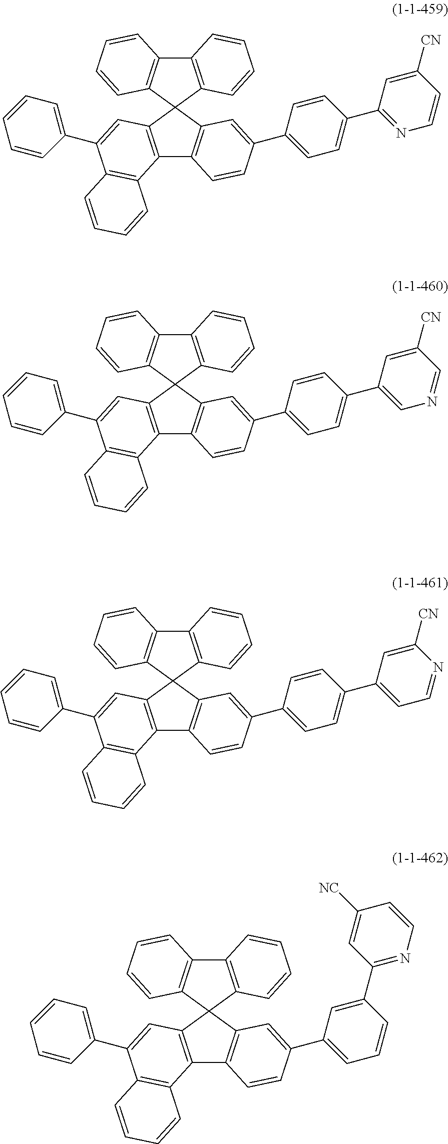 [SMILES: N#Cc1ccnc(-c2ccc(-c3ccc4c(c3)C3(c5ccccc5-c5ccccc53)c3cc(-c5ccccc5)c5ccccc5c3-4)cc2)c1 ; N#Cc1cncc(-c2ccc(-c3ccc4c(c3)C3(c5ccccc5-c5ccccc53)c3cc(-c5ccccc5)c5ccccc5c3-4)cc2)c1 ; N#Cc1cc(-c2ccc(-c3ccc4c(c3)C3(c5ccccc5-c5ccccc53)c3cc(-c5ccccc5)c5ccccc5c3-4)cc2)ccn1 ; N#Cc1ccnc(-c2cccc(-c3ccc4c(c3)C3(c5ccccc5-c5ccccc53)c3cc(-c5ccccc5)c5ccccc5c3-4)c2)c1]

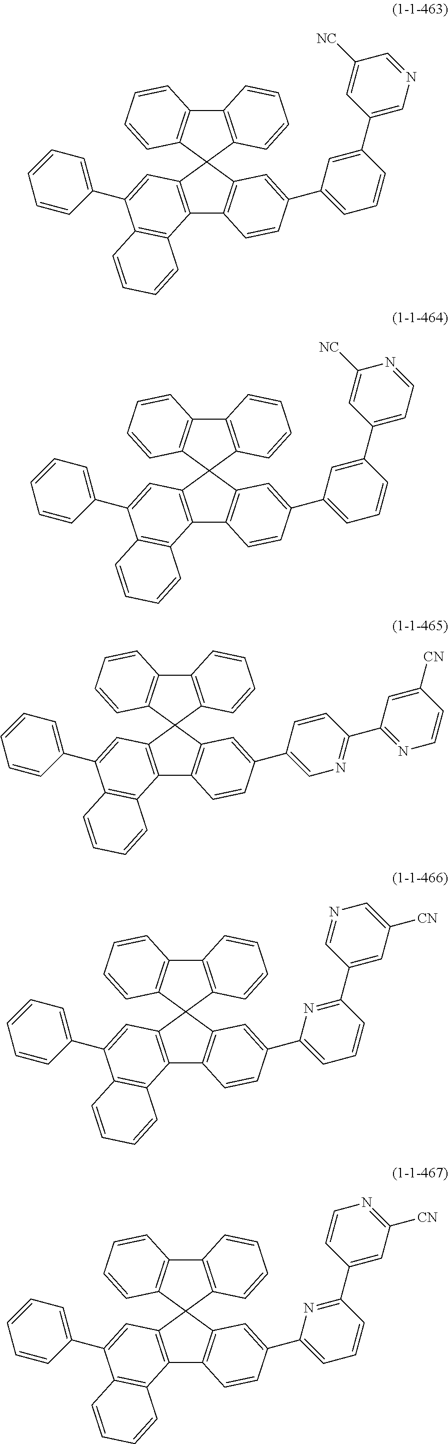 [SMILES: N#Cc1cncc(-c2cccc(-c3ccc4c(c3)C3(c5ccccc5-c5ccccc53)c3cc(-c5ccccc5)c5ccccc5c3-4)c2)c1 ; N#Cc1cc(-c2cccc(-c3ccc4c(c3)C3(c5ccccc5-c5ccccc53)c3cc(-c5ccccc5)c5ccccc5c3-4)c2)ccn1 ; N#Cc1ccnc(-c2ccc(-c3ccc4c(c3)C3(c5ccccc5-c5ccccc53)c3cc(-c5ccccc5)c5ccccc5c3-4)cn2)c1 ; N#Cc1cncc(-c2cccc(-c3ccc4c(c3)C3(c5ccccc5-c5ccccc53)c3cc(-c5ccccc5)c5ccccc5c3-4)n2)c1 ; N#Cc1cc(-c2cccc(-c3ccc4c(c3)C3(c5ccccc5-c5ccccc53)c3cc(-c5ccccc5)c5ccccc5c3-4)n2)ccn1]

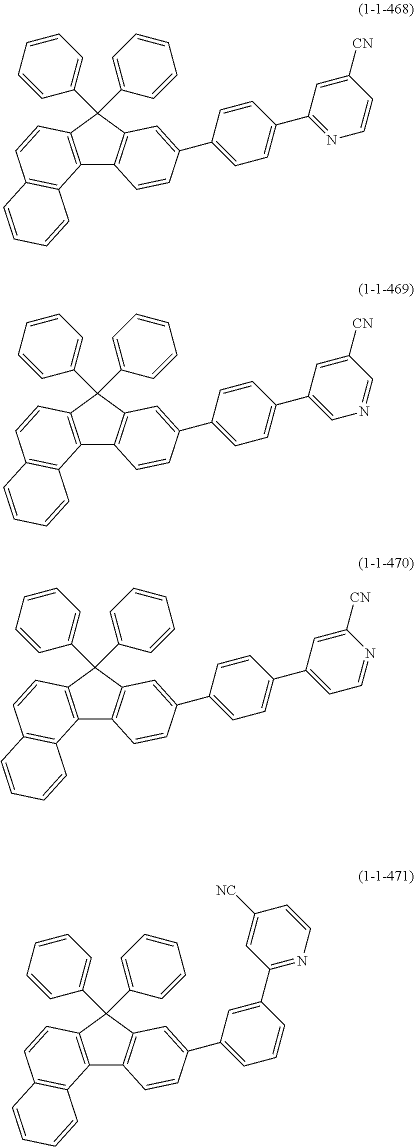 [SMILES: N#Cc1ccnc(-c2ccc(-c3ccc4c(c3)C(c3ccccc3)(c3ccccc3)c3ccc5ccccc5c3-4)cc2)c1 ; N#Cc1cncc(-c2ccc(-c3ccc4c(c3)C(c3ccccc3)(c3ccccc3)c3ccc5ccccc5c3-4)cc2)c1 ; N#Cc1cc(-c2ccc(-c3ccc4c(c3)C(c3ccccc3)(c3ccccc3)c3ccc5ccccc5c3-4)cc2)ccn1 ; N#Cc1ccnc(-c2cccc(-c3ccc4c(c3)C(c3ccccc3)(c3ccccc3)c3ccc5ccccc5c3-4)c2)c1]

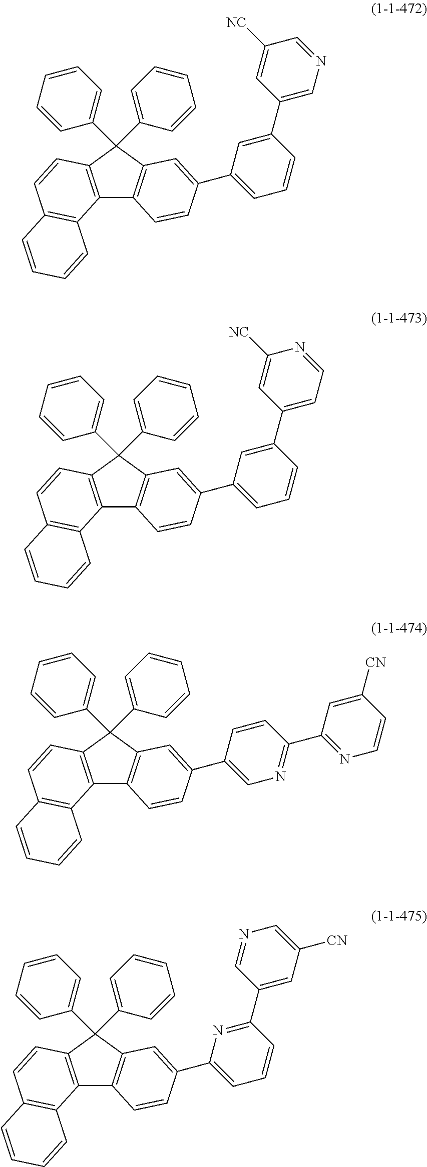 [SMILES: N#Cc1cncc(-c2cccc(-c3ccc4c(c3)C(c3ccccc3)(c3ccccc3)c3ccc5ccccc5c3-4)c2)c1 ; N#Cc1cc(-c2cccc(-c3ccc4c(c3)C(c3ccccc3)(c3ccccc3)c3ccc5ccccc5c3-4)c2)ccn1 ; N#Cc1ccnc(-c2ccc(-c3ccc4c(c3)C(c3ccccc3)(c3ccccc3)c3ccc5ccccc5c3-4)cn2)c1 ; N#Cc1cncc(-c2cccc(-c3ccc4c(c3)C(c3ccccc3)(c3ccccc3)c3ccc5ccccc5c3-4)n2)c1]

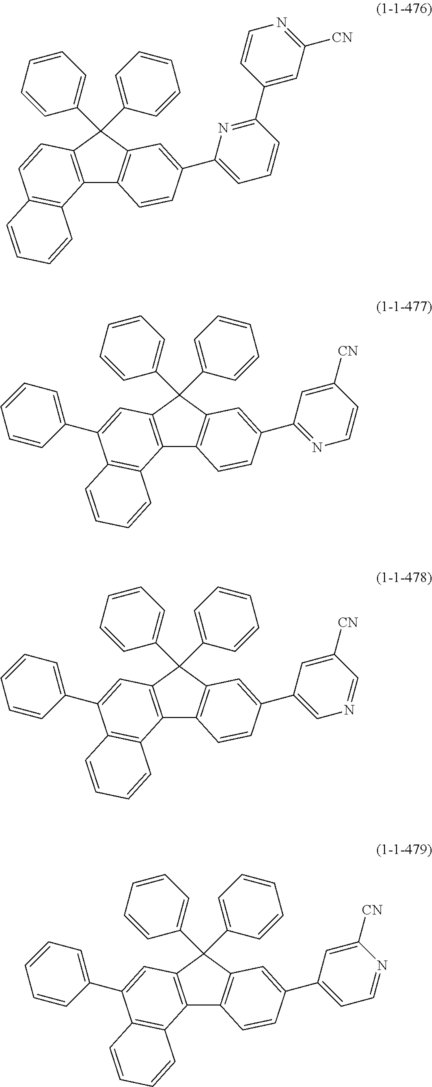 [SMILES: N#Cc1cc(-c2cccc(-c3ccc4c(c3)C(c3ccccc3)(c3ccccc3)c3ccc5ccccc5c3-4)n2)ccn1 ; N#Cc1ccnc(-c2ccc3c(c2)C(c2ccccc2)(c2ccccc2)c2cc(-c4ccccc4)c4ccccc4c2-3)c1 ; N#Cc1cncc(-c2ccc3c(c2)C(c2ccccc2)(c2ccccc2)c2cc(-c4ccccc4)c4ccccc4c2-3)c1 ; N#Cc1cc(-c2ccc3c(c2)C(c2ccccc2)(c2ccccc2)c2cc(-c4ccccc4)c4ccccc4c2-3)ccn1]

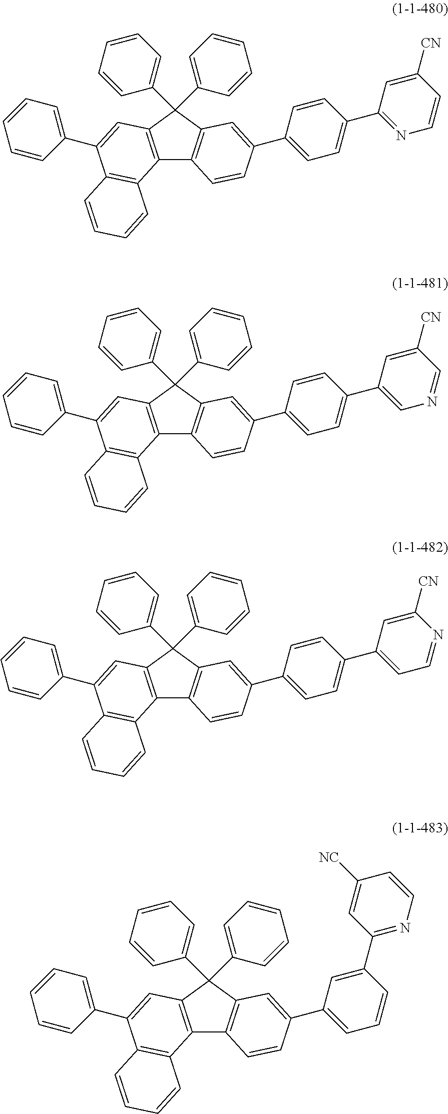 [SMILES: N#Cc1ccnc(-c2ccc(-c3ccc4c(c3)C(c3ccccc3)(c3ccccc3)c3cc(-c5ccccc5)c5ccccc5c3-4)cc2)c1 ; N#Cc1cncc(-c2ccc(-c3ccc4c(c3)C(c3ccccc3)(c3ccccc3)c3cc(-c5ccccc5)c5ccccc5c3-4)cc2)c1 ; N#Cc1cc(-c2ccc(-c3ccc4c(c3)C(c3ccccc3)(c3ccccc3)c3cc(-c5ccccc5)c5ccccc5c3-4)cc2)ccn1 ; N#Cc1ccnc(-c2cccc(-c3ccc4c(c3)C(c3ccccc3)(c3ccccc3)c3cc(-c5ccccc5)c5ccccc5c3-4)c2)c1]

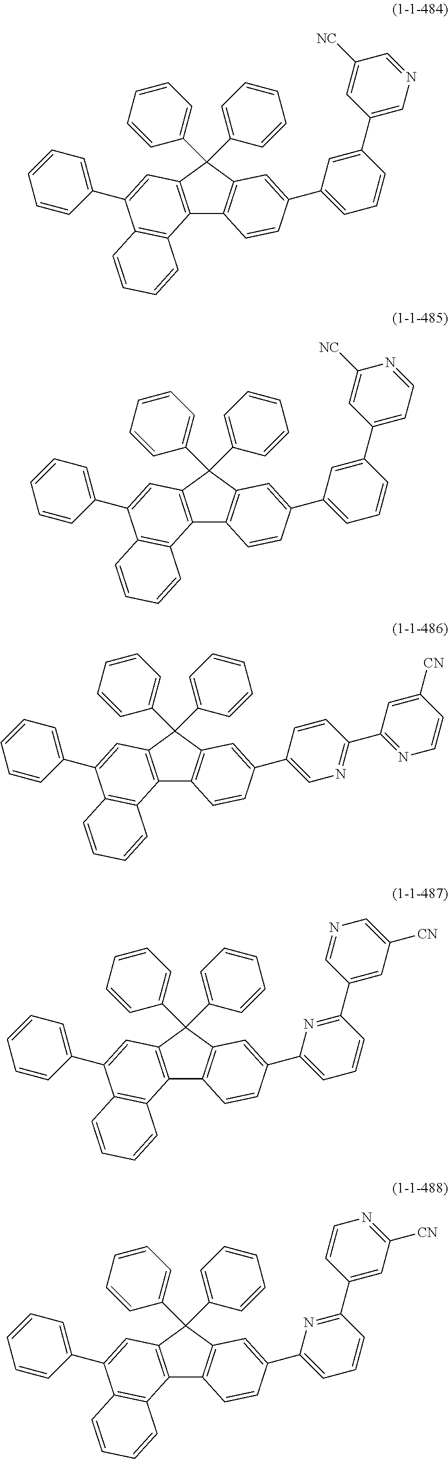 [SMILES: N#Cc1cncc(-c2cccc(-c3ccc4c(c3)C(c3ccccc3)(c3ccccc3)c3cc(-c5ccccc5)c5ccccc5c3-4)c2)c1 ; N#Cc1cc(-c2cccc(-c3ccc4c(c3)C(c3ccccc3)(c3ccccc3)c3cc(-c5ccccc5)c5ccccc5c3-4)c2)ccn1 ; N#Cc1ccnc(-c2ccc(-c3ccc4c(c3)C(c3ccccc3)(c3ccccc3)c3cc(-c5ccccc5)c5ccccc5c3-4)cn2)c1 ; N#Cc1cncc(-c2cccc(-c3ccc4c(c3)C(c3ccccc3)(c3ccccc3)c3cc(-c5ccccc5)c5ccccc5c3-4)n2)c1 ; N#Cc1cc(-c2cccc(-c3ccc4c(c3)C(c3ccccc3)(c3ccccc3)c3cc(-c5ccccc5)c5ccccc5c3-4)n2)ccn1]

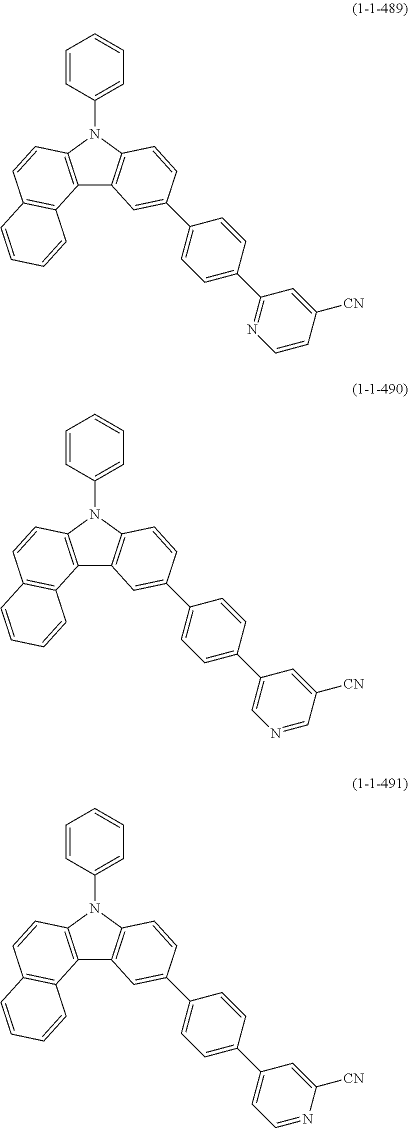 [SMILES: N#Cc1ccnc(-c2ccc(-c3ccc4c(c3)c3c5ccccc5ccc3n4-c3ccccc3)cc2)c1 ; N#Cc1cncc(-c2ccc(-c3ccc4c(c3)c3c5ccccc5ccc3n4-c3ccccc3)cc2)c1 ; N#Cc1cc(-c2ccc(-c3ccc4c(c3)c3c5ccccc5ccc3n4-c3ccccc3)cc2)ccn1]

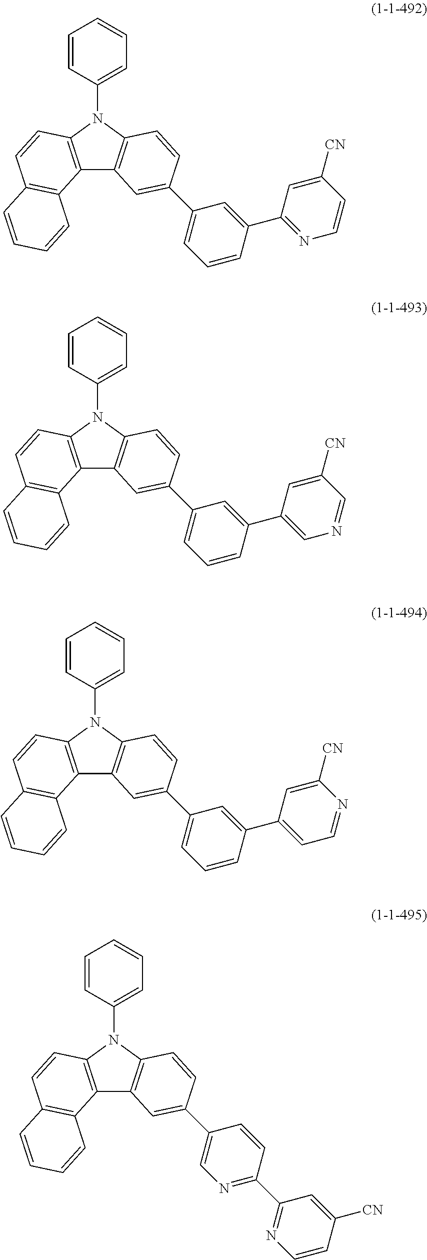 [SMILES: N#Cc1ccnc(-c2cccc(-c3ccc4c(c3)c3c5ccccc5ccc3n4-c3ccccc3)c2)c1 ; N#Cc1cncc(-c2cccc(-c3ccc4c(c3)c3c5ccccc5ccc3n4-c3ccccc3)c2)c1 ; N#Cc1cc(-c2cccc(-c3ccc4c(c3)c3c5ccccc5ccc3n4-c3ccccc3)c2)ccn1 ; N#Cc1ccnc(-c2ccc(-c3ccc4c(c3)c3c5ccccc5ccc3n4-c3ccccc3)cn2)c1]

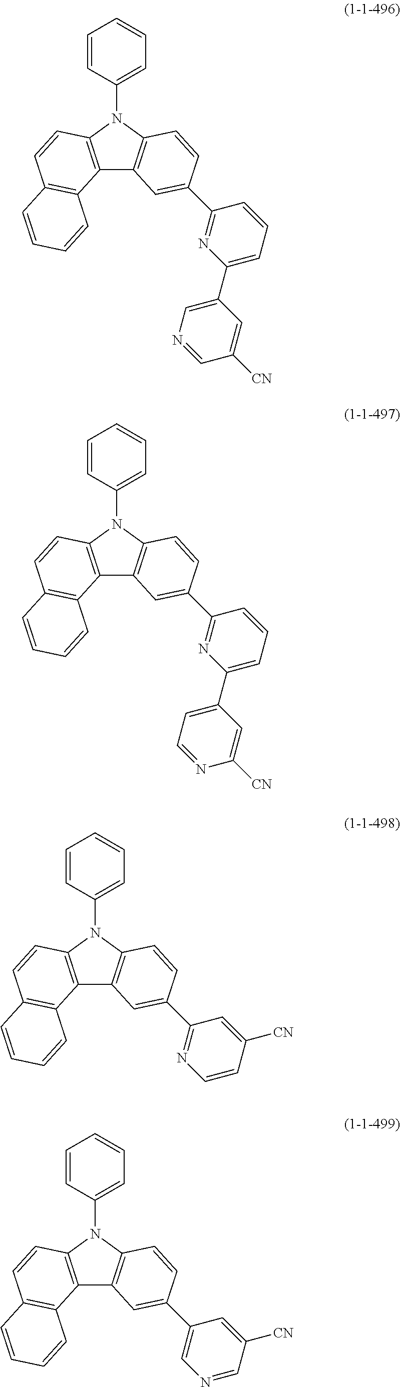 [SMILES: N#Cc1cncc(-c2cccc(-c3ccc4c(c3)c3c5ccccc5ccc3n4-c3ccccc3)n2)c1 ; N#Cc1cc(-c2cccc(-c3ccc4c(c3)c3c5ccccc5ccc3n4-c3ccccc3)n2)ccn1 ; N#Cc1ccnc(-c2ccc3c(c2)c2c4ccccc4ccc2n3-c2ccccc2)c1 ; N#Cc1cncc(-c2ccc3c(c2)c2c4ccccc4ccc2n3-c2ccccc2)c1]

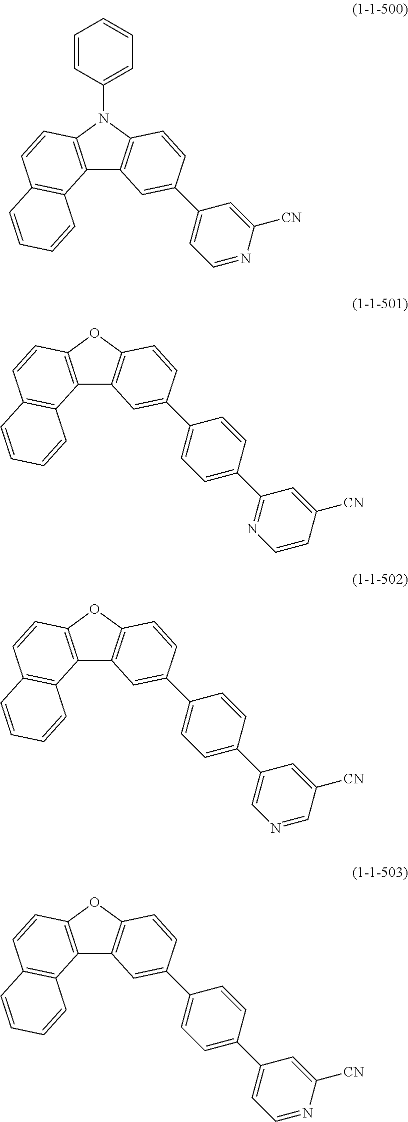 [SMILES: N#Cc1cc(-c2ccc3c(c2)c2c4ccccc4ccc2n3-c2ccccc2)ccn1 ; N#Cc1ccnc(-c2ccc(-c3ccc4oc5ccc6ccccc6c5c4c3)cc2)c1 ; N#Cc1cncc(-c2ccc(-c3ccc4oc5ccc6ccccc6c5c4c3)cc2)c1 ; N#Cc1cc(-c2ccc(-c3ccc4oc5ccc6ccccc6c5c4c3)cc2)ccn1]

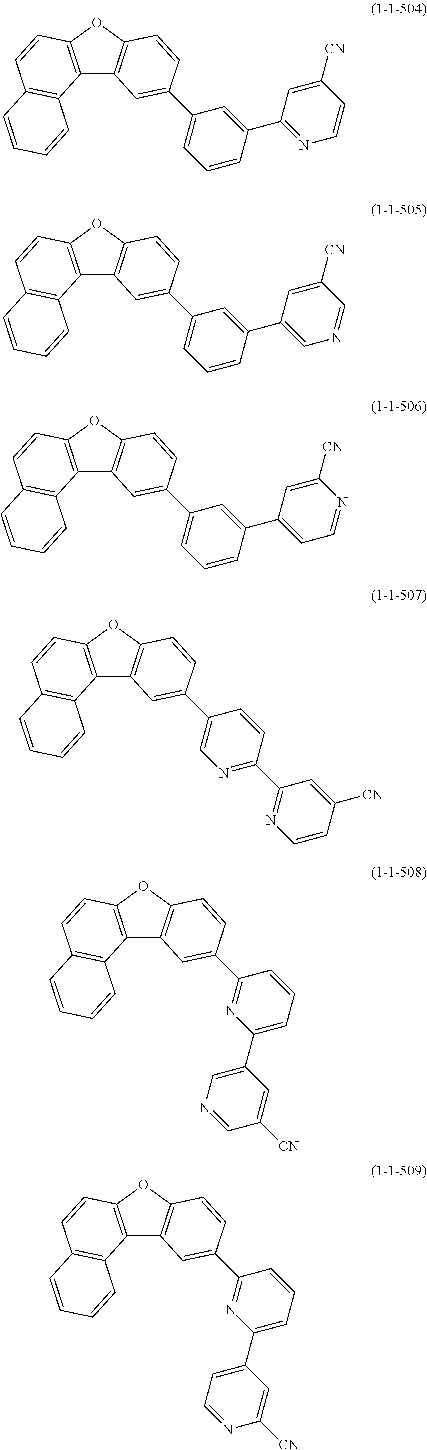 [SMILES: N#Cc1ccnc(-c2cccc(-c3ccc4oc5ccc6ccccc6c5c4c3)c2)c1 ; N#Cc1cncc(-c2cccc(-c3ccc4oc5ccc6ccccc6c5c4c3)c2)c1 ; N#Cc1cc(-c2cccc(-c3ccc4oc5ccc6ccccc6c5c4c3)c2)ccn1 ; N#Cc1ccnc(-c2ccc(-c3ccc4oc5ccc6ccccc6c5c4c3)cn2)c1 ; N#Cc1cncc(-c2cccc(-c3ccc4oc5ccc6ccccc6c5c4c3)n2)c1 ; N#Cc1cc(-c2cccc(-c3ccc4oc5ccc6ccccc6c5c4c3)n2)ccn1]

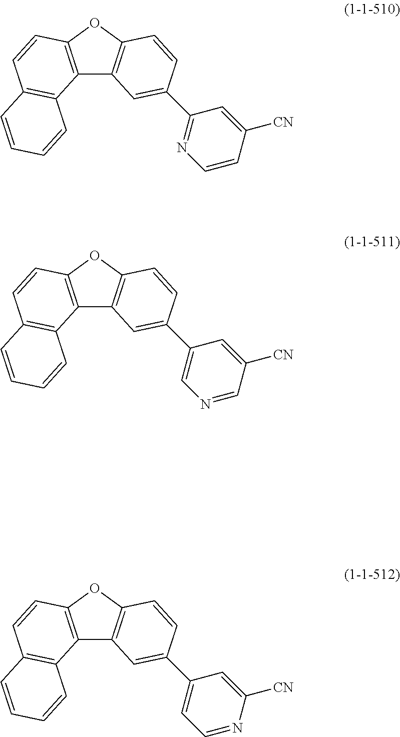 [SMILES: N#Cc1ccnc(-c2ccc3oc4ccc5ccccc5c4c3c2)c1 ; N#Cc1cncc(-c2ccc3oc4ccc5ccccc5c4c3c2)c1 ; N#Cc1cc(-c2ccc3oc4ccc5ccccc5c4c3c2)ccn1]

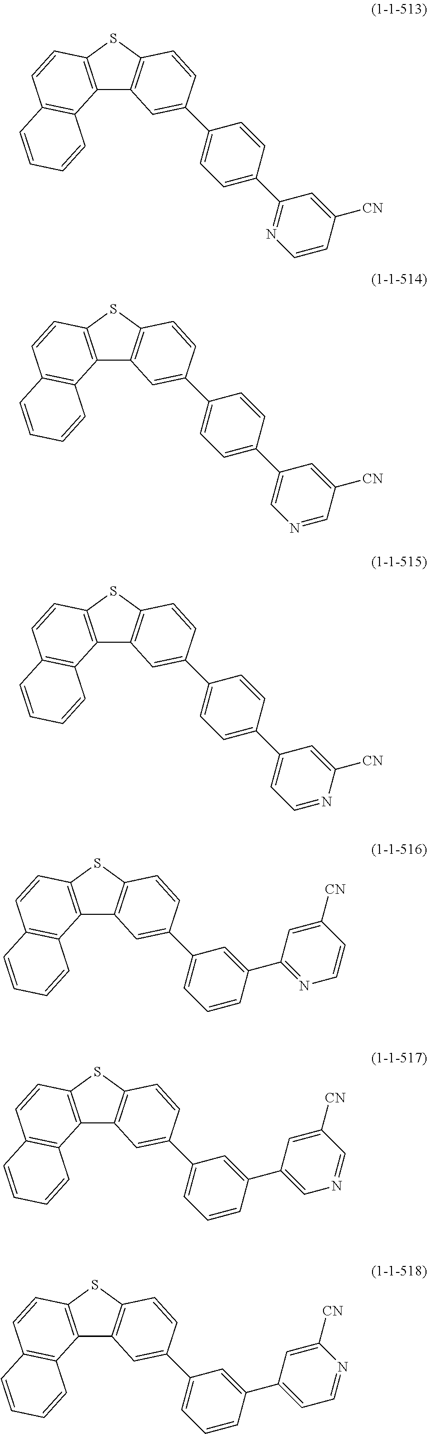 [SMILES: N#Cc1ccnc(-c2ccc(-c3ccc4sc5ccc6ccccc6c5c4c3)cc2)c1 ; N#Cc1cncc(-c2ccc(-c3ccc4sc5ccc6ccccc6c5c4c3)cc2)c1 ; N#Cc1cc(-c2ccc(-c3ccc4sc5ccc6ccccc6c5c4c3)cc2)ccn1 ; N#Cc1ccnc(-c2cccc(-c3ccc4sc5ccc6ccccc6c5c4c3)c2)c1 ; N#Cc1cncc(-c2cccc(-c3ccc4sc5ccc6ccccc6c5c4c3)c2)c1 ; N#Cc1cc(-c2cccc(-c3ccc4sc5ccc6ccccc6c5c4c3)c2)ccn1]

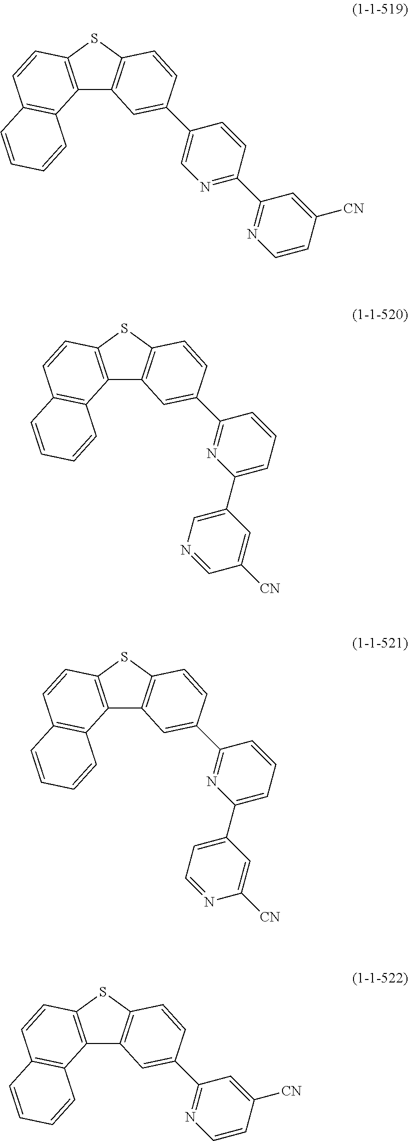 [SMILES: N#Cc1ccnc(-c2ccc(-c3ccc4sc5ccc6ccccc6c5c4c3)cn2)c1 ; N#Cc1cncc(-c2cccc(-c3ccc4sc5ccc6ccccc6c5c4c3)n2)c1 ; N#Cc1cc(-c2cccc(-c3ccc4sc5ccc6ccccc6c5c4c3)n2)ccn1 ; N#Cc1ccnc(-c2ccc3sc4ccc5ccccc5c4c3c2)c1]

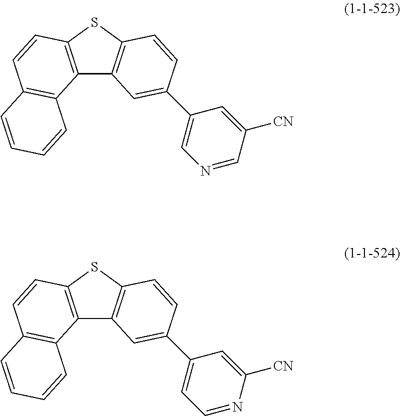 [SMILES: N#Cc1cncc(-c2ccc3sc4ccc5ccccc5c4c3c2)c1 ; N#Cc1cc(-c2ccc3sc4ccc5ccccc5c4c3c2)ccn1]

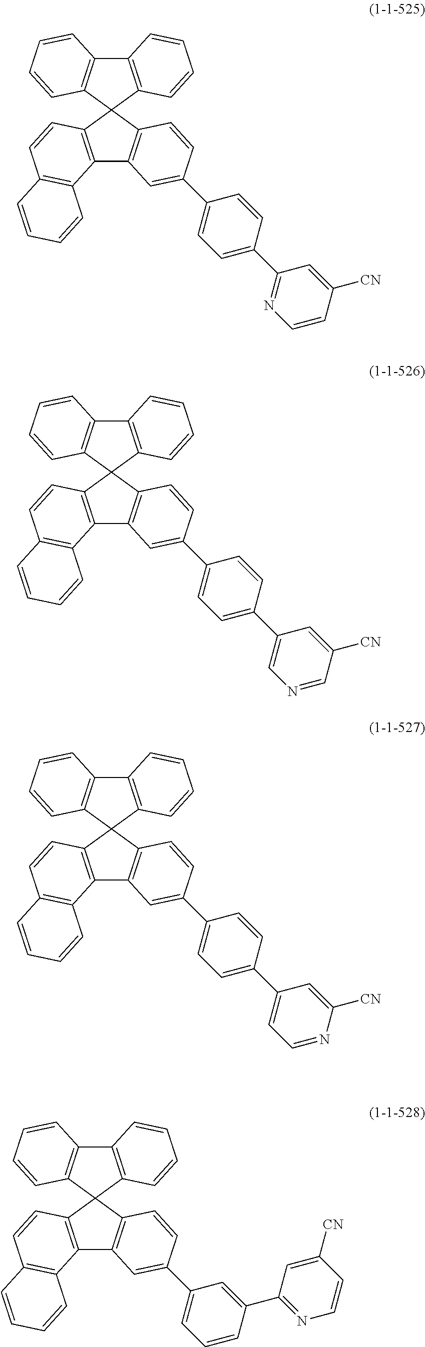 [SMILES: N#Cc1ccnc(-c2ccc(-c3ccc4c(c3)-c3c(ccc5ccccc35)C43c4ccccc4-c4ccccc43)cc2)c1 ; N#Cc1cncc(-c2ccc(-c3ccc4c(c3)-c3c(ccc5ccccc35)C43c4ccccc4-c4ccccc43)cc2)c1 ; N#Cc1cc(-c2ccc(-c3ccc4c(c3)-c3c(ccc5ccccc35)C43c4ccccc4-c4ccccc43)cc2)ccn1 ; N#Cc1ccnc(-c2cccc(-c3ccc4c(c3)-c3c(ccc5ccccc35)C43c4ccccc4-c4ccccc43)c2)c1]

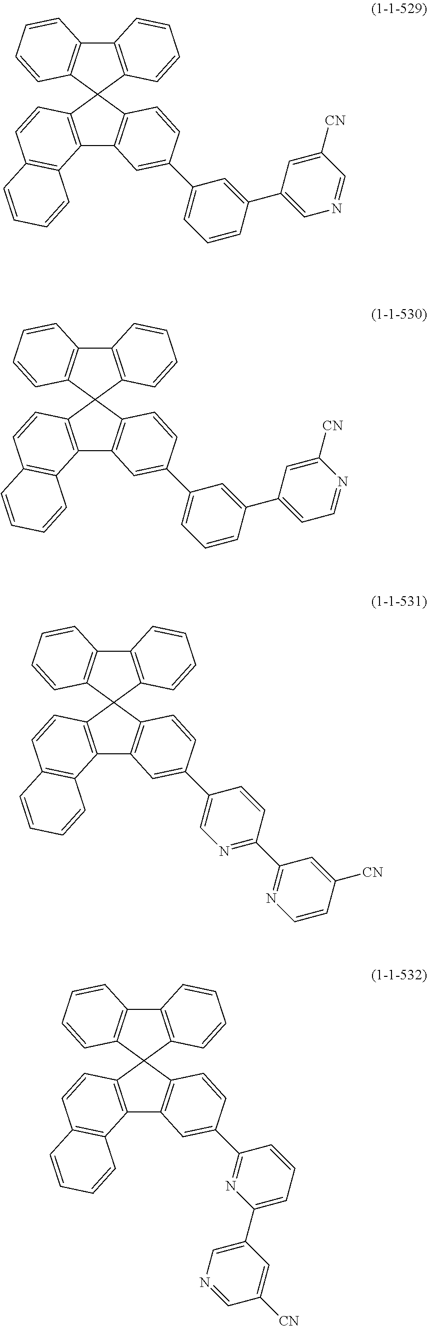 [SMILES: N#Cc1cncc(-c2cccc(-c3ccc4c(c3)-c3c(ccc5ccccc35)C43c4ccccc4-c4ccccc43)c2)c1 ; N#Cc1cc(-c2cccc(-c3ccc4c(c3)-c3c(ccc5ccccc35)C43c4ccccc4-c4ccccc43)c2)ccn1 ; N#Cc1ccnc(-c2ccc(-c3ccc4c(c3)-c3c(ccc5ccccc35)C43c4ccccc4-c4ccccc43)cn2)c1 ; N#Cc1cncc(-c2cccc(-c3ccc4c(c3)-c3c(ccc5ccccc35)C43c4ccccc4-c4ccccc43)n2)c1]

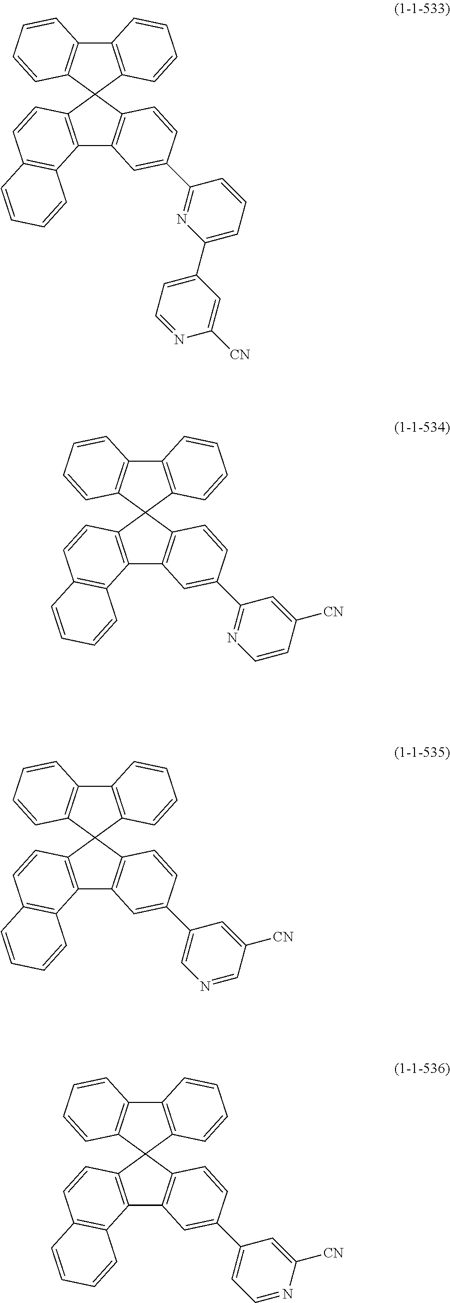 [SMILES: N#Cc1cc(-c2cccc(-c3ccc4c(c3)-c3c(ccc5ccccc35)C43c4ccccc4-c4ccccc43)n2)ccn1 ; N#Cc1ccnc(-c2ccc3c(c2)-c2c(ccc4ccccc24)C32c3ccccc3-c3ccccc32)c1 ; N#Cc1cncc(-c2ccc3c(c2)-c2c(ccc4ccccc24)C32c3ccccc3-c3ccccc32)c1 ; N#Cc1cc(-c2ccc3c(c2)-c2c(ccc4ccccc24)C32c3ccccc3-c3ccccc32)ccn1]

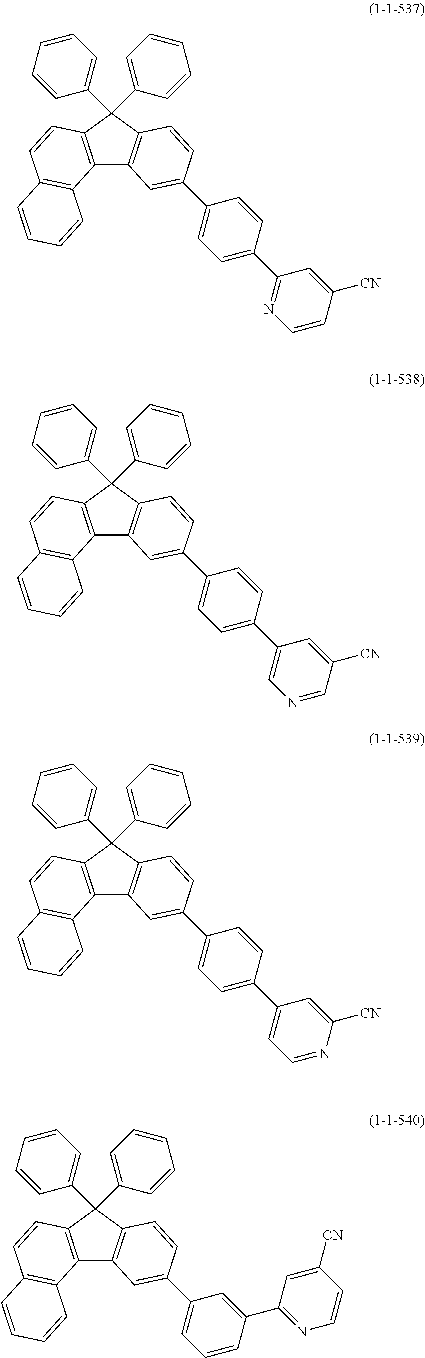 [SMILES: N#Cc1ccnc(-c2ccc(-c3ccc4c(c3)-c3c(ccc5ccccc35)C4(c3ccccc3)c3ccccc3)cc2)c1 ; N#Cc1cncc(-c2ccc(-c3ccc4c(c3)-c3c(ccc5ccccc35)C4(c3ccccc3)c3ccccc3)cc2)c1 ; N#Cc1cc(-c2ccc(-c3ccc4c(c3)-c3c(ccc5ccccc35)C4(c3ccccc3)c3ccccc3)cc2)ccn1 ; N#Cc1ccnc(-c2cccc(-c3ccc4c(c3)-c3c(ccc5ccccc35)C4(c3ccccc3)c3ccccc3)c2)c1]

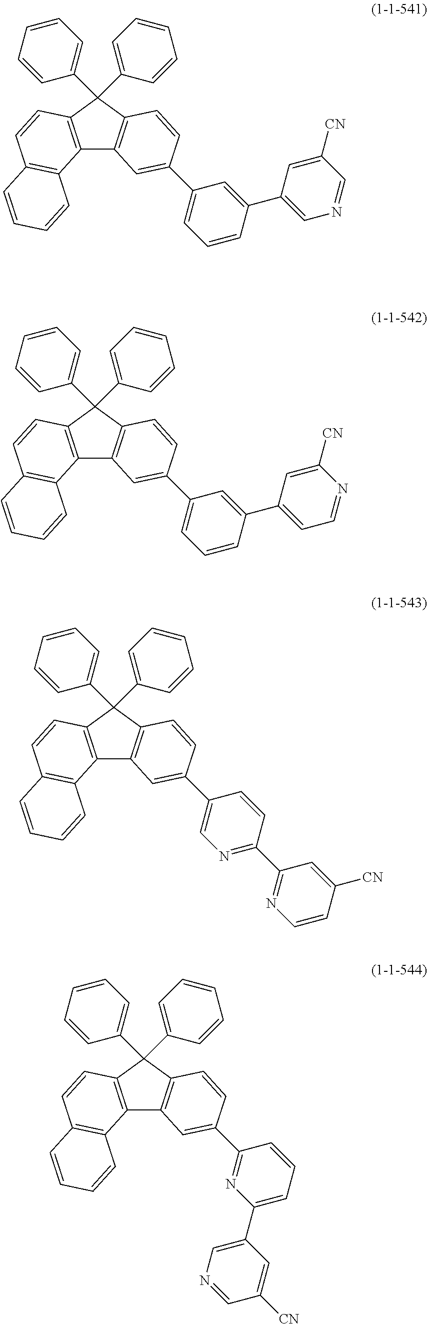 [SMILES: N#Cc1cncc(-c2cccc(-c3ccc4c(c3)-c3c(ccc5ccccc35)C4(c3ccccc3)c3ccccc3)c2)c1 ; N#Cc1cc(-c2cccc(-c3ccc4c(c3)-c3c(ccc5ccccc35)C4(c3ccccc3)c3ccccc3)c2)ccn1 ; N#Cc1ccnc(-c2ccc(-c3ccc4c(c3)-c3c(ccc5ccccc35)C4(c3ccccc3)c3ccccc3)cn2)c1 ; N#Cc1cncc(-c2cccc(-c3ccc4c(c3)-c3c(ccc5ccccc35)C4(c3ccccc3)c3ccccc3)n2)c1]

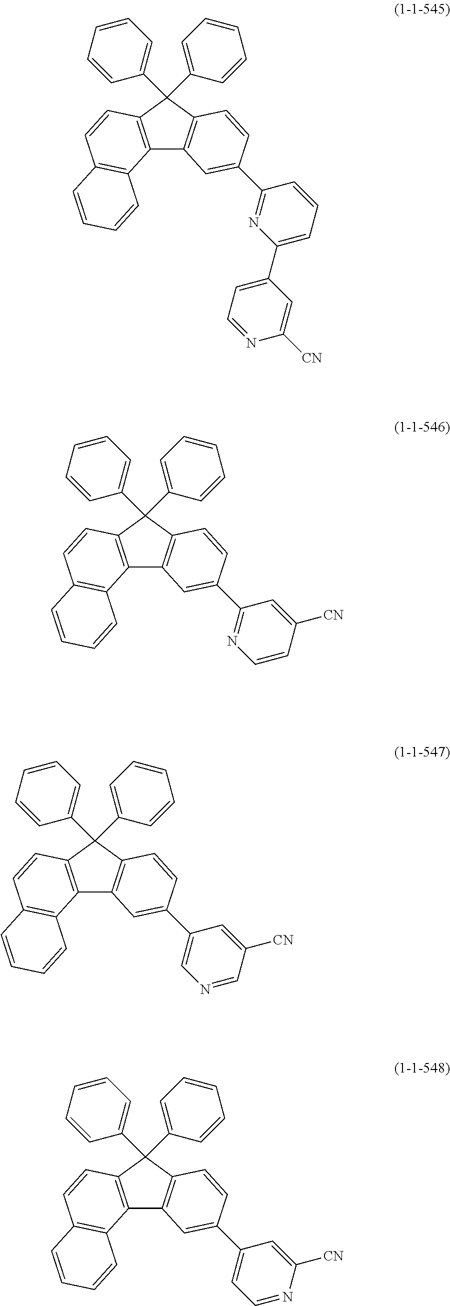 [SMILES: N#Cc1cc(-c2cccc(-c3ccc4c(c3)-c3c(ccc5ccccc35)C4(c3ccccc3)c3ccccc3)n2)ccn1 ; N#Cc1ccnc(-c2ccc3c(c2)-c2c(ccc4ccccc24)C3(c2ccccc2)c2ccccc2)c1 ; N#Cc1cncc(-c2ccc3c(c2)-c2c(ccc4ccccc24)C3(c2ccccc2)c2ccccc2)c1 ; N#Cc1cc(-c2ccc3c(c2)-c2c(ccc4ccccc24)C3(c2ccccc2)c2ccccc2)ccn1]

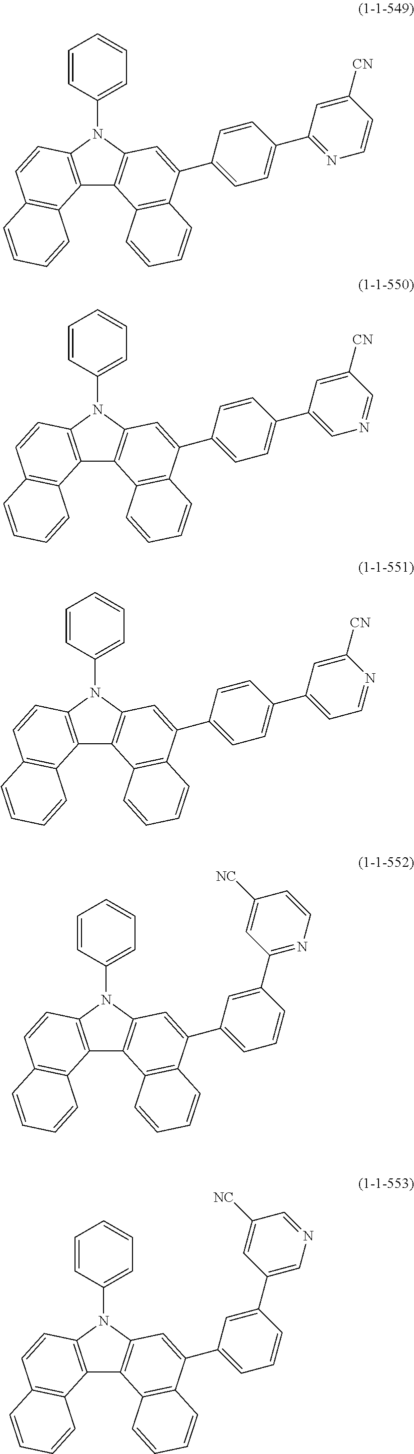 [SMILES: N#Cc1ccnc(-c2ccc(-c3cc4c(c5ccccc35)c3c5ccccc5ccc3n4-c3ccccc3)cc2)c1 ; N#Cc1cncc(-c2ccc(-c3cc4c(c5ccccc35)c3c5ccccc5ccc3n4-c3ccccc3)cc2)c1 ; N#Cc1cc(-c2ccc(-c3cc4c(c5ccccc35)c3c5ccccc5ccc3n4-c3ccccc3)cc2)ccn1 ; N#Cc1ccnc(-c2cccc(-c3cc4c(c5ccccc35)c3c5ccccc5ccc3n4-c3ccccc3)c2)c1 ; N#Cc1cncc(-c2cccc(-c3cc4c(c5ccccc35)c3c5ccccc5ccc3n4-c3ccccc3)c2)c1]

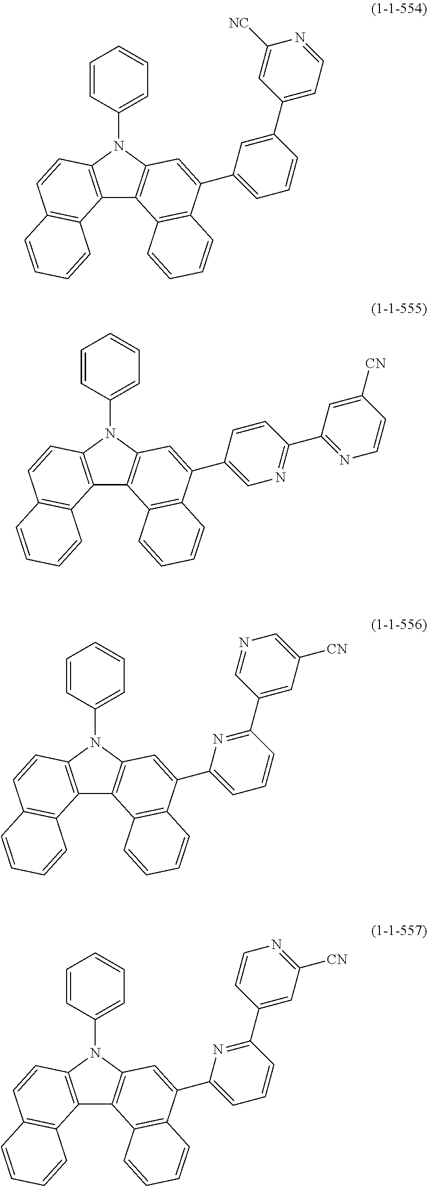 [SMILES: N#Cc1cc(-c2cccc(-c3cc4c(c5ccccc35)c3c5ccccc5ccc3n4-c3ccccc3)c2)ccn1 ; N#Cc1ccnc(-c2ccc(-c3cc4c(c5ccccc35)c3c5ccccc5ccc3n4-c3ccccc3)cn2)c1 ; N#Cc1cncc(-c2cccc(-c3cc4c(c5ccccc35)c3c5ccccc5ccc3n4-c3ccccc3)n2)c1 ; N#Cc1cc(-c2cccc(-c3cc4c(c5ccccc35)c3c5ccccc5ccc3n4-c3ccccc3)n2)ccn1]

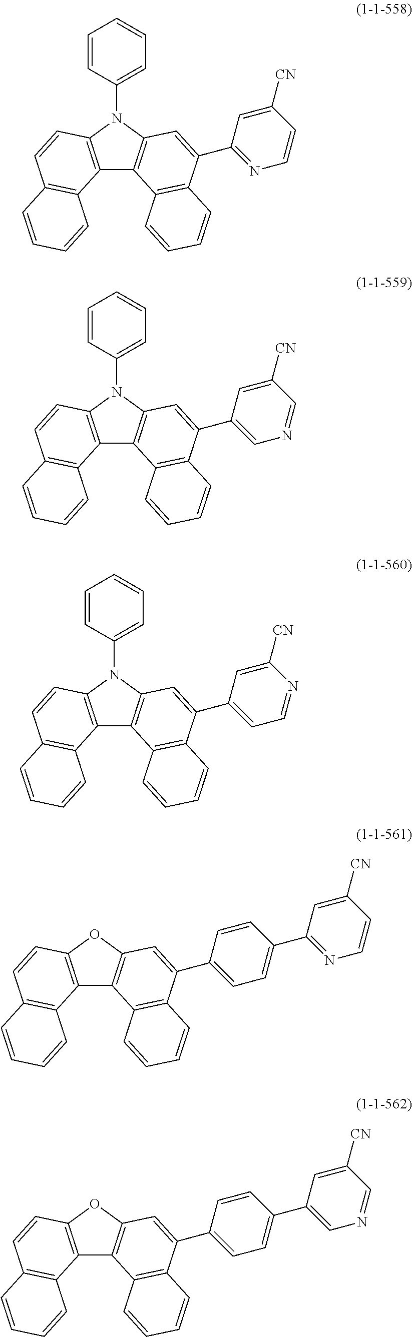 [SMILES: N#Cc1ccnc(-c2cc3c(c4ccccc24)c2c4ccccc4ccc2n3-c2ccccc2)c1 ; N#Cc1cncc(-c2cc3c(c4ccccc24)c2c4ccccc4ccc2n3-c2ccccc2)c1 ; N#Cc1cc(-c2cc3c(c4ccccc24)c2c4ccccc4ccc2n3-c2ccccc2)ccn1 ; N#Cc1ccnc(-c2ccc(-c3cc4oc5ccc6ccccc6c5c4c4ccccc34)cc2)c1 ; N#Cc1cncc(-c2ccc(-c3cc4oc5ccc6ccccc6c5c4c4ccccc34)cc2)c1]

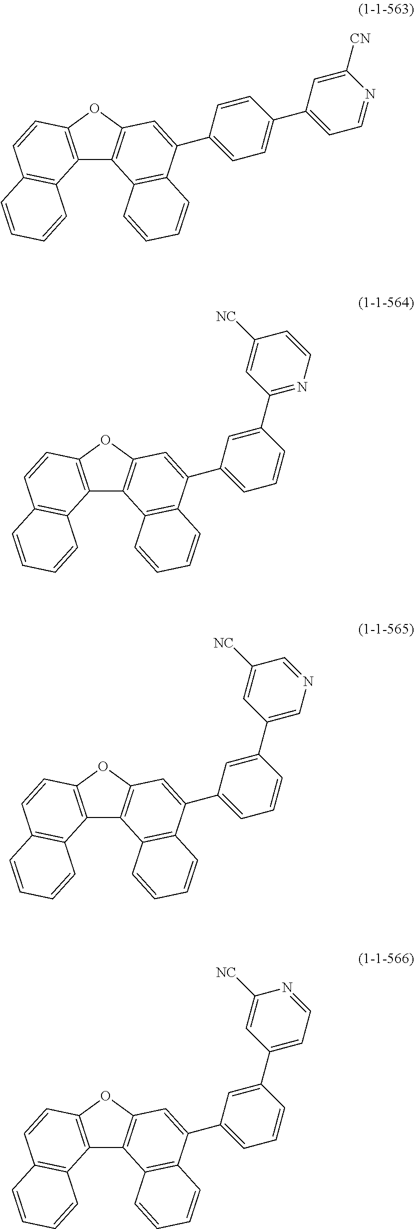 [SMILES: N#Cc1cc(-c2ccc(-c3cc4oc5ccc6ccccc6c5c4c4ccccc34)cc2)ccn1 ; N#Cc1ccnc(-c2cccc(-c3cc4oc5ccc6ccccc6c5c4c4ccccc34)c2)c1 ; N#Cc1cncc(-c2cccc(-c3cc4oc5ccc6ccccc6c5c4c4ccccc34)c2)c1 ; N#Cc1cc(-c2cccc(-c3cc4oc5ccc6ccccc6c5c4c4ccccc34)c2)ccn1]

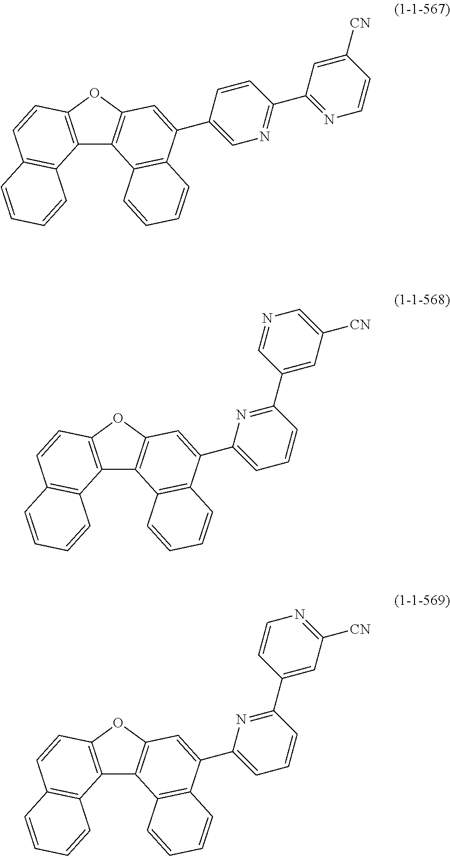 [SMILES: N#Cc1ccnc(-c2ccc(-c3cc4oc5ccc6ccccc6c5c4c4ccccc34)cn2)c1 ; N#Cc1cncc(-c2cccc(-c3cc4oc5ccc6ccccc6c5c4c4ccccc34)n2)c1 ; N#Cc1cc(-c2cccc(-c3cc4oc5ccc6ccccc6c5c4c4ccccc34)n2)ccn1]

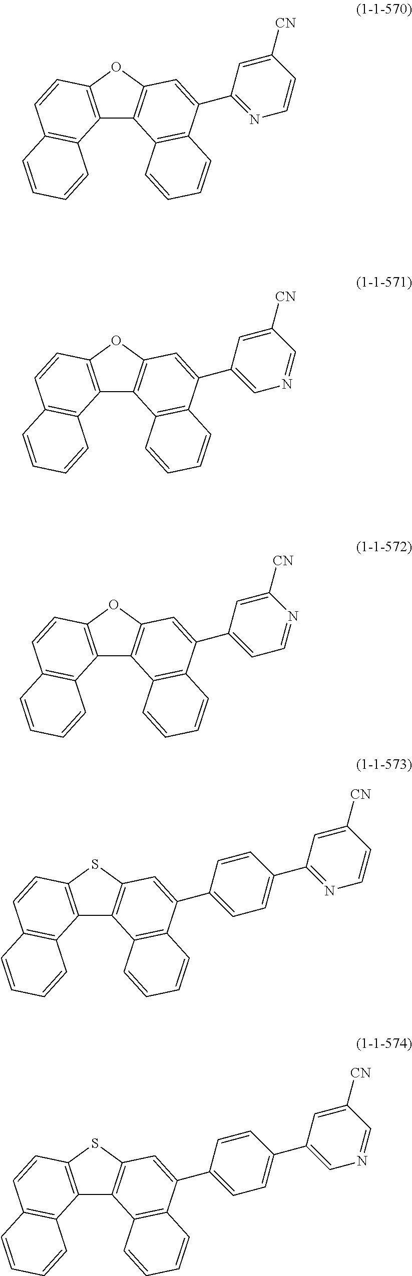 [SMILES: N#Cc1ccnc(-c2cc3oc4ccc5ccccc5c4c3c3ccccc23)c1 ; N#Cc1cncc(-c2cc3oc4ccc5ccccc5c4c3c3ccccc23)c1 ; N#Cc1cc(-c2cc3oc4ccc5ccccc5c4c3c3ccccc23)ccn1 ; N#Cc1ccnc(-c2ccc(-c3cc4sc5ccc6ccccc6c5c4c4ccccc34)cc2)c1 ; N#Cc1cncc(-c2ccc(-c3cc4sc5ccc6ccccc6c5c4c4ccccc34)cc2)c1]

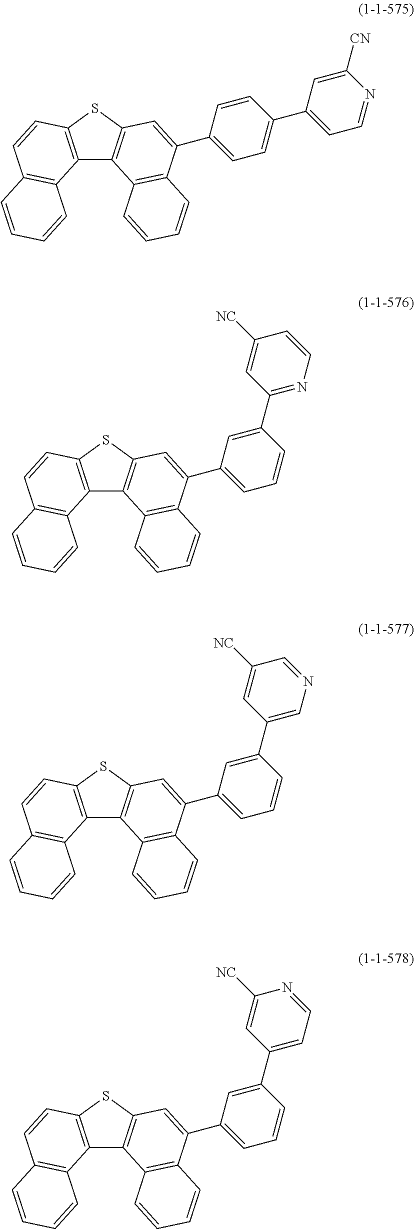 [SMILES: N#Cc1cc(-c2ccc(-c3cc4sc5ccc6ccccc6c5c4c4ccccc34)cc2)ccn1 ; N#Cc1ccnc(-c2cccc(-c3cc4sc5ccc6ccccc6c5c4c4ccccc34)c2)c1 ; N#Cc1cncc(-c2cccc(-c3cc4sc5ccc6ccccc6c5c4c4ccccc34)c2)c1 ; N#Cc1cc(-c2cccc(-c3cc4sc5ccc6ccccc6c5c4c4ccccc34)c2)ccn1]

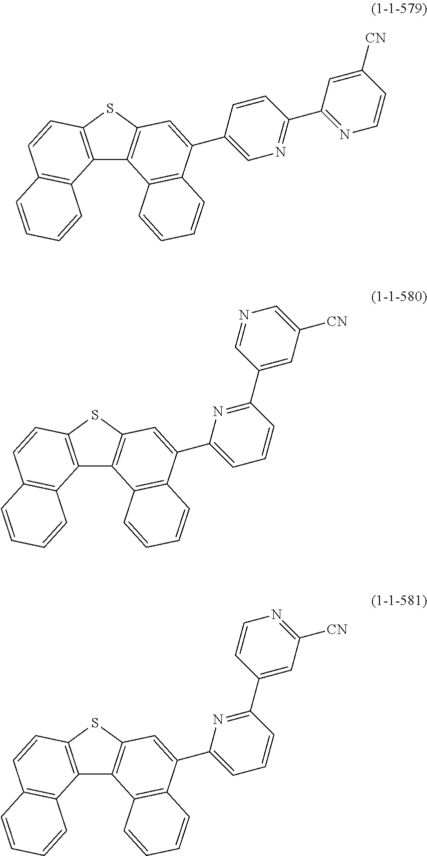 [SMILES: N#Cc1ccnc(-c2ccc(-c3cc4sc5ccc6ccccc6c5c4c4ccccc34)cn2)c1 ; N#Cc1cncc(-c2cccc(-c3cc4sc5ccc6ccccc6c5c4c4ccccc34)n2)c1 ; N#Cc1cc(-c2cccc(-c3cc4sc5ccc6ccccc6c5c4c4ccccc34)n2)ccn1]

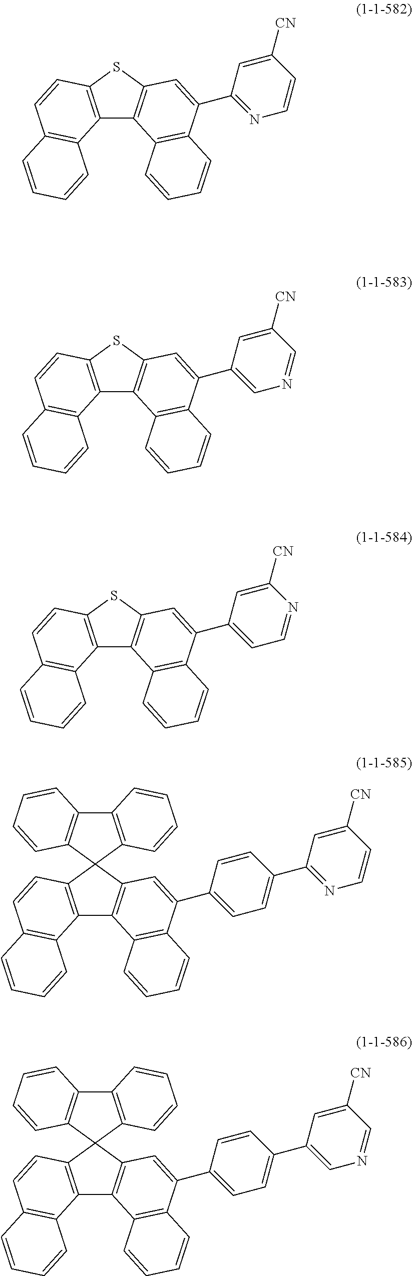 [SMILES: N#Cc1ccnc(-c2cc3sc4ccc5ccccc5c4c3c3ccccc23)c1 ; N#Cc1cncc(-c2cc3sc4ccc5ccccc5c4c3c3ccccc23)c1 ; N#Cc1cc(-c2cc3sc4ccc5ccccc5c4c3c3ccccc23)ccn1 ; N#Cc1ccnc(-c2ccc(-c3cc4c(c5ccccc35)-c3c(ccc5ccccc35)C43c4ccccc4-c4ccccc43)cc2)c1 ; N#Cc1cncc(-c2ccc(-c3cc4c(c5ccccc35)-c3c(ccc5ccccc35)C43c4ccccc4-c4ccccc43)cc2)c1]

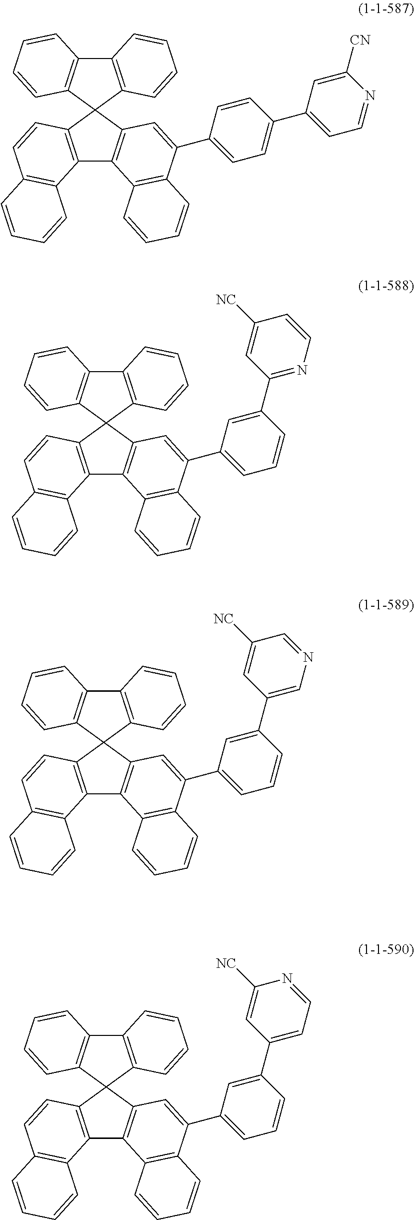 [SMILES: N#Cc1cc(-c2ccc(-c3cc4c(c5ccccc35)-c3c(ccc5ccccc35)C43c4ccccc4-c4ccccc43)cc2)ccn1 ; N#Cc1ccnc(-c2cccc(-c3cc4c(c5ccccc35)-c3c(ccc5ccccc35)C43c4ccccc4-c4ccccc43)c2)c1 ; N#Cc1cncc(-c2cccc(-c3cc4c(c5ccccc35)-c3c(ccc5ccccc35)C43c4ccccc4-c4ccccc43)c2)c1 ; N#Cc1cc(-c2cccc(-c3cc4c(c5ccccc35)-c3c(ccc5ccccc35)C43c4ccccc4-c4ccccc43)c2)ccn1]

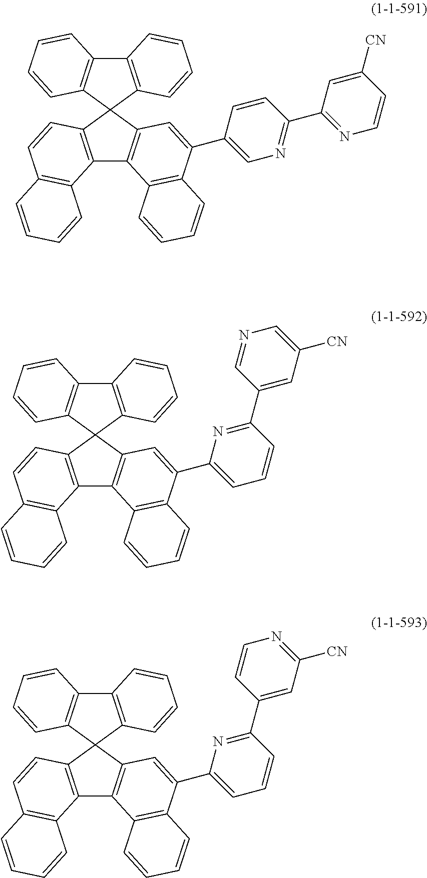 [SMILES: N#Cc1ccnc(-c2ccc(-c3cc4c(c5ccccc35)-c3c(ccc5ccccc35)C43c4ccccc4-c4ccccc43)cn2)c1 ; N#Cc1cncc(-c2cccc(-c3cc4c(c5ccccc35)-c3c(ccc5ccccc35)C43c4ccccc4-c4ccccc43)n2)c1 ; N#Cc1cc(-c2cccc(-c3cc4c(c5ccccc35)-c3c(ccc5ccccc35)C43c4ccccc4-c4ccccc43)n2)ccn1]

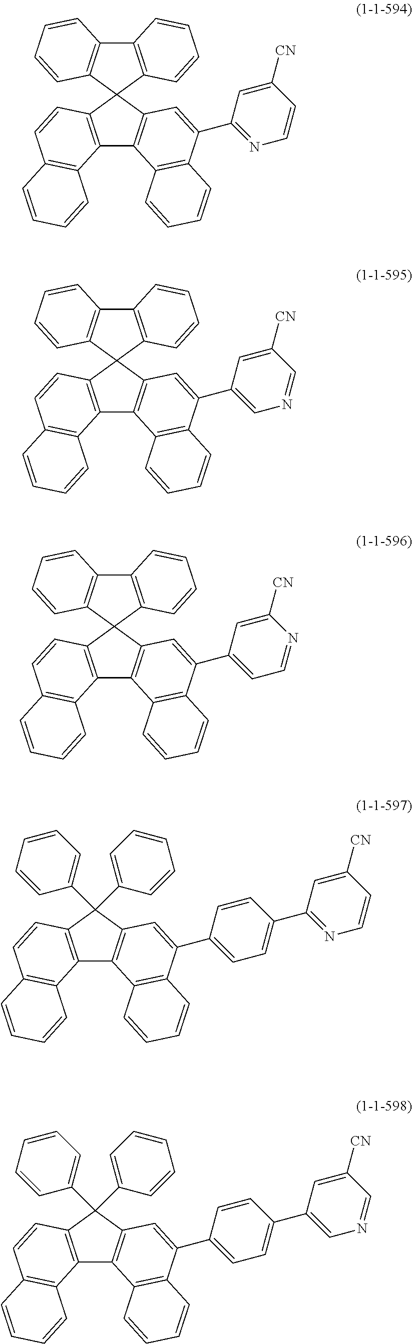 [SMILES: N#Cc1ccnc(-c2cc3c(c4ccccc24)-c2c(ccc4ccccc24)C32c3ccccc3-c3ccccc32)c1 ; N#Cc1cncc(-c2cc3c(c4ccccc24)-c2c(ccc4ccccc24)C32c3ccccc3-c3ccccc32)c1 ; N#Cc1cc(-c2cc3c(c4ccccc24)-c2c(ccc4ccccc24)C32c3ccccc3-c3ccccc32)ccn1 ; N#Cc1ccnc(-c2ccc(-c3cc4c(c5ccccc35)-c3c(ccc5ccccc35)C4(c3ccccc3)c3ccccc3)cc2)c1 ; N#Cc1cncc(-c2ccc(-c3cc4c(c5ccccc35)-c3c(ccc5ccccc35)C4(c3ccccc3)c3ccccc3)cc2)c1]

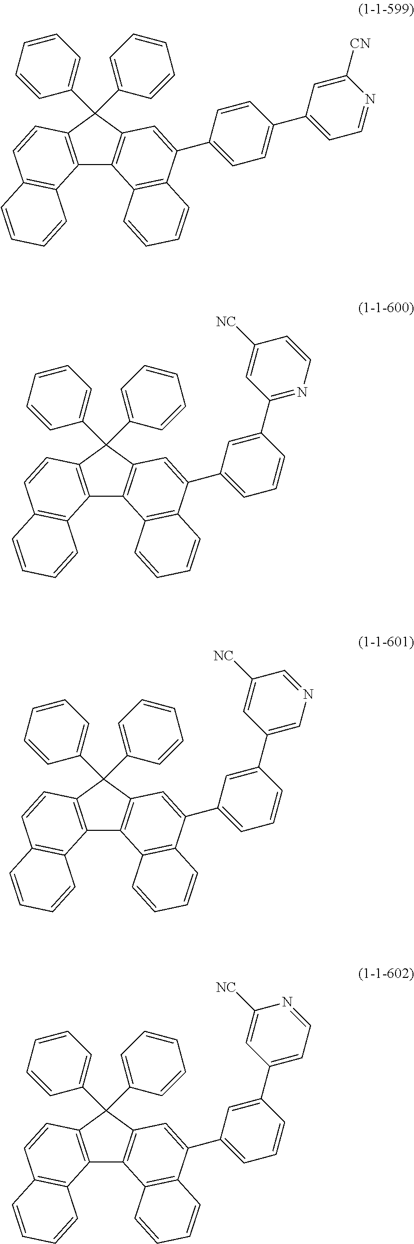 [SMILES: N#Cc1cc(-c2ccc(-c3cc4c(c5ccccc35)-c3c(ccc5ccccc35)C4(c3ccccc3)c3ccccc3)cc2)ccn1 ; N#Cc1ccnc(-c2cccc(-c3cc4c(c5ccccc35)-c3c(ccc5ccccc35)C4(c3ccccc3)c3ccccc3)c2)c1 ; N#Cc1cncc(-c2cccc(-c3cc4c(c5ccccc35)-c3c(ccc5ccccc35)C4(c3ccccc3)c3ccccc3)c2)c1 ; N#Cc1cc(-c2cccc(-c3cc4c(c5ccccc35)-c3c(ccc5ccccc35)C4(c3ccccc3)c3ccccc3)c2)ccn1]

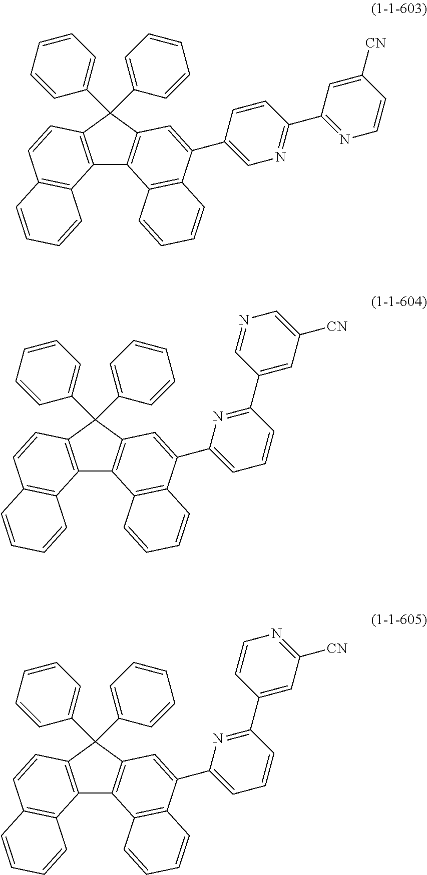 [SMILES: N#Cc1ccnc(-c2ccc(-c3cc4c(c5ccccc35)-c3c(ccc5ccccc35)C4(c3ccccc3)c3ccccc3)cn2)c1 ; N#Cc1cncc(-c2cccc(-c3cc4c(c5ccccc35)-c3c(ccc5ccccc35)C4(c3ccccc3)c3ccccc3)n2)c1 ; N#Cc1cc(-c2cccc(-c3cc4c(c5ccccc35)-c3c(ccc5ccccc35)C4(c3ccccc3)c3ccccc3)n2)ccn1]

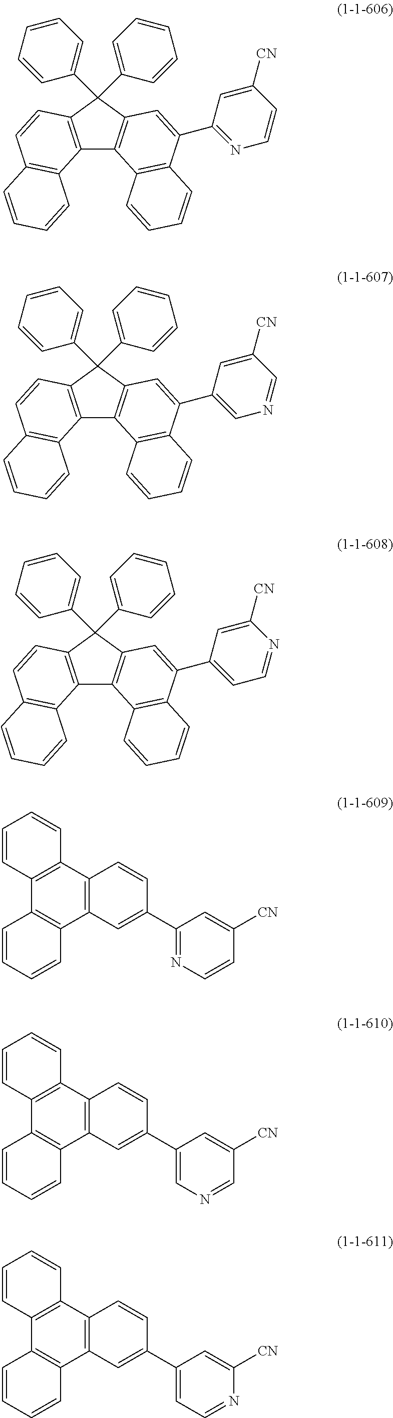 [SMILES: N#Cc1ccnc(-c2cc3c(c4ccccc24)-c2c(ccc4ccccc24)C3(c2ccccc2)c2ccccc2)c1 ; N#Cc1cncc(-c2cc3c(c4ccccc24)-c2c(ccc4ccccc24)C3(c2ccccc2)c2ccccc2)c1 ; N#Cc1cc(-c2cc3c(c4ccccc24)-c2c(ccc4ccccc24)C3(c2ccccc2)c2ccccc2)ccn1 ; N#Cc1ccnc(-c2ccc3c4ccccc4c4ccccc4c3c2)c1 ; N#Cc1cncc(-c2ccc3c4ccccc4c4ccccc4c3c2)c1 ; N#Cc1cc(-c2ccc3c4ccccc4c4ccccc4c3c2)ccn1]

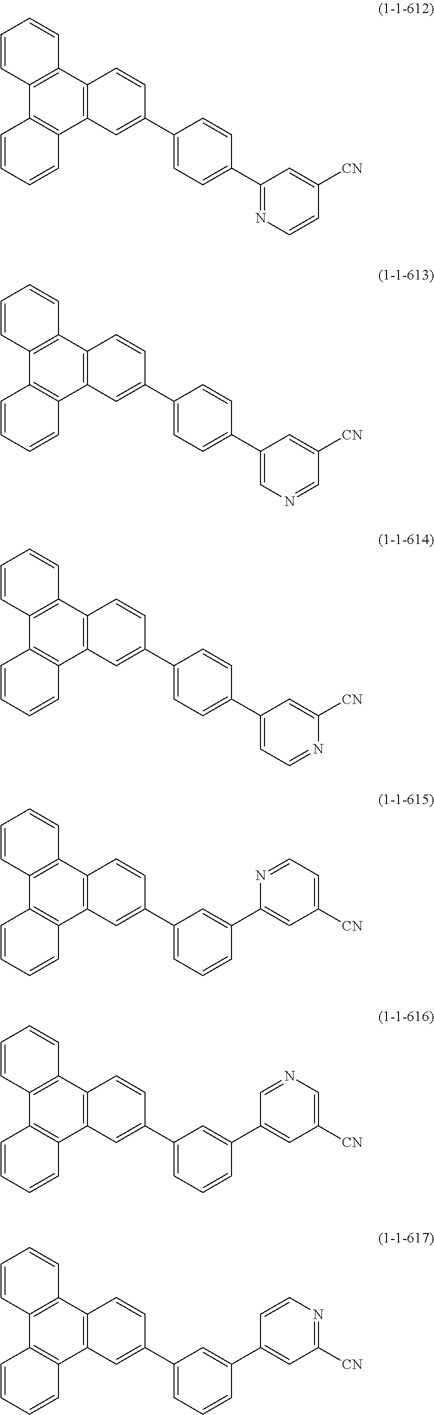 [SMILES: N#Cc1ccnc(-c2ccc(-c3ccc4c5ccccc5c5ccccc5c4c3)cc2)c1 ; N#Cc1cncc(-c2ccc(-c3ccc4c5ccccc5c5ccccc5c4c3)cc2)c1 ; N#Cc1cc(-c2ccc(-c3ccc4c5ccccc5c5ccccc5c4c3)cc2)ccn1 ; N#Cc1ccnc(-c2cccc(-c3ccc4c5ccccc5c5ccccc5c4c3)c2)c1 ; N#Cc1cncc(-c2cccc(-c3ccc4c5ccccc5c5ccccc5c4c3)c2)c1 ; N#Cc1cc(-c2cccc(-c3ccc4c5ccccc5c5ccccc5c4c3)c2)ccn1]

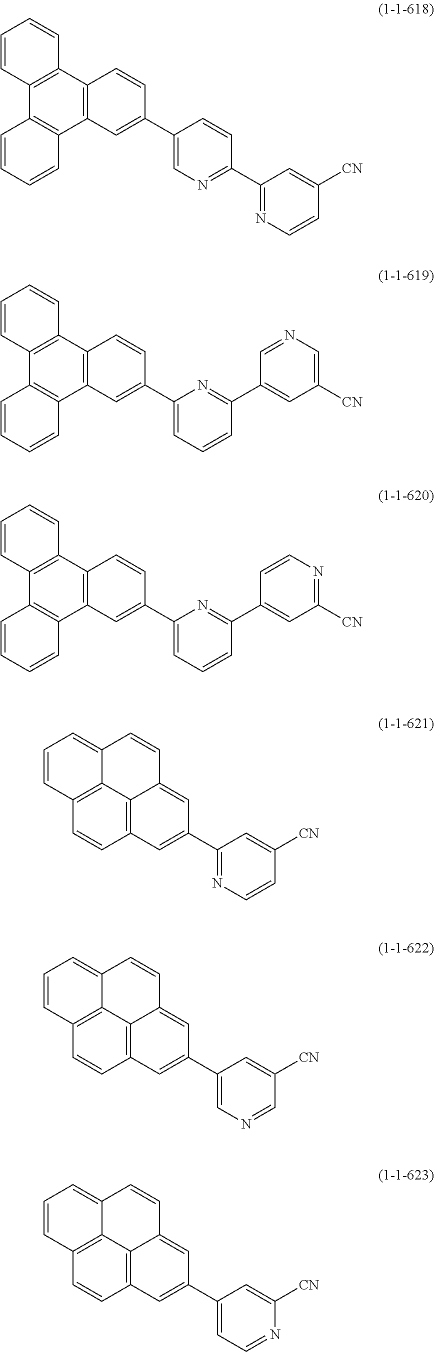 [SMILES: N#Cc1ccnc(-c2ccc(-c3ccc4c5ccccc5c5ccccc5c4c3)cn2)c1 ; N#Cc1cncc(-c2cccc(-c3ccc4c5ccccc5c5ccccc5c4c3)n2)c1 ; N#Cc1cc(-c2cccc(-c3ccc4c5ccccc5c5ccccc5c4c3)n2)ccn1 ; N#Cc1ccnc(-c2cc3ccc4cccc5ccc(c2)c3c45)c1 ; N#Cc1cncc(-c2cc3ccc4cccc5ccc(c2)c3c45)c1 ; N#Cc1cc(-c2cc3ccc4cccc5ccc(c2)c3c45)ccn1]

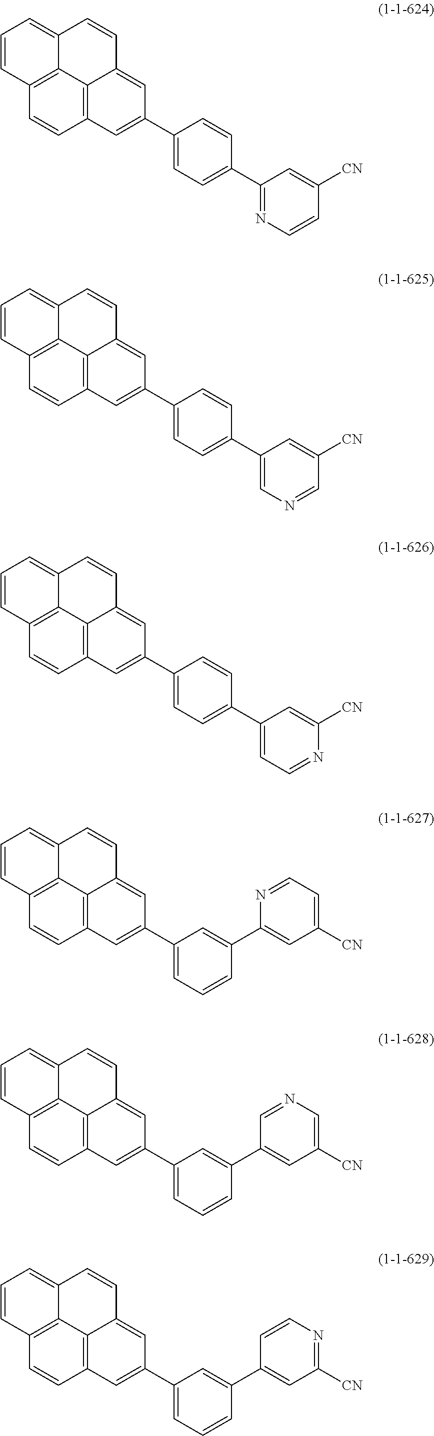 [SMILES: N#Cc1ccnc(-c2ccc(-c3cc4ccc5cccc6ccc(c3)c4c56)cc2)c1 ; N#Cc1cncc(-c2ccc(-c3cc4ccc5cccc6ccc(c3)c4c56)cc2)c1 ; N#Cc1cc(-c2ccc(-c3cc4ccc5cccc6ccc(c3)c4c56)cc2)ccn1 ; N#Cc1ccnc(-c2cccc(-c3cc4ccc5cccc6ccc(c3)c4c56)c2)c1 ; N#Cc1cncc(-c2cccc(-c3cc4ccc5cccc6ccc(c3)c4c56)c2)c1 ; N#Cc1cc(-c2cccc(-c3cc4ccc5cccc6ccc(c3)c4c56)c2)ccn1]

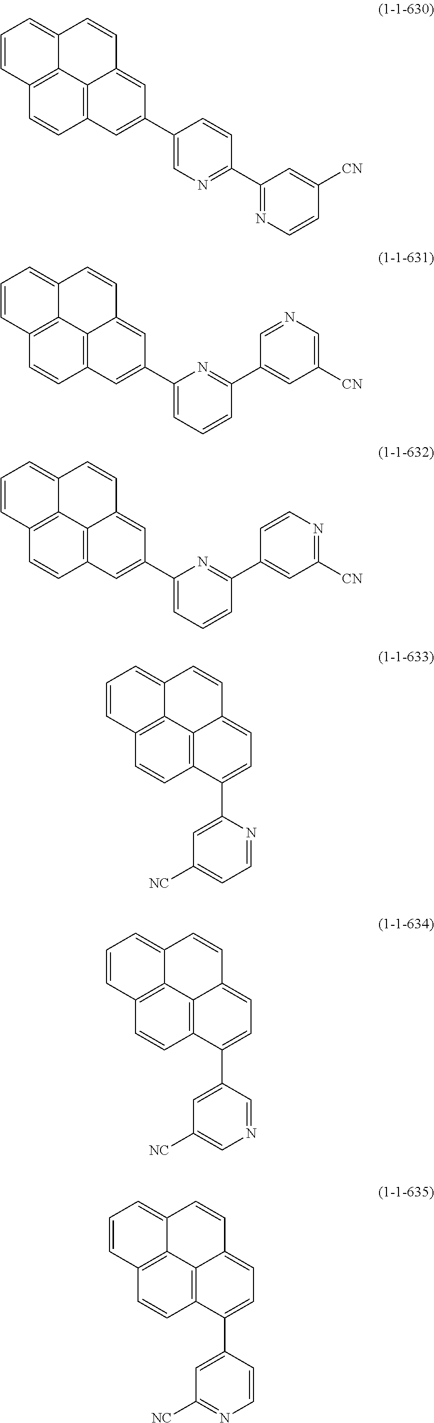

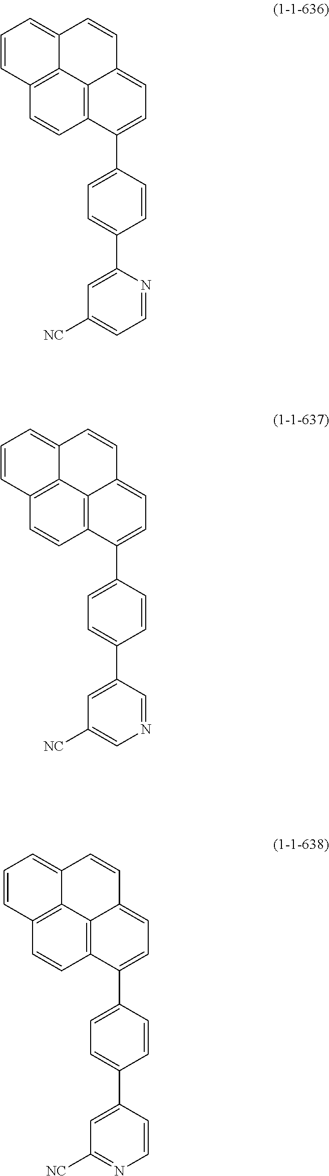 [SMILES: N#Cc1ccnc(-c2ccc(-c3ccc4ccc5cccc6ccc3c4c56)cc2)c1 ; N#Cc1cncc(-c2ccc(-c3ccc4ccc5cccc6ccc3c4c56)cc2)c1 ; N#Cc1cc(-c2ccc(-c3ccc4ccc5cccc6ccc3c4c56)cc2)ccn1]

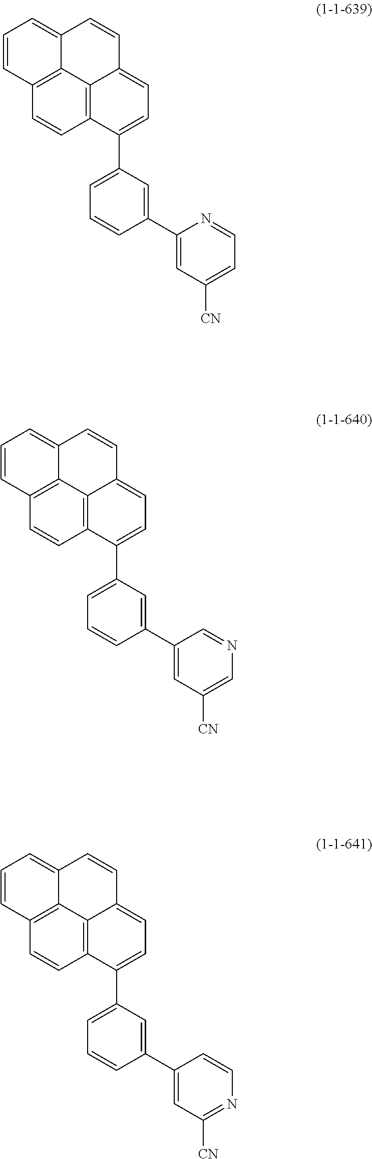 [SMILES: N#Cc1ccnc(-c2cccc(-c3ccc4ccc5cccc6ccc3c4c56)c2)c1 ; N#Cc1cncc(-c2cccc(-c3ccc4ccc5cccc6ccc3c4c56)c2)c1 ; N#Cc1cc(-c2cccc(-c3ccc4ccc5cccc6ccc3c4c56)c2)ccn1]

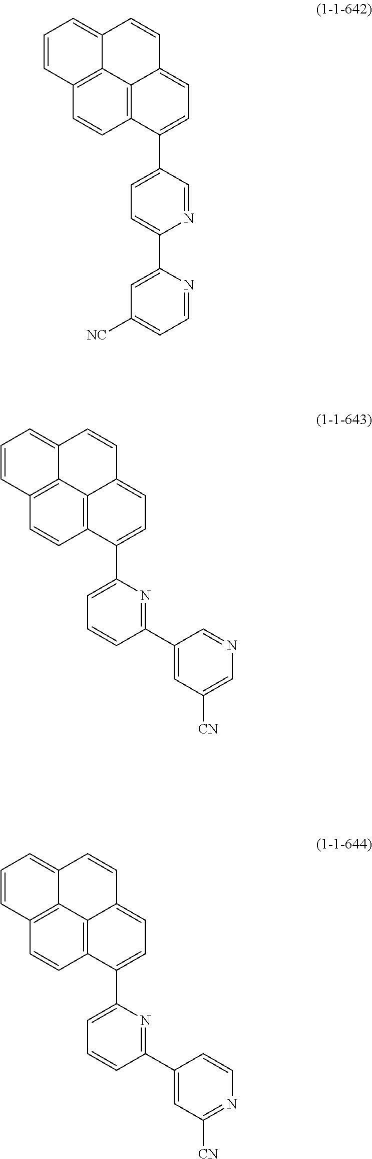 [SMILES: N#Cc1ccnc(-c2ccc(-c3ccc4ccc5cccc6ccc3c4c56)cn2)c1 ; N#Cc1cncc(-c2cccc(-c3ccc4ccc5cccc6ccc3c4c56)n2)c1 ; N#Cc1cc(-c2cccc(-c3ccc4ccc5cccc6ccc3c4c56)n2)ccn1]

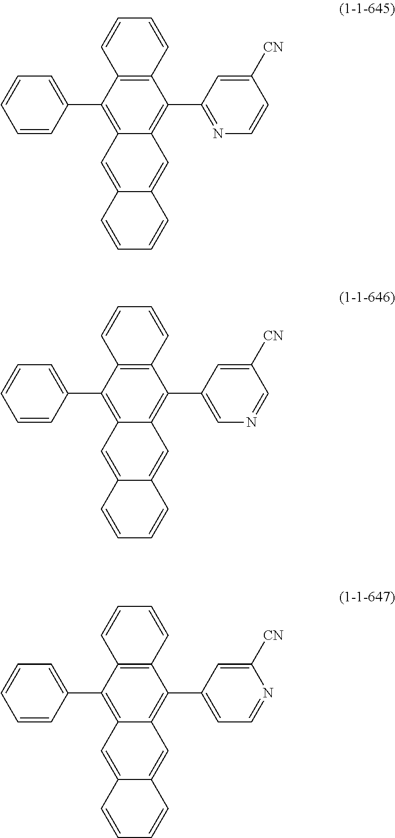 [SMILES: N#Cc1ccnc(-c2c3ccccc3c(-c3ccccc3)c3cc4ccccc4cc23)c1 ; N#Cc1cncc(-c2c3ccccc3c(-c3ccccc3)c3cc4ccccc4cc23)c1 ; N#Cc1cc(-c2c3ccccc3c(-c3ccccc3)c3cc4ccccc4cc23)ccn1]

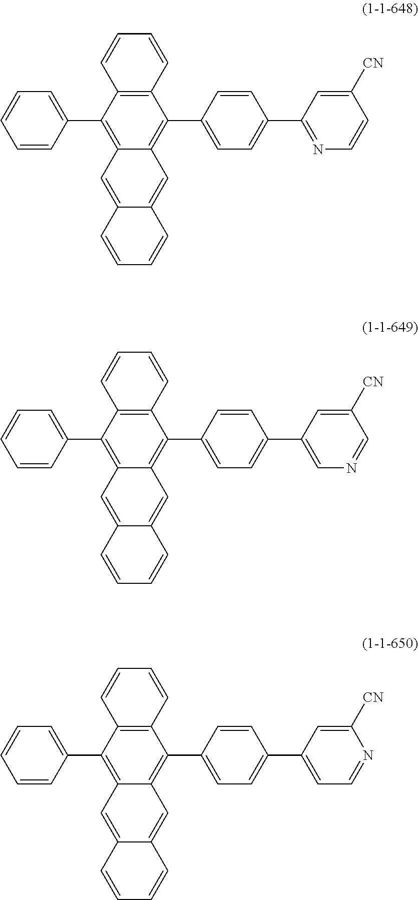 [SMILES: N#Cc1ccnc(-c2ccc(-c3c4ccccc4c(-c4ccccc4)c4cc5ccccc5cc34)cc2)c1 ; N#Cc1cncc(-c2ccc(-c3c4ccccc4c(-c4ccccc4)c4cc5ccccc5cc34)cc2)c1 ; N#Cc1cc(-c2ccc(-c3c4ccccc4c(-c4ccccc4)c4cc5ccccc5cc34)cc2)ccn1]

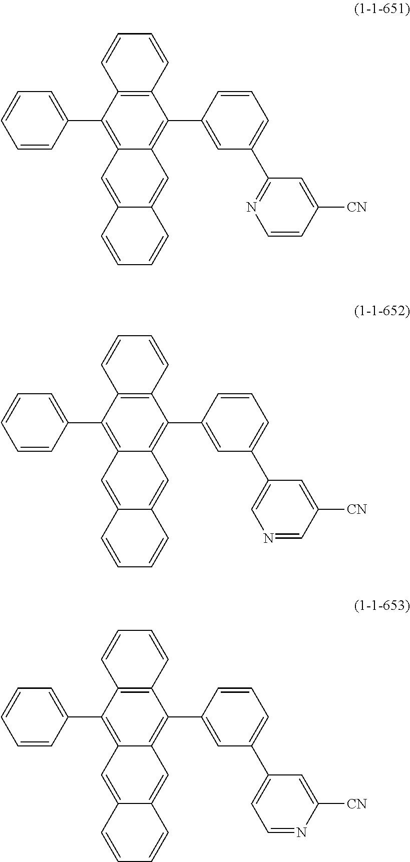 [SMILES: N#Cc1ccnc(-c2cccc(-c3c4ccccc4c(-c4ccccc4)c4cc5ccccc5cc34)c2)c1 ; N#Cc1cncc(-c2cccc(-c3c4ccccc4c(-c4ccccc4)c4cc5ccccc5cc34)c2)c1 ; N#Cc1cc(-c2cccc(-c3c4ccccc4c(-c4ccccc4)c4cc5ccccc5cc34)c2)ccn1]

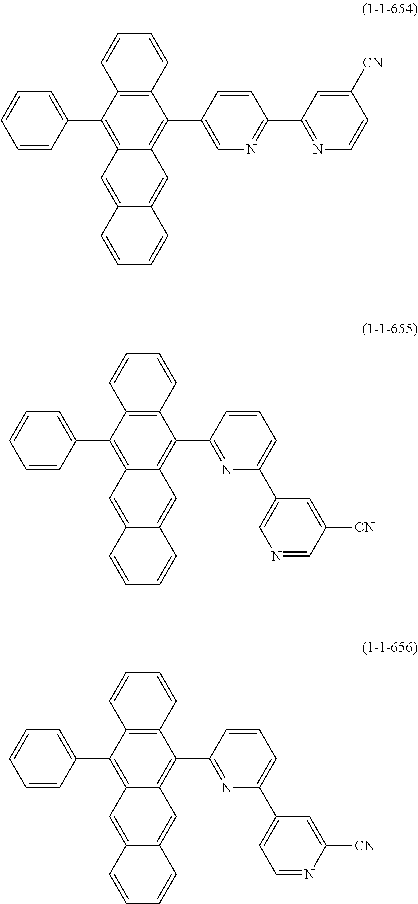 [SMILES: N#Cc1ccnc(-c2ccc(-c3c4ccccc4c(-c4ccccc4)c4cc5ccccc5cc34)cn2)c1 ; N#Cc1cncc(-c2cccc(-c3c4ccccc4c(-c4ccccc4)c4cc5ccccc5cc34)n2)c1 ; N#Cc1cc(-c2cccc(-c3c4ccccc4c(-c4ccccc4)c4cc5ccccc5cc34)n2)ccn1]

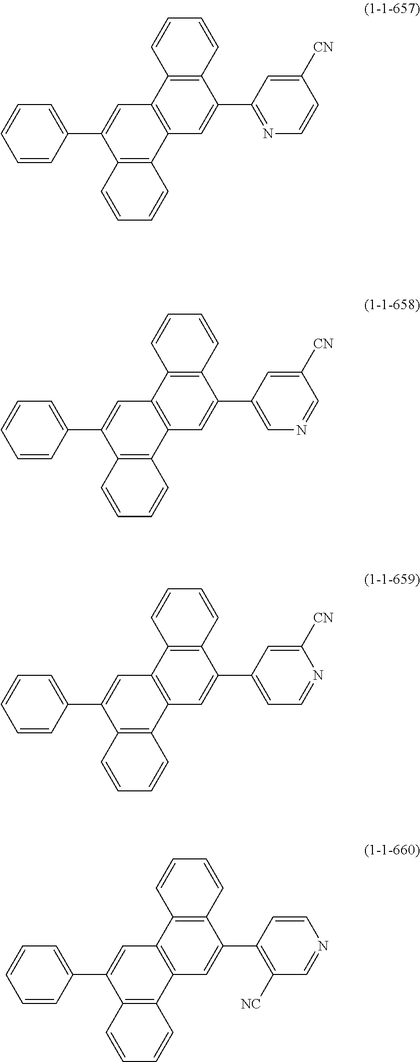 [SMILES: N#Cc1ccnc(-c2cc3c4ccccc4c(-c4ccccc4)cc3c3ccccc23)c1 ; N#Cc1cncc(-c2cc3c4ccccc4c(-c4ccccc4)cc3c3ccccc23)c1 ; N#Cc1cc(-c2cc3c4ccccc4c(-c4ccccc4)cc3c3ccccc23)ccn1 ; N#Cc1cnccc1-c1cc2c3ccccc3c(-c3ccccc3)cc2c2ccccc12]

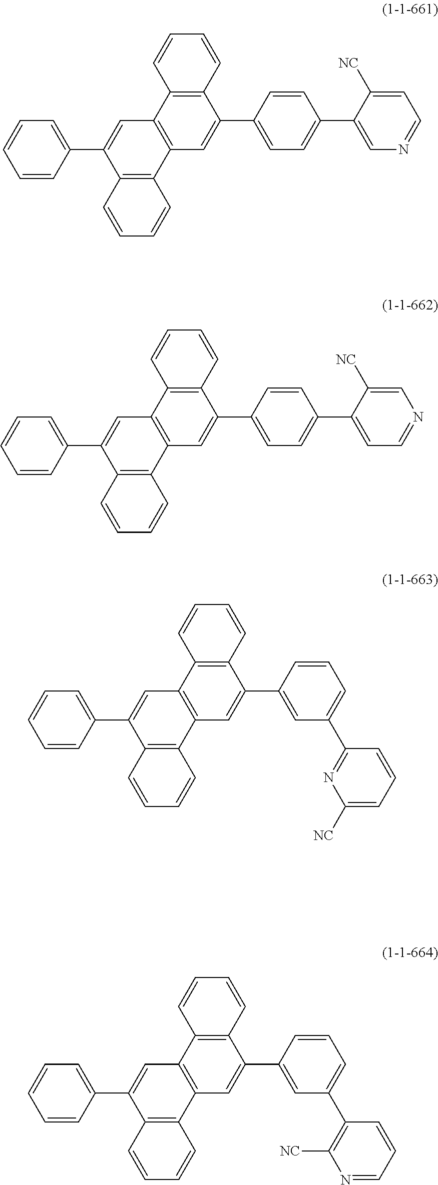 [SMILES: N#Cc1ccncc1-c1ccc(-c2cc3c4ccccc4c(-c4ccccc4)cc3c3ccccc23)cc1 ; N#Cc1cnccc1-c1ccc(-c2cc3c4ccccc4c(-c4ccccc4)cc3c3ccccc23)cc1 ; N#Cc1cccc(-c2cccc(-c3cc4c5ccccc5c(-c5ccccc5)cc4c4ccccc34)c2)n1 ; N#Cc1ncccc1-c1cccc(-c2cc3c4ccccc4c(-c4ccccc4)cc3c3ccccc23)c1]

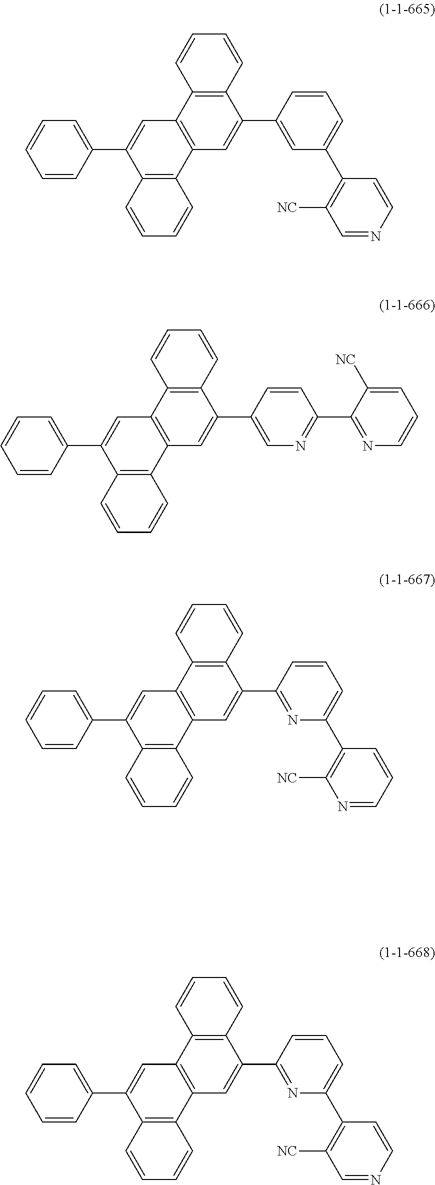 [SMILES: N#Cc1cnccc1-c1cccc(-c2cc3c4ccccc4c(-c4ccccc4)cc3c3ccccc23)c1 ; N#Cc1cccnc1-c1ccc(-c2cc3c4ccccc4c(-c4ccccc4)cc3c3ccccc23)cn1 ; N#Cc1ncccc1-c1cccc(-c2cc3c4ccccc4c(-c4ccccc4)cc3c3ccccc23)n1 ; N#Cc1cnccc1-c1cccc(-c2cc3c4ccccc4c(-c4ccccc4)cc3c3ccccc23)n1]

Specific examples of the compound represented by formula (1) when m=2 are shown in the following formulae (1-2-1) to (1-2-515).

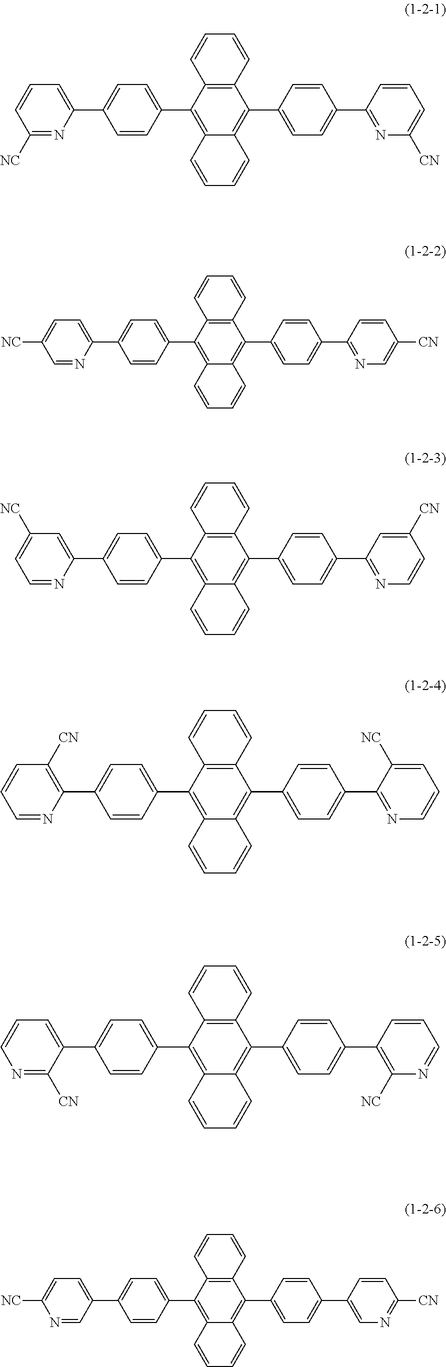 [SMILES: N#Cc1cccc(-c2ccc(-c3c4ccccc4c(-c4ccc(-c5cccc(C#N)n5)cc4)c4ccccc34)cc2)n1 ; N#Cc1ccc(-c2ccc(-c3c4ccccc4c(-c4ccc(-c5ccc(C#N)cn5)cc4)c4ccccc34)cc2)nc1 ; N#Cc1ccnc(-c2ccc(-c3c4ccccc4c(-c4ccc(-c5cc(C#N)ccn5)cc4)c4ccccc34)cc2)c1 ; N#Cc1cccnc1-c1ccc(-c2c3ccccc3c(-c3ccc(-c4ncccc4C#N)cc3)c3ccccc23)cc1 ; N#Cc1ncccc1-c1ccc(-c2c3ccccc3c(-c3ccc(-c4cccnc4C#N)cc3)c3ccccc23)cc1 ; N#Cc1ccc(-c2ccc(-c3c4ccccc4c(-c4ccc(-c5ccc(C#N)nc5)cc4)c4ccccc34)cc2)cn1]

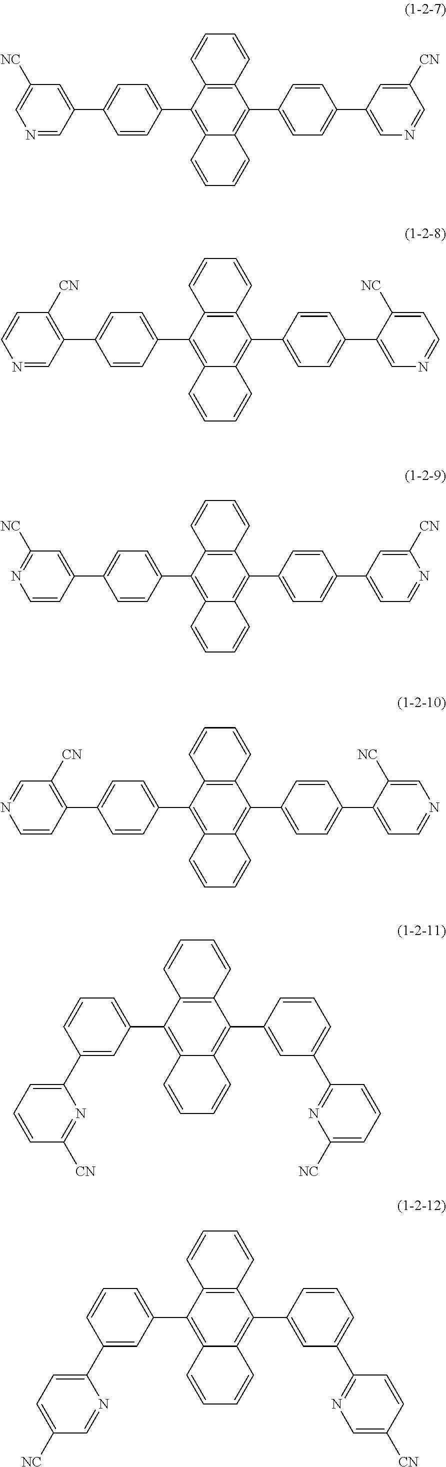 [SMILES: N#Cc1cncc(-c2ccc(-c3c4ccccc4c(-c4ccc(-c5cncc(C#N)c5)cc4)c4ccccc34)cc2)c1 ; N#Cc1ccncc1-c1ccc(-c2c3ccccc3c(-c3ccc(-c4cnccc4C#N)cc3)c3ccccc23)cc1 ; N#Cc1cc(-c2ccc(-c3c4ccccc4c(-c4ccc(-c5ccnc(C#N)c5)cc4)c4ccccc34)cc2)ccn1 ; N#Cc1cnccc1-c1ccc(-c2c3ccccc3c(-c3ccc(-c4ccncc4C#N)cc3)c3ccccc23)cc1 ; N#Cc1cccc(-c2cccc(-c3c4ccccc4c(-c4cccc(-c5cccc(C#N)n5)c4)c4ccccc34)c2)n1 ; N#Cc1ccc(-c2cccc(-c3c4ccccc4c(-c4cccc(-c5ccc(C#N)cn5)c4)c4ccccc34)c2)nc1]

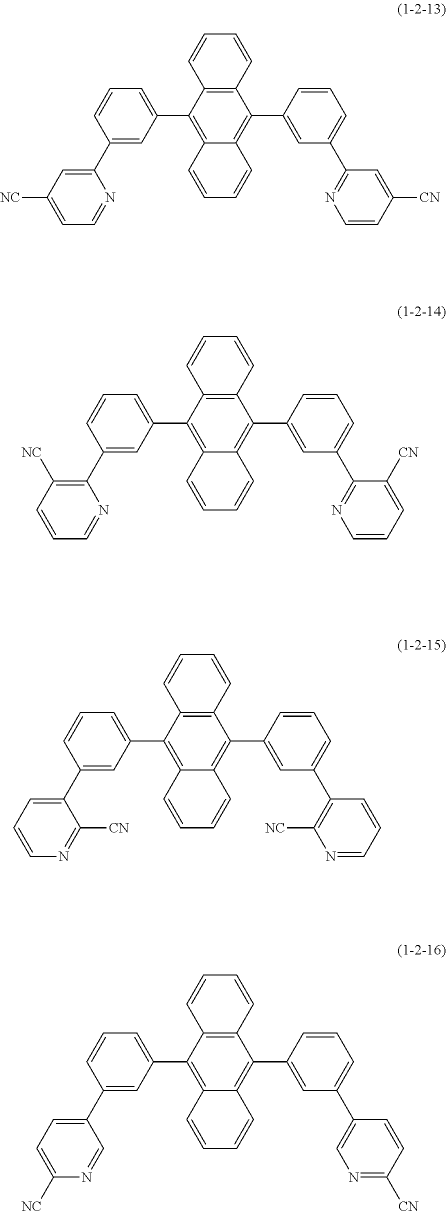 [SMILES: N#Cc1ccnc(-c2cccc(-c3c4ccccc4c(-c4cccc(-c5cc(C#N)ccn5)c4)c4ccccc34)c2)c1 ; N#Cc1cccnc1-c1cccc(-c2c3ccccc3c(-c3cccc(-c4ncccc4C#N)c3)c3ccccc23)c1 ; N#Cc1ncccc1-c1cccc(-c2c3ccccc3c(-c3cccc(-c4cccnc4C#N)c3)c3ccccc23)c1 ; N#Cc1ccc(-c2cccc(-c3c4ccccc4c(-c4cccc(-c5ccc(C#N)nc5)c4)c4ccccc34)c2)cn1]

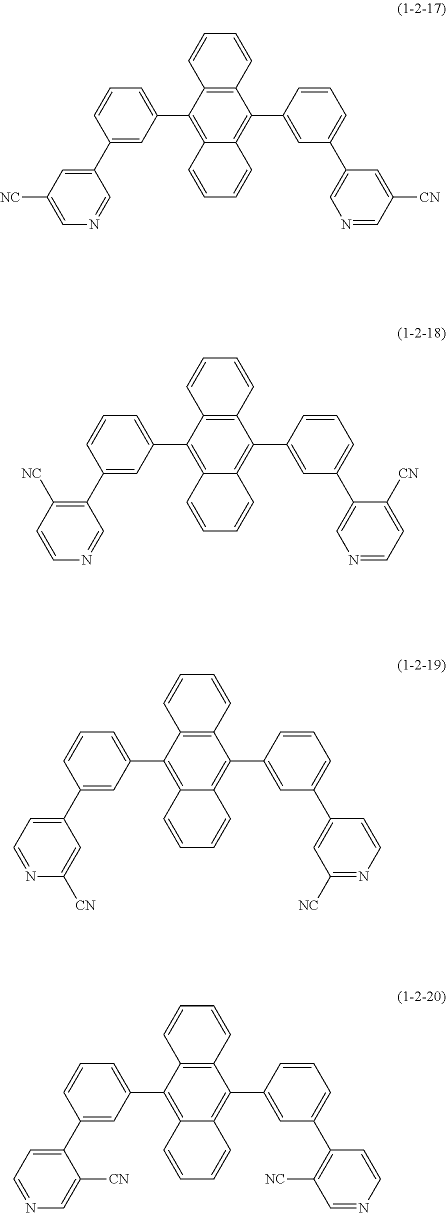 [SMILES: N#Cc1cncc(-c2cccc(-c3c4ccccc4c(-c4cccc(-c5cncc(C#N)c5)c4)c4ccccc34)c2)c1 ; N#Cc1ccncc1-c1cccc(-c2c3ccccc3c(-c3cccc(-c4cnccc4C#N)c3)c3ccccc23)c1 ; N#Cc1cc(-c2cccc(-c3c4ccccc4c(-c4cccc(-c5ccnc(C#N)c5)c4)c4ccccc34)c2)ccn1 ; N#Cc1cnccc1-c1cccc(-c2c3ccccc3c(-c3cccc(-c4ccncc4C#N)c3)c3ccccc23)c1]

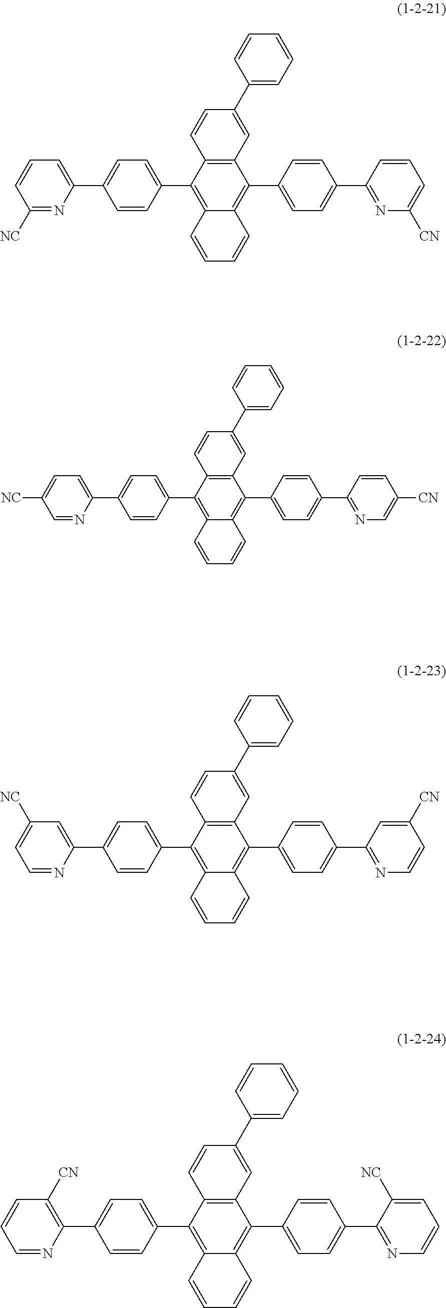 [SMILES: N#Cc1cccc(-c2ccc(-c3c4ccccc4c(-c4ccc(-c5cccc(C#N)n5)cc4)c4cc(-c5ccccc5)ccc34)cc2)n1 ; N#Cc1ccc(-c2ccc(-c3c4ccccc4c(-c4ccc(-c5ccc(C#N)cn5)cc4)c4cc(-c5ccccc5)ccc34)cc2)nc1 ; N#Cc1ccnc(-c2ccc(-c3c4ccccc4c(-c4ccc(-c5cc(C#N)ccn5)cc4)c4cc(-c5ccccc5)ccc34)cc2)c1 ; N#Cc1cccnc1-c1ccc(-c2c3ccccc3c(-c3ccc(-c4ncccc4C#N)cc3)c3cc(-c4ccccc4)ccc23)cc1]

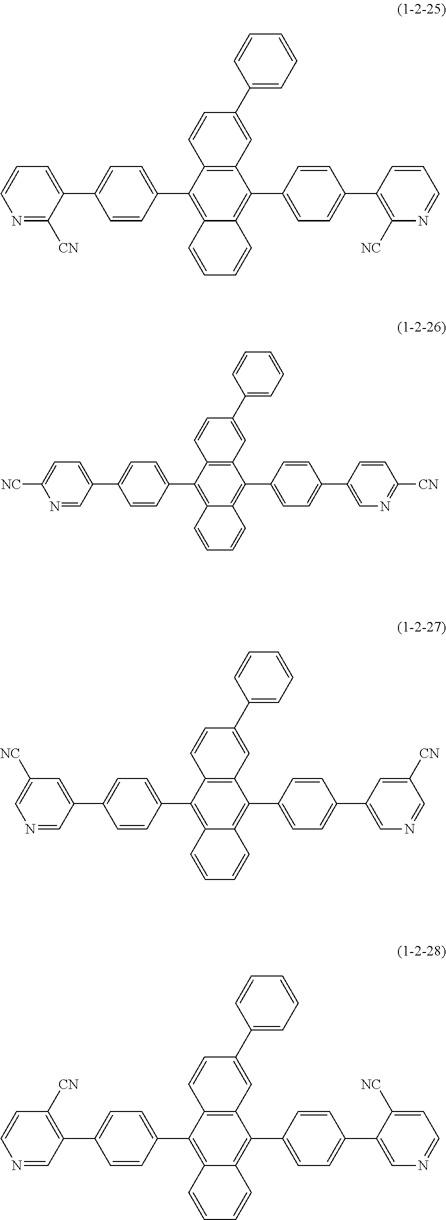 [SMILES: N#Cc1ncccc1-c1ccc(-c2c3ccccc3c(-c3ccc(-c4cccnc4C#N)cc3)c3cc(-c4ccccc4)ccc23)cc1 ; N#Cc1ccc(-c2ccc(-c3c4ccccc4c(-c4ccc(-c5ccc(C#N)nc5)cc4)c4cc(-c5ccccc5)ccc34)cc2)cn1 ; N#Cc1cncc(-c2ccc(-c3c4ccccc4c(-c4ccc(-c5cncc(C#N)c5)cc4)c4cc(-c5ccccc5)ccc34)cc2)c1 ; N#Cc1ccncc1-c1ccc(-c2c3ccccc3c(-c3ccc(-c4cnccc4C#N)cc3)c3cc(-c4ccccc4)ccc23)cc1]

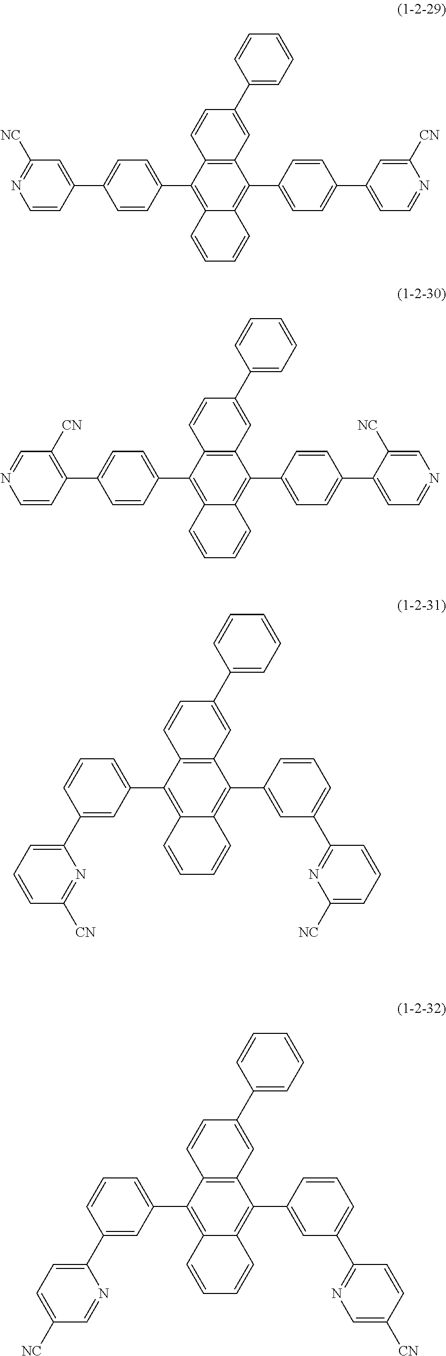 [SMILES: N#Cc1cc(-c2ccc(-c3c4ccccc4c(-c4ccc(-c5ccnc(C#N)c5)cc4)c4cc(-c5ccccc5)ccc34)cc2)ccn1 ; N#Cc1cnccc1-c1ccc(-c2c3ccccc3c(-c3ccc(-c4ccncc4C#N)cc3)c3cc(-c4ccccc4)ccc23)cc1 ; N#Cc1cccc(-c2cccc(-c3c4ccccc4c(-c4cccc(-c5cccc(C#N)n5)c4)c4cc(-c5ccccc5)ccc34)c2)n1 ; N#Cc1ccc(-c2cccc(-c3c4ccccc4c(-c4cccc(-c5ccc(C#N)cn5)c4)c4cc(-c5ccccc5)ccc34)c2)nc1]

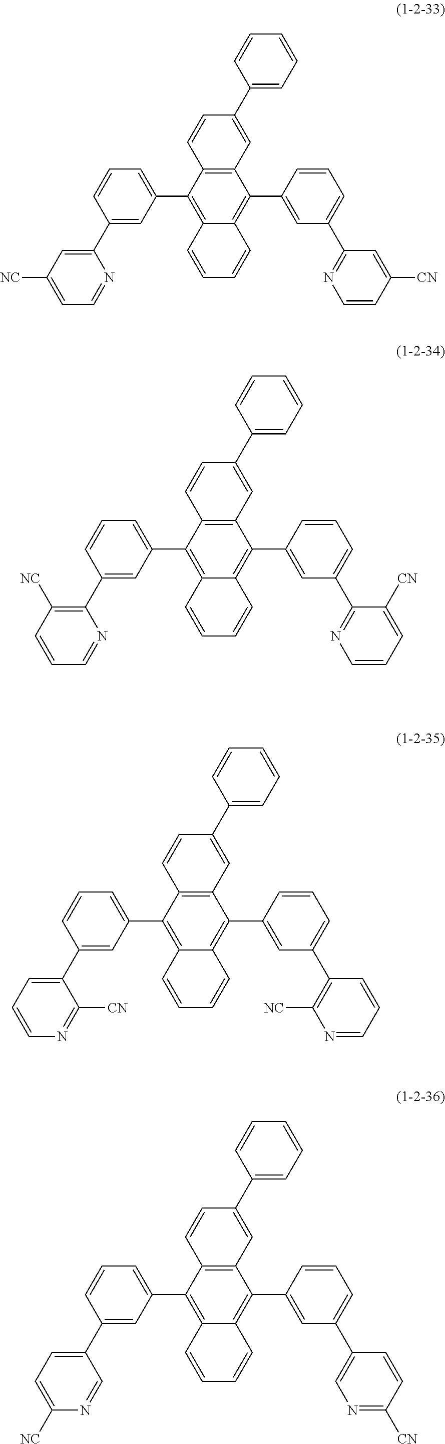 [SMILES: N#Cc1ccnc(-c2cccc(-c3c4ccccc4c(-c4cccc(-c5cc(C#N)ccn5)c4)c4cc(-c5ccccc5)ccc34)c2)c1 ; N#Cc1cccnc1-c1cccc(-c2c3ccccc3c(-c3cccc(-c4ncccc4C#N)c3)c3cc(-c4ccccc4)ccc23)c1 ; N#Cc1ncccc1-c1cccc(-c2c3ccccc3c(-c3cccc(-c4cccnc4C#N)c3)c3cc(-c4ccccc4)ccc23)c1 ; N#Cc1ccc(-c2cccc(-c3c4ccccc4c(-c4cccc(-c5ccc(C#N)nc5)c4)c4cc(-c5ccccc5)ccc34)c2)cn1]

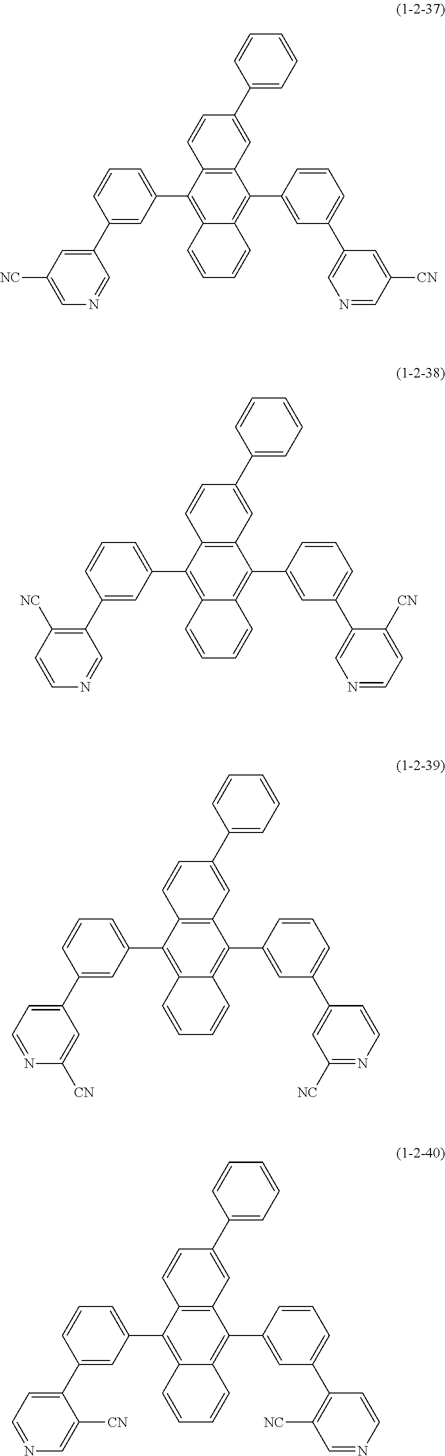 [SMILES: N#Cc1cncc(-c2cccc(-c3c4ccccc4c(-c4cccc(-c5cncc(C#N)c5)c4)c4cc(-c5ccccc5)ccc34)c2)c1 ; N#Cc1ccncc1-c1cccc(-c2c3ccccc3c(-c3cccc(-c4cnccc4C#N)c3)c3cc(-c4ccccc4)ccc23)c1 ; N#Cc1cc(-c2cccc(-c3c4ccccc4c(-c4cccc(-c5ccnc(C#N)c5)c4)c4cc(-c5ccccc5)ccc34)c2)ccn1 ; N#Cc1cnccc1-c1cccc(-c2c3ccccc3c(-c3cccc(-c4ccncc4C#N)c3)c3cc(-c4ccccc4)ccc23)c1]

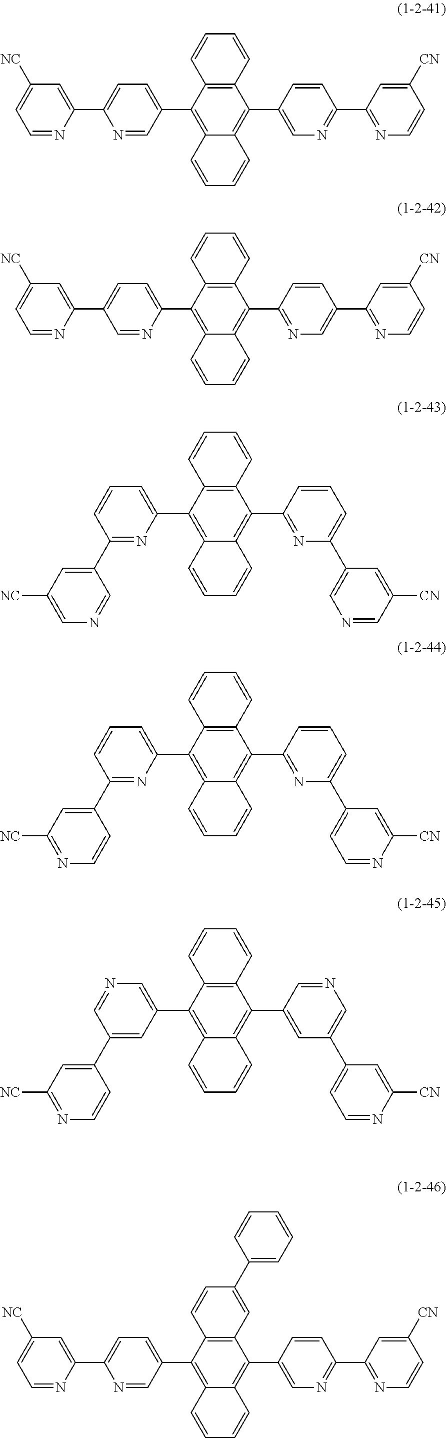 [SMILES: N#Cc1ccnc(-c2ccc(-c3c4ccccc4c(-c4ccc(-c5cc(C#N)ccn5)nc4)c4ccccc34)cn2)c1 ; N#Cc1ccnc(-c2ccc(-c3c4ccccc4c(-c4ccc(-c5cc(C#N)ccn5)cn4)c4ccccc34)nc2)c1 ; N#Cc1cncc(-c2cccc(-c3c4ccccc4c(-c4cccc(-c5cncc(C#N)c5)n4)c4ccccc34)n2)c1 ; N#Cc1cc(-c2cccc(-c3c4ccccc4c(-c4cccc(-c5ccnc(C#N)c5)n4)c4ccccc34)n2)ccn1 ; N#Cc1cc(-c2cncc(-c3c4ccccc4c(-c4cncc(-c5ccnc(C#N)c5)c4)c4ccccc34)c2)ccn1 ; N#Cc1ccnc(-c2ccc(-c3c4ccccc4c(-c4ccc(-c5cc(C#N)ccn5)nc4)c4cc(-c5ccccc5)ccc34)cn2)c1]

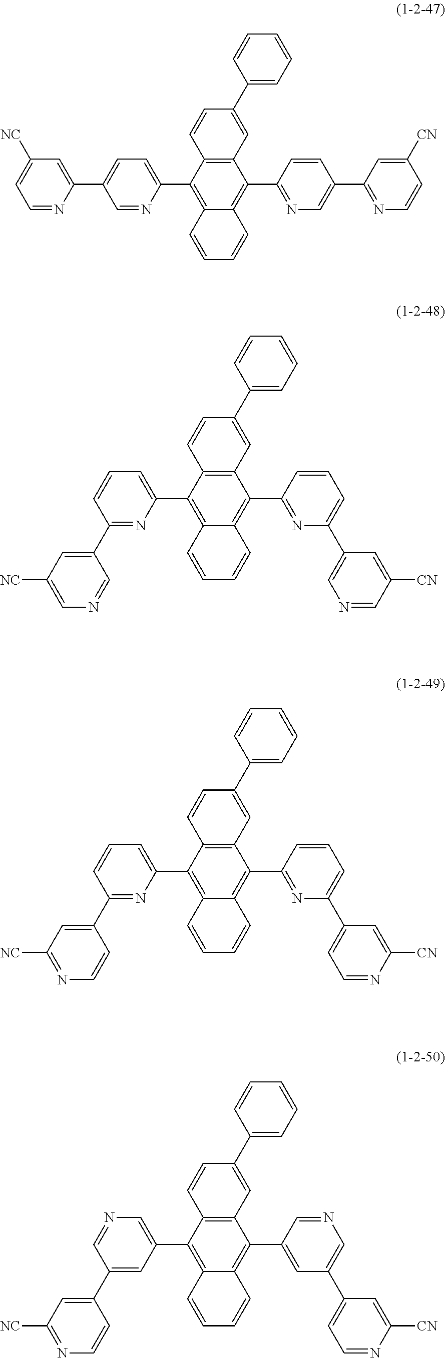 [SMILES: N#Cc1ccnc(-c2ccc(-c3c4ccccc4c(-c4ccc(-c5cc(C#N)ccn5)cn4)c4cc(-c5ccccc5)ccc34)nc2)c1 ; N#Cc1cncc(-c2cccc(-c3c4ccccc4c(-c4cccc(-c5cncc(C#N)c5)n4)c4cc(-c5ccccc5)ccc34)n2)c1 ; N#Cc1cc(-c2cccc(-c3c4ccccc4c(-c4cccc(-c5ccnc(C#N)c5)n4)c4cc(-c5ccccc5)ccc34)n2)ccn1 ; N#Cc1cc(-c2cncc(-c3c4ccccc4c(-c4cncc(-c5ccnc(C#N)c5)c4)c4cc(-c5ccccc5)ccc34)c2)ccn1]

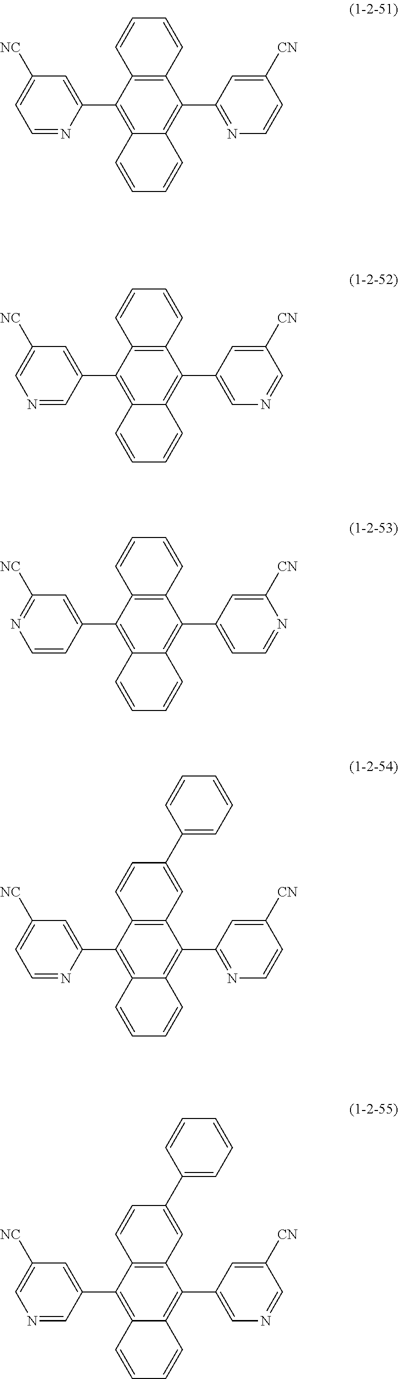 [SMILES: N#Cc1ccnc(-c2c3ccccc3c(-c3cc(C#N)ccn3)c3ccccc23)c1 ; N#Cc1cncc(-c2c3ccccc3c(-c3cncc(C#N)c3)c3ccccc23)c1 ; N#Cc1cc(-c2c3ccccc3c(-c3ccnc(C#N)c3)c3ccccc23)ccn1 ; N#Cc1ccnc(-c2c3ccccc3c(-c3cc(C#N)ccn3)c3cc(-c4ccccc4)ccc23)c1 ; N#Cc1cncc(-c2c3ccccc3c(-c3cncc(C#N)c3)c3cc(-c4ccccc4)ccc23)c1]

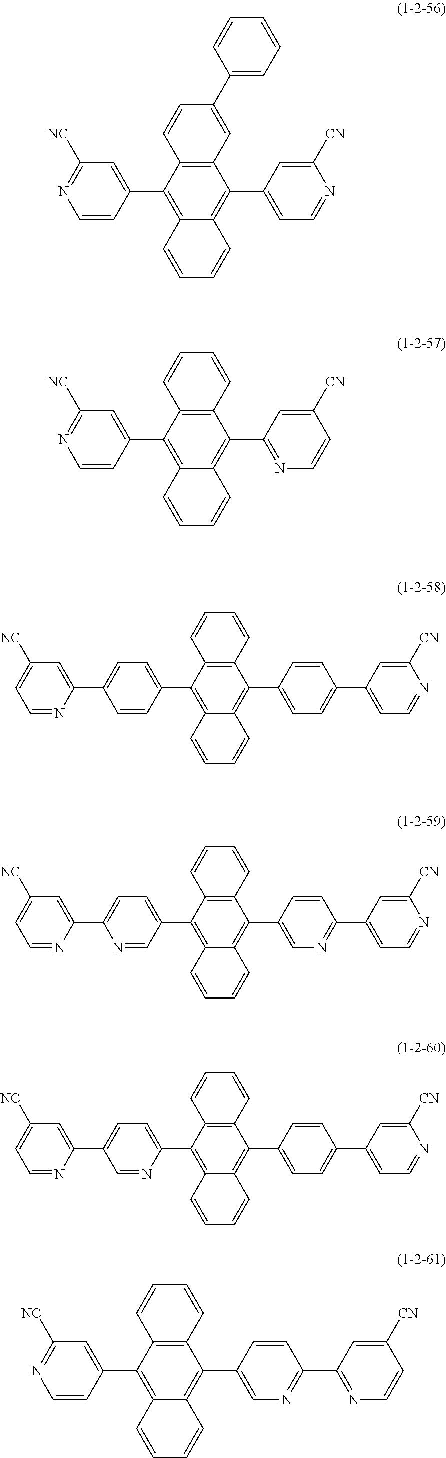 [SMILES: N#Cc1cc(-c2c3ccccc3c(-c3ccnc(C#N)c3)c3cc(-c4ccccc4)ccc23)ccn1 ; N#Cc1ccnc(-c2c3ccccc3c(-c3ccnc(C#N)c3)c3ccccc23)c1 ; N#Cc1ccnc(-c2ccc(-c3c4ccccc4c(-c4ccc(-c5ccnc(C#N)c5)cc4)c4ccccc34)cc2)c1 ; N#Cc1ccnc(-c2ccc(-c3c4ccccc4c(-c4ccc(-c5ccnc(C#N)c5)nc4)c4ccccc34)cn2)c1 ; N#Cc1ccnc(-c2ccc(-c3c4ccccc4c(-c4ccc(-c5ccnc(C#N)c5)cc4)c4ccccc34)nc2)c1 ; N#Cc1ccnc(-c2ccc(-c3c4ccccc4c(-c4ccnc(C#N)c4)c4ccccc34)cn2)c1]

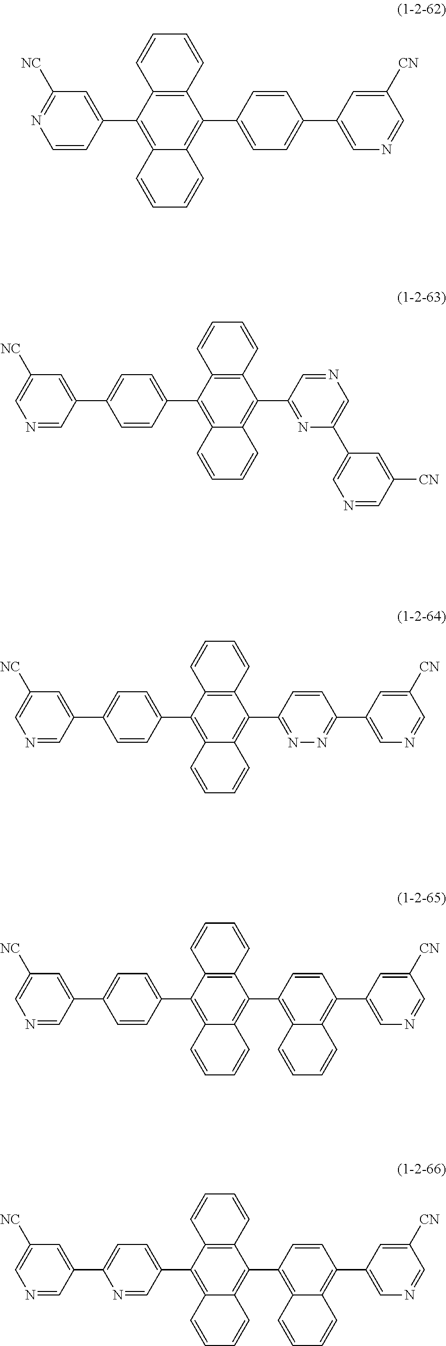 [SMILES: N#Cc1cncc(-c2ccc(-c3c4ccccc4c(-c4ccnc(C#N)c4)c4ccccc34)cc2)c1 ; N#Cc1cncc(-c2ccc(-c3c4ccccc4c(-c4cncc(-c5cncc(C#N)c5)n4)c4ccccc34)cc2)c1 ; N#Cc1cncc(-c2ccc(-c3c4ccccc4c(-c4ccc(-c5cncc(C#N)c5)nn4)c4ccccc34)cc2)c1 ; N#Cc1cncc(-c2ccc(-c3c4ccccc4c(-c4ccc(-c5cncc(C#N)c5)c5ccccc45)c4ccccc34)cc2)c1 ; N#Cc1cncc(-c2ccc(-c3c4ccccc4c(-c4ccc(-c5cncc(C#N)c5)c5ccccc45)c4ccccc34)cn2)c1]

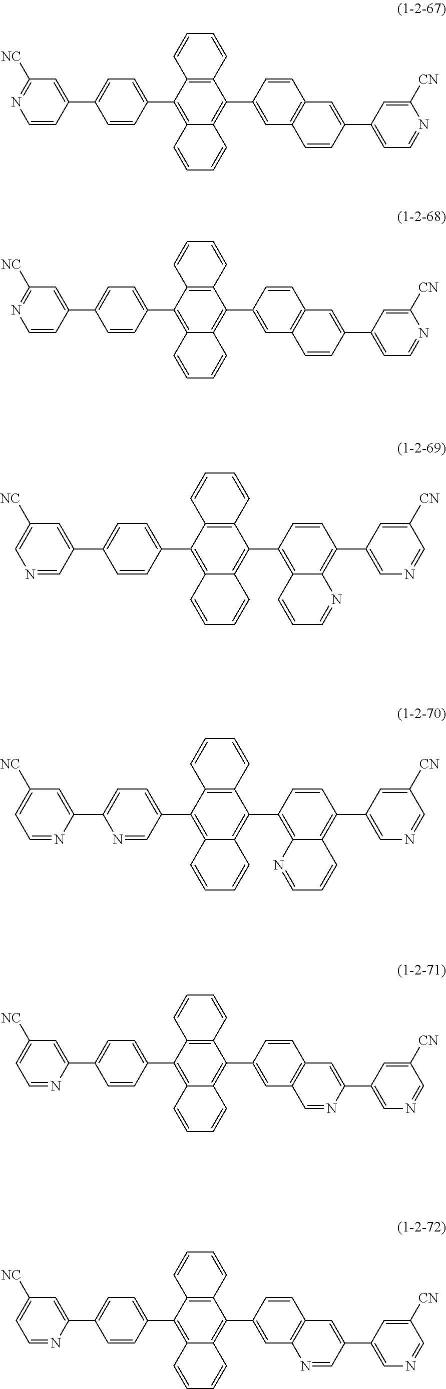 [SMILES: N#Cc1cc(-c2ccc(-c3c4ccccc4c(-c4ccc5cc(-c6ccnc(C#N)c6)ccc5c4)c4ccccc34)cc2)ccn1 ; N#Cc1cc(-c2ccc(-c3c4ccccc4c(-c4ccc5cc(-c6ccnc(C#N)c6)ccc5c4)c4ccccc34)cc2)ccn1 ; N#Cc1cncc(-c2ccc(-c3c4ccccc4c(-c4ccc(-c5cncc(C#N)c5)c5ncccc45)c4ccccc34)cc2)c1 ; N#Cc1cncc(-c2ccc(-c3c4ccccc4c(-c4ccc(-c5cc(C#N)ccn5)nc4)c4ccccc34)c3ncccc23)c1 ; N#Cc1cncc(-c2cc3ccc(-c4c5ccccc5c(-c5ccc(-c6cc(C#N)ccn6)cc5)c5ccccc45)cc3cn2)c1 ; N#Cc1cncc(-c2cnc3cc(-c4c5ccccc5c(-c5ccc(-c6cc(C#N)ccn6)cc5)c5ccccc45)ccc3c2)c1]

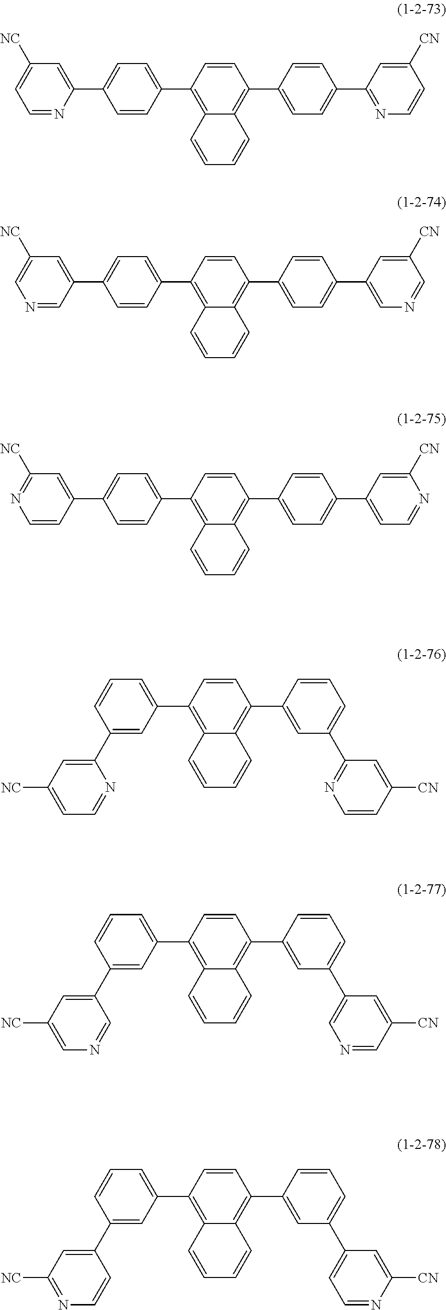 [SMILES: N#Cc1ccnc(-c2ccc(-c3ccc(-c4ccc(-c5cc(C#N)ccn5)cc4)c4ccccc34)cc2)c1 ; N#Cc1cncc(-c2ccc(-c3ccc(-c4ccc(-c5cncc(C#N)c5)cc4)c4ccccc34)cc2)c1 ; N#Cc1cc(-c2ccc(-c3ccc(-c4ccc(-c5ccnc(C#N)c5)cc4)c4ccccc34)cc2)ccn1 ; N#Cc1ccnc(-c2cccc(-c3ccc(-c4cccc(-c5cc(C#N)ccn5)c4)c4ccccc34)c2)c1 ; N#Cc1cncc(-c2cccc(-c3ccc(-c4cccc(-c5cncc(C#N)c5)c4)c4ccccc34)c2)c1 ; N#Cc1cc(-c2cccc(-c3ccc(-c4cccc(-c5ccnc(C#N)c5)c4)c4ccccc34)c2)ccn1]

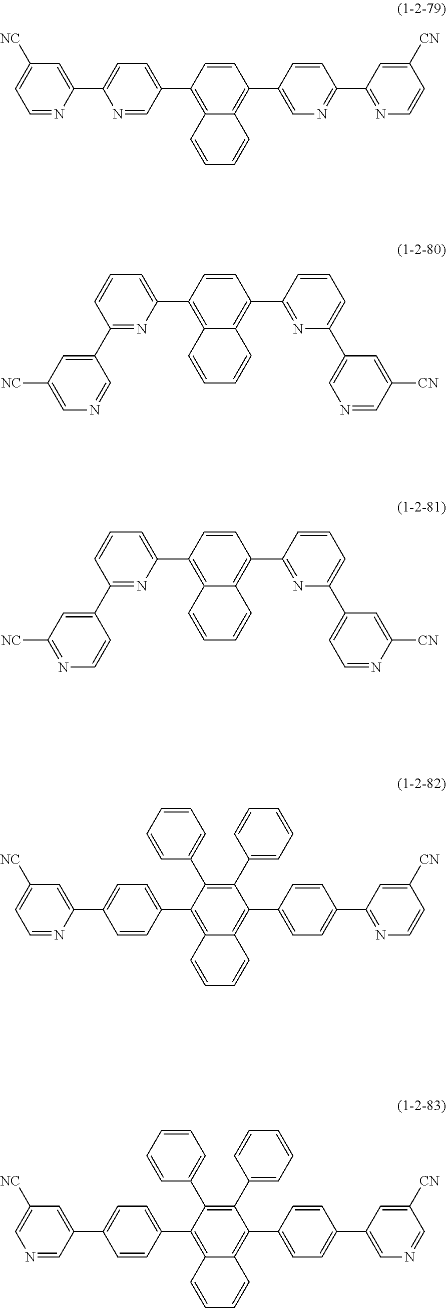 [SMILES: N#Cc1ccnc(-c2ccc(-c3ccc(-c4ccc(-c5cc(C#N)ccn5)nc4)c4ccccc34)cn2)c1 ; N#Cc1cncc(-c2cccc(-c3ccc(-c4cccc(-c5cncc(C#N)c5)n4)c4ccccc34)n2)c1 ; N#Cc1cc(-c2cccc(-c3ccc(-c4cccc(-c5ccnc(C#N)c5)n4)c4ccccc34)n2)ccn1 ; N#Cc1ccnc(-c2ccc(-c3c(-c4ccccc4)c(-c4ccccc4)c(-c4ccc(-c5cc(C#N)ccn5)cc4)c4ccccc34)cc2)c1 ; N#Cc1cncc(-c2ccc(-c3c(-c4ccccc4)c(-c4ccccc4)c(-c4ccc(-c5cncc(C#N)c5)cc4)c4ccccc34)cc2)c1]

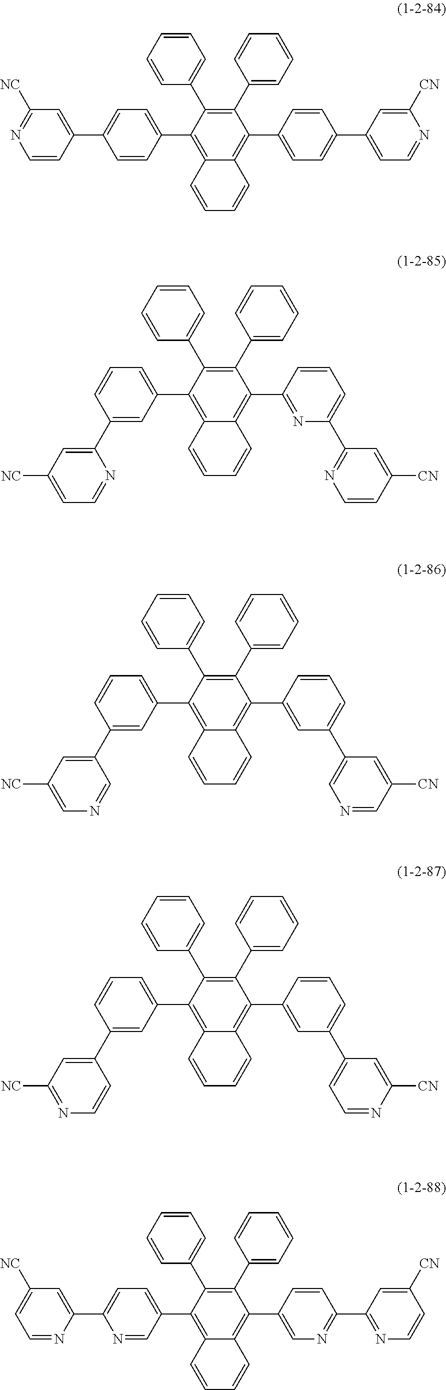 [SMILES: N#Cc1cc(-c2ccc(-c3c(-c4ccccc4)c(-c4ccccc4)c(-c4ccc(-c5ccnc(C#N)c5)cc4)c4ccccc34)cc2)ccn1 ; N#Cc1ccnc(-c2cccc(-c3c(-c4ccccc4)c(-c4ccccc4)c(-c4cccc(-c5cc(C#N)ccn5)n4)c4ccccc34)c2)c1 ; N#Cc1cncc(-c2cccc(-c3c(-c4ccccc4)c(-c4ccccc4)c(-c4cccc(-c5cncc(C#N)c5)c4)c4ccccc34)c2)c1 ; N#Cc1cc(-c2cccc(-c3c(-c4ccccc4)c(-c4ccccc4)c(-c4cccc(-c5ccnc(C#N)c5)c4)c4ccccc34)c2)ccn1 ; N#Cc1ccnc(-c2ccc(-c3c(-c4ccccc4)c(-c4ccccc4)c(-c4ccc(-c5cc(C#N)ccn5)nc4)c4ccccc34)cn2)c1]

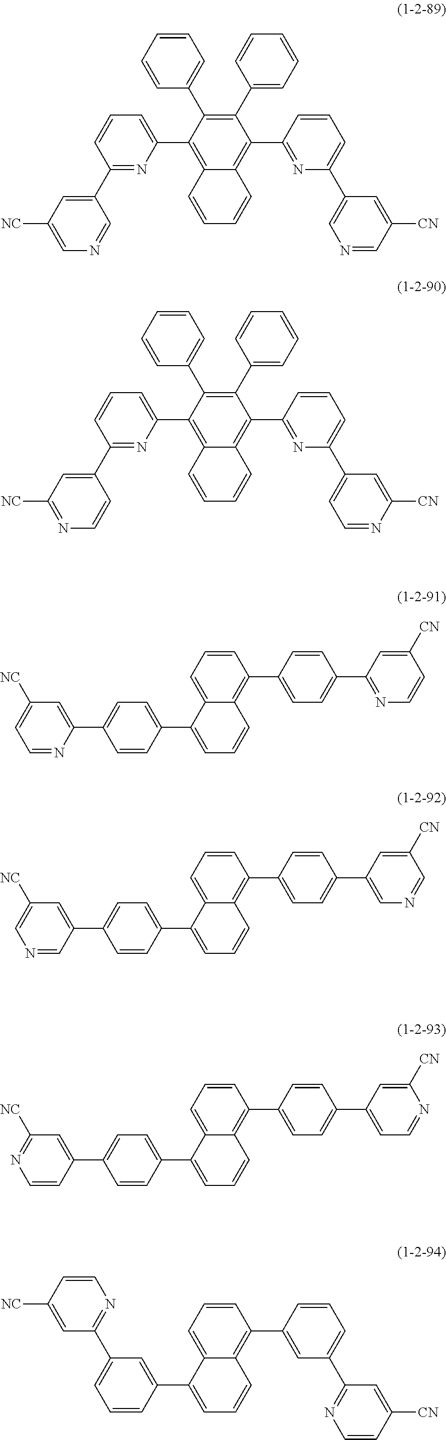 [SMILES: N#Cc1cncc(-c2cccc(-c3c(-c4ccccc4)c(-c4ccccc4)c(-c4cccc(-c5cncc(C#N)c5)n4)c4ccccc34)n2)c1 ; N#Cc1cc(-c2cccc(-c3c(-c4ccccc4)c(-c4ccccc4)c(-c4cccc(-c5ccnc(C#N)c5)n4)c4ccccc34)n2)ccn1 ; N#Cc1ccnc(-c2ccc(-c3cccc4c(-c5ccc(-c6cc(C#N)ccn6)cc5)cccc34)cc2)c1 ; N#Cc1cncc(-c2ccc(-c3cccc4c(-c5ccc(-c6cncc(C#N)c6)cc5)cccc34)cc2)c1 ; N#Cc1cc(-c2ccc(-c3cccc4c(-c5ccc(-c6ccnc(C#N)c6)cc5)cccc34)cc2)ccn1 ; N#Cc1ccnc(-c2cccc(-c3cccc4c(-c5cccc(-c6cc(C#N)ccn6)c5)cccc34)c2)c1]

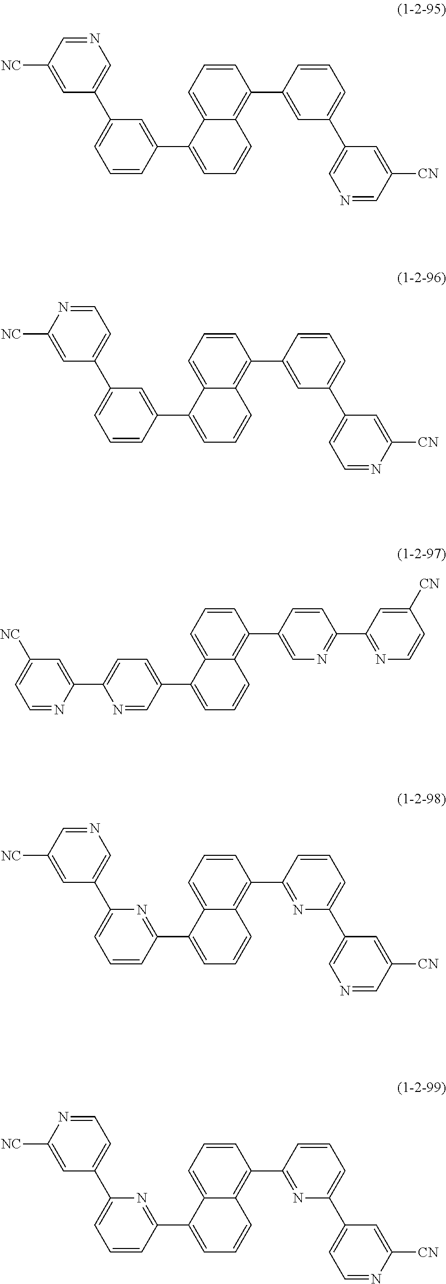 [SMILES: N#Cc1cncc(-c2cccc(-c3cccc4c(-c5cccc(-c6cncc(C#N)c6)c5)cccc34)c2)c1 ; N#Cc1cc(-c2cccc(-c3cccc4c(-c5cccc(-c6ccnc(C#N)c6)c5)cccc34)c2)ccn1 ; N#Cc1ccnc(-c2ccc(-c3cccc4c(-c5ccc(-c6cc(C#N)ccn6)nc5)cccc34)cn2)c1 ; N#Cc1cncc(-c2cccc(-c3cccc4c(-c5cccc(-c6cncc(C#N)c6)n5)cccc34)n2)c1 ; N#Cc1cc(-c2cccc(-c3cccc4c(-c5cccc(-c6ccnc(C#N)c6)n5)cccc34)n2)ccn1]

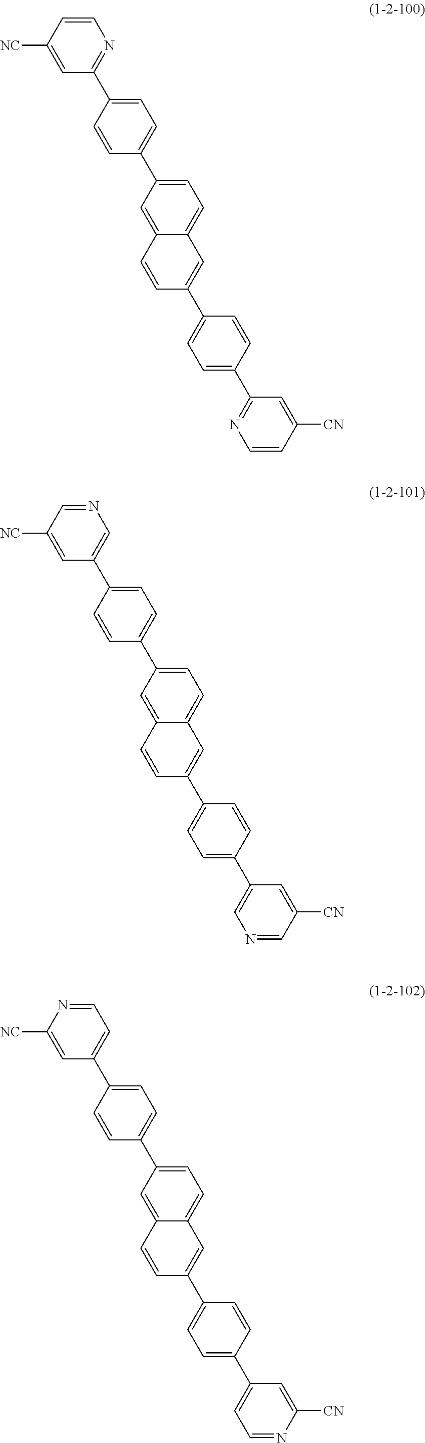 [SMILES: N#Cc1ccnc(-c2ccc(-c3ccc4cc(-c5ccc(-c6cc(C#N)ccn6)cc5)ccc4c3)cc2)c1 ; N#Cc1cncc(-c2ccc(-c3ccc4cc(-c5ccc(-c6cncc(C#N)c6)cc5)ccc4c3)cc2)c1 ; N#Cc1cc(-c2ccc(-c3ccc4cc(-c5ccc(-c6ccnc(C#N)c6)cc5)ccc4c3)cc2)ccn1]

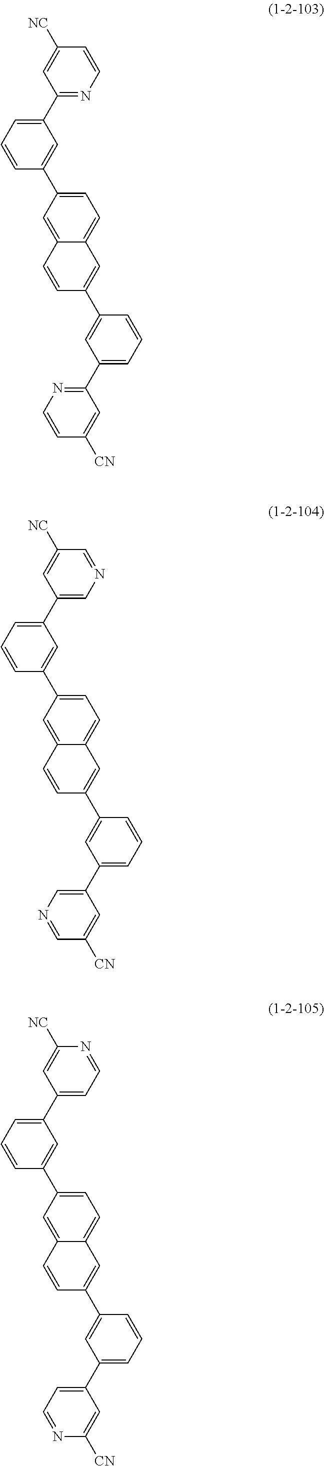 [SMILES: N#Cc1ccnc(-c2cccc(-c3ccc4cc(-c5cccc(-c6cc(C#N)ccn6)c5)ccc4c3)c2)c1 ; N#Cc1cncc(-c2cccc(-c3ccc4cc(-c5cccc(-c6cncc(C#N)c6)c5)ccc4c3)c2)c1 ; N#Cc1cc(-c2cccc(-c3ccc4cc(-c5cccc(-c6ccnc(C#N)c6)c5)ccc4c3)c2)ccn1]

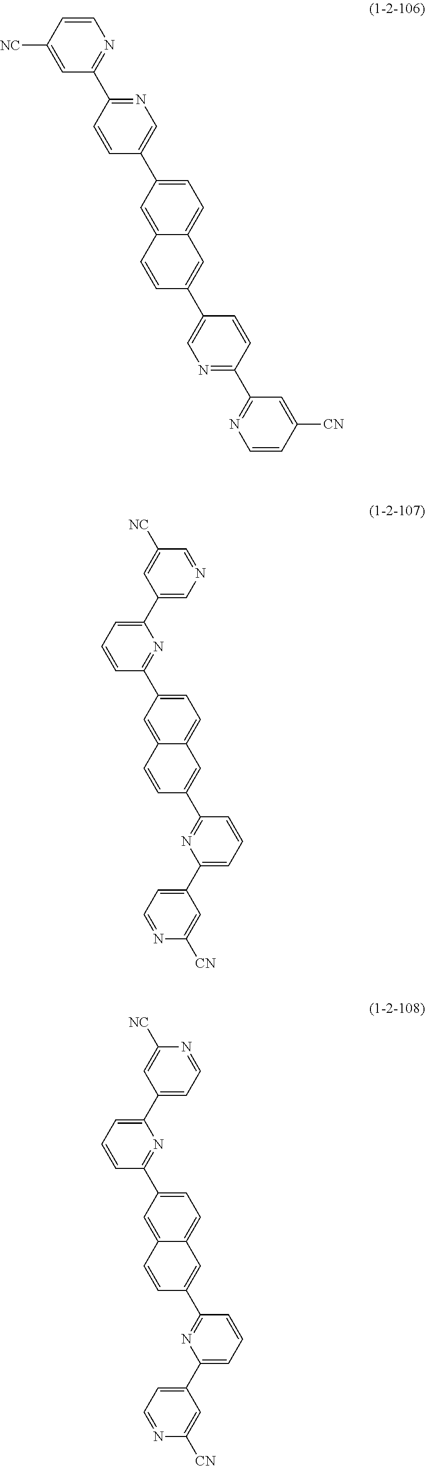 [SMILES: N#Cc1ccnc(-c2ccc(-c3ccc4cc(-c5ccc(-c6cc(C#N)ccn6)nc5)ccc4c3)cn2)c1 ; N#Cc1cncc(-c2cccc(-c3ccc4cc(-c5cccc(-c6ccnc(C#N)c6)n5)ccc4c3)n2)c1 ; N#Cc1cc(-c2cccc(-c3ccc4cc(-c5cccc(-c6ccnc(C#N)c6)n5)ccc4c3)n2)ccn1]

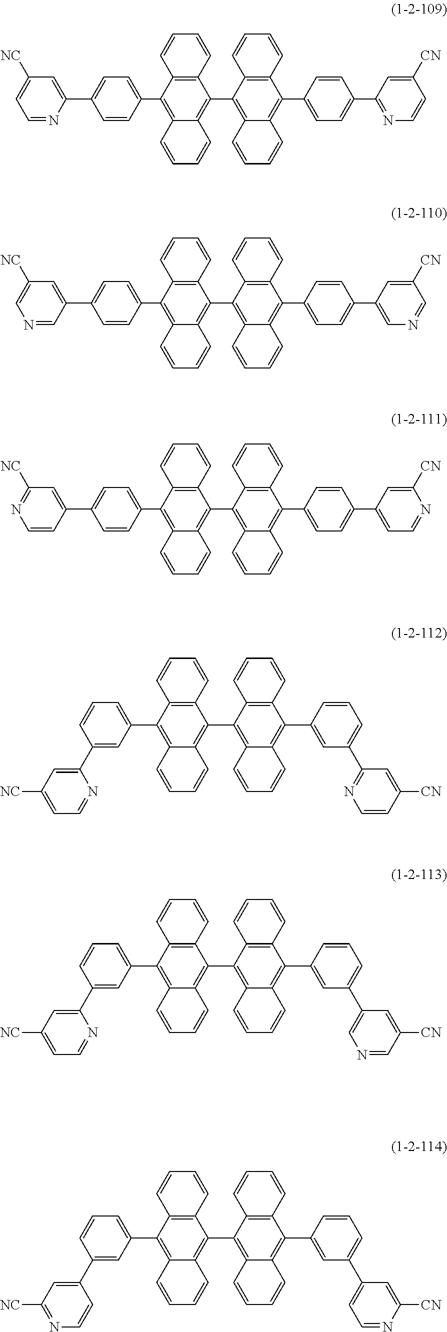

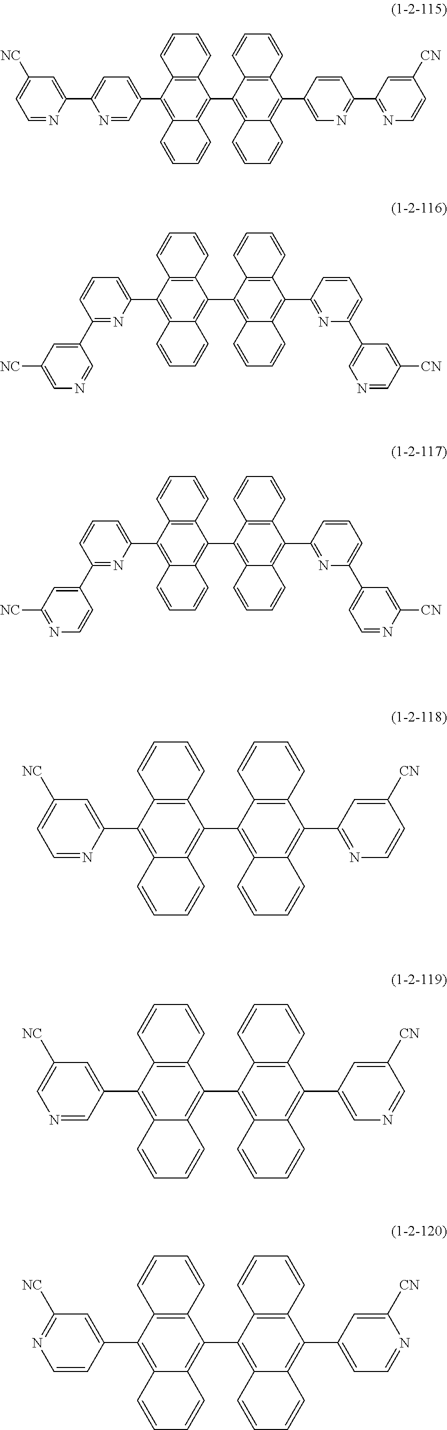

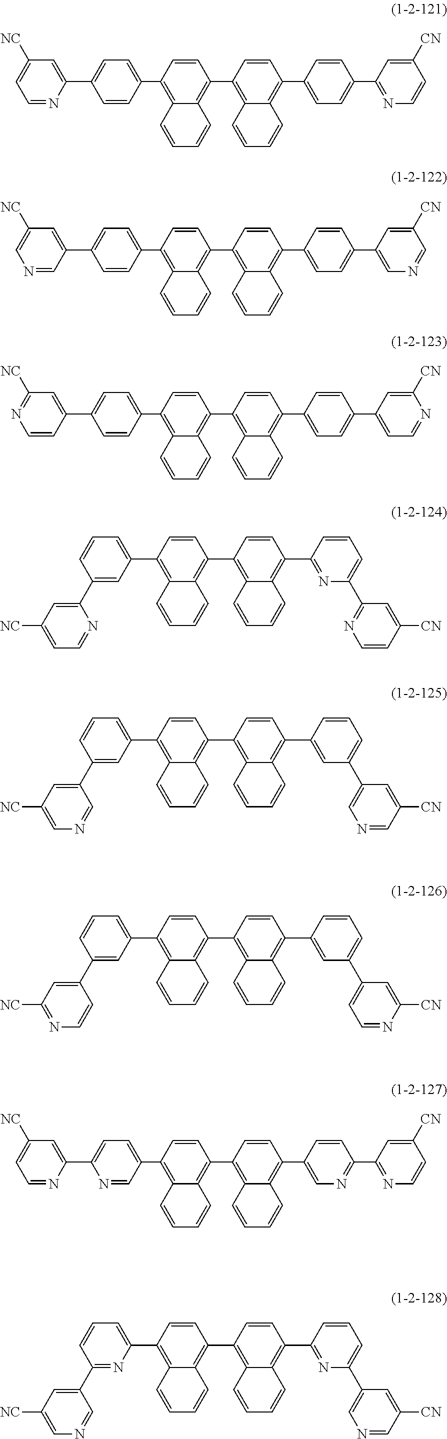

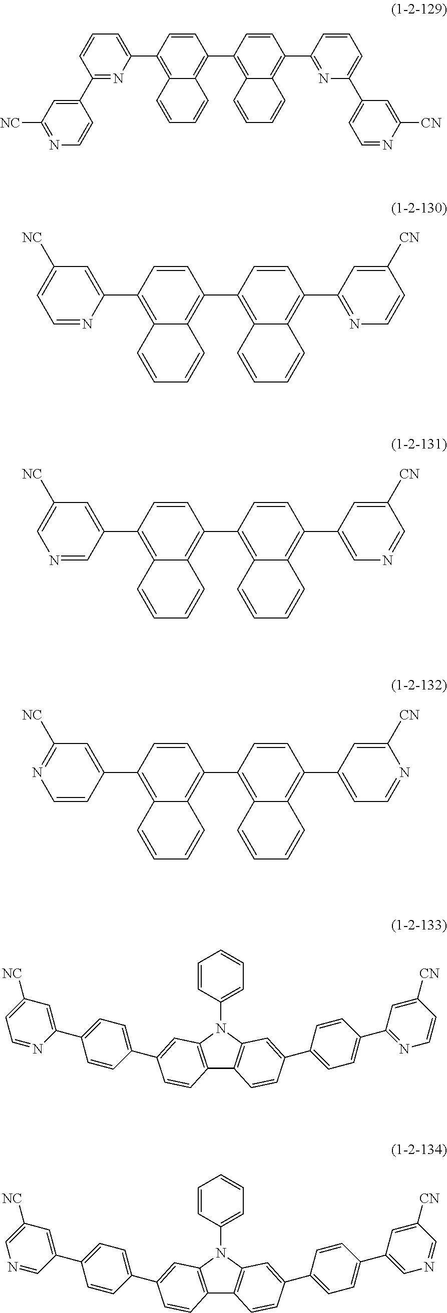 [SMILES: N#Cc1cc(-c2cccc(-c3ccc(-c4ccc(-c5cccc(-c6ccnc(C#N)c6)n5)c5ccccc45)c4ccccc34)n2)ccn1 ; N#Cc1ccnc(-c2ccc(-c3ccc(-c4cc(C#N)ccn4)c4ccccc34)c3ccccc23)c1 ; N#Cc1cncc(-c2ccc(-c3ccc(-c4cncc(C#N)c4)c4ccccc34)c3ccccc23)c1 ; N#Cc1cc(-c2ccc(-c3ccc(-c4ccnc(C#N)c4)c4ccccc34)c3ccccc23)ccn1 ; N#Cc1ccnc(-c2ccc(-c3ccc4c5ccc(-c6ccc(-c7cc(C#N)ccn7)cc6)cc5n(-c5ccccc5)c4c3)cc2)c1 ; N#Cc1cncc(-c2ccc(-c3ccc4c5ccc(-c6ccc(-c7cncc(C#N)c7)cc6)cc5n(-c5ccccc5)c4c3)cc2)c1]

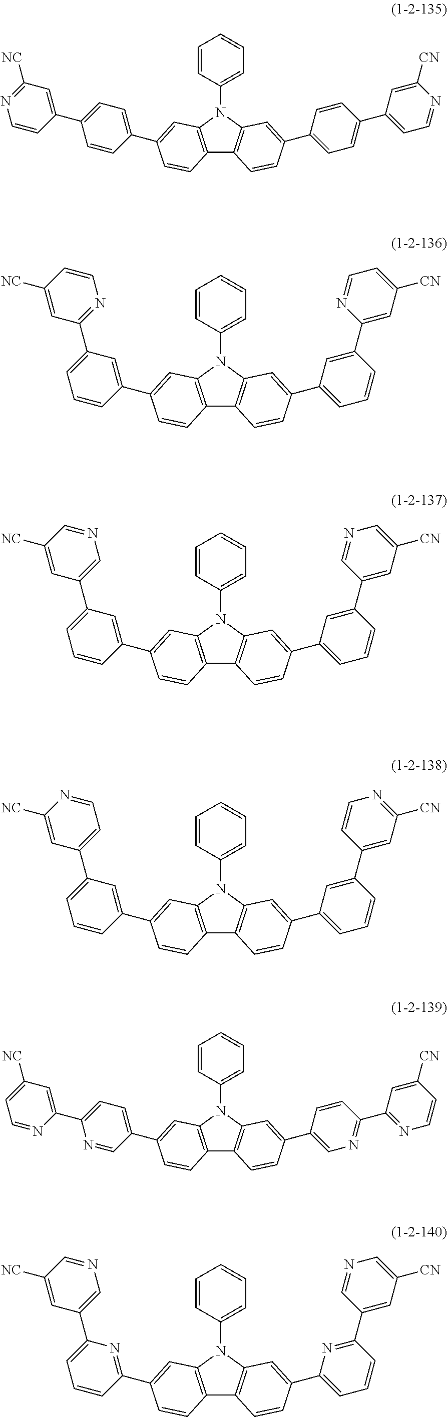

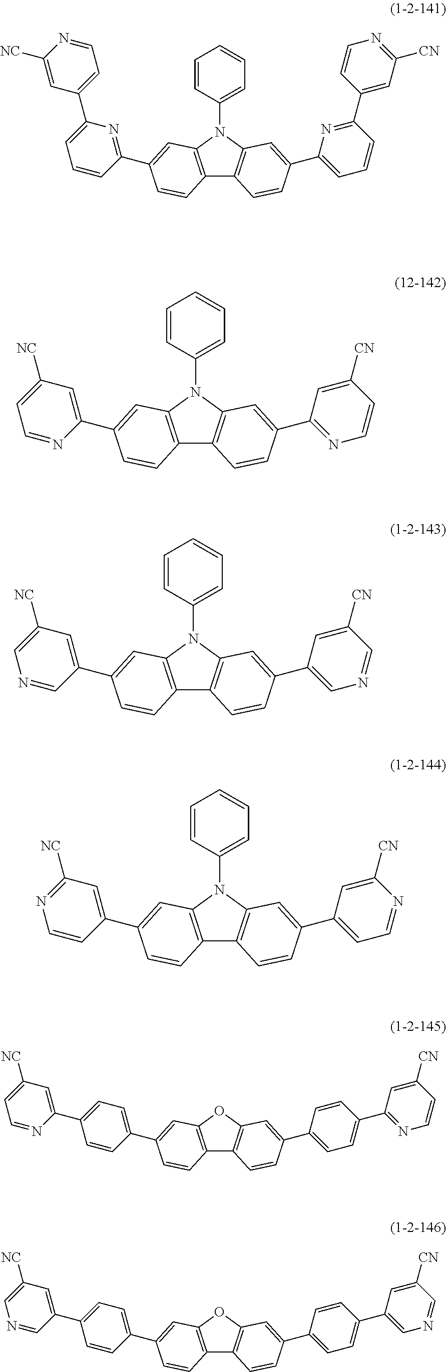 [SMILES: N#Cc1cc(-c2cccc(-c3ccc4c5ccc(-c6cccc(-c7ccnc(C#N)c7)n6)cc5n(-c5ccccc5)c4c3)n2)ccn1 ; N#Cc1ccnc(-c2ccc3c4ccc(-c5cc(C#N)ccn5)cc4n(-c4ccccc4)c3c2)c1 ; N#Cc1cncc(-c2ccc3c4ccc(-c5cncc(C#N)c5)cc4n(-c4ccccc4)c3c2)c1 ; N#Cc1cc(-c2ccc3c4ccc(-c5ccnc(C#N)c5)cc4n(-c4ccccc4)c3c2)ccn1 ; N#Cc1ccnc(-c2ccc(-c3ccc4c(c3)oc3cc(-c5ccc(-c6cc(C#N)ccn6)cc5)ccc34)cc2)c1 ; N#Cc1cncc(-c2ccc(-c3ccc4c(c3)oc3cc(-c5ccc(-c6cncc(C#N)c6)cc5)ccc34)cc2)c1]

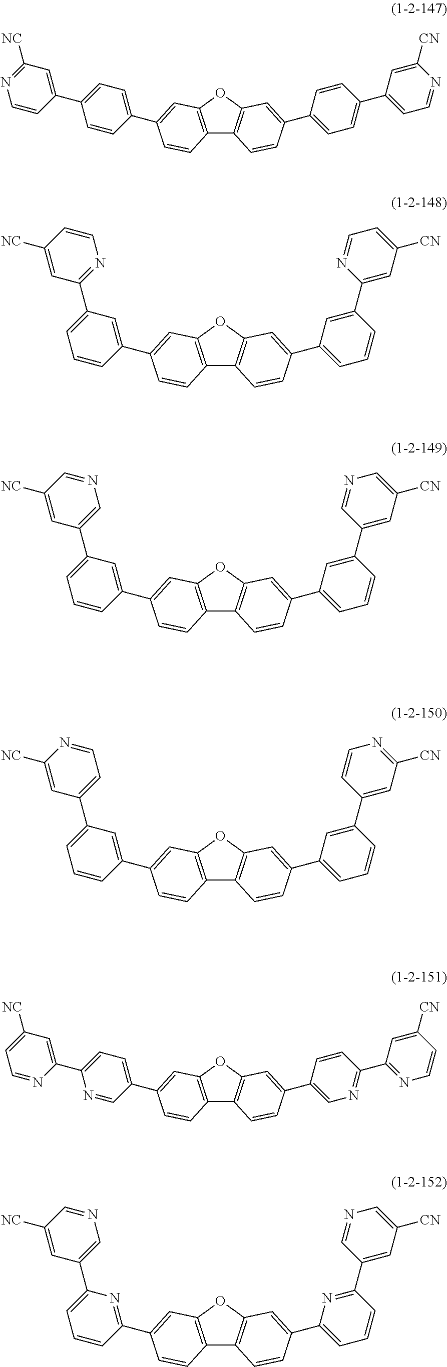 [SMILES: N#Cc1cc(-c2ccc(-c3ccc4c(c3)oc3cc(-c5ccc(-c6ccnc(C#N)c6)cc5)ccc34)cc2)ccn1 ; N#Cc1ccnc(-c2cccc(-c3ccc4c(c3)oc3cc(-c5cccc(-c6cc(C#N)ccn6)c5)ccc34)c2)c1 ; N#Cc1cncc(-c2cccc(-c3ccc4c(c3)oc3cc(-c5cccc(-c6cncc(C#N)c6)c5)ccc34)c2)c1 ; N#Cc1cc(-c2cccc(-c3ccc4c(c3)oc3cc(-c5cccc(-c6ccnc(C#N)c6)c5)ccc34)c2)ccn1 ; N#Cc1ccnc(-c2ccc(-c3ccc4c(c3)oc3cc(-c5ccc(-c6cc(C#N)ccn6)nc5)ccc34)cn2)c1 ; N#Cc1cncc(-c2cccc(-c3ccc4c(c3)oc3cc(-c5cccc(-c6cncc(C#N)c6)n5)ccc34)n2)c1]

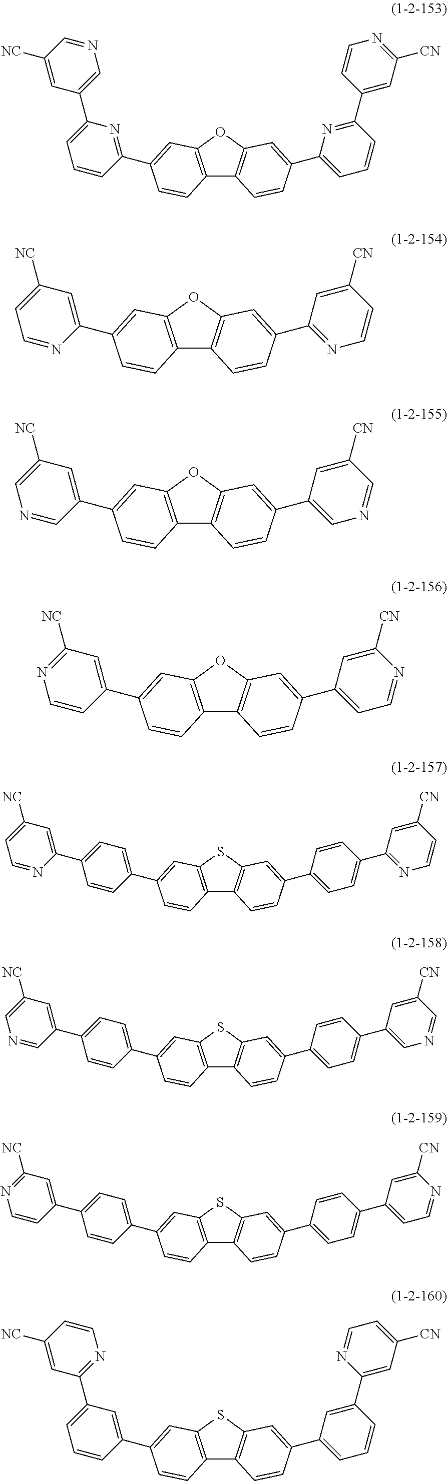

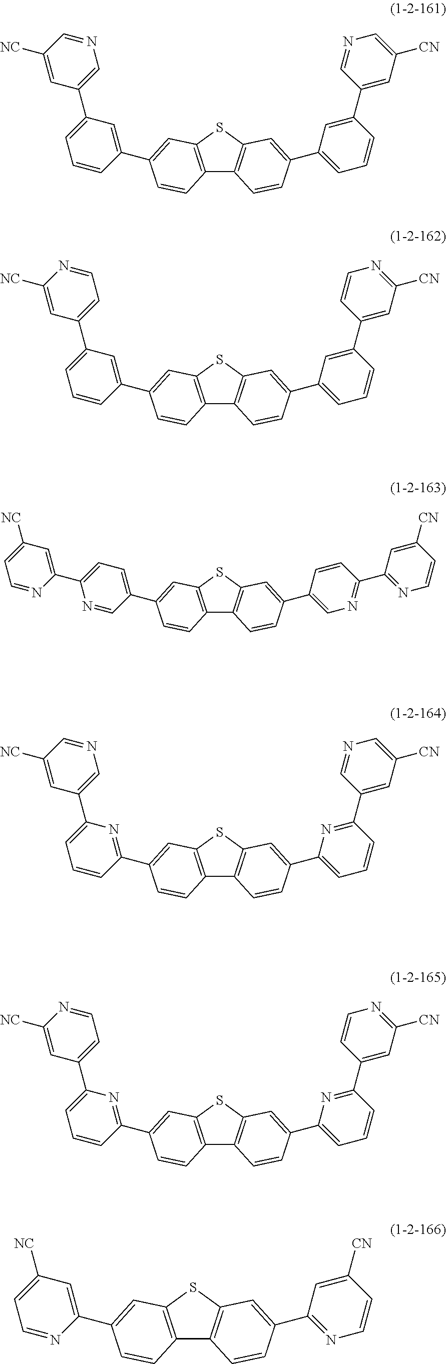 [SMILES: N#Cc1cncc(-c2cccc(-c3ccc4c(c3)sc3cc(-c5cccc(-c6cncc(C#N)c6)c5)ccc34)c2)c1 ; N#Cc1cc(-c2cccc(-c3ccc4c(c3)sc3cc(-c5cccc(-c6ccnc(C#N)c6)c5)ccc34)c2)ccn1 ; N#Cc1ccnc(-c2ccc(-c3ccc4c(c3)sc3cc(-c5ccc(-c6cc(C#N)ccn6)nc5)ccc34)cn2)c1 ; N#Cc1cncc(-c2cccc(-c3ccc4c(c3)sc3cc(-c5cccc(-c6cncc(C#N)c6)n5)ccc34)n2)c1 ; N#Cc1cc(-c2cccc(-c3ccc4c(c3)sc3cc(-c5cccc(-c6ccnc(C#N)c6)n5)ccc34)n2)ccn1 ; N#Cc1ccnc(-c2ccc3c(c2)sc2cc(-c4cc(C#N)ccn4)ccc23)c1]

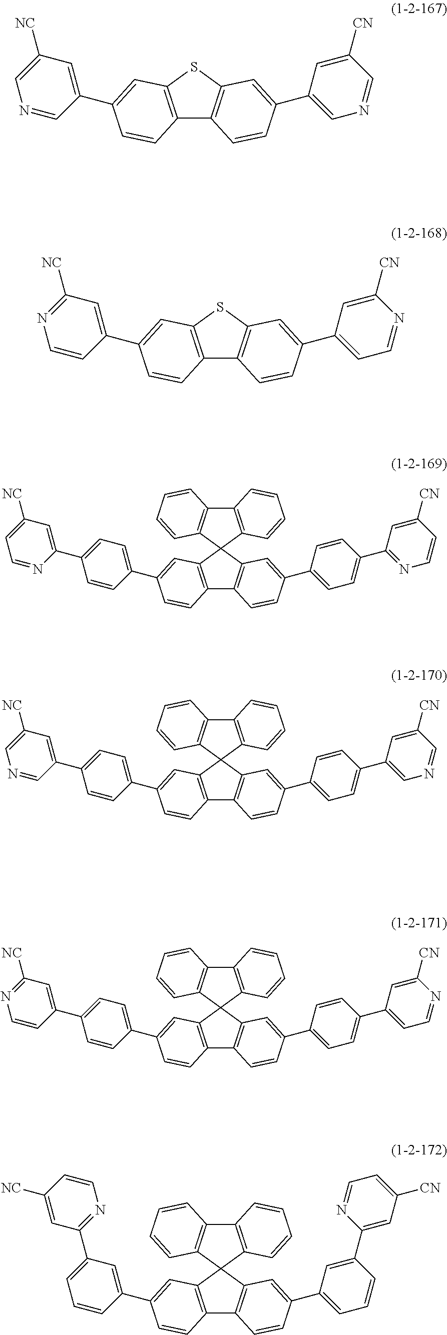 [SMILES: N#Cc1cncc(-c2ccc3c(c2)sc2cc(-c4cncc(C#N)c4)ccc23)c1 ; N#Cc1cc(-c2ccc3c(c2)sc2cc(-c4ccnc(C#N)c4)ccc23)ccn1 ; N#Cc1ccnc(-c2ccc(-c3ccc4c(c3)C3(c5ccccc5-c5ccccc53)c3cc(-c5ccc(-c6cc(C#N)ccn6)cc5)ccc3-4)cc2)c1 ; N#Cc1cncc(-c2ccc(-c3ccc4c(c3)C3(c5ccccc5-c5ccccc53)c3cc(-c5ccc(-c6cncc(C#N)c6)cc5)ccc3-4)cc2)c1 ; N#Cc1cc(-c2ccc(-c3ccc4c(c3)C3(c5ccccc5-c5ccccc53)c3cc(-c5ccc(-c6ccnc(C#N)c6)cc5)ccc3-4)cc2)ccn1 ; N#Cc1ccnc(-c2cccc(-c3ccc4c(c3)C3(c5ccccc5-c5ccccc53)c3cc(-c5cccc(-c6cc(C#N)ccn6)c5)ccc3-4)c2)c1]

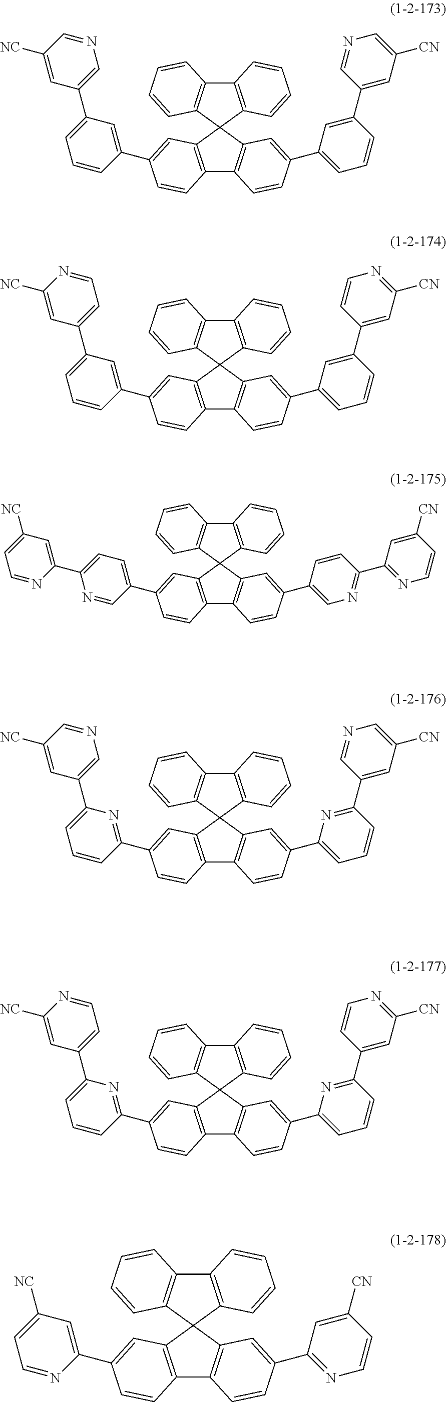

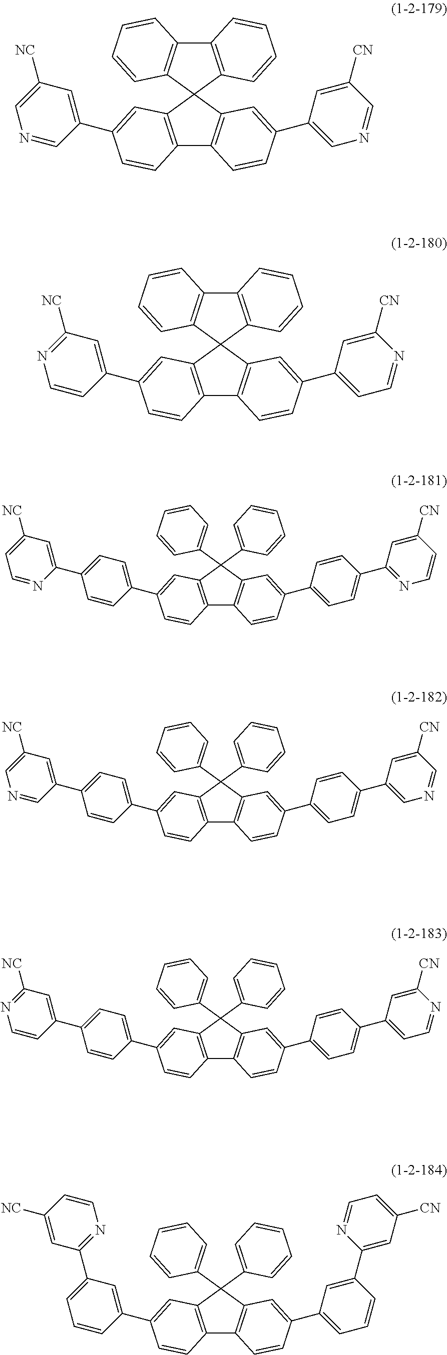

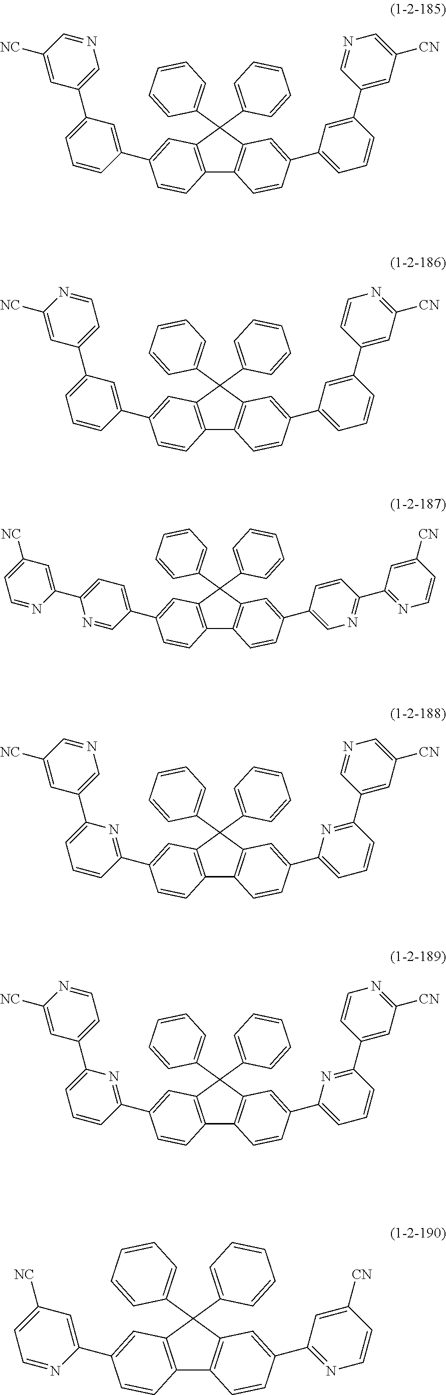

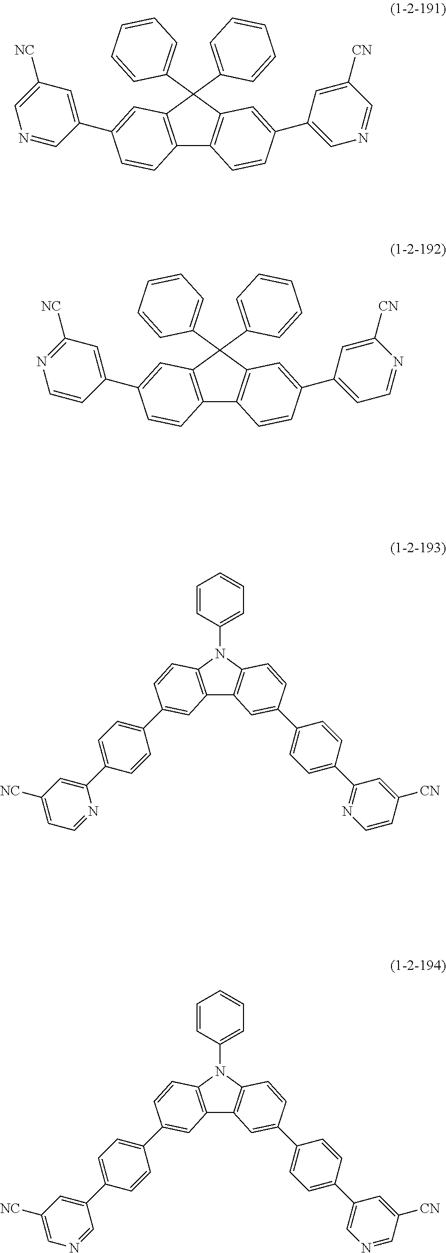 [SMILES: N#Cc1cncc(-c2ccc3c(c2)C(c2ccccc2)(c2ccccc2)c2cc(-c4cncc(C#N)c4)ccc2-3)c1 ; N#Cc1cc(-c2ccc3c(c2)C(c2ccccc2)(c2ccccc2)c2cc(-c4ccnc(C#N)c4)ccc2-3)ccn1 ; N#Cc1ccnc(-c2ccc(-c3ccc4c(c3)c3cc(-c5ccc(-c6cc(C#N)ccn6)cc5)ccc3n4-c3ccccc3)cc2)c1 ; N#Cc1cncc(-c2ccc(-c3ccc4c(c3)c3cc(-c5ccc(-c6cncc(C#N)c6)cc5)ccc3n4-c3ccccc3)cc2)c1]

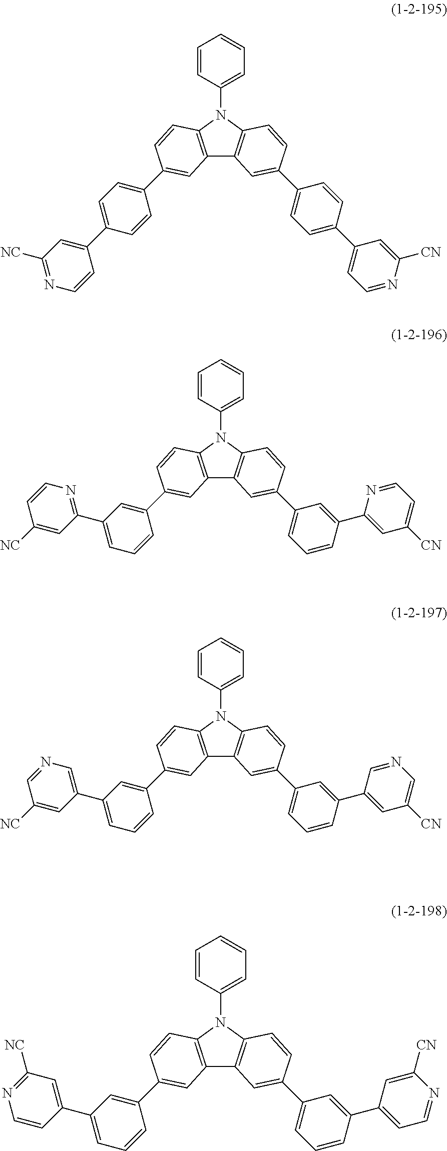 [SMILES: N#Cc1cc(-c2ccc(-c3ccc4c(c3)c3cc(-c5ccc(-c6ccnc(C#N)c6)cc5)ccc3n4-c3ccccc3)cc2)ccn1 ; N#Cc1ccnc(-c2cccc(-c3ccc4c(c3)c3cc(-c5cccc(-c6cc(C#N)ccn6)c5)ccc3n4-c3ccccc3)c2)c1 ; N#Cc1cncc(-c2cccc(-c3ccc4c(c3)c3cc(-c5cccc(-c6cncc(C#N)c6)c5)ccc3n4-c3ccccc3)c2)c1 ; N#Cc1cc(-c2cccc(-c3ccc4c(c3)c3cc(-c5cccc(-c6ccnc(C#N)c6)c5)ccc3n4-c3ccccc3)c2)ccn1]

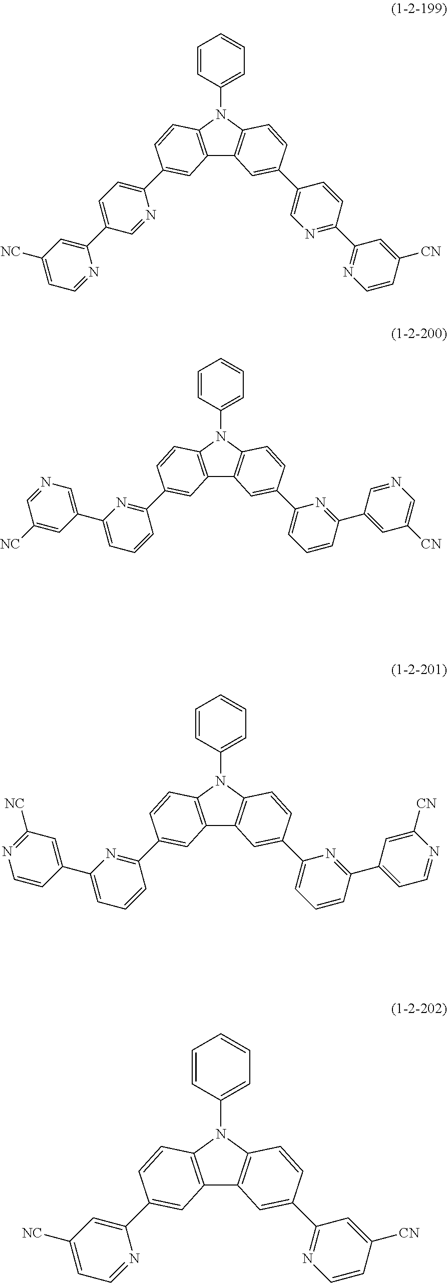 [SMILES: N#Cc1ccnc(-c2ccc(-c3ccc4c(c3)c3cc(-c5ccc(-c6cc(C#N)ccn6)nc5)ccc3n4-c3ccccc3)nc2)c1 ; N#Cc1cncc(-c2cccc(-c3ccc4c(c3)c3cc(-c5cccc(-c6cncc(C#N)c6)n5)ccc3n4-c3ccccc3)n2)c1 ; N#Cc1cc(-c2cccc(-c3ccc4c(c3)c3cc(-c5cccc(-c6ccnc(C#N)c6)n5)ccc3n4-c3ccccc3)n2)ccn1 ; N#Cc1ccnc(-c2ccc3c(c2)c2cc(-c4cc(C#N)ccn4)ccc2n3-c2ccccc2)c1]

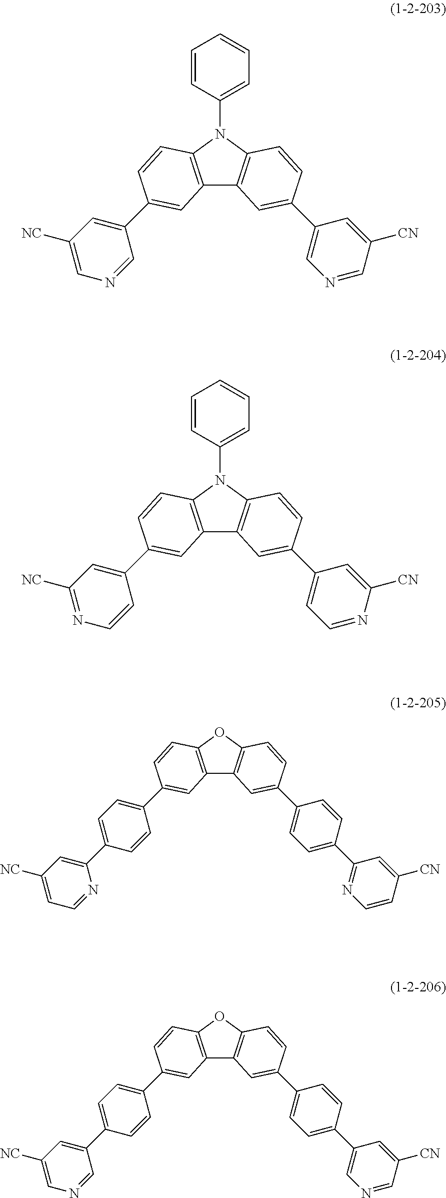 [SMILES: N#Cc1cncc(-c2ccc3c(c2)c2cc(-c4cncc(C#N)c4)ccc2n3-c2ccccc2)c1 ; N#Cc1cc(-c2ccc3c(c2)c2cc(-c4ccnc(C#N)c4)ccc2n3-c2ccccc2)ccn1 ; N#Cc1ccnc(-c2ccc(-c3ccc4oc5ccc(-c6ccc(-c7cc(C#N)ccn7)cc6)cc5c4c3)cc2)c1 ; N#Cc1cncc(-c2ccc(-c3ccc4oc5ccc(-c6ccc(-c7cncc(C#N)c7)cc6)cc5c4c3)cc2)c1]

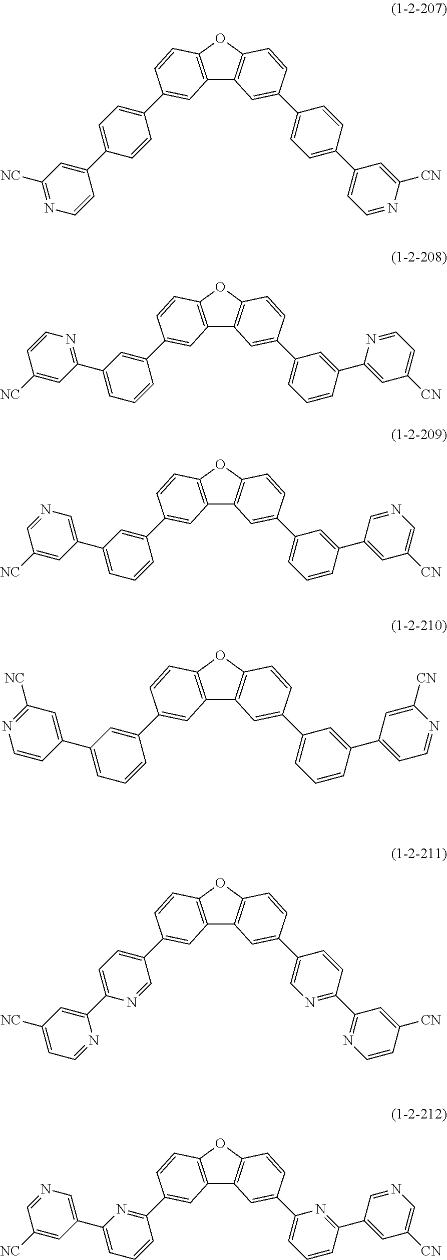 [SMILES: N#Cc1cc(-c2ccc(-c3ccc4oc5ccc(-c6ccc(-c7ccnc(C#N)c7)cc6)cc5c4c3)cc2)ccn1 ; N#Cc1ccnc(-c2cccc(-c3ccc4oc5ccc(-c6cccc(-c7cc(C#N)ccn7)c6)cc5c4c3)c2)c1 ; N#Cc1cncc(-c2cccc(-c3ccc4oc5ccc(-c6cccc(-c7cncc(C#N)c7)c6)cc5c4c3)c2)c1 ; N#Cc1cc(-c2cccc(-c3ccc4oc5ccc(-c6cccc(-c7ccnc(C#N)c7)c6)cc5c4c3)c2)ccn1 ; N#Cc1ccnc(-c2ccc(-c3ccc4oc5ccc(-c6ccc(-c7cc(C#N)ccn7)nc6)cc5c4c3)cn2)c1 ; N#Cc1cncc(-c2cccc(-c3ccc4oc5ccc(-c6cccc(-c7cncc(C#N)c7)n6)cc5c4c3)n2)c1]

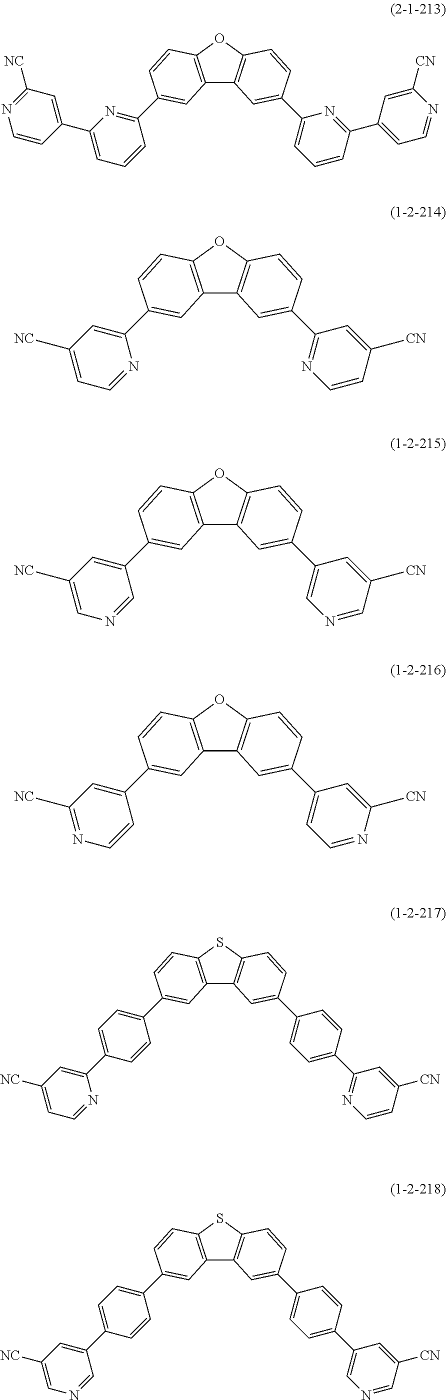 [SMILES: N#Cc1cc(-c2cccc(-c3ccc4oc5ccc(-c6cccc(-c7ccnc(C#N)c7)n6)cc5c4c3)n2)ccn1 ; N#Cc1ccnc(-c2ccc3oc4ccc(-c5cc(C#N)ccn5)cc4c3c2)c1 ; N#Cc1cncc(-c2ccc3oc4ccc(-c5cncc(C#N)c5)cc4c3c2)c1 ; N#Cc1cc(-c2ccc3oc4ccc(-c5ccnc(C#N)c5)cc4c3c2)ccn1 ; N#Cc1ccnc(-c2ccc(-c3ccc4sc5ccc(-c6ccc(-c7cc(C#N)ccn7)cc6)cc5c4c3)cc2)c1 ; N#Cc1cncc(-c2ccc(-c3ccc4sc5ccc(-c6ccc(-c7cncc(C#N)c7)cc6)cc5c4c3)cc2)c1]

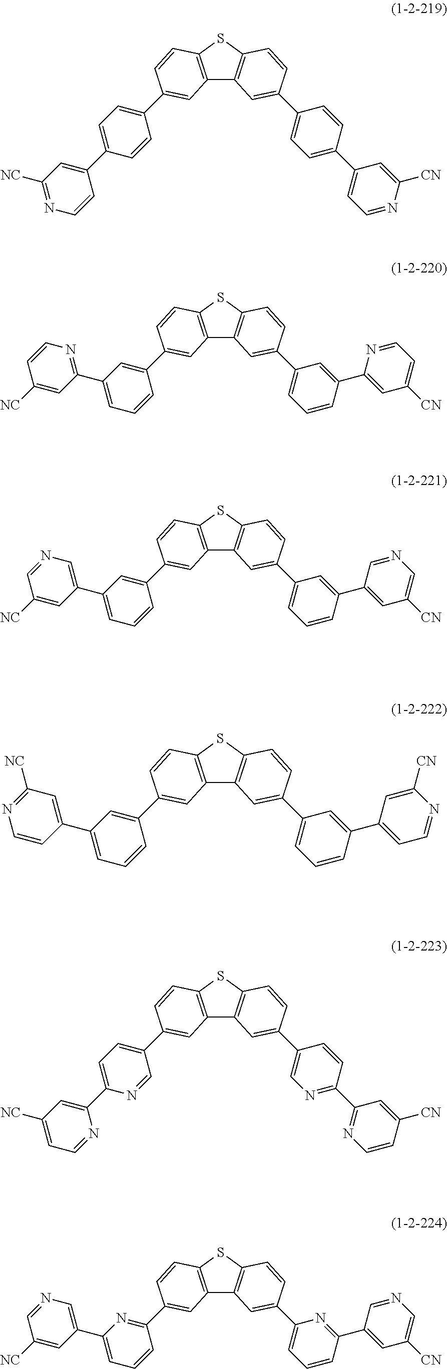 [SMILES: N#Cc1cc(-c2ccc(-c3ccc4sc5ccc(-c6ccc(-c7ccnc(C#N)c7)cc6)cc5c4c3)cc2)ccn1 ; N#Cc1ccnc(-c2cccc(-c3ccc4sc5ccc(-c6cccc(-c7cc(C#N)ccn7)c6)cc5c4c3)c2)c1 ; N#Cc1cncc(-c2cccc(-c3ccc4sc5ccc(-c6cccc(-c7cncc(C#N)c7)c6)cc5c4c3)c2)c1 ; N#Cc1cc(-c2cccc(-c3ccc4sc5ccc(-c6cccc(-c7ccnc(C#N)c7)c6)cc5c4c3)c2)ccn1 ; N#Cc1ccnc(-c2ccc(-c3ccc4sc5ccc(-c6ccc(-c7cc(C#N)ccn7)nc6)cc5c4c3)cn2)c1 ; N#Cc1cncc(-c2cccc(-c3ccc4sc5ccc(-c6cccc(-c7cncc(C#N)c7)n6)cc5c4c3)n2)c1]

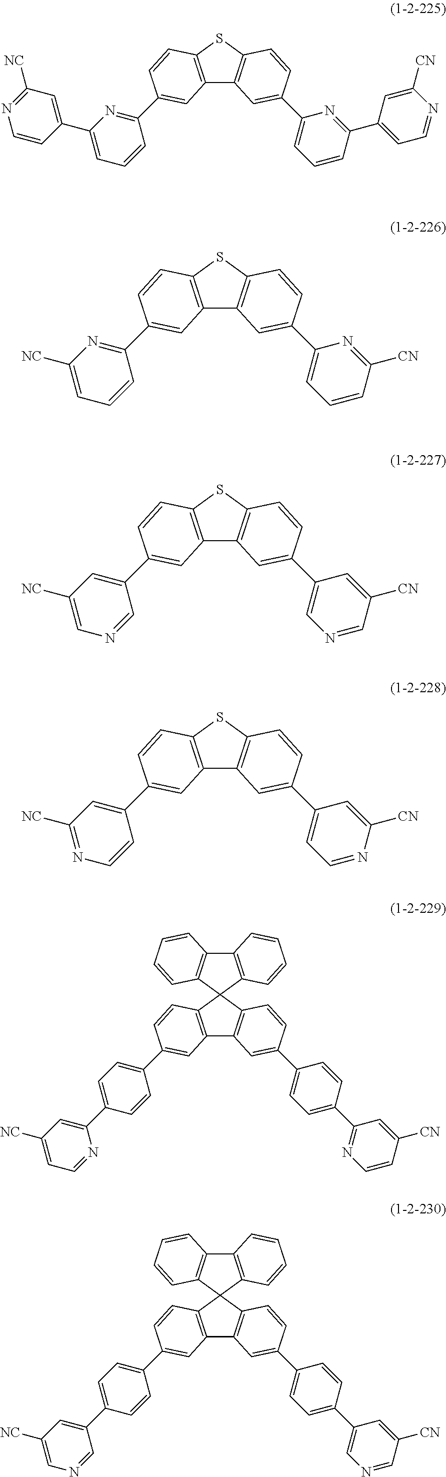 [SMILES: N#Cc1cc(-c2cccc(-c3ccc4sc5ccc(-c6cccc(-c7ccnc(C#N)c7)n6)cc5c4c3)n2)ccn1 ; N#Cc1cccc(-c2ccc3sc4ccc(-c5cccc(C#N)n5)cc4c3c2)n1 ; N#Cc1cncc(-c2ccc3sc4ccc(-c5cncc(C#N)c5)cc4c3c2)c1 ; N#Cc1cc(-c2ccc3sc4ccc(-c5ccnc(C#N)c5)cc4c3c2)ccn1 ; N#Cc1ccnc(-c2ccc(-c3ccc4c(c3)-c3cc(-c5ccc(-c6cc(C#N)ccn6)cc5)ccc3C43c4ccccc4-c4ccccc43)cc2)c1 ; N#Cc1cncc(-c2ccc(-c3ccc4c(c3)-c3cc(-c5ccc(-c6cncc(C#N)c6)cc5)ccc3C43c4ccccc4-c4ccccc43)cc2)c1]

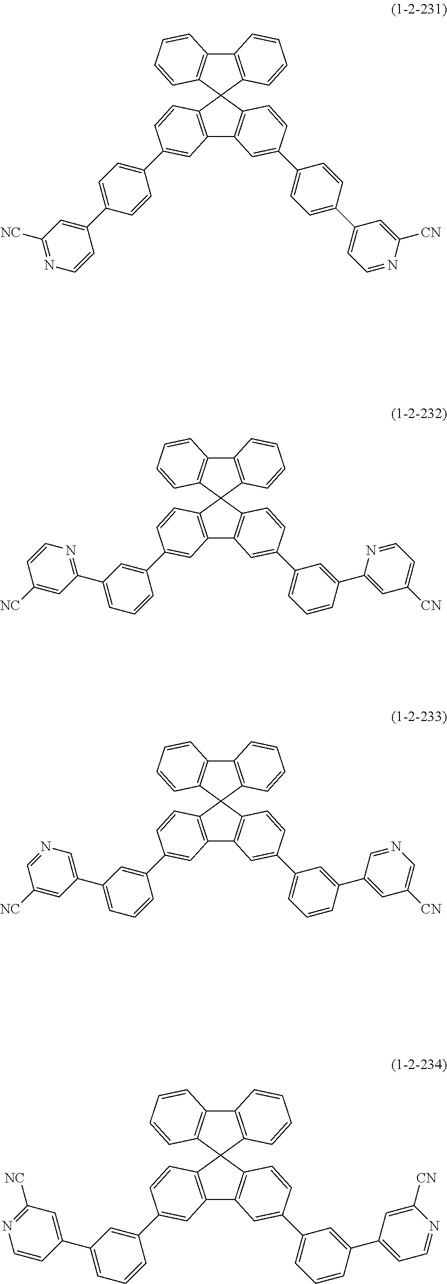 [SMILES: N#Cc1cc(-c2ccc(-c3ccc4c(c3)-c3cc(-c5ccc(-c6ccnc(C#N)c6)cc5)ccc3C43c4ccccc4-c4ccccc43)cc2)ccn1 ; N#Cc1ccnc(-c2cccc(-c3ccc4c(c3)-c3cc(-c5cccc(-c6cc(C#N)ccn6)c5)ccc3C43c4ccccc4-c4ccccc43)c2)c1 ; N#Cc1cncc(-c2cccc(-c3ccc4c(c3)-c3cc(-c5cccc(-c6cncc(C#N)c6)c5)ccc3C43c4ccccc4-c4ccccc43)c2)c1 ; N#Cc1cc(-c2cccc(-c3ccc4c(c3)-c3cc(-c5cccc(-c6ccnc(C#N)c6)c5)ccc3C43c4ccccc4-c4ccccc43)c2)ccn1]

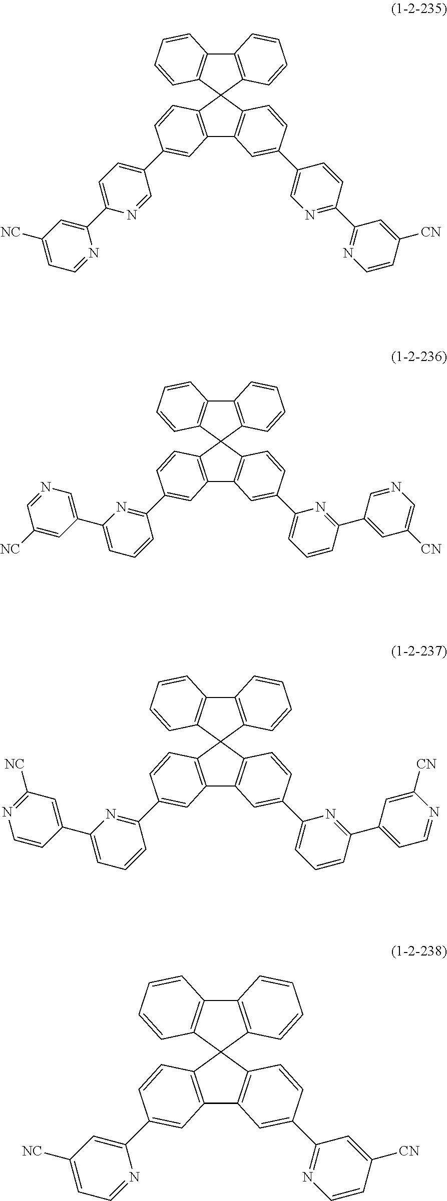 [SMILES: N#Cc1ccnc(-c2ccc(-c3ccc4c(c3)-c3cc(-c5ccc(-c6cc(C#N)ccn6)nc5)ccc3C43c4ccccc4-c4ccccc43)cn2)c1 ; N#Cc1cncc(-c2cccc(-c3ccc4c(c3)-c3cc(-c5cccc(-c6cncc(C#N)c6)n5)ccc3C43c4ccccc4-c4ccccc43)n2)c1 ; N#Cc1cc(-c2cccc(-c3ccc4c(c3)-c3cc(-c5cccc(-c6ccnc(C#N)c6)n5)ccc3C43c4ccccc4-c4ccccc43)n2)ccn1 ; N#Cc1ccnc(-c2ccc3c(c2)-c2cc(-c4cc(C#N)ccn4)ccc2C32c3ccccc3-c3ccccc32)c1]

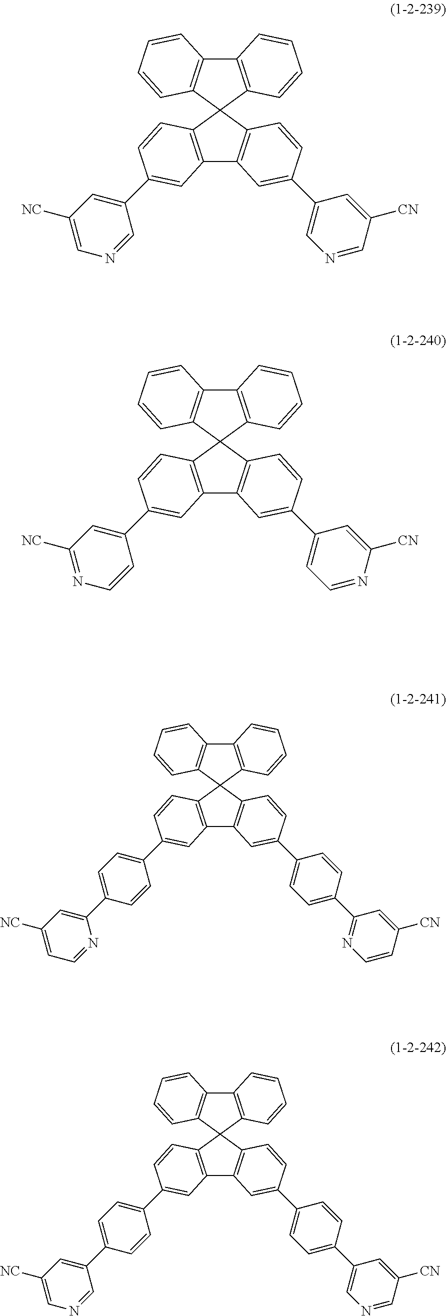 [SMILES: N#Cc1cncc(-c2ccc3c(c2)-c2cc(-c4cncc(C#N)c4)ccc2C32c3ccccc3-c3ccccc32)c1 ; N#Cc1cc(-c2ccc3c(c2)-c2cc(-c4ccnc(C#N)c4)ccc2C32c3ccccc3-c3ccccc32)ccn1 ; N#Cc1ccnc(-c2ccc(-c3ccc4c(c3)-c3cc(-c5ccc(-c6cc(C#N)ccn6)cc5)ccc3C43c4ccccc4-c4ccccc43)cc2)c1 ; N#Cc1cncc(-c2ccc(-c3ccc4c(c3)-c3cc(-c5ccc(-c6cncc(C#N)c6)cc5)ccc3C43c4ccccc4-c4ccccc43)cc2)c1]

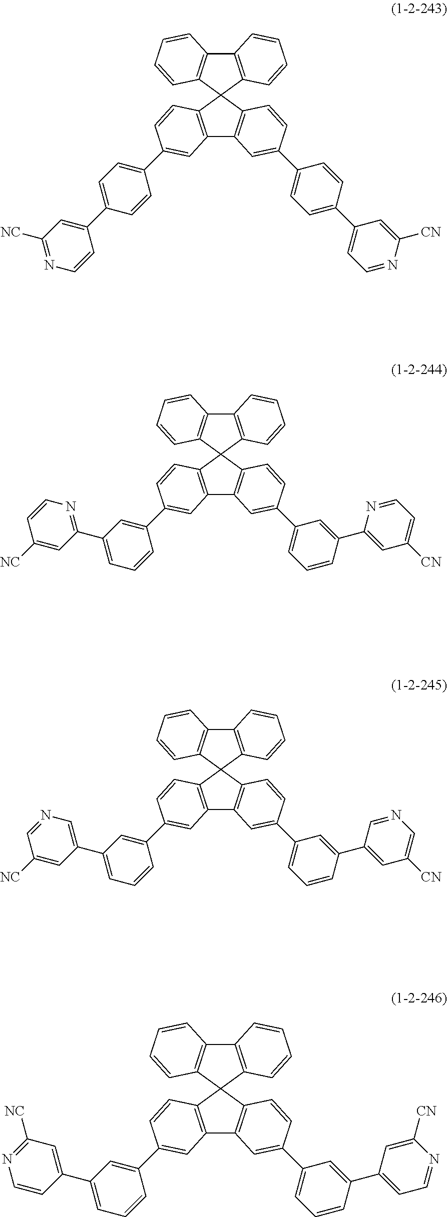 [SMILES: N#Cc1cc(-c2ccc(-c3ccc4c(c3)-c3cc(-c5ccc(-c6ccnc(C#N)c6)cc5)ccc3C43c4ccccc4-c4ccccc43)cc2)ccn1 ; N#Cc1ccnc(-c2cccc(-c3ccc4c(c3)-c3cc(-c5cccc(-c6cc(C#N)ccn6)c5)ccc3C43c4ccccc4-c4ccccc43)c2)c1 ; N#Cc1cncc(-c2cccc(-c3ccc4c(c3)-c3cc(-c5cccc(-c6cncc(C#N)c6)c5)ccc3C43c4ccccc4-c4ccccc43)c2)c1 ; N#Cc1cc(-c2cccc(-c3ccc4c(c3)-c3cc(-c5cccc(-c6ccnc(C#N)c6)c5)ccc3C43c4ccccc4-c4ccccc43)c2)ccn1]

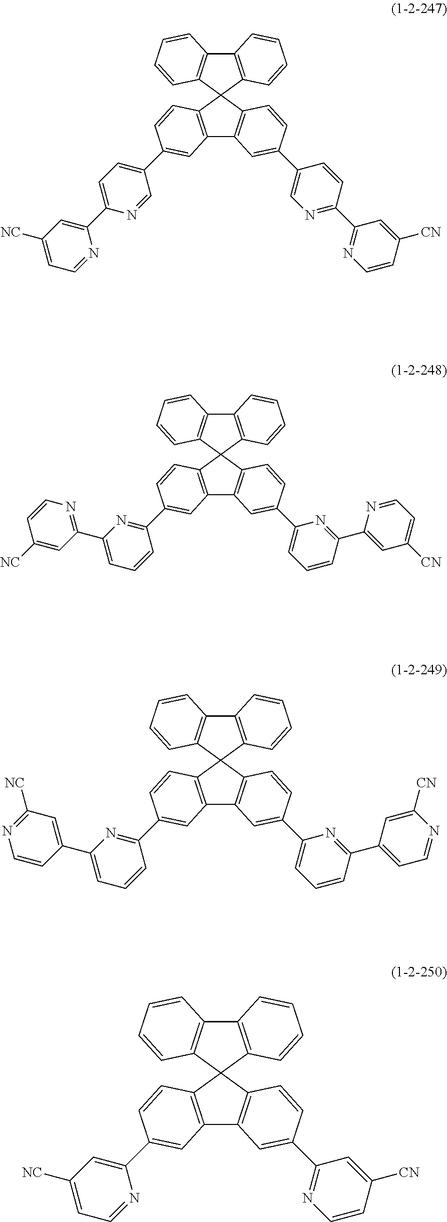 [SMILES: N#Cc1ccnc(-c2ccc(-c3ccc4c(c3)-c3cc(-c5ccc(-c6cc(C#N)ccn6)nc5)ccc3C43c4ccccc4-c4ccccc43)cn2)c1 ; N#Cc1ccnc(-c2cccc(-c3ccc4c(c3)-c3cc(-c5cccc(-c6cc(C#N)ccn6)n5)ccc3C43c4ccccc4-c4ccccc43)n2)c1 ; N#Cc1cc(-c2cccc(-c3ccc4c(c3)-c3cc(-c5cccc(-c6ccnc(C#N)c6)n5)ccc3C43c4ccccc4-c4ccccc43)n2)ccn1 ; N#Cc1ccnc(-c2ccc3c(c2)-c2cc(-c4cc(C#N)ccn4)ccc2C32c3ccccc3-c3ccccc32)c1]

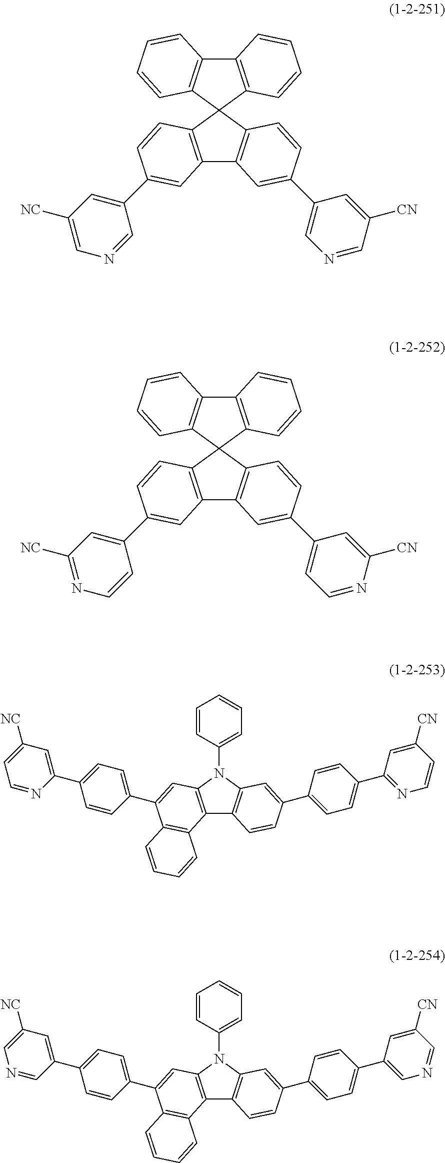 [SMILES: N#Cc1cncc(-c2ccc3c(c2)-c2cc(-c4cncc(C#N)c4)ccc2C32c3ccccc3-c3ccccc32)c1 ; N#Cc1cc(-c2ccc3c(c2)-c2cc(-c4ccnc(C#N)c4)ccc2C32c3ccccc3-c3ccccc32)ccn1 ; N#Cc1ccnc(-c2ccc(-c3ccc4c5c6ccccc6c(-c6ccc(-c7cc(C#N)ccn7)cc6)cc5n(-c5ccccc5)c4c3)cc2)c1 ; N#Cc1cncc(-c2ccc(-c3ccc4c5c6ccccc6c(-c6ccc(-c7cncc(C#N)c7)cc6)cc5n(-c5ccccc5)c4c3)cc2)c1]

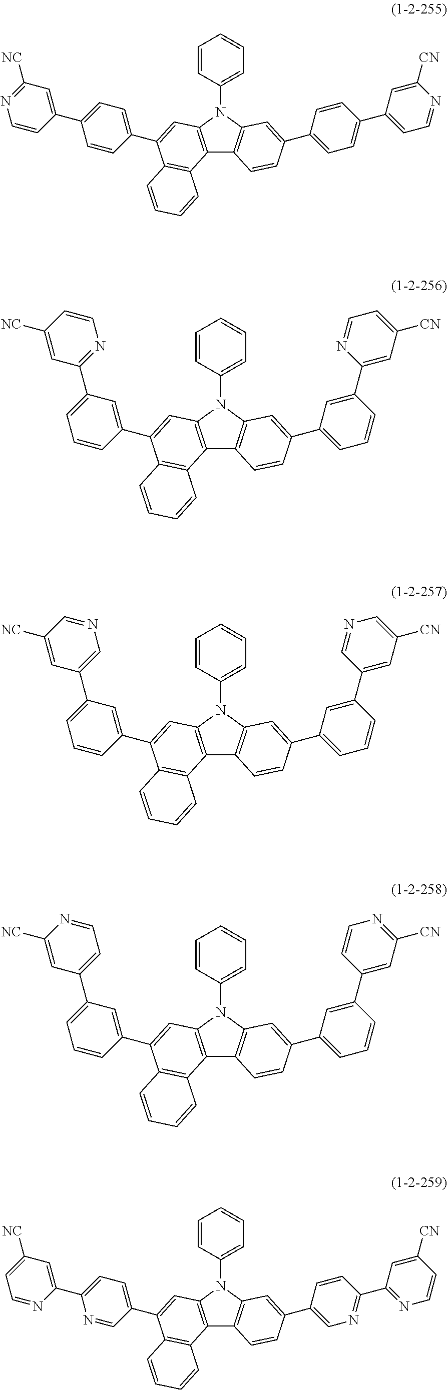 [SMILES: N#Cc1cc(-c2ccc(-c3ccc4c5c6ccccc6c(-c6ccc(-c7ccnc(C#N)c7)cc6)cc5n(-c5ccccc5)c4c3)cc2)ccn1 ; N#Cc1ccnc(-c2cccc(-c3ccc4c5c6ccccc6c(-c6cccc(-c7cc(C#N)ccn7)c6)cc5n(-c5ccccc5)c4c3)c2)c1 ; N#Cc1cncc(-c2cccc(-c3ccc4c5c6ccccc6c(-c6cccc(-c7cncc(C#N)c7)c6)cc5n(-c5ccccc5)c4c3)c2)c1 ; N#Cc1cc(-c2cccc(-c3ccc4c5c6ccccc6c(-c6cccc(-c7ccnc(C#N)c7)c6)cc5n(-c5ccccc5)c4c3)c2)ccn1 ; N#Cc1ccnc(-c2ccc(-c3ccc4c5c6ccccc6c(-c6ccc(-c7cc(C#N)ccn7)nc6)cc5n(-c5ccccc5)c4c3)cn2)c1]

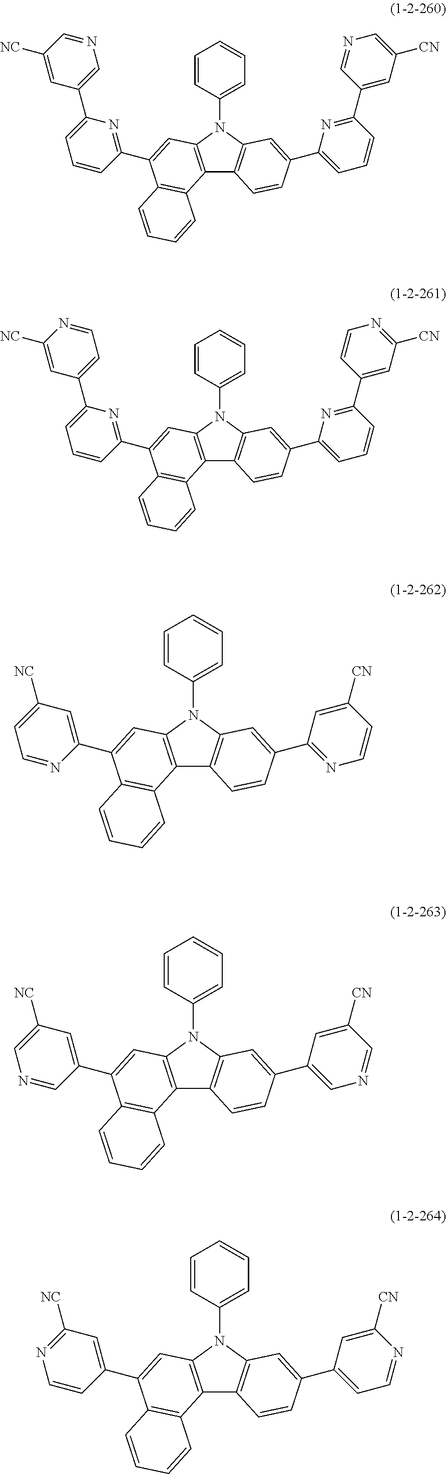 [SMILES: N#Cc1cncc(-c2cccc(-c3ccc4c5c6ccccc6c(-c6cccc(-c7cncc(C#N)c7)n6)cc5n(-c5ccccc5)c4c3)n2)c1 ; N#Cc1cc(-c2cccc(-c3ccc4c5c6ccccc6c(-c6cccc(-c7ccnc(C#N)c7)n6)cc5n(-c5ccccc5)c4c3)n2)ccn1 ; N#Cc1ccnc(-c2ccc3c4c5ccccc5c(-c5cc(C#N)ccn5)cc4n(-c4ccccc4)c3c2)c1 ; N#Cc1cncc(-c2ccc3c4c5ccccc5c(-c5cncc(C#N)c5)cc4n(-c4ccccc4)c3c2)c1 ; N#Cc1cc(-c2ccc3c4c5ccccc5c(-c5ccnc(C#N)c5)cc4n(-c4ccccc4)c3c2)ccn1]

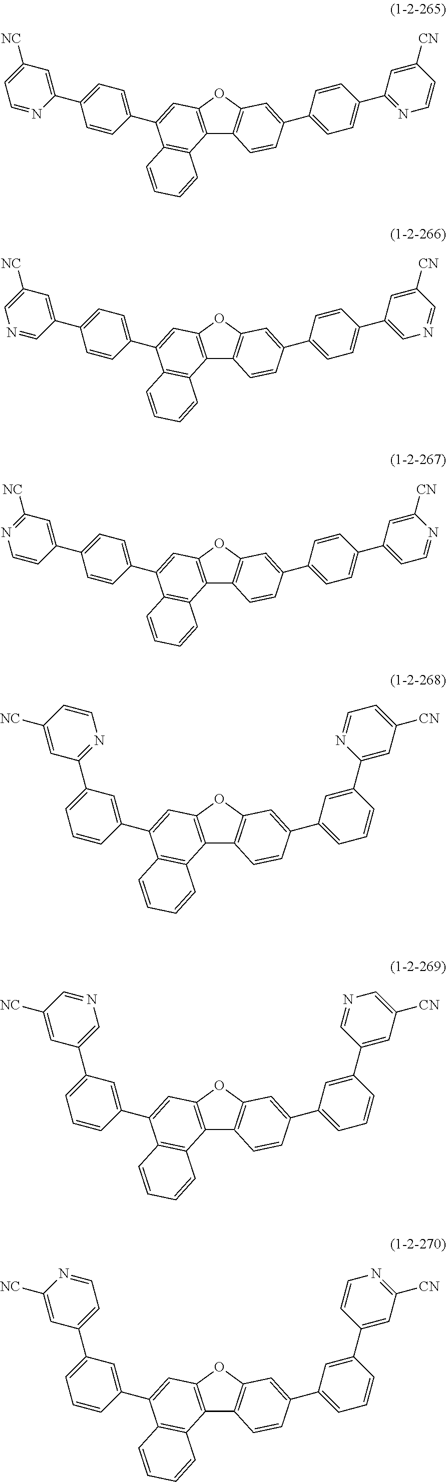 [SMILES: N#Cc1ccnc(-c2ccc(-c3ccc4c(c3)oc3cc(-c5ccc(-c6cc(C#N)ccn6)cc5)c5ccccc5c34)cc2)c1 ; N#Cc1cncc(-c2ccc(-c3ccc4c(c3)oc3cc(-c5ccc(-c6cncc(C#N)c6)cc5)c5ccccc5c34)cc2)c1 ; N#Cc1cc(-c2ccc(-c3ccc4c(c3)oc3cc(-c5ccc(-c6ccnc(C#N)c6)cc5)c5ccccc5c34)cc2)ccn1 ; N#Cc1ccnc(-c2cccc(-c3ccc4c(c3)oc3cc(-c5cccc(-c6cc(C#N)ccn6)c5)c5ccccc5c34)c2)c1 ; N#Cc1cncc(-c2cccc(-c3ccc4c(c3)oc3cc(-c5cccc(-c6cncc(C#N)c6)c5)c5ccccc5c34)c2)c1 ; N#Cc1cc(-c2cccc(-c3ccc4c(c3)oc3cc(-c5cccc(-c6ccnc(C#N)c6)c5)c5ccccc5c34)c2)ccn1]

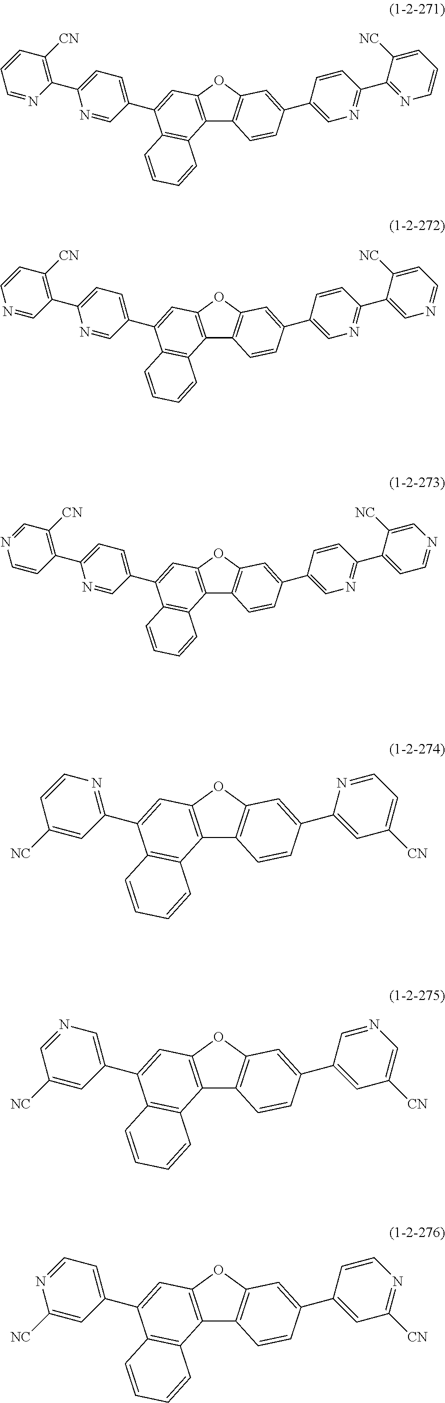 [SMILES: N#Cc1cccnc1-c1ccc(-c2ccc3c(c2)oc2cc(-c4ccc(-c5ncccc5C#N)nc4)c4ccccc4c23)cn1 ; N#Cc1ccncc1-c1ccc(-c2ccc3c(c2)oc2cc(-c4ccc(-c5cnccc5C#N)nc4)c4ccccc4c23)cn1 ; N#Cc1cnccc1-c1ccc(-c2ccc3c(c2)oc2cc(-c4ccc(-c5ccncc5C#N)nc4)c4ccccc4c23)cn1 ; N#Cc1ccnc(-c2ccc3c(c2)oc2cc(-c4cc(C#N)ccn4)c4ccccc4c23)c1 ; N#Cc1cncc(-c2ccc3c(c2)oc2cc(-c4cncc(C#N)c4)c4ccccc4c23)c1 ; N#Cc1cc(-c2ccc3c(c2)oc2cc(-c4ccnc(C#N)c4)c4ccccc4c23)ccn1]

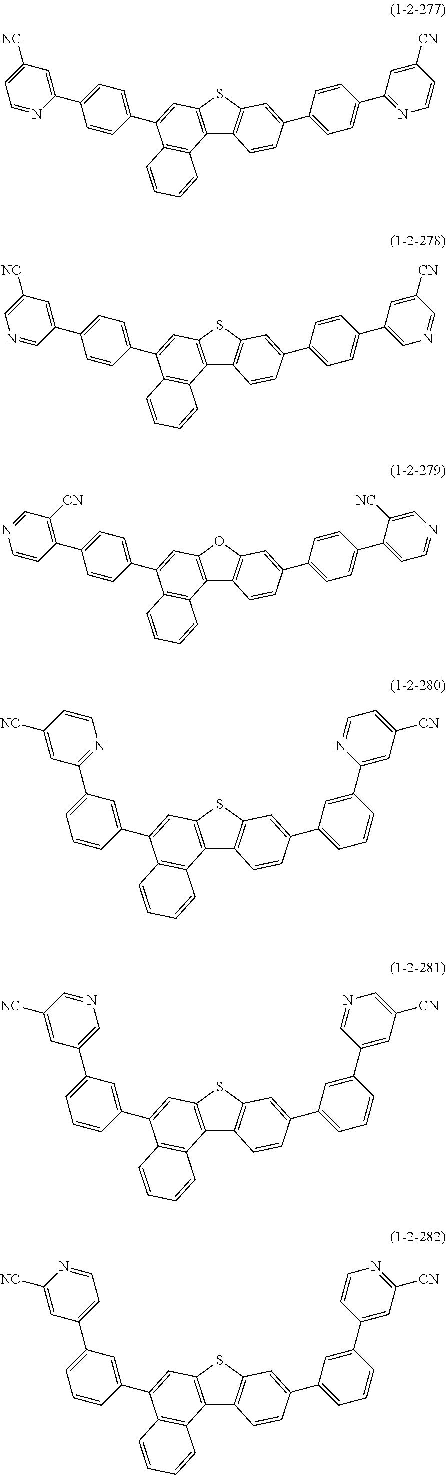 [SMILES: N#Cc1ccnc(-c2ccc(-c3ccc4c(c3)sc3cc(-c5ccc(-c6cc(C#N)ccn6)cc5)c5ccccc5c34)cc2)c1 ; N#Cc1cncc(-c2ccc(-c3ccc4c(c3)sc3cc(-c5ccc(-c6cncc(C#N)c6)cc5)c5ccccc5c34)cc2)c1 ; N#Cc1cnccc1-c1ccc(-c2ccc3c(c2)oc2cc(-c4ccc(-c5ccncc5C#N)cc4)c4ccccc4c23)cc1 ; N#Cc1ccnc(-c2cccc(-c3ccc4c(c3)sc3cc(-c5cccc(-c6cc(C#N)ccn6)c5)c5ccccc5c34)c2)c1 ; N#Cc1cncc(-c2cccc(-c3ccc4c(c3)sc3cc(-c5cccc(-c6cncc(C#N)c6)c5)c5ccccc5c34)c2)c1 ; N#Cc1cc(-c2cccc(-c3ccc4c(c3)sc3cc(-c5cccc(-c6ccnc(C#N)c6)c5)c5ccccc5c34)c2)ccn1]

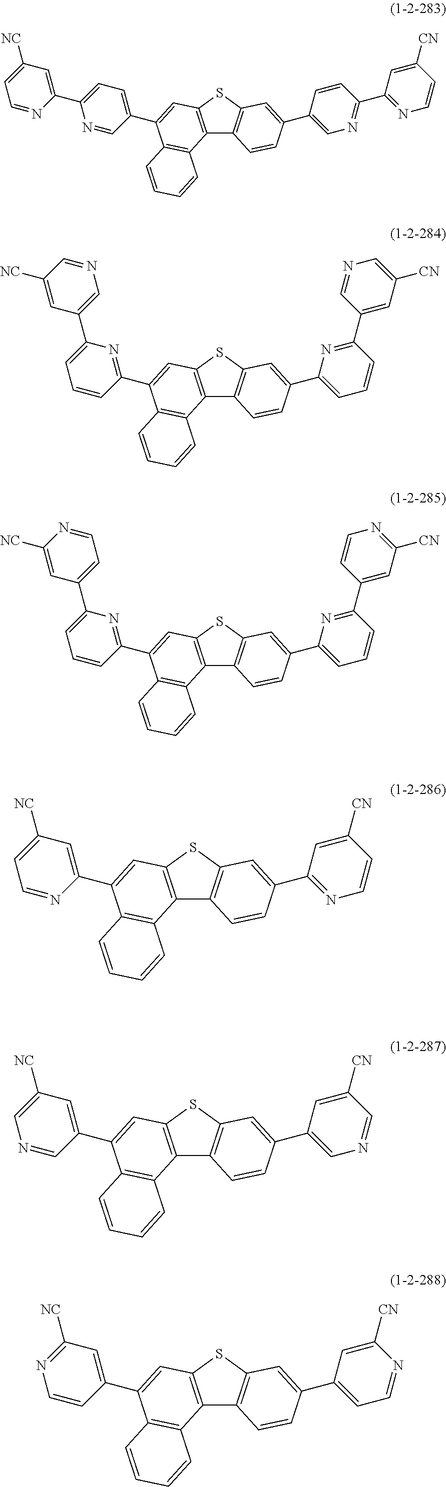 [SMILES: N#Cc1ccnc(-c2ccc(-c3ccc4c(c3)sc3cc(-c5ccc(-c6cc(C#N)ccn6)nc5)c5ccccc5c34)cn2)c1 ; N#Cc1cncc(-c2cccc(-c3ccc4c(c3)sc3cc(-c5cccc(-c6cncc(C#N)c6)n5)c5ccccc5c34)n2)c1 ; N#Cc1cc(-c2cccc(-c3ccc4c(c3)sc3cc(-c5cccc(-c6ccnc(C#N)c6)n5)c5ccccc5c34)n2)ccn1 ; N#Cc1ccnc(-c2ccc3c(c2)sc2cc(-c4cc(C#N)ccn4)c4ccccc4c23)c1 ; N#Cc1cncc(-c2ccc3c(c2)sc2cc(-c4cncc(C#N)c4)c4ccccc4c23)c1 ; N#Cc1cc(-c2ccc3c(c2)sc2cc(-c4ccnc(C#N)c4)c4ccccc4c23)ccn1]

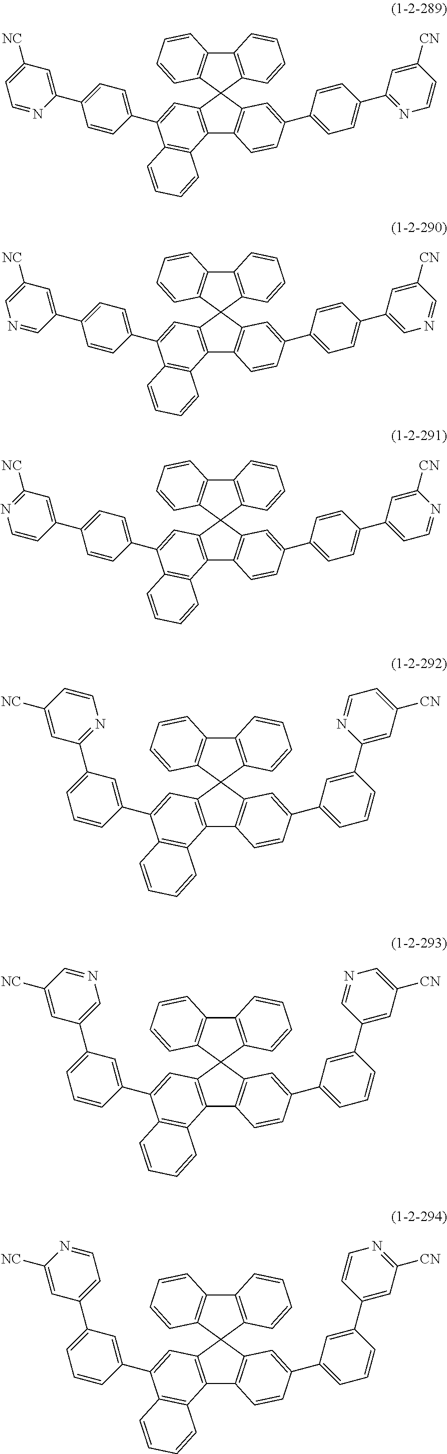

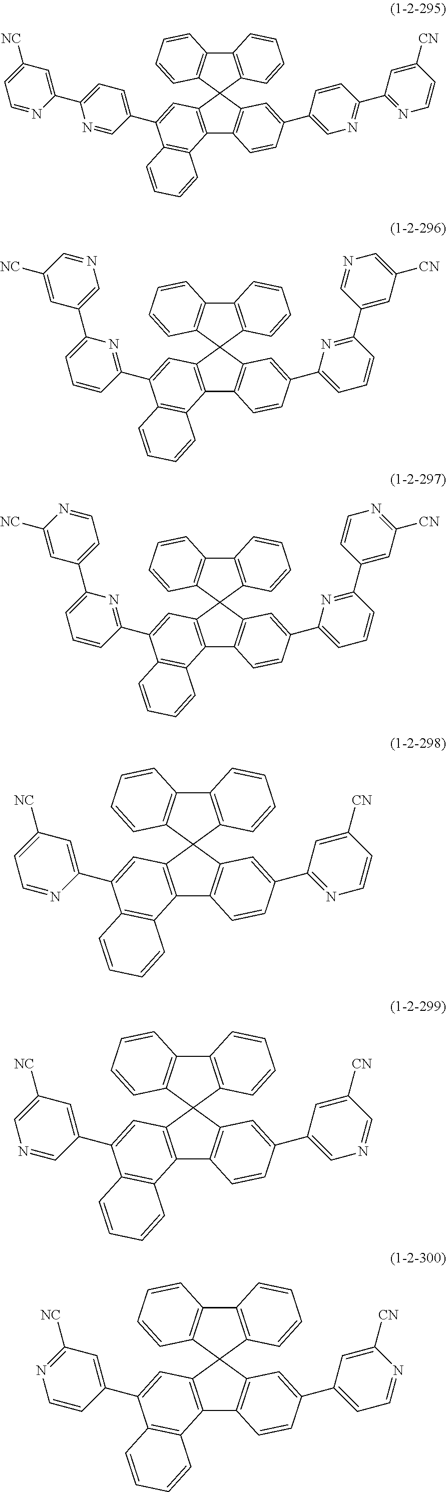

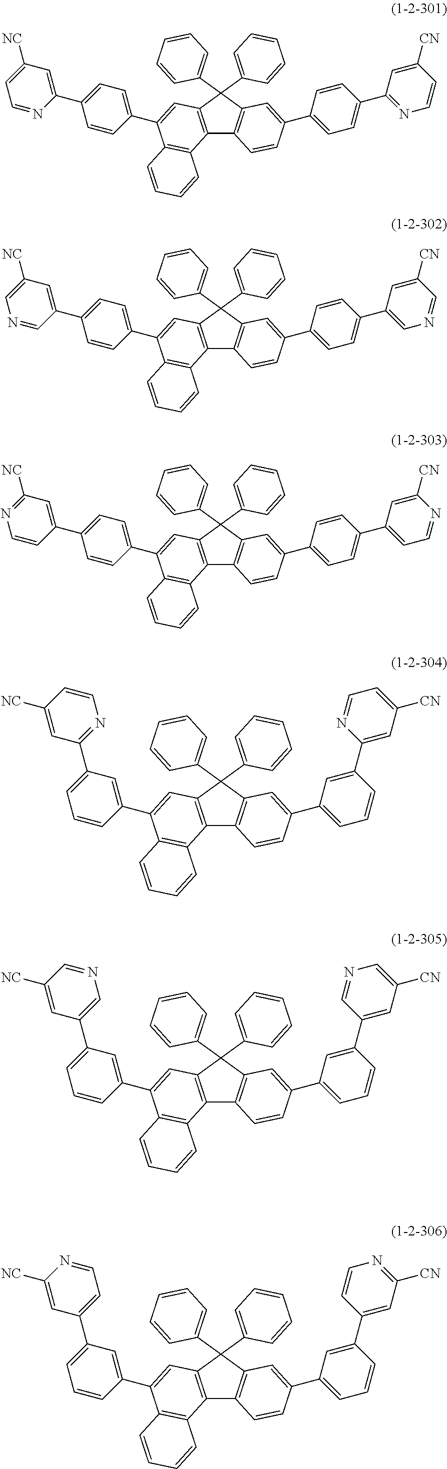

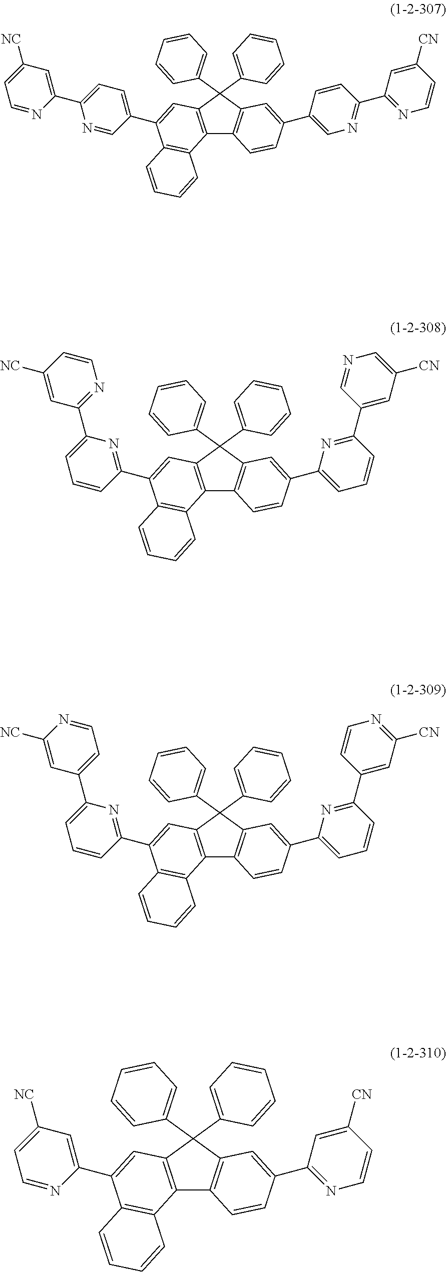 [SMILES: N#Cc1ccnc(-c2ccc(-c3ccc4c(c3)C(c3ccccc3)(c3ccccc3)c3cc(-c5ccc(-c6cc(C#N)ccn6)nc5)c5ccccc5c3-4)cn2)c1 ; N#Cc1cncc(-c2cccc(-c3ccc4c(c3)C(c3ccccc3)(c3ccccc3)c3cc(-c5cccc(-c6cc(C#N)ccn6)n5)c5ccccc5c3-4)n2)c1 ; N#Cc1cc(-c2cccc(-c3ccc4c(c3)C(c3ccccc3)(c3ccccc3)c3cc(-c5cccc(-c6ccnc(C#N)c6)n5)c5ccccc5c3-4)n2)ccn1 ; N#Cc1ccnc(-c2ccc3c(c2)C(c2ccccc2)(c2ccccc2)c2cc(-c4cc(C#N)ccn4)c4ccccc4c2-3)c1]

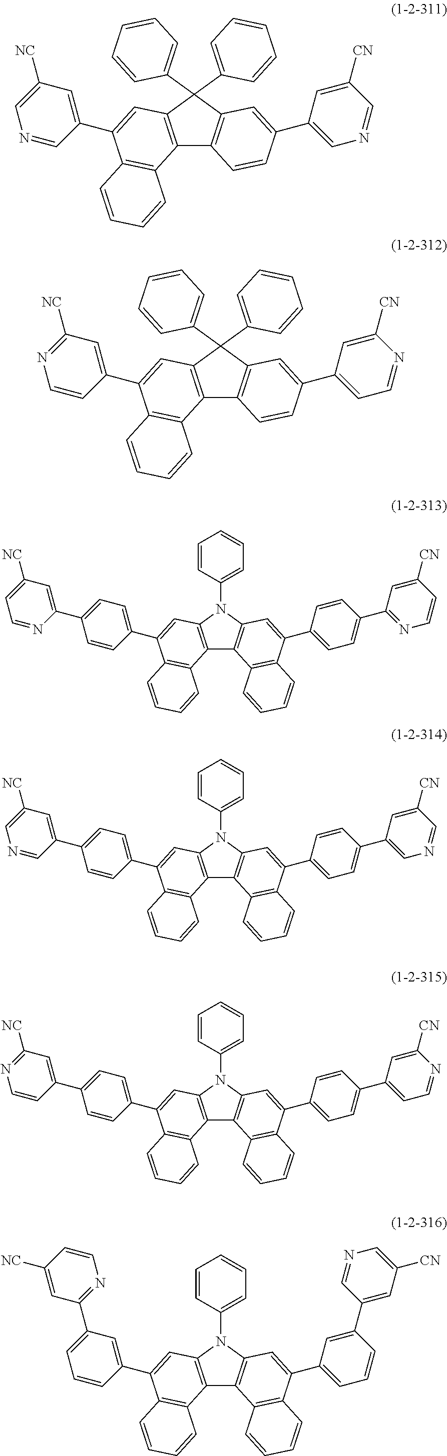

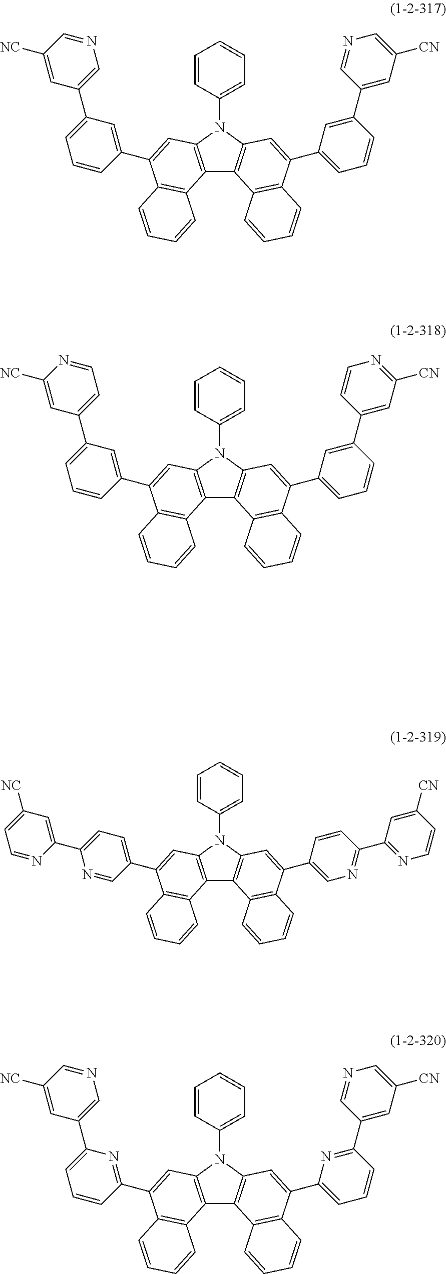 [SMILES: N#Cc1cncc(-c2cccc(-c3cc4c(c5ccccc35)c3c5ccccc5c(-c5cccc(-c6cncc(C#N)c6)c5)cc3n4-c3ccccc3)c2)c1 ; N#Cc1cc(-c2cccc(-c3cc4c(c5ccccc35)c3c5ccccc5c(-c5cccc(-c6ccnc(C#N)c6)c5)cc3n4-c3ccccc3)c2)ccn1 ; N#Cc1ccnc(-c2ccc(-c3cc4c(c5ccccc35)c3c5ccccc5c(-c5ccc(-c6cc(C#N)ccn6)nc5)cc3n4-c3ccccc3)cn2)c1 ; N#Cc1cncc(-c2cccc(-c3cc4c(c5ccccc35)c3c5ccccc5c(-c5cccc(-c6cncc(C#N)c6)n5)cc3n4-c3ccccc3)n2)c1]

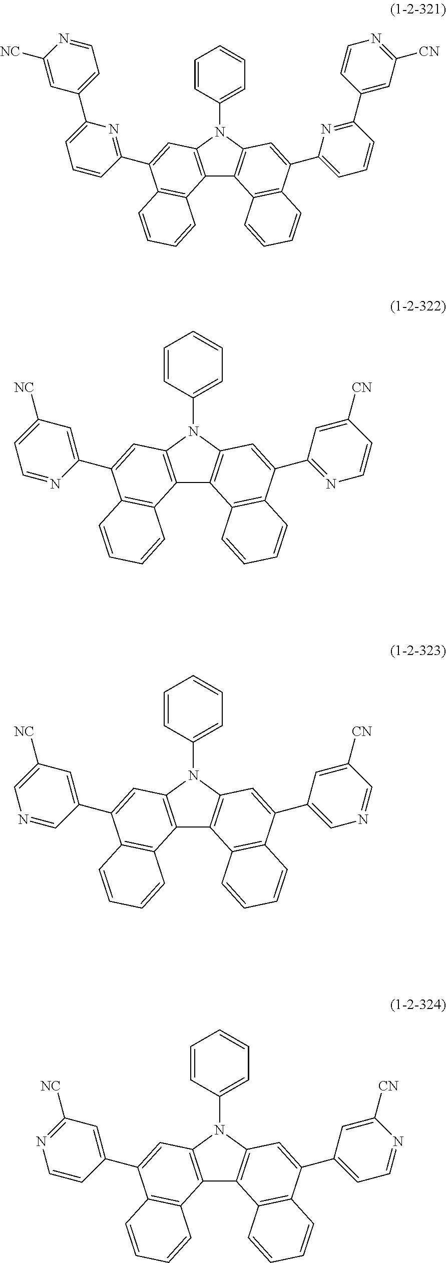 [SMILES: N#Cc1cc(-c2cccc(-c3cc4c(c5ccccc35)c3c5ccccc5c(-c5cccc(-c6ccnc(C#N)c6)n5)cc3n4-c3ccccc3)n2)ccn1 ; N#Cc1ccnc(-c2cc3c(c4ccccc24)c2c4ccccc4c(-c4cc(C#N)ccn4)cc2n3-c2ccccc2)c1 ; N#Cc1cncc(-c2cc3c(c4ccccc24)c2c4ccccc4c(-c4cncc(C#N)c4)cc2n3-c2ccccc2)c1 ; N#Cc1cc(-c2cc3c(c4ccccc24)c2c4ccccc4c(-c4ccnc(C#N)c4)cc2n3-c2ccccc2)ccn1]

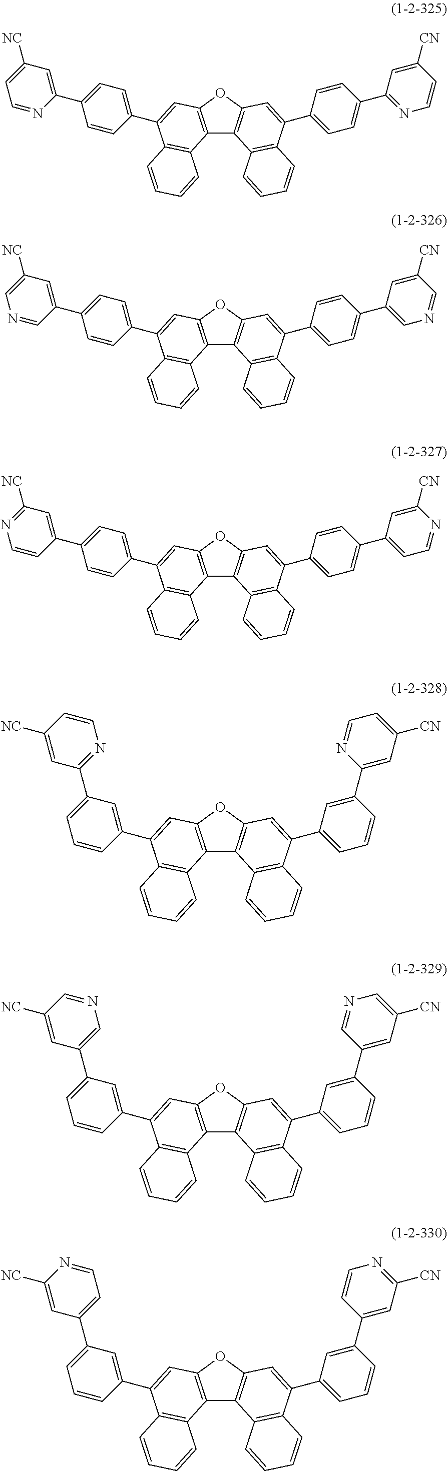

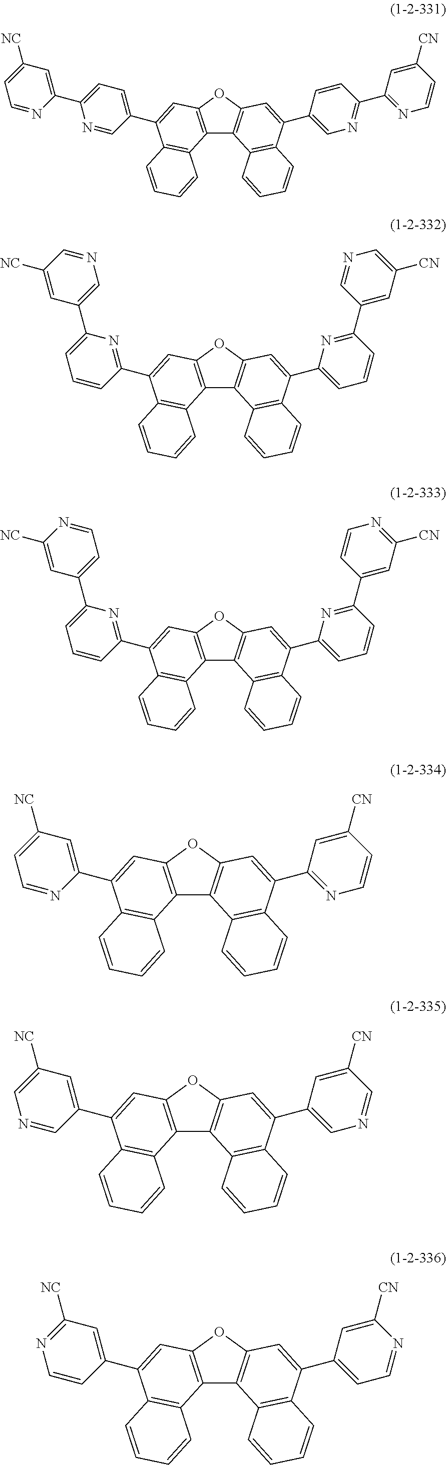 [SMILES: N#Cc1ccnc(-c2ccc(-c3cc4oc5cc(-c6ccc(-c7cc(C#N)ccn7)nc6)c6ccccc6c5c4c4ccccc34)cn2)c1 ; N#Cc1cncc(-c2cccc(-c3cc4oc5cc(-c6cccc(-c7cncc(C#N)c7)n6)c6ccccc6c5c4c4ccccc34)n2)c1 ; N#Cc1cc(-c2cccc(-c3cc4oc5cc(-c6cccc(-c7ccnc(C#N)c7)n6)c6ccccc6c5c4c4ccccc34)n2)ccn1 ; N#Cc1ccnc(-c2cc3oc4cc(-c5cc(C#N)ccn5)c5ccccc5c4c3c3ccccc23)c1 ; N#Cc1cncc(-c2cc3oc4cc(-c5cncc(C#N)c5)c5ccccc5c4c3c3ccccc23)c1 ; N#Cc1cc(-c2cc3oc4cc(-c5ccnc(C#N)c5)c5ccccc5c4c3c3ccccc23)ccn1]

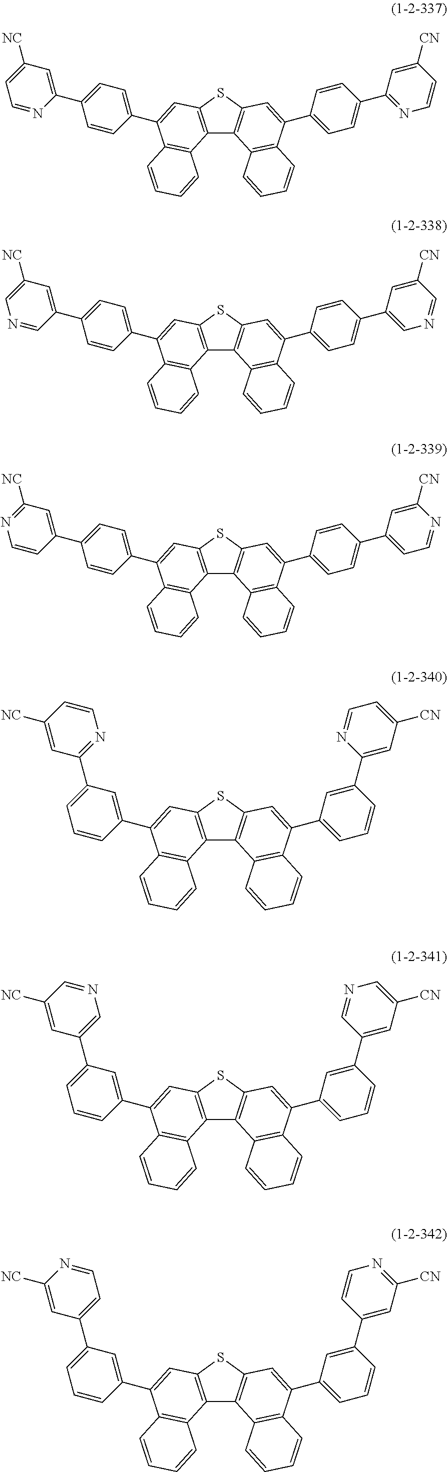

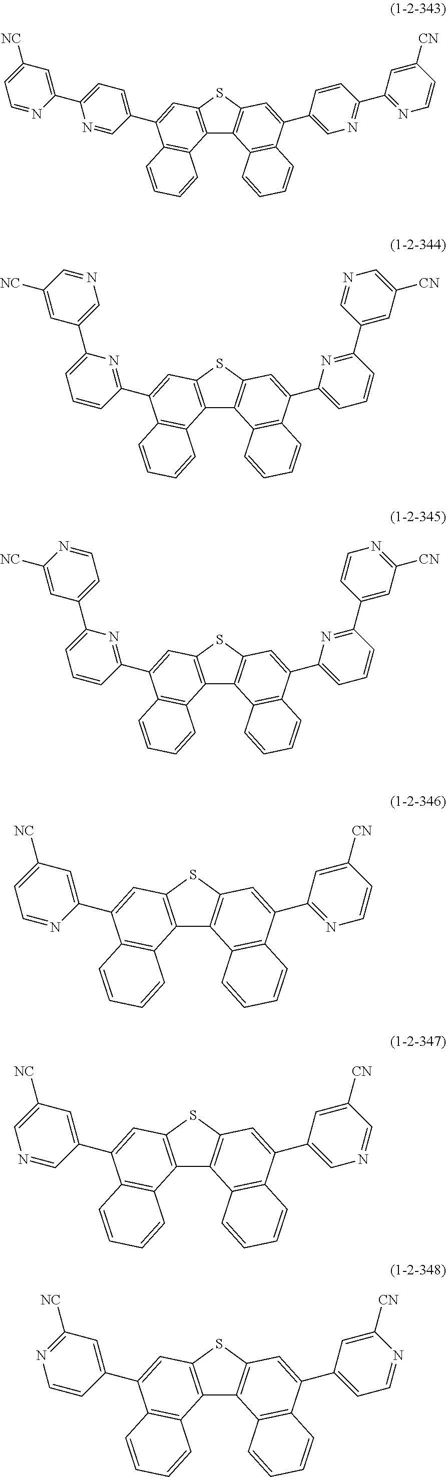 [SMILES: N#Cc1ccnc(-c2ccc(-c3cc4sc5cc(-c6ccc(-c7cc(C#N)ccn7)nc6)c6ccccc6c5c4c4ccccc34)cn2)c1 ; N#Cc1cncc(-c2cccc(-c3cc4sc5cc(-c6cccc(-c7cncc(C#N)c7)n6)c6ccccc6c5c4c4ccccc34)n2)c1 ; N#Cc1cc(-c2cccc(-c3cc4sc5cc(-c6cccc(-c7ccnc(C#N)c7)n6)c6ccccc6c5c4c4ccccc34)n2)ccn1 ; N#Cc1ccnc(-c2cc3sc4cc(-c5cc(C#N)ccn5)c5ccccc5c4c3c3ccccc23)c1 ; N#Cc1cncc(-c2cc3sc4cc(-c5cncc(C#N)c5)c5ccccc5c4c3c3ccccc23)c1 ; N#Cc1cc(-c2cc3sc4cc(-c5ccnc(C#N)c5)c5ccccc5c4c3c3ccccc23)ccn1]

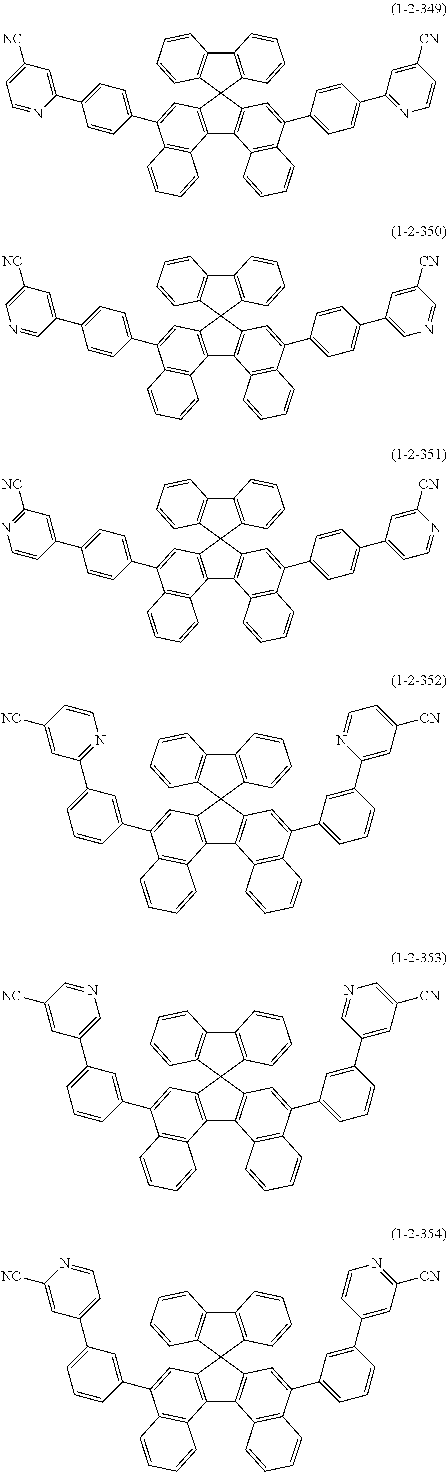

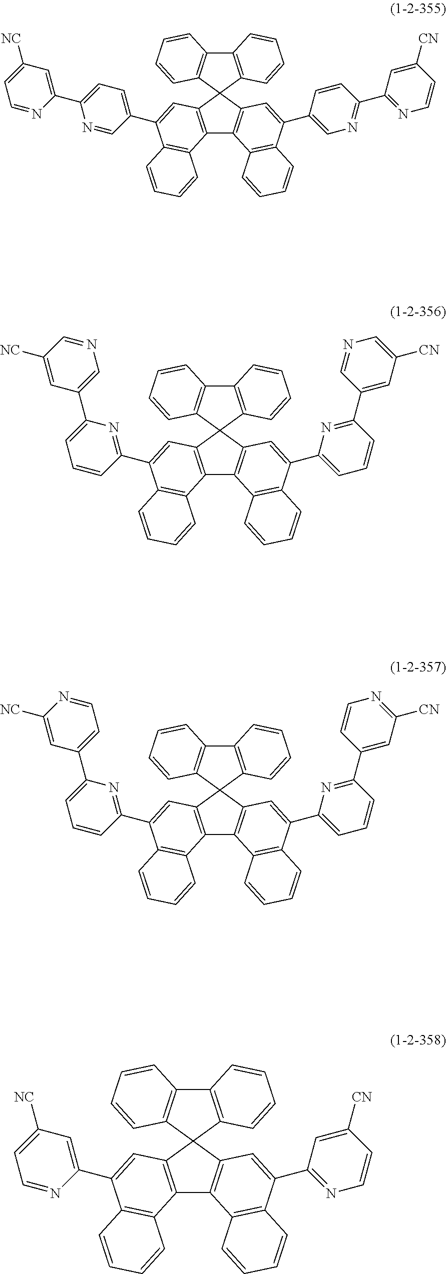 [SMILES: N#Cc1ccnc(-c2ccc(-c3cc4c(c5ccccc35)-c3c(cc(-c5ccc(-c6cc(C#N)ccn6)nc5)c5ccccc35)C43c4ccccc4-c4ccccc43)cn2)c1 ; N#Cc1cncc(-c2cccc(-c3cc4c(c5ccccc35)-c3c(cc(-c5cccc(-c6cncc(C#N)c6)n5)c5ccccc35)C43c4ccccc4-c4ccccc43)n2)c1 ; N#Cc1cc(-c2cccc(-c3cc4c(c5ccccc35)-c3c(cc(-c5cccc(-c6ccnc(C#N)c6)n5)c5ccccc35)C43c4ccccc4-c4ccccc43)n2)ccn1 ; N#Cc1ccnc(-c2cc3c(c4ccccc24)-c2c(cc(-c4cc(C#N)ccn4)c4ccccc24)C32c3ccccc3-c3ccccc32)c1]

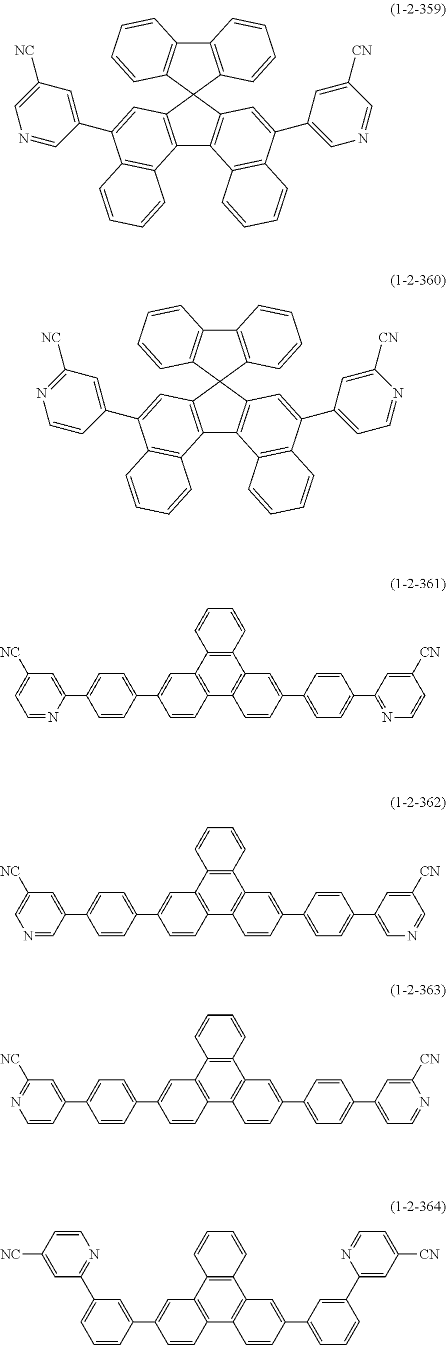 [SMILES: N#Cc1cncc(-c2cc3c(c4ccccc24)-c2c(cc(-c4cncc(C#N)c4)c4ccccc24)C32c3ccccc3-c3ccccc32)c1 ; N#Cc1cc(-c2cc3c(c4ccccc24)-c2c(cc(-c4ccnc(C#N)c4)c4ccccc24)C32c3ccccc3-c3ccccc32)ccn1 ; N#Cc1ccnc(-c2ccc(-c3ccc4c5ccc(-c6ccc(-c7cc(C#N)ccn7)cc6)cc5c5ccccc5c4c3)cc2)c1 ; N#Cc1cncc(-c2ccc(-c3ccc4c5ccc(-c6ccc(-c7cncc(C#N)c7)cc6)cc5c5ccccc5c4c3)cc2)c1 ; N#Cc1cc(-c2ccc(-c3ccc4c5ccc(-c6ccc(-c7ccnc(C#N)c7)cc6)cc5c5ccccc5c4c3)cc2)ccn1 ; N#Cc1ccnc(-c2cccc(-c3ccc4c5ccc(-c6cccc(-c7cc(C#N)ccn7)c6)cc5c5ccccc5c4c3)c2)c1]

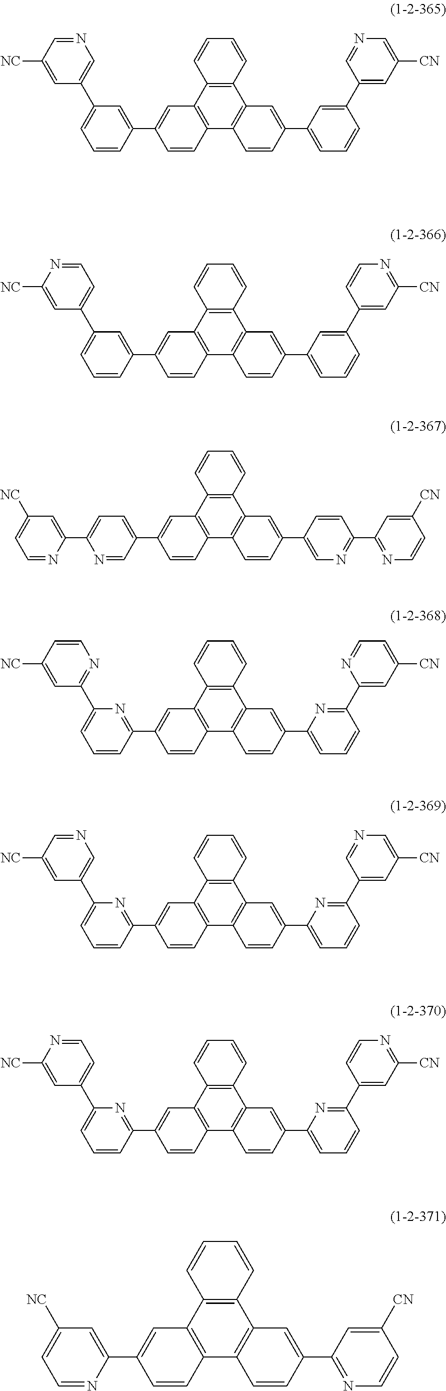

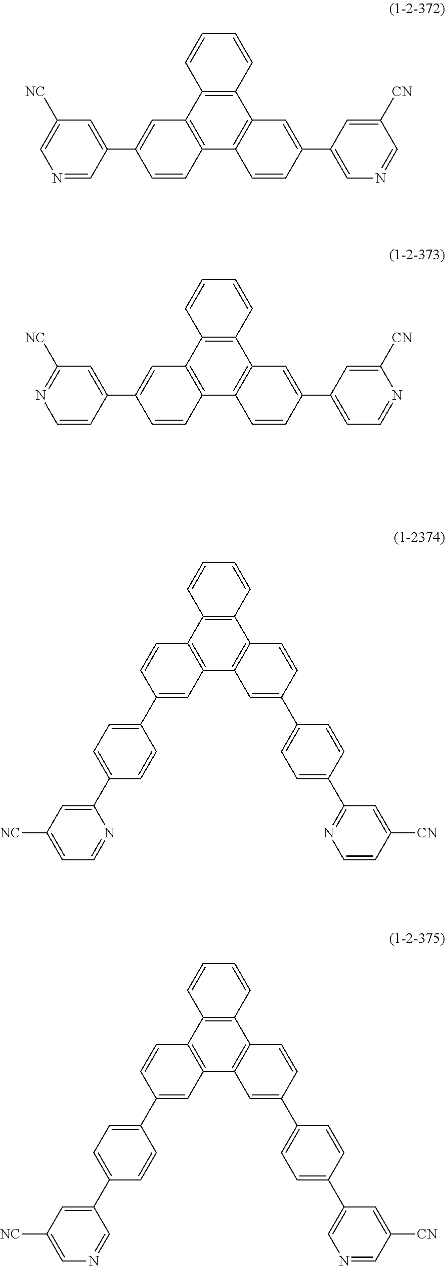 [SMILES: N#Cc1cncc(-c2ccc3c4ccc(-c5cncc(C#N)c5)cc4c4ccccc4c3c2)c1 ; N#Cc1cc(-c2ccc3c4ccc(-c5ccnc(C#N)c5)cc4c4ccccc4c3c2)ccn1 ; N#Cc1ccnc(-c2ccc(-c3ccc4c5ccccc5c5ccc(-c6ccc(-c7cc(C#N)ccn7)cc6)cc5c4c3)cc2)c1 ; N#Cc1cncc(-c2ccc(-c3ccc4c5ccccc5c5ccc(-c6ccc(-c7cncc(C#N)c7)cc6)cc5c4c3)cc2)c1]

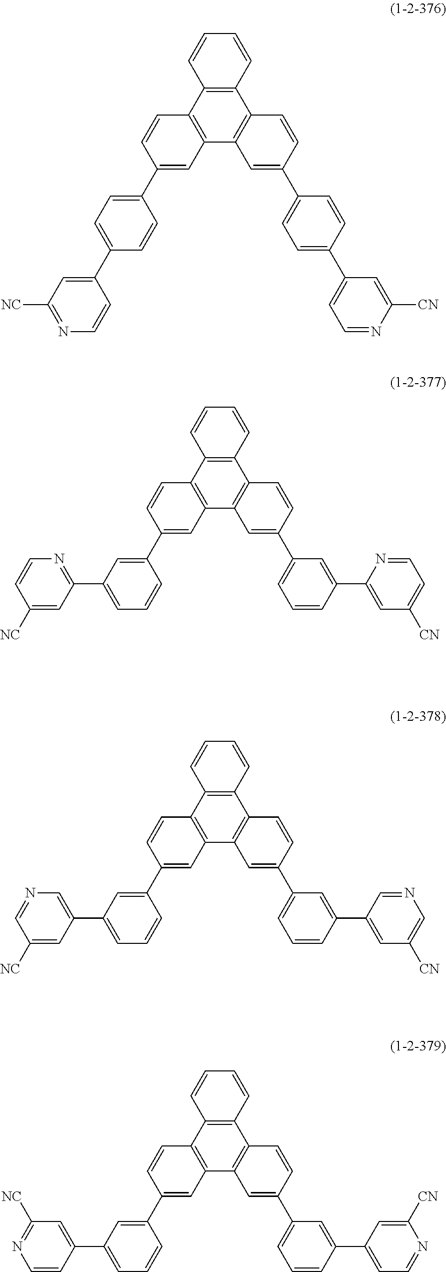 [SMILES: N#Cc1cc(-c2ccc(-c3ccc4c5ccccc5c5ccc(-c6ccc(-c7ccnc(C#N)c7)cc6)cc5c4c3)cc2)ccn1 ; N#Cc1ccnc(-c2cccc(-c3ccc4c5ccccc5c5ccc(-c6cccc(-c7cc(C#N)ccn7)c6)cc5c4c3)c2)c1 ; N#Cc1cncc(-c2cccc(-c3ccc4c5ccccc5c5ccc(-c6cccc(-c7cncc(C#N)c7)c6)cc5c4c3)c2)c1 ; N#Cc1cc(-c2cccc(-c3ccc4c5ccccc5c5ccc(-c6cccc(-c7ccnc(C#N)c7)c6)cc5c4c3)c2)ccn1]

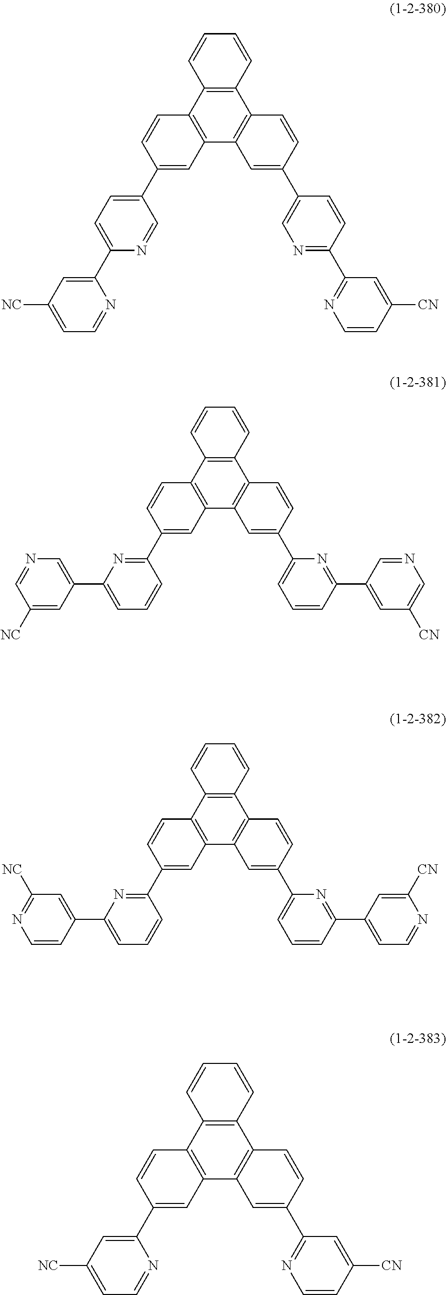 [SMILES: N#Cc1ccnc(-c2ccc(-c3ccc4c5ccccc5c5ccc(-c6ccc(-c7cc(C#N)ccn7)nc6)cc5c4c3)cn2)c1 ; N#Cc1cncc(-c2cccc(-c3ccc4c5ccccc5c5ccc(-c6cccc(-c7cncc(C#N)c7)n6)cc5c4c3)n2)c1 ; N#Cc1cc(-c2cccc(-c3ccc4c5ccccc5c5ccc(-c6cccc(-c7ccnc(C#N)c7)n6)cc5c4c3)n2)ccn1 ; N#Cc1ccnc(-c2ccc3c4ccccc4c4ccc(-c5cc(C#N)ccn5)cc4c3c2)c1]

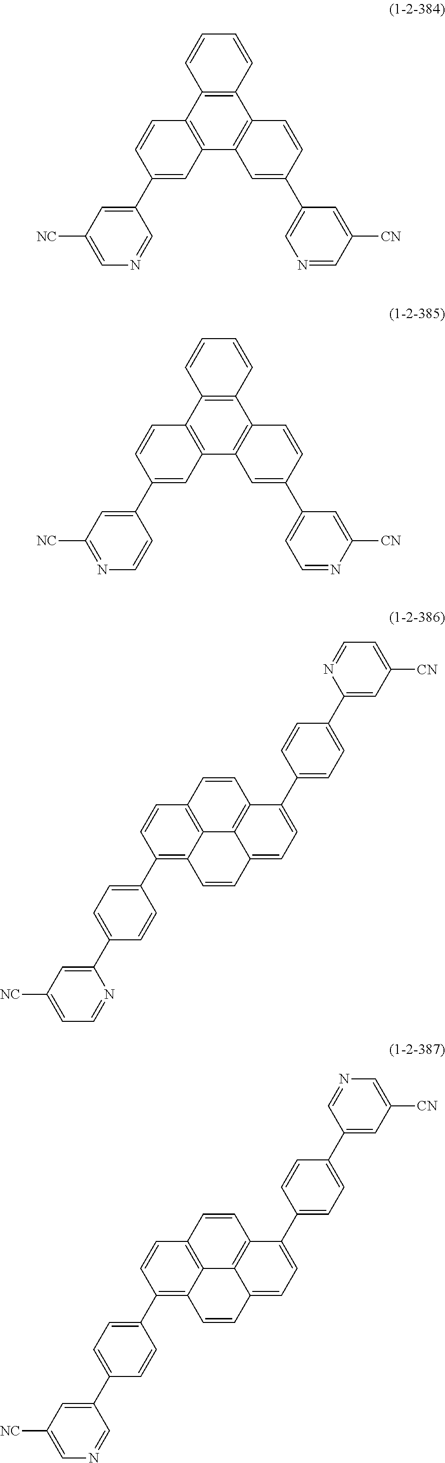 [SMILES: N#Cc1cncc(-c2ccc3c4ccccc4c4ccc(-c5cncc(C#N)c5)cc4c3c2)c1 ; N#Cc1cc(-c2ccc3c4ccccc4c4ccc(-c5ccnc(C#N)c5)cc4c3c2)ccn1 ; N#Cc1ccnc(-c2ccc(-c3ccc4ccc5c(-c6ccc(-c7cc(C#N)ccn7)cc6)ccc6ccc3c4c65)cc2)c1 ; N#Cc1cncc(-c2ccc(-c3ccc4ccc5c(-c6ccc(-c7cncc(C#N)c7)cc6)ccc6ccc3c4c65)cc2)c1]

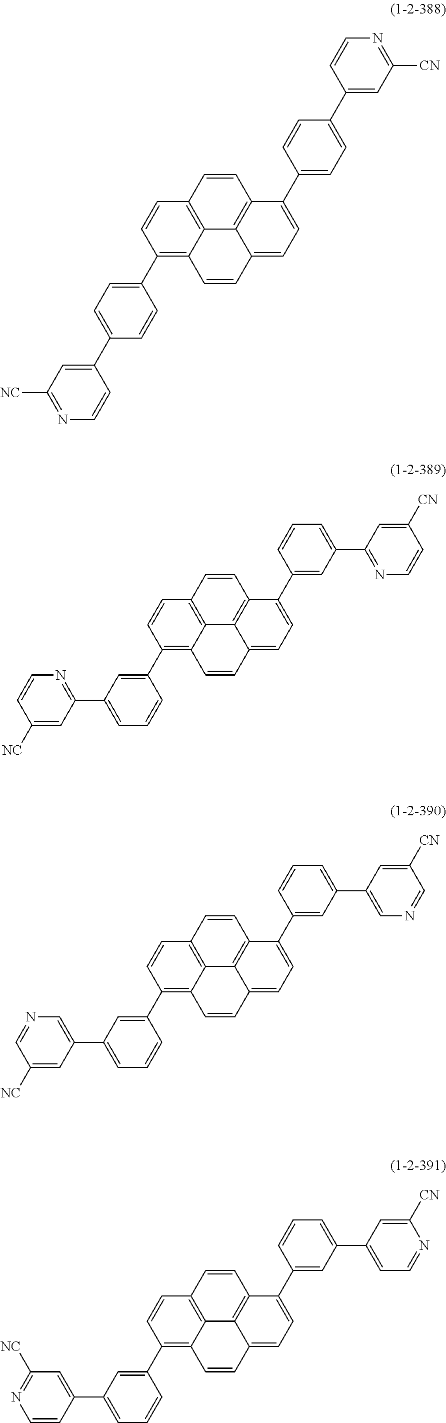 [SMILES: N#Cc1cc(-c2ccc(-c3ccc4ccc5c(-c6ccc(-c7ccnc(C#N)c7)cc6)ccc6ccc3c4c65)cc2)ccn1 ; N#Cc1ccnc(-c2cccc(-c3ccc4ccc5c(-c6cccc(-c7cc(C#N)ccn7)c6)ccc6ccc3c4c65)c2)c1 ; N#Cc1cncc(-c2cccc(-c3ccc4ccc5c(-c6cccc(-c7cncc(C#N)c7)c6)ccc6ccc3c4c65)c2)c1 ; N#Cc1cc(-c2cccc(-c3ccc4ccc5c(-c6cccc(-c7ccnc(C#N)c7)c6)ccc6ccc3c4c65)c2)ccn1]

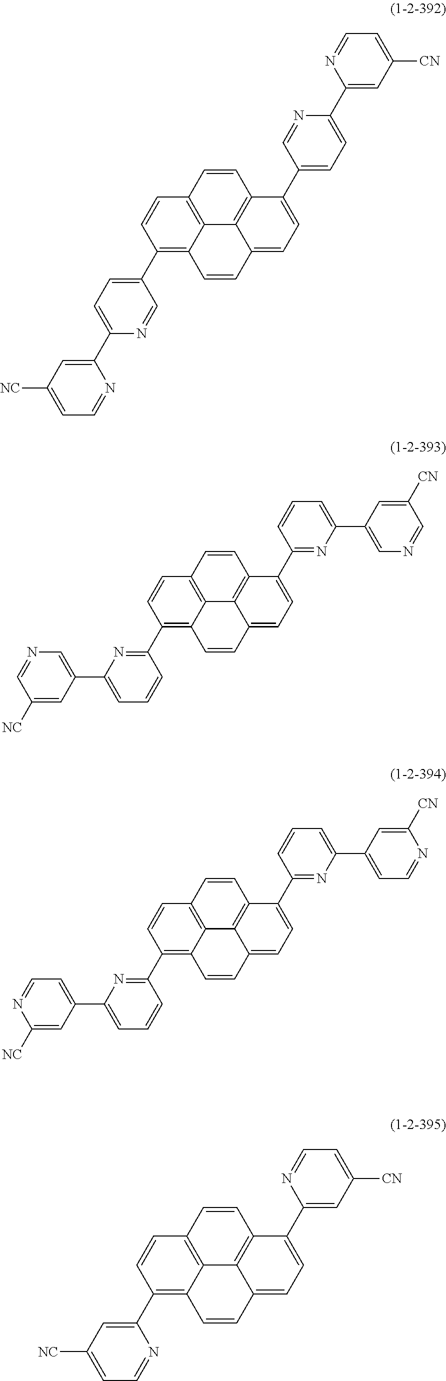 [SMILES: N#Cc1ccnc(-c2ccc(-c3ccc4ccc5c(-c6ccc(-c7cc(C#N)ccn7)nc6)ccc6ccc3c4c65)cn2)c1 ; N#Cc1cncc(-c2cccc(-c3ccc4ccc5c(-c6cccc(-c7cncc(C#N)c7)n6)ccc6ccc3c4c65)n2)c1 ; N#Cc1cc(-c2cccc(-c3ccc4ccc5c(-c6cccc(-c7ccnc(C#N)c7)n6)ccc6ccc3c4c65)n2)ccn1 ; N#Cc1ccnc(-c2ccc3ccc4c(-c5cc(C#N)ccn5)ccc5ccc2c3c54)c1]

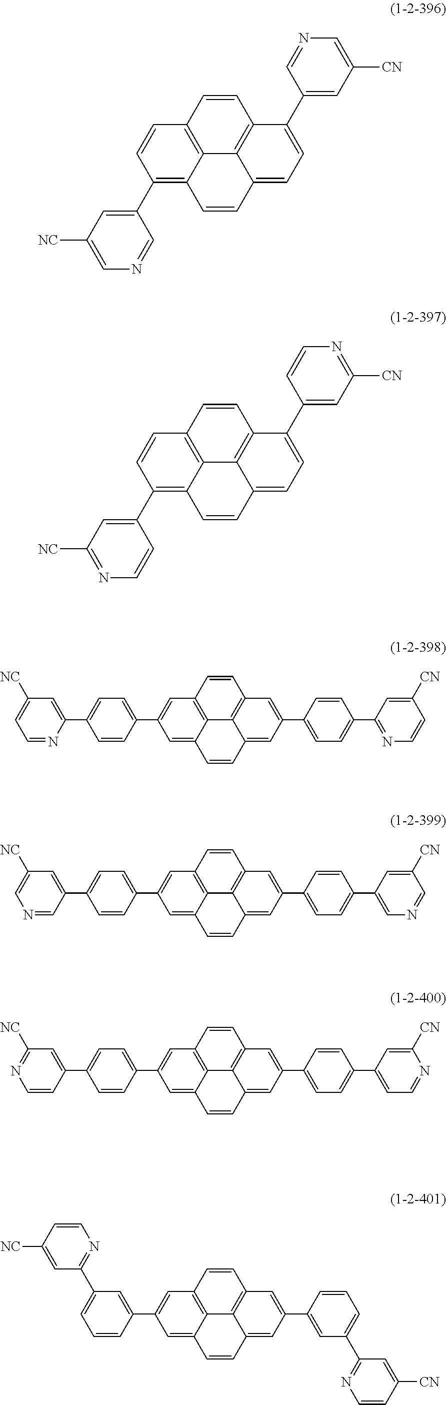 [SMILES: N#Cc1cncc(-c2ccc3ccc4c(-c5cncc(C#N)c5)ccc5ccc2c3c54)c1 ; N#Cc1cc(-c2ccc3ccc4c(-c5ccnc(C#N)c5)ccc5ccc2c3c54)ccn1 ; N#Cc1ccnc(-c2ccc(-c3cc4ccc5cc(-c6ccc(-c7cc(C#N)ccn7)cc6)cc6ccc(c3)c4c56)cc2)c1 ; N#Cc1cncc(-c2ccc(-c3cc4ccc5cc(-c6ccc(-c7cncc(C#N)c7)cc6)cc6ccc(c3)c4c56)cc2)c1 ; N#Cc1cc(-c2ccc(-c3cc4ccc5cc(-c6ccc(-c7ccnc(C#N)c7)cc6)cc6ccc(c3)c4c56)cc2)ccn1 ; N#Cc1ccnc(-c2cccc(-c3cc4ccc5cc(-c6cccc(-c7cc(C#N)ccn7)c6)cc6ccc(c3)c4c56)c2)c1]

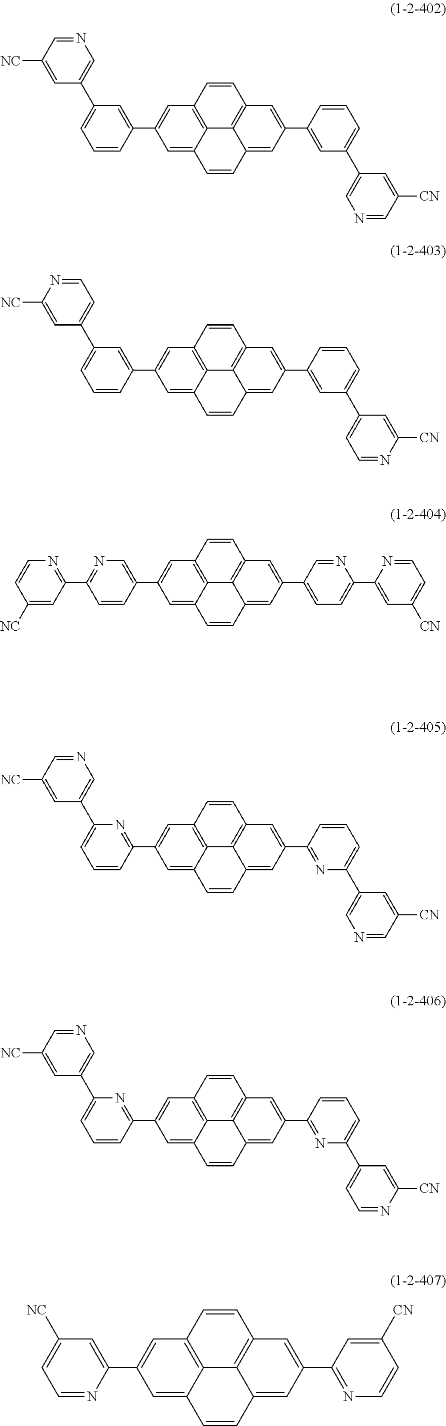 [SMILES: N#Cc1cncc(-c2cccc(-c3cc4ccc5cc(-c6cccc(-c7cncc(C#N)c7)c6)cc6ccc(c3)c4c56)c2)c1 ; N#Cc1cc(-c2cccc(-c3cc4ccc5cc(-c6cccc(-c7ccnc(C#N)c7)c6)cc6ccc(c3)c4c56)c2)ccn1 ; N#Cc1ccnc(-c2ccc(-c3cc4ccc5cc(-c6ccc(-c7cc(C#N)ccn7)nc6)cc6ccc(c3)c4c56)cn2)c1 ; N#Cc1cncc(-c2cccc(-c3cc4ccc5cc(-c6cccc(-c7cncc(C#N)c7)n6)cc6ccc(c3)c4c56)n2)c1 ; N#Cc1cncc(-c2cccc(-c3cc4ccc5cc(-c6cccc(-c7ccnc(C#N)c7)n6)cc6ccc(c3)c4c56)n2)c1 ; N#Cc1ccnc(-c2cc3ccc4cc(-c5cc(C#N)ccn5)cc5ccc(c2)c3c45)c1]

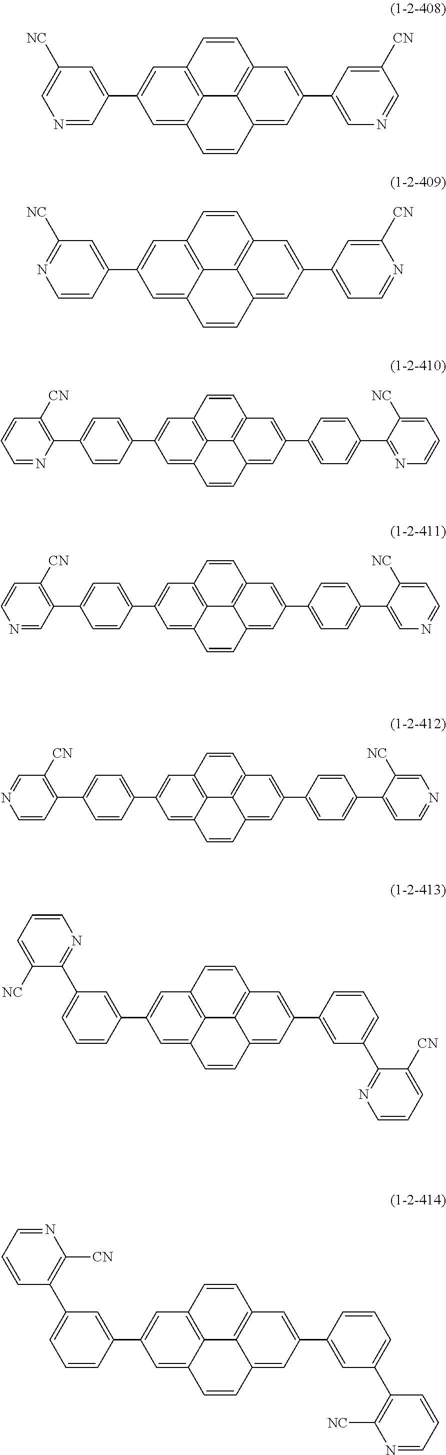

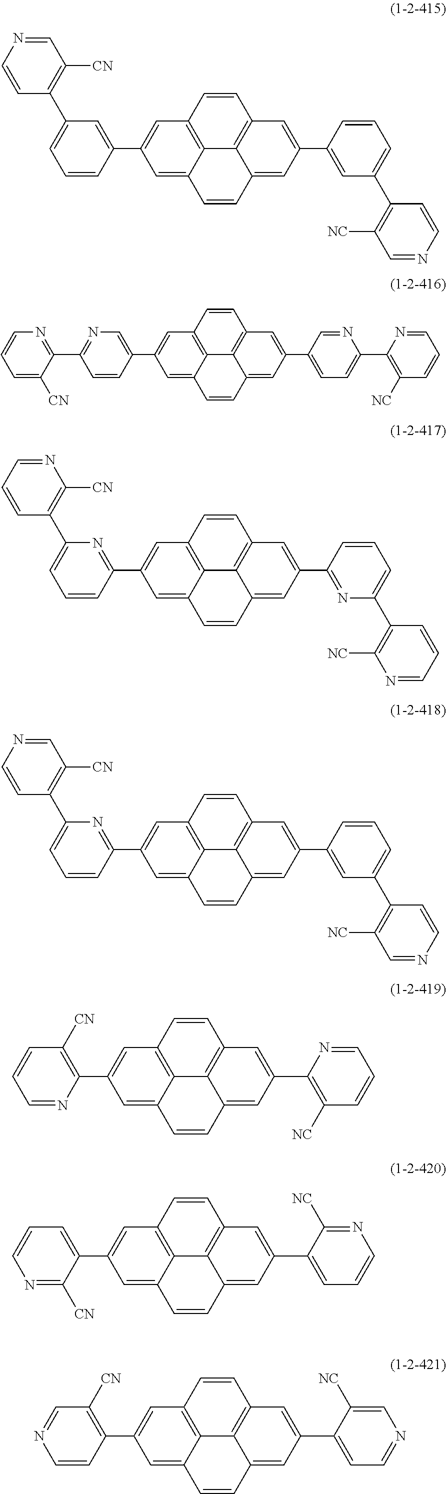

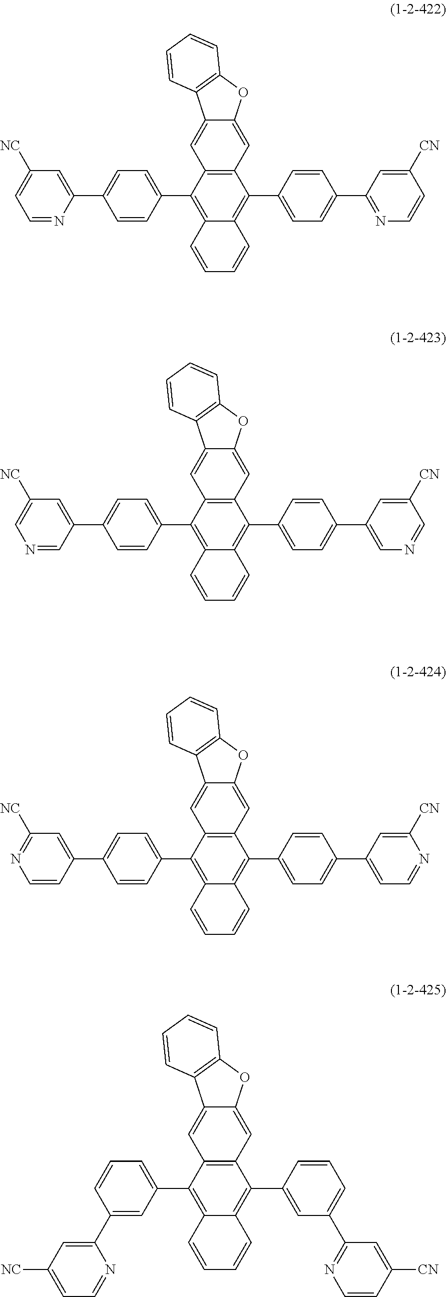 [SMILES: N#Cc1ccnc(-c2ccc(-c3c4ccccc4c(-c4ccc(-c5cc(C#N)ccn5)cc4)c4cc5c(cc34)oc3ccccc35)cc2)c1 ; N#Cc1cncc(-c2ccc(-c3c4ccccc4c(-c4ccc(-c5cncc(C#N)c5)cc4)c4cc5c(cc34)oc3ccccc35)cc2)c1 ; N#Cc1cc(-c2ccc(-c3c4ccccc4c(-c4ccc(-c5ccnc(C#N)c5)cc4)c4cc5c(cc34)oc3ccccc35)cc2)ccn1 ; N#Cc1ccnc(-c2cccc(-c3c4ccccc4c(-c4cccc(-c5cc(C#N)ccn5)c4)c4cc5c(cc34)oc3ccccc35)c2)c1]

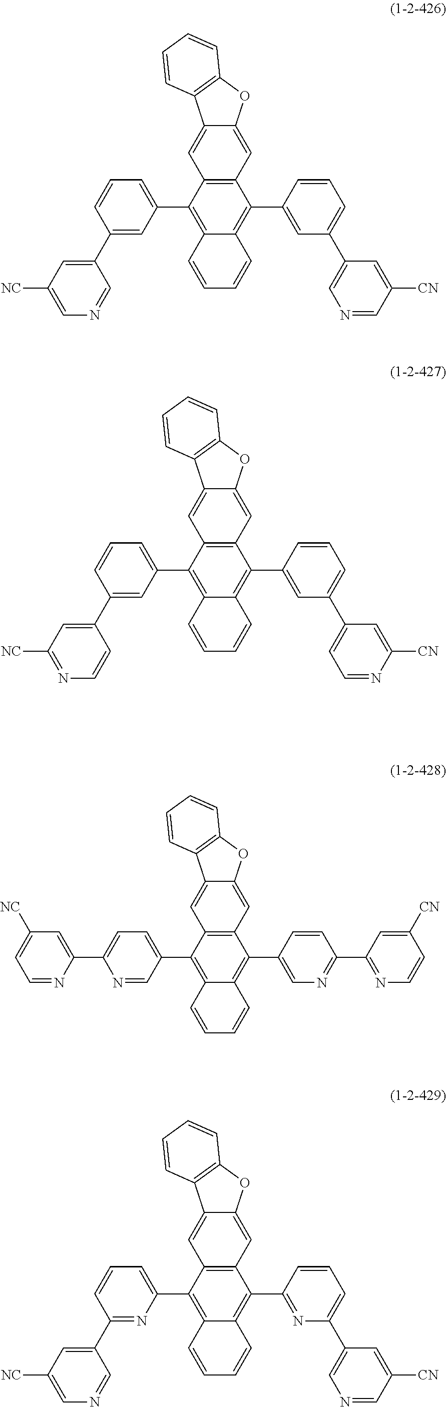 [SMILES: N#Cc1cncc(-c2cccc(-c3c4ccccc4c(-c4cccc(-c5cncc(C#N)c5)c4)c4cc5c(cc34)oc3ccccc35)c2)c1 ; N#Cc1cc(-c2cccc(-c3c4ccccc4c(-c4cccc(-c5ccnc(C#N)c5)c4)c4cc5c(cc34)oc3ccccc35)c2)ccn1 ; N#Cc1ccnc(-c2ccc(-c3c4ccccc4c(-c4ccc(-c5cc(C#N)ccn5)nc4)c4cc5c(cc34)oc3ccccc35)cn2)c1 ; N#Cc1cncc(-c2cccc(-c3c4ccccc4c(-c4cccc(-c5cncc(C#N)c5)n4)c4cc5c(cc34)oc3ccccc35)n2)c1]

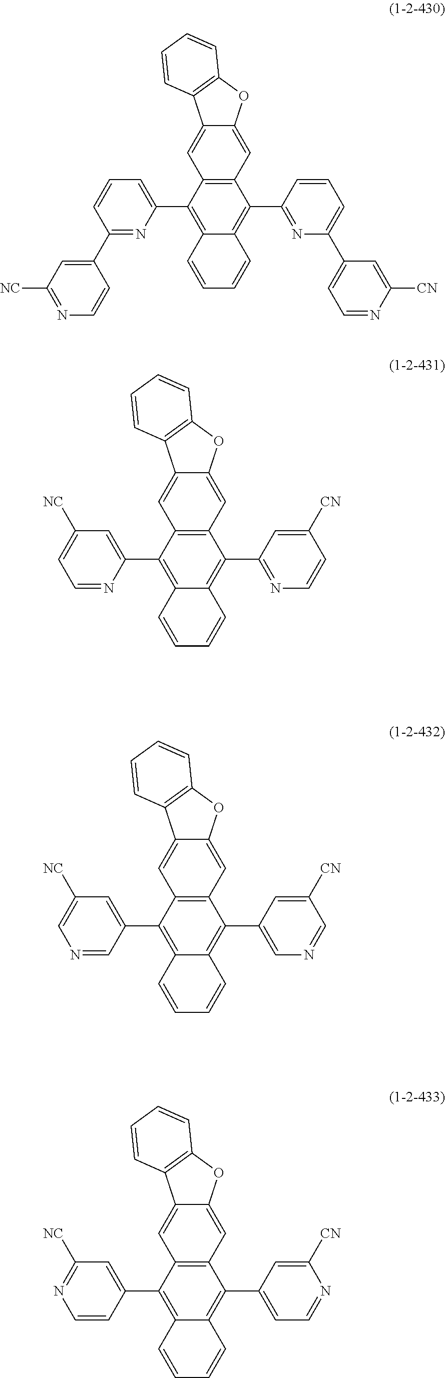 [SMILES: N#Cc1cc(-c2cccc(-c3c4ccccc4c(-c4cccc(-c5ccnc(C#N)c5)n4)c4cc5c(cc34)oc3ccccc35)n2)ccn1 ; N#Cc1ccnc(-c2c3ccccc3c(-c3cc(C#N)ccn3)c3cc4c(cc23)oc2ccccc24)c1 ; N#Cc1cncc(-c2c3ccccc3c(-c3cncc(C#N)c3)c3cc4c(cc23)oc2ccccc24)c1 ; N#Cc1cc(-c2c3ccccc3c(-c3ccnc(C#N)c3)c3cc4c(cc23)oc2ccccc24)ccn1]

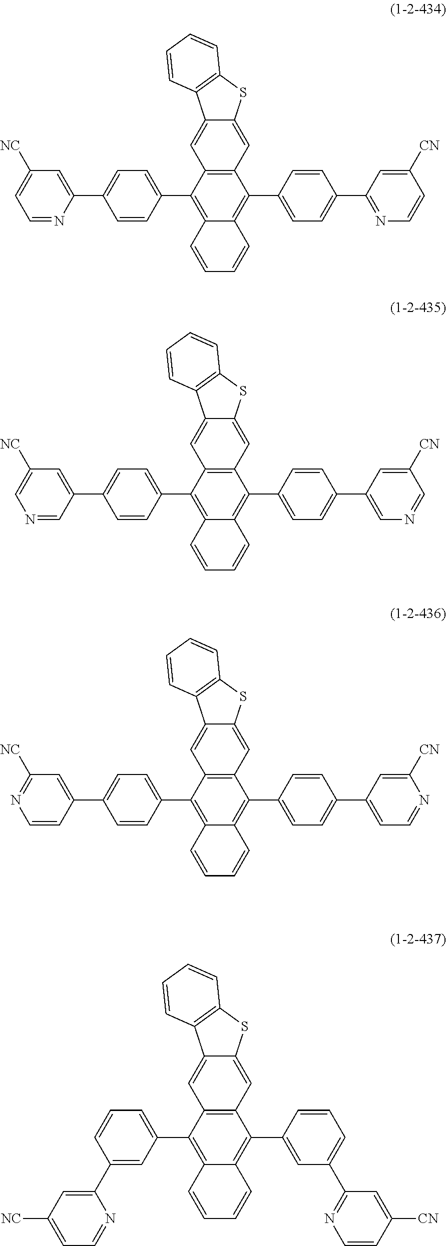 [SMILES: N#Cc1ccnc(-c2ccc(-c3c4ccccc4c(-c4ccc(-c5cc(C#N)ccn5)cc4)c4cc5c(cc34)sc3ccccc35)cc2)c1 ; N#Cc1cncc(-c2ccc(-c3c4ccccc4c(-c4ccc(-c5cncc(C#N)c5)cc4)c4cc5c(cc34)sc3ccccc35)cc2)c1 ; N#Cc1cc(-c2ccc(-c3c4ccccc4c(-c4ccc(-c5ccnc(C#N)c5)cc4)c4cc5c(cc34)sc3ccccc35)cc2)ccn1 ; N#Cc1ccnc(-c2cccc(-c3c4ccccc4c(-c4cccc(-c5cc(C#N)ccn5)c4)c4cc5c(cc34)sc3ccccc35)c2)c1]

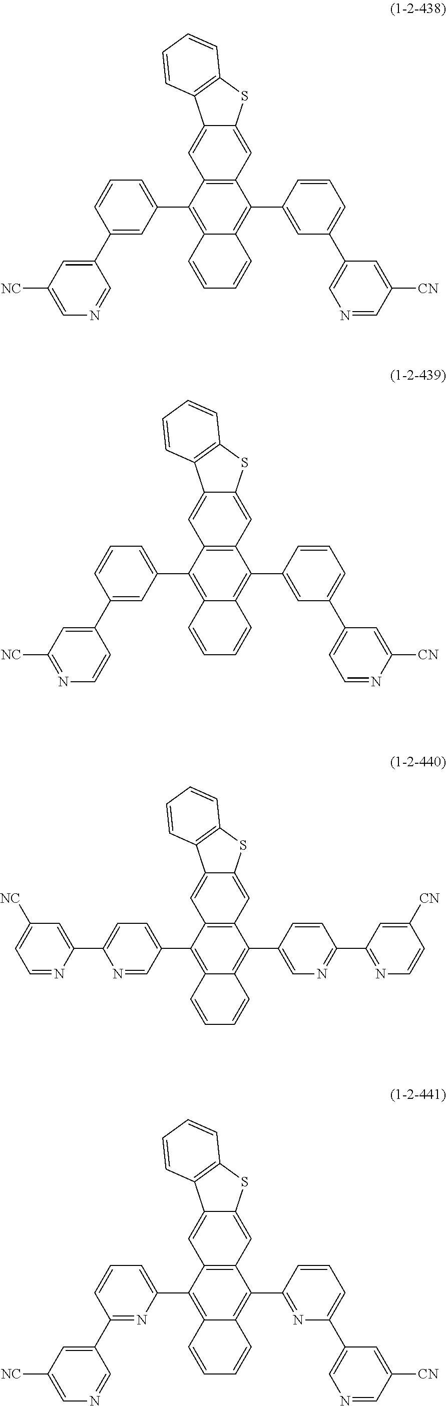 [SMILES: N#Cc1cncc(-c2cccc(-c3c4ccccc4c(-c4cccc(-c5cncc(C#N)c5)c4)c4cc5c(cc34)sc3ccccc35)c2)c1 ; N#Cc1cc(-c2cccc(-c3c4ccccc4c(-c4cccc(-c5ccnc(C#N)c5)c4)c4cc5c(cc34)sc3ccccc35)c2)ccn1 ; N#Cc1ccnc(-c2ccc(-c3c4ccccc4c(-c4ccc(-c5cc(C#N)ccn5)nc4)c4cc5c(cc34)sc3ccccc35)cn2)c1 ; N#Cc1cncc(-c2cccc(-c3c4ccccc4c(-c4cccc(-c5cncc(C#N)c5)n4)c4cc5c(cc34)sc3ccccc35)n2)c1]

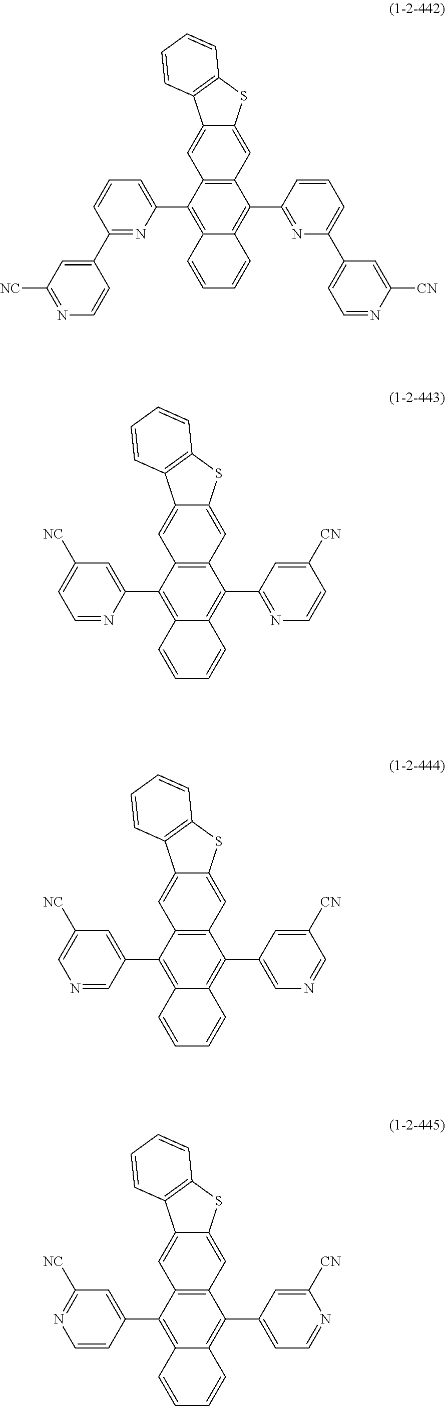 [SMILES: N#Cc1cc(-c2cccc(-c3c4ccccc4c(-c4cccc(-c5ccnc(C#N)c5)n4)c4cc5c(cc34)sc3ccccc35)n2)ccn1 ; N#Cc1ccnc(-c2c3ccccc3c(-c3cc(C#N)ccn3)c3cc4c(cc23)sc2ccccc24)c1 ; N#Cc1cncc(-c2c3ccccc3c(-c3cncc(C#N)c3)c3cc4c(cc23)sc2ccccc24)c1 ; N#Cc1cc(-c2c3ccccc3c(-c3ccnc(C#N)c3)c3cc4c(cc23)sc2ccccc24)ccn1]

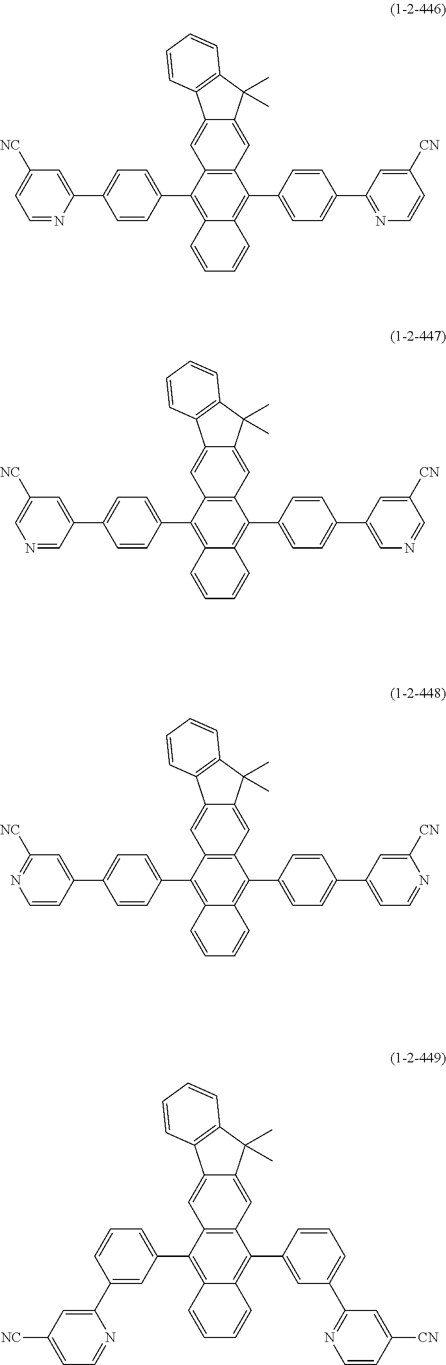 [SMILES: CC1(C)c2ccccc2-c2cc3c(-c4ccc(-c5cc(C#N)ccn5)cc4)c4ccccc4c(-c4ccc(-c5cc(C#N)ccn5)cc4)c3cc21 ; CC1(C)c2ccccc2-c2cc3c(-c4ccc(-c5cncc(C#N)c5)cc4)c4ccccc4c(-c4ccc(-c5cncc(C#N)c5)cc4)c3cc21 ; CC1(C)c2ccccc2-c2cc3c(-c4ccc(-c5ccnc(C#N)c5)cc4)c4ccccc4c(-c4ccc(-c5ccnc(C#N)c5)cc4)c3cc21 ; CC1(C)c2ccccc2-c2cc3c(-c4cccc(-c5cc(C#N)ccn5)c4)c4ccccc4c(-c4cccc(-c5cc(C#N)ccn5)c4)c3cc21]

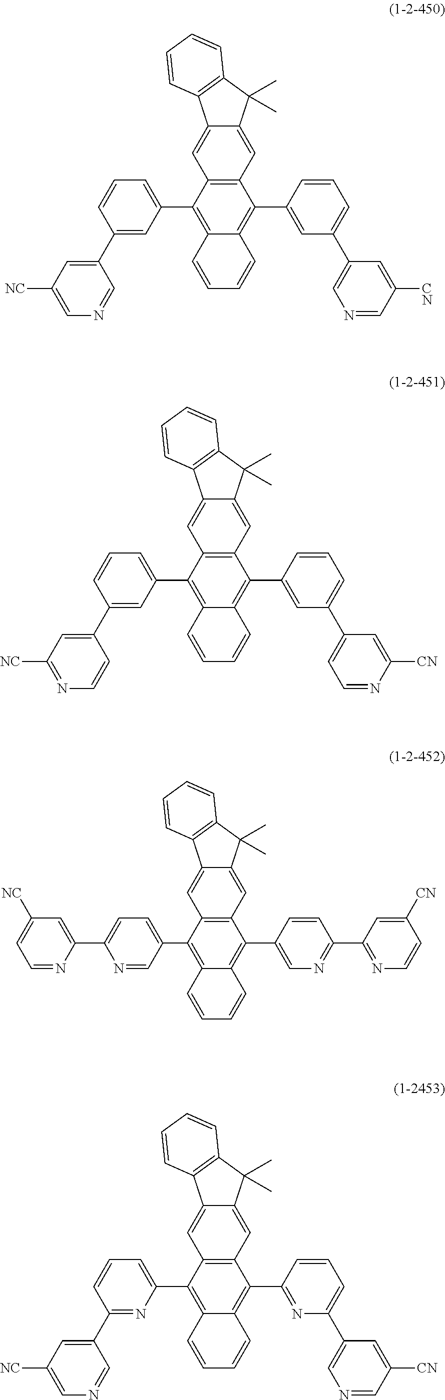 [SMILES: CC1(C)c2ccccc2-c2cc3c(-c4cccc(-c5cncc(C#N)c5)c4)c4ccccc4c(-c4cccc(-c5cncc(C#N)c5)c4)c3cc21 ; CC1(C)c2ccccc2-c2cc3c(-c4cccc(-c5ccnc(C#N)c5)c4)c4ccccc4c(-c4cccc(-c5ccnc(C#N)c5)c4)c3cc21 ; CC1(C)c2ccccc2-c2cc3c(-c4ccc(-c5cc(C#N)ccn5)nc4)c4ccccc4c(-c4ccc(-c5cc(C#N)ccn5)nc4)c3cc21 ; CC1(C)c2ccccc2-c2cc3c(-c4cccc(-c5cncc(C#N)c5)n4)c4ccccc4c(-c4cccc(-c5cncc(C#N)c5)n4)c3cc21]

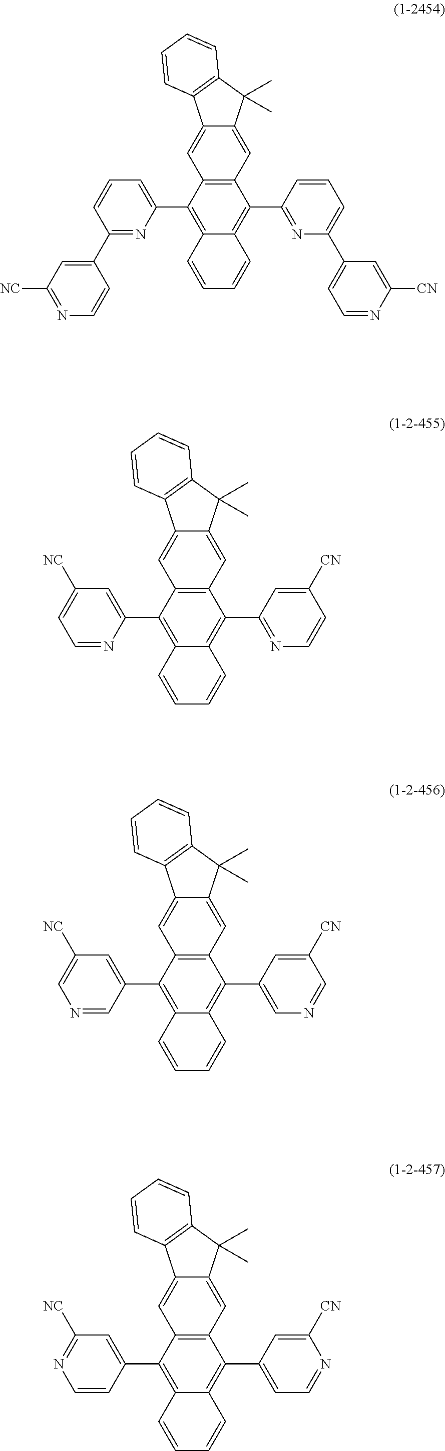 [SMILES: CC1(C)c2ccccc2-c2cc3c(-c4cccc(-c5ccnc(C#N)c5)n4)c4ccccc4c(-c4cccc(-c5ccnc(C#N)c5)n4)c3cc21 ; CC1(C)c2ccccc2-c2cc3c(-c4cc(C#N)ccn4)c4ccccc4c(-c4cc(C#N)ccn4)c3cc21 ; CC1(C)c2ccccc2-c2cc3c(-c4cncc(C#N)c4)c4ccccc4c(-c4cncc(C#N)c4)c3cc21 ; CC1(C)c2ccccc2-c2cc3c(-c4ccnc(C#N)c4)c4ccccc4c(-c4ccnc(C#N)c4)c3cc21]

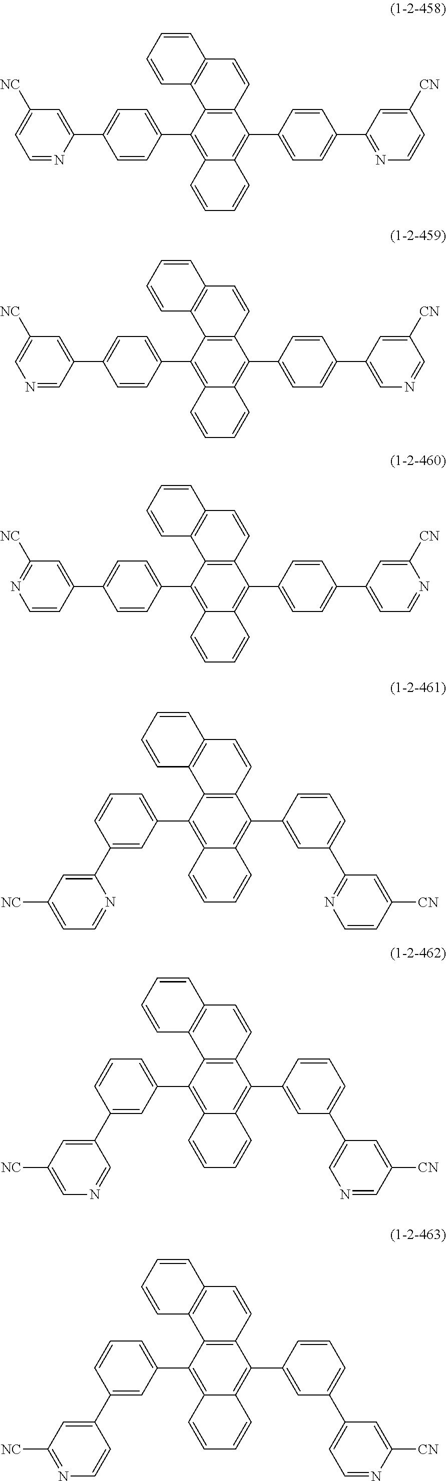 [SMILES: N#Cc1ccnc(-c2ccc(-c3c4ccccc4c(-c4ccc(-c5cc(C#N)ccn5)cc4)c4c3ccc3ccccc34)cc2)c1 ; N#Cc1cncc(-c2ccc(-c3c4ccccc4c(-c4ccc(-c5cncc(C#N)c5)cc4)c4c3ccc3ccccc34)cc2)c1 ; N#Cc1cc(-c2ccc(-c3c4ccccc4c(-c4ccc(-c5ccnc(C#N)c5)cc4)c4c3ccc3ccccc34)cc2)ccn1 ; N#Cc1ccnc(-c2cccc(-c3c4ccccc4c(-c4cccc(-c5cc(C#N)ccn5)c4)c4c3ccc3ccccc34)c2)c1 ; N#Cc1cncc(-c2cccc(-c3c4ccccc4c(-c4cccc(-c5cncc(C#N)c5)c4)c4c3ccc3ccccc34)c2)c1 ; N#Cc1cc(-c2cccc(-c3c4ccccc4c(-c4cccc(-c5ccnc(C#N)c5)c4)c4c3ccc3ccccc34)c2)ccn1]

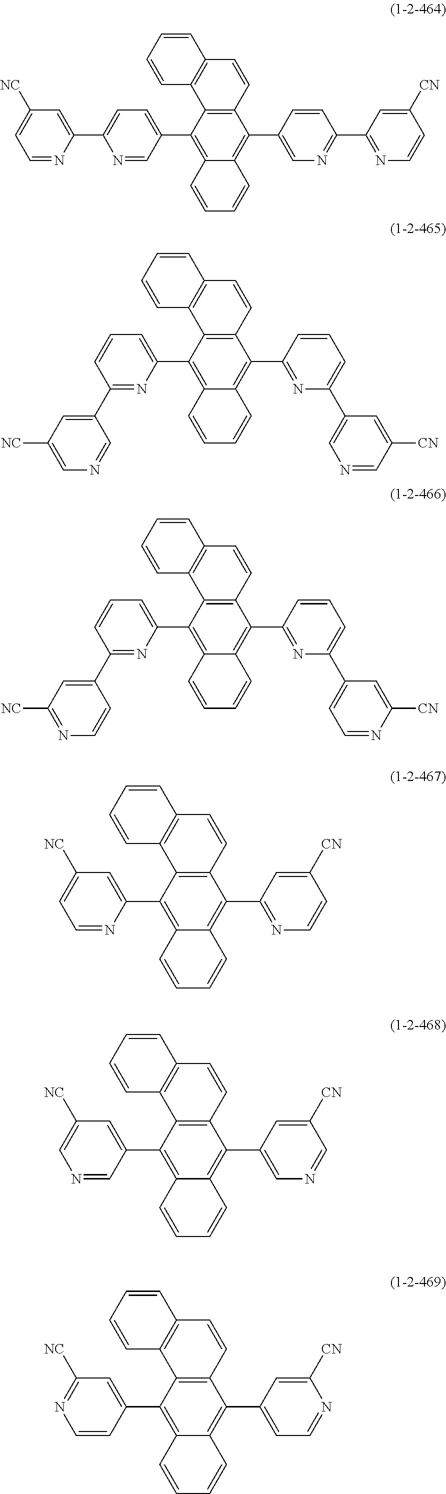 [SMILES: N#Cc1ccnc(-c2ccc(-c3c4ccccc4c(-c4ccc(-c5cc(C#N)ccn5)nc4)c4c3ccc3ccccc34)cn2)c1 ; N#Cc1cncc(-c2cccc(-c3c4ccccc4c(-c4cccc(-c5cncc(C#N)c5)n4)c4c3ccc3ccccc34)n2)c1 ; N#Cc1cc(-c2cccc(-c3c4ccccc4c(-c4cccc(-c5ccnc(C#N)c5)n4)c4c3ccc3ccccc34)n2)ccn1 ; N#Cc1ccnc(-c2c3ccccc3c(-c3cc(C#N)ccn3)c3c2ccc2ccccc23)c1 ; N#Cc1cncc(-c2c3ccccc3c(-c3cncc(C#N)c3)c3c2ccc2ccccc23)c1 ; N#Cc1cc(-c2c3ccccc3c(-c3ccnc(C#N)c3)c3c2ccc2ccccc23)ccn1]

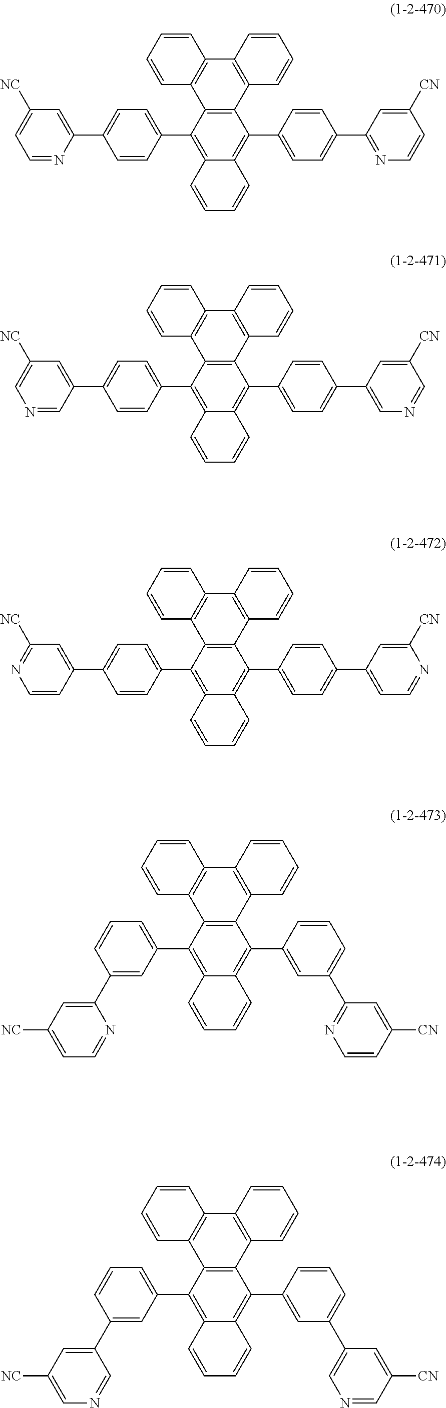 [SMILES: N#Cc1ccnc(-c2ccc(-c3c4ccccc4c(-c4ccc(-c5cc(C#N)ccn5)cc4)c4c5ccccc5c5ccccc5c34)cc2)c1 ; N#Cc1cncc(-c2ccc(-c3c4ccccc4c(-c4ccc(-c5cncc(C#N)c5)cc4)c4c5ccccc5c5ccccc5c34)cc2)c1 ; N#Cc1cc(-c2ccc(-c3c4ccccc4c(-c4ccc(-c5ccnc(C#N)c5)cc4)c4c5ccccc5c5ccccc5c34)cc2)ccn1 ; N#Cc1ccnc(-c2cccc(-c3c4ccccc4c(-c4cccc(-c5cc(C#N)ccn5)c4)c4c5ccccc5c5ccccc5c34)c2)c1 ; N#Cc1cncc(-c2cccc(-c3c4ccccc4c(-c4cccc(-c5cncc(C#N)c5)c4)c4c5ccccc5c5ccccc5c34)c2)c1]

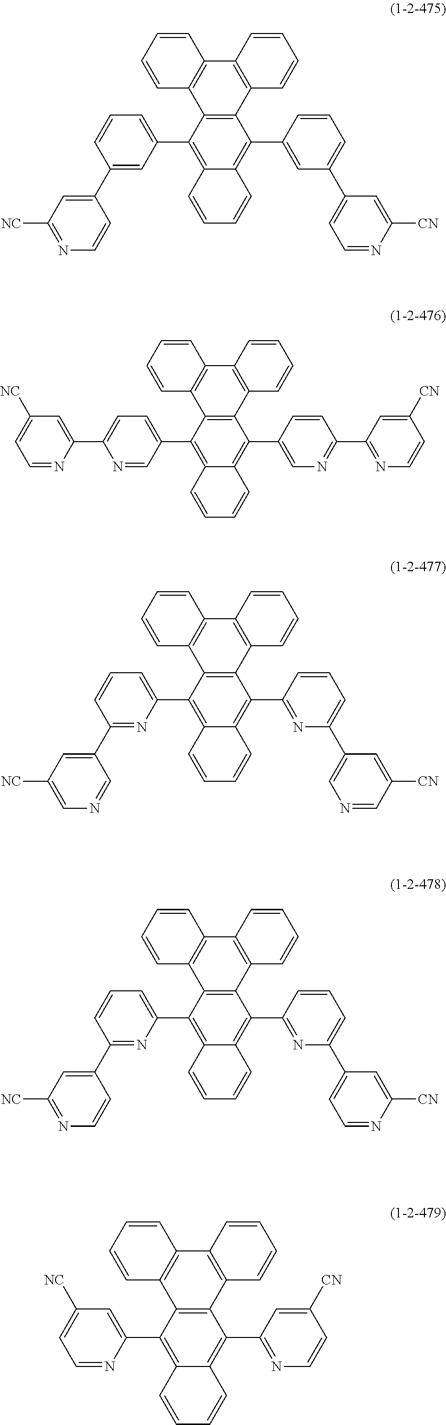 [SMILES: N#Cc1cc(-c2cccc(-c3c4ccccc4c(-c4cccc(-c5ccnc(C#N)c5)c4)c4c5ccccc5c5ccccc5c34)c2)ccn1 ; N#Cc1ccnc(-c2ccc(-c3c4ccccc4c(-c4ccc(-c5cc(C#N)ccn5)nc4)c4c5ccccc5c5ccccc5c34)cn2)c1 ; N#Cc1cncc(-c2cccc(-c3c4ccccc4c(-c4cccc(-c5cncc(C#N)c5)n4)c4c5ccccc5c5ccccc5c34)n2)c1 ; N#Cc1cc(-c2cccc(-c3c4ccccc4c(-c4cccc(-c5ccnc(C#N)c5)n4)c4c5ccccc5c5ccccc5c34)n2)ccn1 ; N#Cc1ccnc(-c2c3ccccc3c(-c3cc(C#N)ccn3)c3c4ccccc4c4ccccc4c23)c1]

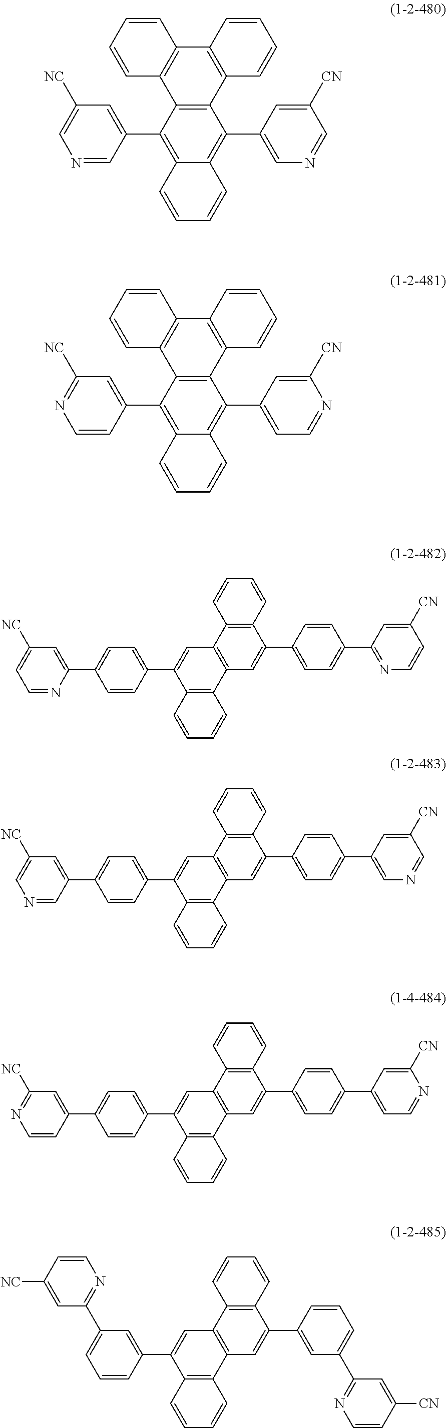 [SMILES: N#Cc1cncc(-c2c3ccccc3c(-c3cncc(C#N)c3)c3c4ccccc4c4ccccc4c23)c1 ; N#Cc1cc(-c2c3ccccc3c(-c3ccnc(C#N)c3)c3c4ccccc4c4ccccc4c23)ccn1 ; N#Cc1ccnc(-c2ccc(-c3cc4c5ccccc5c(-c5ccc(-c6cc(C#N)ccn6)cc5)cc4c4ccccc34)cc2)c1 ; N#Cc1cncc(-c2ccc(-c3cc4c5ccccc5c(-c5ccc(-c6cncc(C#N)c6)cc5)cc4c4ccccc34)cc2)c1 ; N#Cc1cc(-c2ccc(-c3cc4c5ccccc5c(-c5ccc(-c6ccnc(C#N)c6)cc5)cc4c4ccccc34)cc2)ccn1 ; N#Cc1ccnc(-c2cccc(-c3cc4c5ccccc5c(-c5cccc(-c6cc(C#N)ccn6)c5)cc4c4ccccc34)c2)c1]

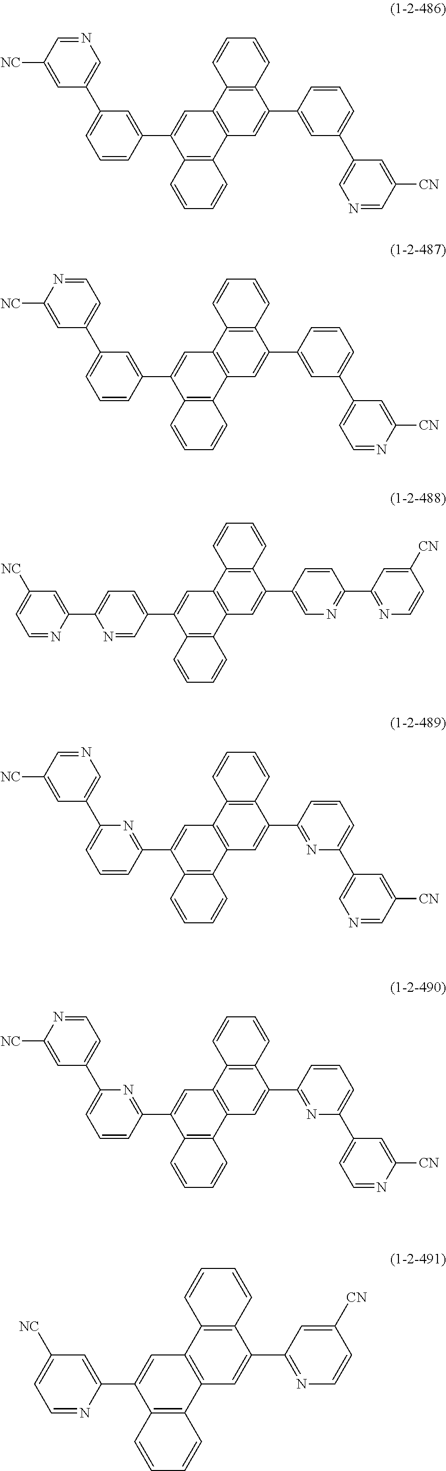 [SMILES: N#Cc1cncc(-c2cccc(-c3cc4c5ccccc5c(-c5cccc(-c6cncc(C#N)c6)c5)cc4c4ccccc34)c2)c1 ; N#Cc1cc(-c2cccc(-c3cc4c5ccccc5c(-c5cccc(-c6ccnc(C#N)c6)c5)cc4c4ccccc34)c2)ccn1 ; N#Cc1ccnc(-c2ccc(-c3cc4c5ccccc5c(-c5ccc(-c6cc(C#N)ccn6)nc5)cc4c4ccccc34)cn2)c1 ; N#Cc1cncc(-c2cccc(-c3cc4c5ccccc5c(-c5cccc(-c6cncc(C#N)c6)n5)cc4c4ccccc34)n2)c1 ; N#Cc1cc(-c2cccc(-c3cc4c5ccccc5c(-c5cccc(-c6ccnc(C#N)c6)n5)cc4c4ccccc34)n2)ccn1 ; N#Cc1ccnc(-c2cc3c4ccccc4c(-c4cc(C#N)ccn4)cc3c3ccccc23)c1]

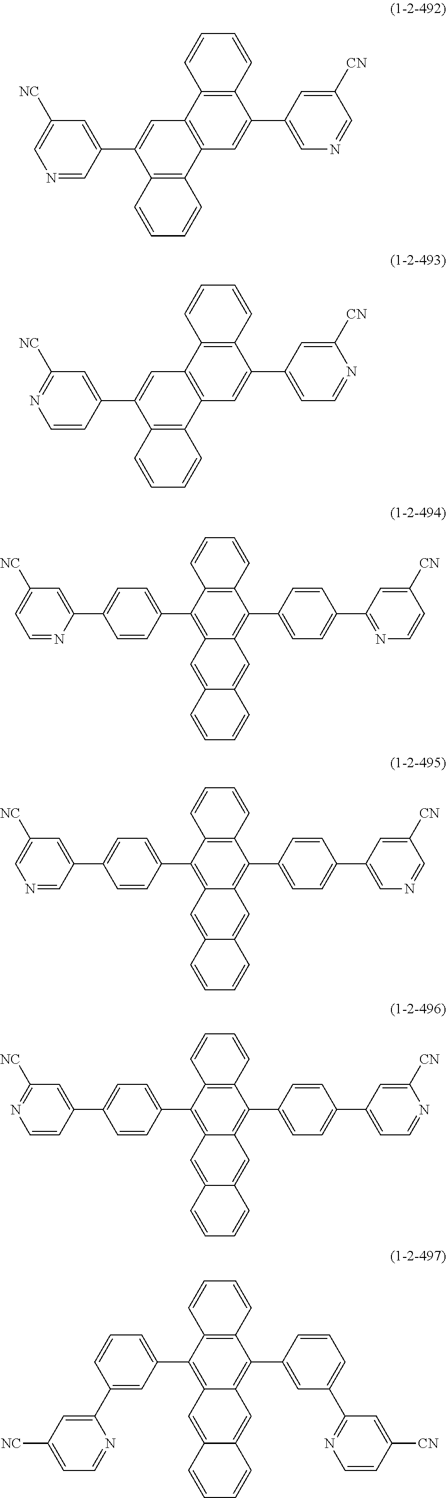 [SMILES: N#Cc1cncc(-c2cc3c4ccccc4c(-c4cncc(C#N)c4)cc3c3ccccc23)c1 ; N#Cc1cc(-c2cc3c4ccccc4c(-c4ccnc(C#N)c4)cc3c3ccccc23)ccn1 ; N#Cc1ccnc(-c2ccc(-c3c4ccccc4c(-c4ccc(-c5cc(C#N)ccn5)cc4)c4cc5ccccc5cc34)cc2)c1 ; N#Cc1cncc(-c2ccc(-c3c4ccccc4c(-c4ccc(-c5cncc(C#N)c5)cc4)c4cc5ccccc5cc34)cc2)c1 ; N#Cc1cc(-c2ccc(-c3c4ccccc4c(-c4ccc(-c5ccnc(C#N)c5)cc4)c4cc5ccccc5cc34)cc2)ccn1 ; N#Cc1ccnc(-c2cccc(-c3c4ccccc4c(-c4cccc(-c5cc(C#N)ccn5)c4)c4cc5ccccc5cc34)c2)c1]

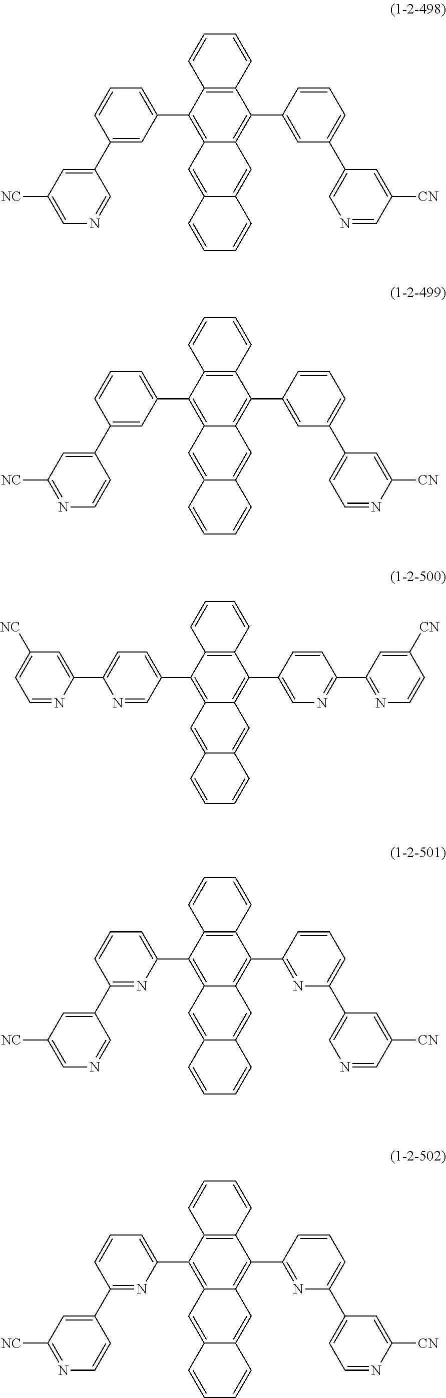 [SMILES: N#Cc1cncc(-c2cccc(-c3c4ccccc4c(-c4cccc(-c5cncc(C#N)c5)c4)c4cc5ccccc5cc34)c2)c1 ; N#Cc1cc(-c2cccc(-c3c4ccccc4c(-c4cccc(-c5ccnc(C#N)c5)c4)c4cc5ccccc5cc34)c2)ccn1 ; N#Cc1ccnc(-c2ccc(-c3c4ccccc4c(-c4ccc(-c5cc(C#N)ccn5)nc4)c4cc5ccccc5cc34)cn2)c1 ; N#Cc1cncc(-c2cccc(-c3c4ccccc4c(-c4cccc(-c5cncc(C#N)c5)n4)c4cc5ccccc5cc34)n2)c1 ; N#Cc1cc(-c2cccc(-c3c4ccccc4c(-c4cccc(-c5ccnc(C#N)c5)n4)c4cc5ccccc5cc34)n2)ccn1]

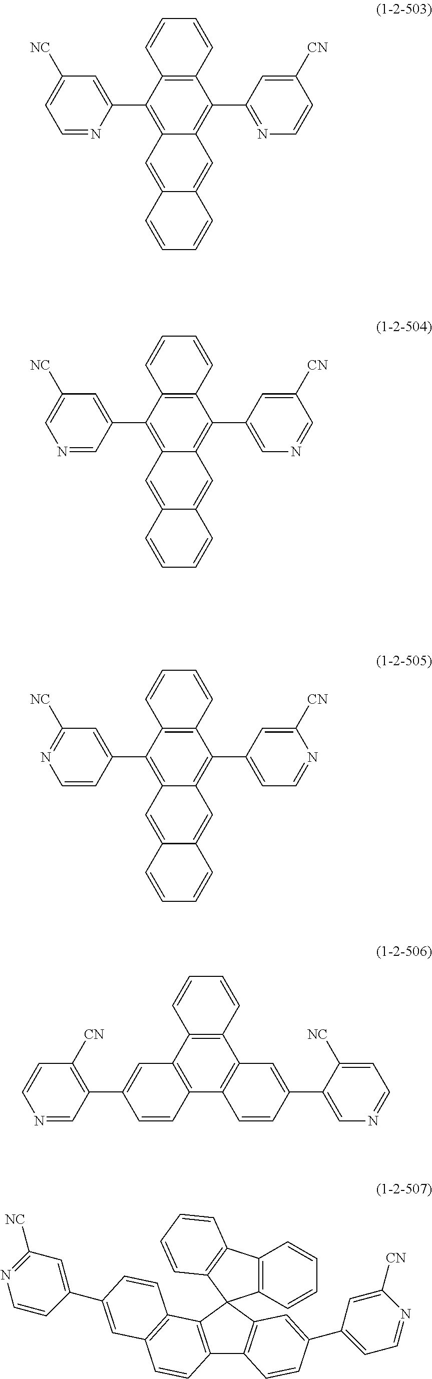 [SMILES: N#Cc1ccnc(-c2c3ccccc3c(-c3cc(C#N)ccn3)c3cc4ccccc4cc23)c1 ; N#Cc1cncc(-c2c3ccccc3c(-c3cncc(C#N)c3)c3cc4ccccc4cc23)c1 ; N#Cc1cc(-c2c3ccccc3c(-c3ccnc(C#N)c3)c3cc4ccccc4cc23)ccn1 ; N#Cc1ccncc1-c1ccc2c3ccc(-c4cnccc4C#N)cc3c3ccccc3c2c1 ; N#Cc1cc(-c2ccc3c(c2)C2(c4ccccc4-c4ccccc42)c2c-3ccc3cc(-c4ccnc(C#N)c4)ccc23)ccn1]

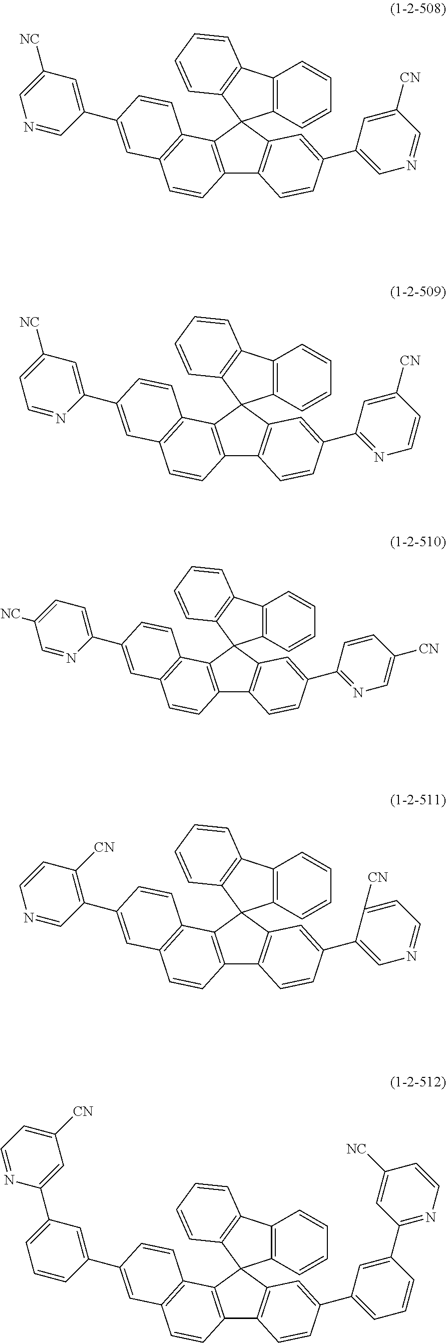

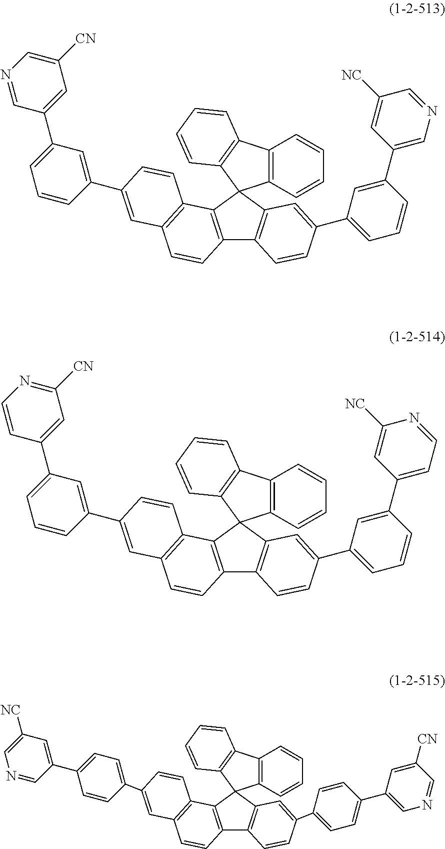 [SMILES: N#Cc1cncc(-c2cccc(-c3ccc4c(c3)C3(c5ccccc5-c5ccccc53)c3c-4ccc4cc(-c5cccc(-c6cncc(C#N)c6)c5)ccc34)c2)c1 ; N#Cc1cc(-c2cccc(-c3ccc4c(c3)C3(c5ccccc5-c5ccccc53)c3c-4ccc4cc(-c5cccc(-c6ccnc(C#N)c6)c5)ccc34)c2)ccn1 ; N#Cc1cncc(-c2ccc(-c3ccc4c(c3)C3(c5ccccc5-c5ccccc53)c3c-4ccc4cc(-c5ccc(-c6cncc(C#N)c6)cc5)ccc34)cc2)c1]

Specific examples of the compound represented by formula (1) when m=3 are shown in the following formulae (1-3-1) to (1-3-60).

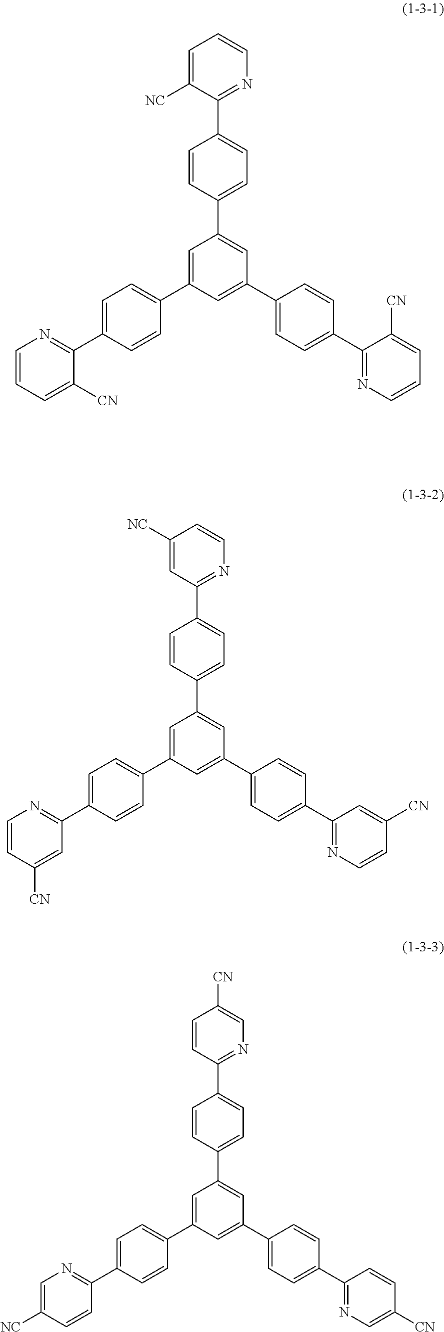 [SMILES: N#Cc1cccnc1-c1ccc(-c2cc(-c3ccc(-c4ncccc4C#N)cc3)cc(-c3ccc(-c4ncccc4C#N)cc3)c2)cc1 ; N#Cc1ccnc(-c2ccc(-c3cc(-c4ccc(-c5cc(C#N)ccn5)cc4)cc(-c4ccc(-c5cc(C#N)ccn5)cc4)c3)cc2)c1 ; N#Cc1ccc(-c2ccc(-c3cc(-c4ccc(-c5ccc(C#N)cn5)cc4)cc(-c4ccc(-c5ccc(C#N)cn5)cc4)c3)cc2)nc1]

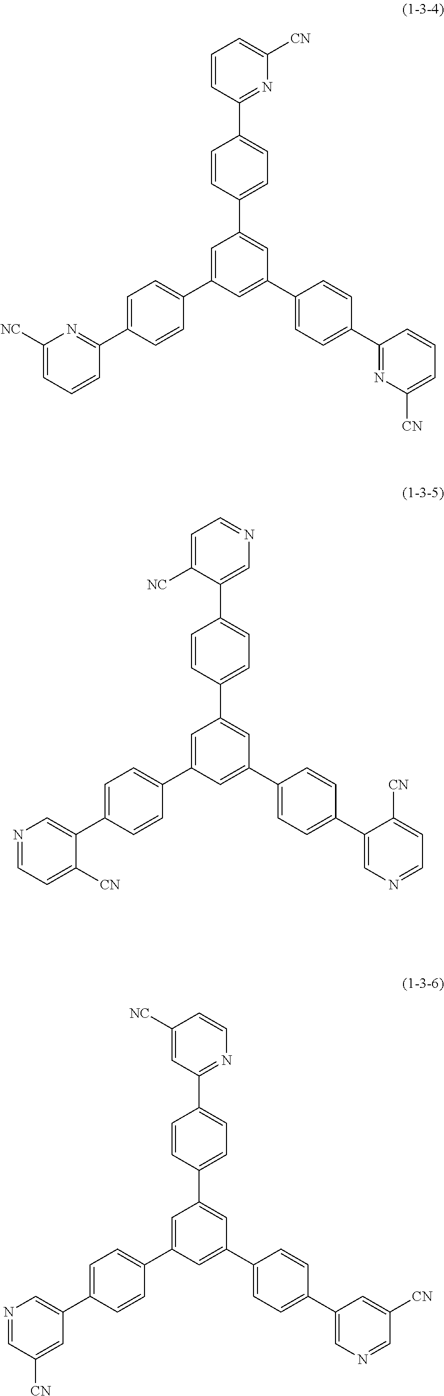 [SMILES: N#Cc1cccc(-c2ccc(-c3cc(-c4ccc(-c5cccc(C#N)n5)cc4)cc(-c4ccc(-c5cccc(C#N)n5)cc4)c3)cc2)n1 ; N#Cc1ccncc1-c1ccc(-c2cc(-c3ccc(-c4cnccc4C#N)cc3)cc(-c3ccc(-c4cnccc4C#N)cc3)c2)cc1 ; N#Cc1cncc(-c2ccc(-c3cc(-c4ccc(-c5cncc(C#N)c5)cc4)cc(-c4ccc(-c5cc(C#N)ccn5)cc4)c3)cc2)c1]

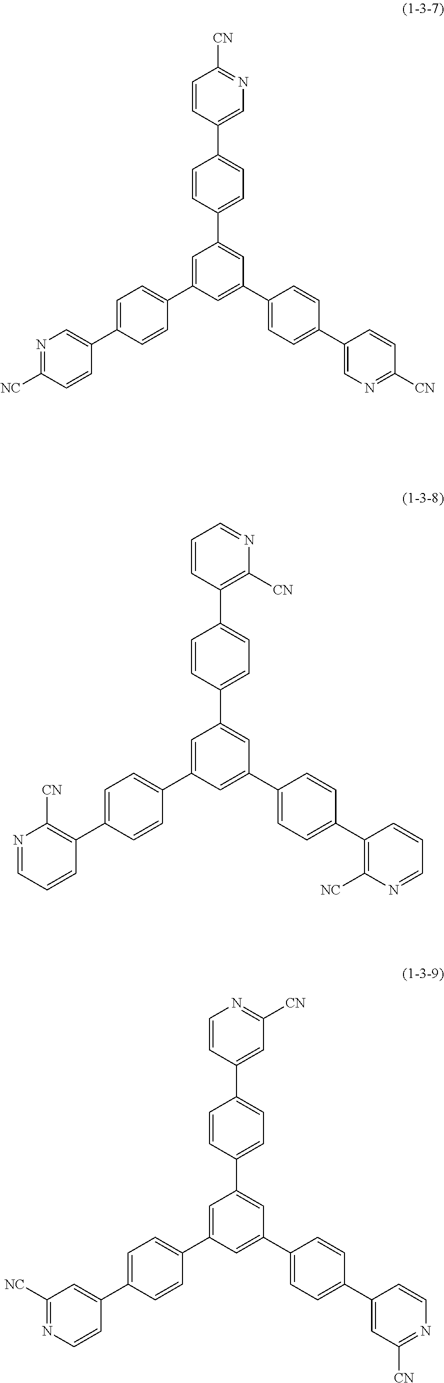 [SMILES: N#Cc1ccc(-c2ccc(-c3cc(-c4ccc(-c5ccc(C#N)nc5)cc4)cc(-c4ccc(-c5ccc(C#N)nc5)cc4)c3)cc2)cn1 ; N#Cc1ncccc1-c1ccc(-c2cc(-c3ccc(-c4cccnc4C#N)cc3)cc(-c3ccc(-c4cccnc4C#N)cc3)c2)cc1 ; N#Cc1cc(-c2ccc(-c3cc(-c4ccc(-c5ccnc(C#N)c5)cc4)cc(-c4ccc(-c5ccnc(C#N)c5)cc4)c3)cc2)ccn1]

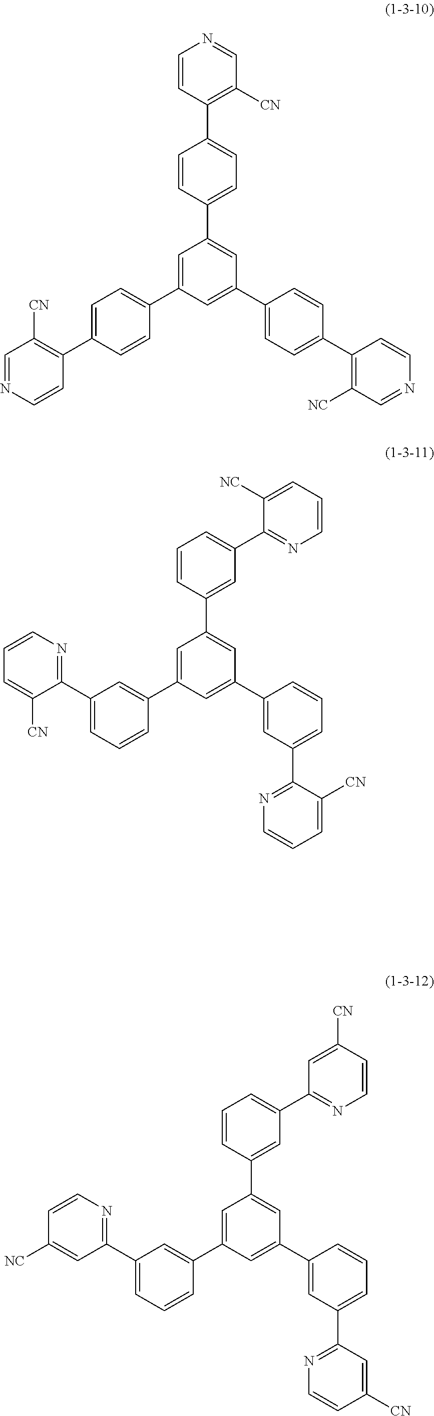 [SMILES: N#Cc1cnccc1-c1ccc(-c2cc(-c3ccc(-c4ccncc4C#N)cc3)cc(-c3ccc(-c4ccncc4C#N)cc3)c2)cc1 ; N#Cc1cccnc1-c1cccc(-c2cc(-c3cccc(-c4ncccc4C#N)c3)cc(-c3cccc(-c4ncccc4C#N)c3)c2)c1 ; N#Cc1ccnc(-c2cccc(-c3cc(-c4cccc(-c5cc(C#N)ccn5)c4)cc(-c4cccc(-c5cc(C#N)ccn5)c4)c3)c2)c1]

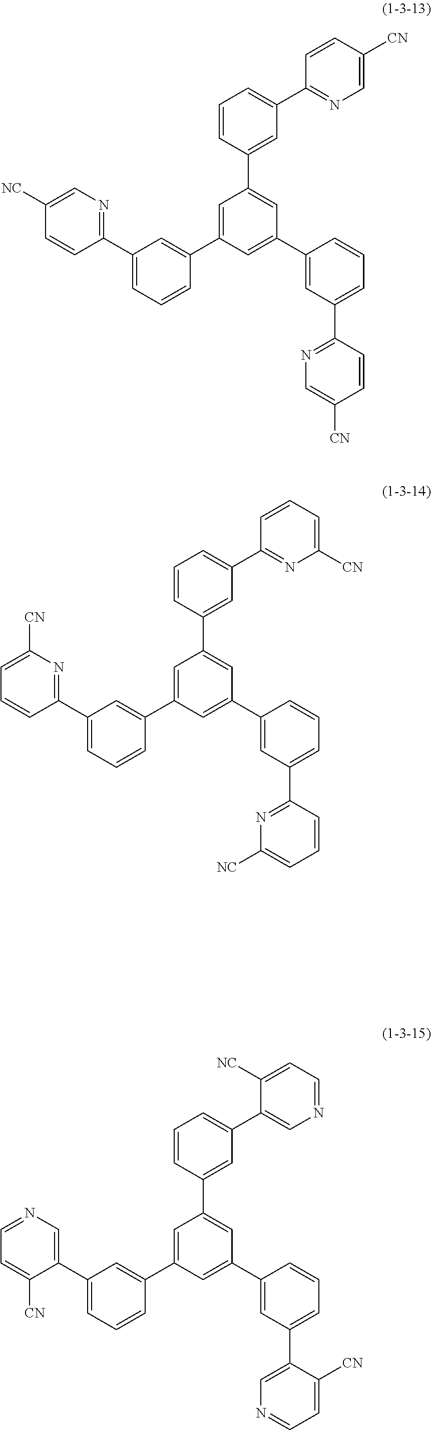 [SMILES: N#Cc1ccc(-c2cccc(-c3cc(-c4cccc(-c5ccc(C#N)cn5)c4)cc(-c4cccc(-c5ccc(C#N)cn5)c4)c3)c2)nc1 ; N#Cc1cccc(-c2cccc(-c3cc(-c4cccc(-c5cccc(C#N)n5)c4)cc(-c4cccc(-c5cccc(C#N)n5)c4)c3)c2)n1 ; N#Cc1ccncc1-c1cccc(-c2cc(-c3cccc(-c4cnccc4C#N)c3)cc(-c3cccc(-c4cnccc4C#N)c3)c2)c1]

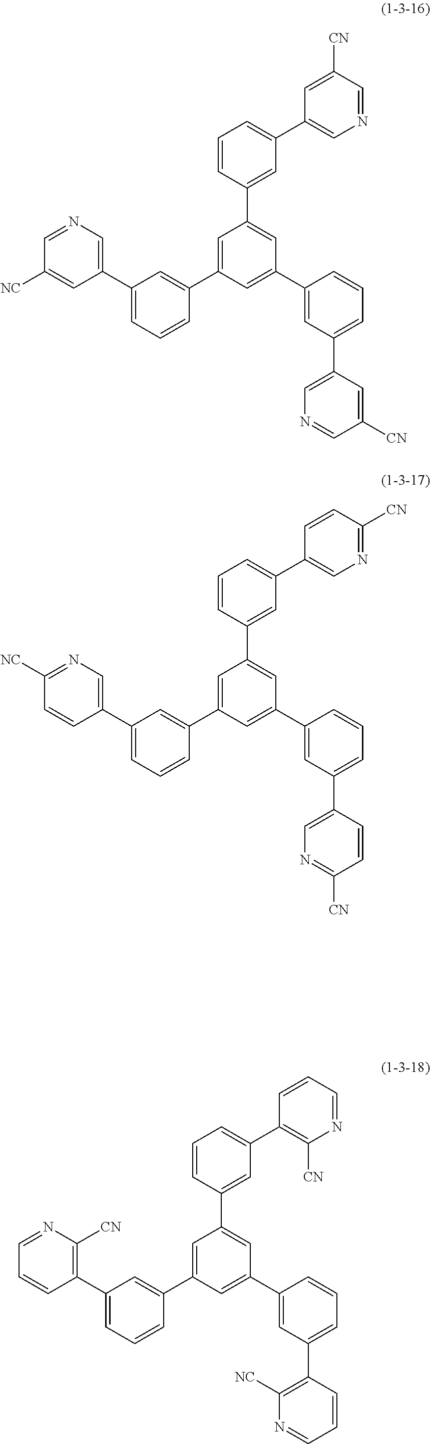 [SMILES: N#Cc1cncc(-c2cccc(-c3cc(-c4cccc(-c5cncc(C#N)c5)c4)cc(-c4cccc(-c5cncc(C#N)c5)c4)c3)c2)c1 ; N#Cc1ccc(-c2cccc(-c3cc(-c4cccc(-c5ccc(C#N)nc5)c4)cc(-c4cccc(-c5ccc(C#N)nc5)c4)c3)c2)cn1 ; N#Cc1ncccc1-c1cccc(-c2cc(-c3cccc(-c4cccnc4C#N)c3)cc(-c3cccc(-c4cccnc4C#N)c3)c2)c1]

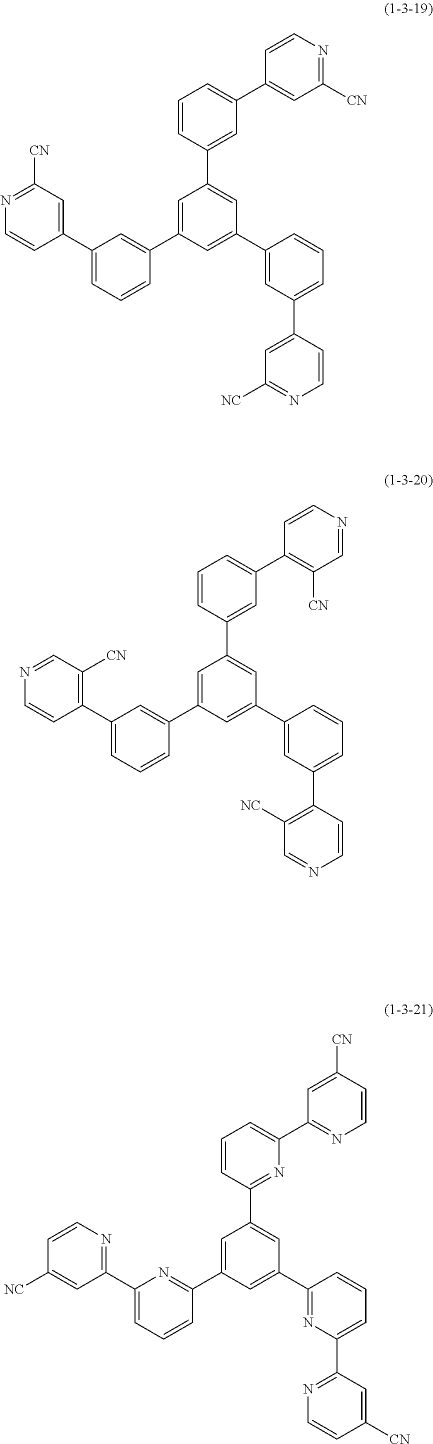 [SMILES: N#Cc1cc(-c2cccc(-c3cc(-c4cccc(-c5ccnc(C#N)c5)c4)cc(-c4cccc(-c5ccnc(C#N)c5)c4)c3)c2)ccn1 ; N#Cc1cnccc1-c1cccc(-c2cc(-c3cccc(-c4ccncc4C#N)c3)cc(-c3cccc(-c4ccncc4C#N)c3)c2)c1 ; N#Cc1ccnc(-c2cccc(-c3cc(-c4cccc(-c5cc(C#N)ccn5)n4)cc(-c4cccc(-c5cc(C#N)ccn5)n4)c3)n2)c1]

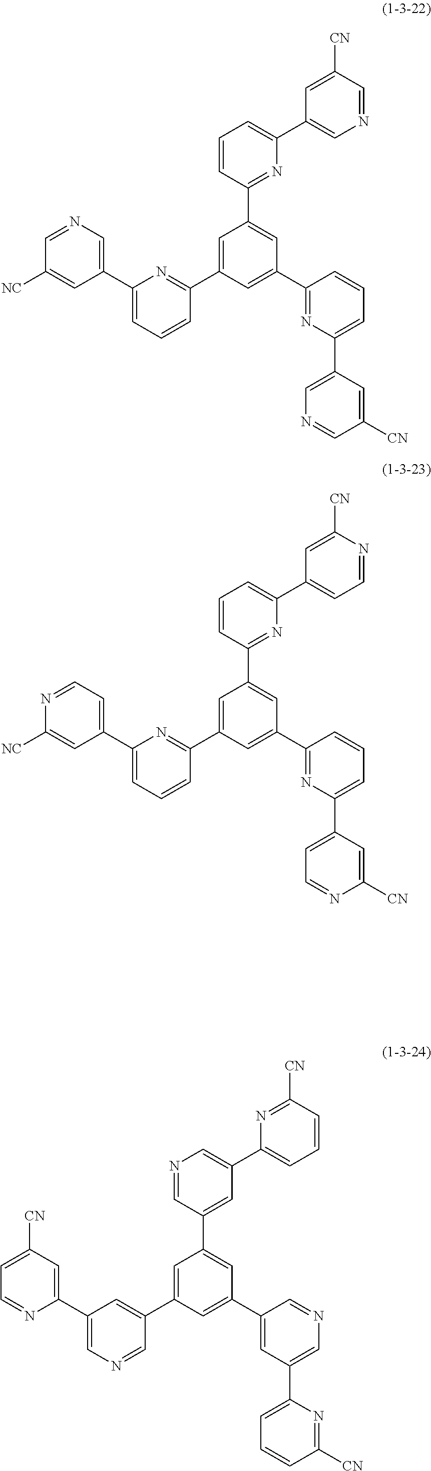 [SMILES: N#Cc1cncc(-c2cccc(-c3cc(-c4cccc(-c5cncc(C#N)c5)n4)cc(-c4cccc(-c5cncc(C#N)c5)n4)c3)n2)c1 ; N#Cc1cc(-c2cccc(-c3cc(-c4cccc(-c5ccnc(C#N)c5)n4)cc(-c4cccc(-c5ccnc(C#N)c5)n4)c3)n2)ccn1 ; N#Cc1ccnc(-c2cncc(-c3cc(-c4cncc(-c5cccc(C#N)n5)c4)cc(-c4cncc(-c5cccc(C#N)n5)c4)c3)c2)c1]

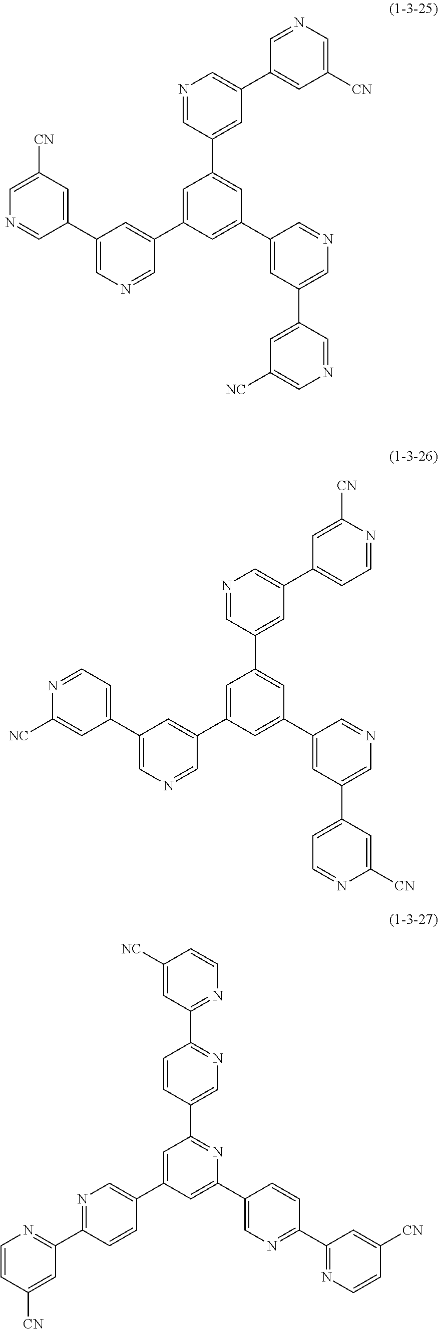 [SMILES: N#Cc1cncc(-c2cncc(-c3cc(-c4cncc(-c5cncc(C#N)c5)c4)cc(-c4cncc(-c5cncc(C#N)c5)c4)c3)c2)c1 ; N#Cc1cc(-c2cncc(-c3cc(-c4cncc(-c5ccnc(C#N)c5)c4)cc(-c4cncc(-c5ccnc(C#N)c5)c4)c3)c2)ccn1 ; N#Cc1ccnc(-c2ccc(-c3cc(-c4ccc(-c5cc(C#N)ccn5)nc4)nc(-c4ccc(-c5cc(C#N)ccn5)nc4)c3)cn2)c1]

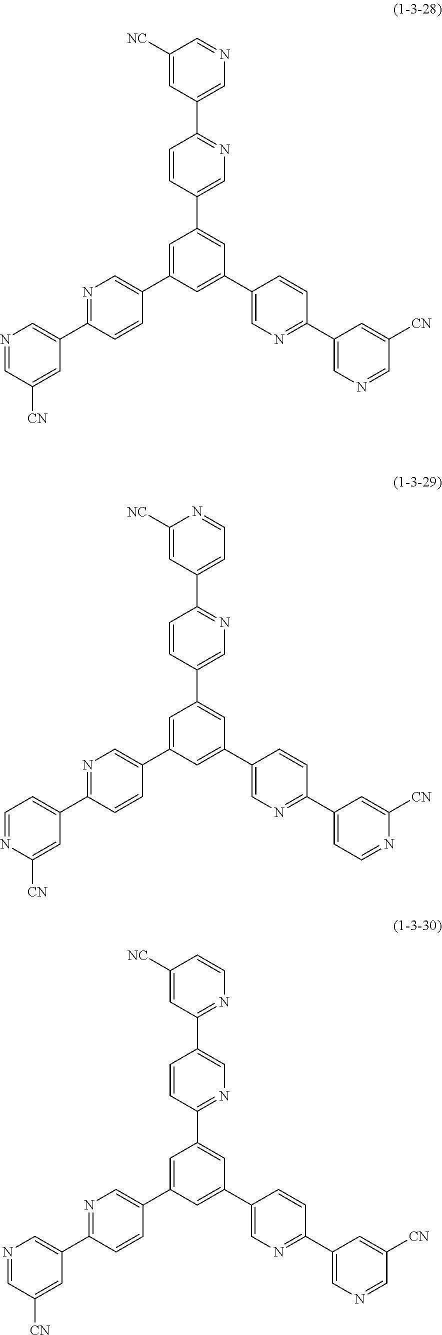 [SMILES: N#Cc1cncc(-c2ccc(-c3cc(-c4ccc(-c5cncc(C#N)c5)nc4)cc(-c4ccc(-c5cncc(C#N)c5)nc4)c3)cn2)c1 ; N#Cc1cc(-c2ccc(-c3cc(-c4ccc(-c5ccnc(C#N)c5)nc4)cc(-c4ccc(-c5ccnc(C#N)c5)nc4)c3)cn2)ccn1 ; N#Cc1cncc(-c2ccc(-c3cc(-c4ccc(-c5cncc(C#N)c5)nc4)cc(-c4ccc(-c5cc(C#N)ccn5)cn4)c3)cn2)c1]

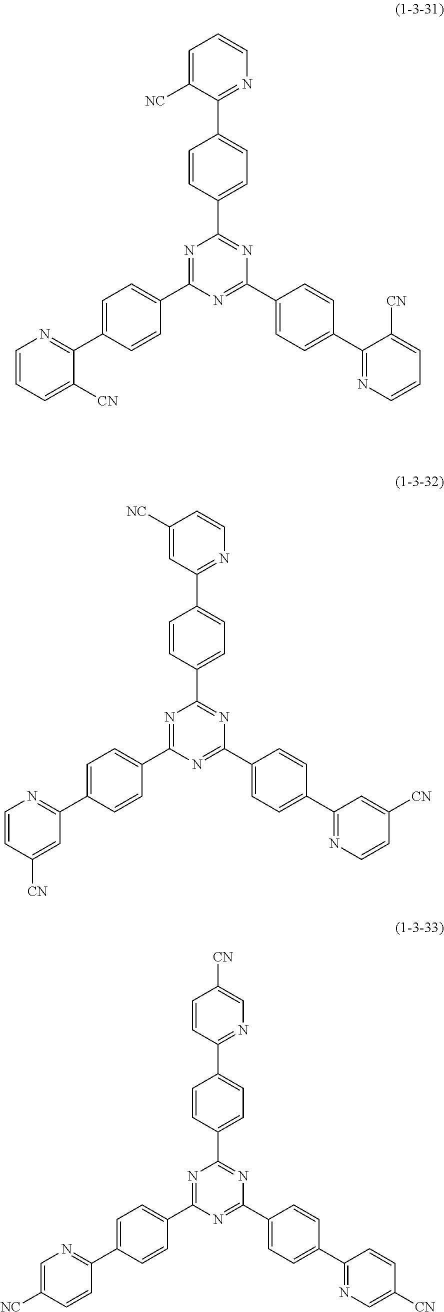 [SMILES: N#Cc1cccnc1-c1ccc(-c2nc(-c3ccc(-c4ncccc4C#N)cc3)nc(-c3ccc(-c4ncccc4C#N)cc3)n2)cc1 ; N#Cc1ccnc(-c2ccc(-c3nc(-c4ccc(-c5cc(C#N)ccn5)cc4)nc(-c4ccc(-c5cc(C#N)ccn5)cc4)n3)cc2)c1 ; N#Cc1ccc(-c2ccc(-c3nc(-c4ccc(-c5ccc(C#N)cn5)cc4)nc(-c4ccc(-c5ccc(C#N)cn5)cc4)n3)cc2)nc1]

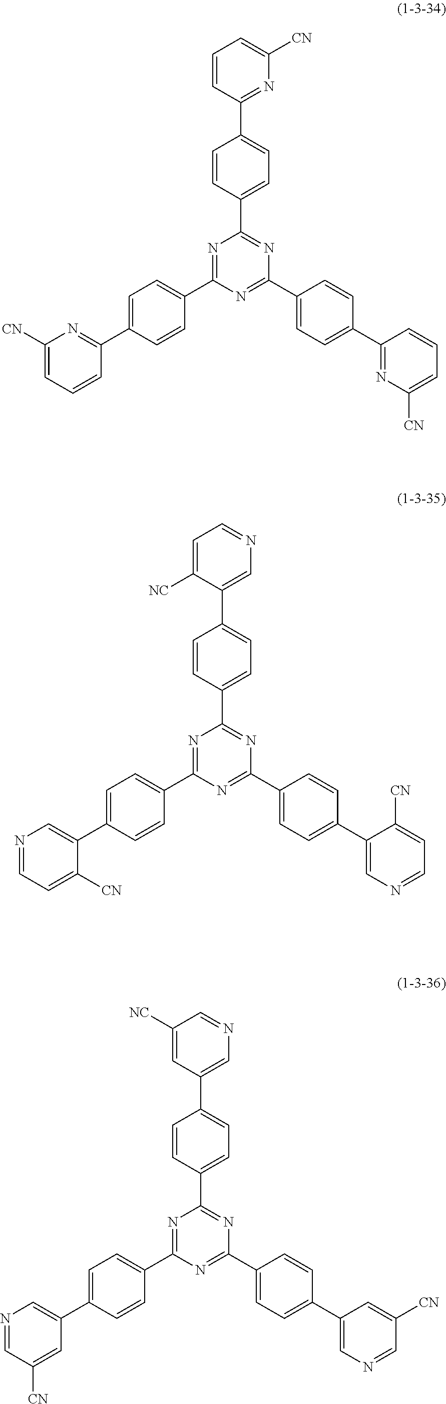

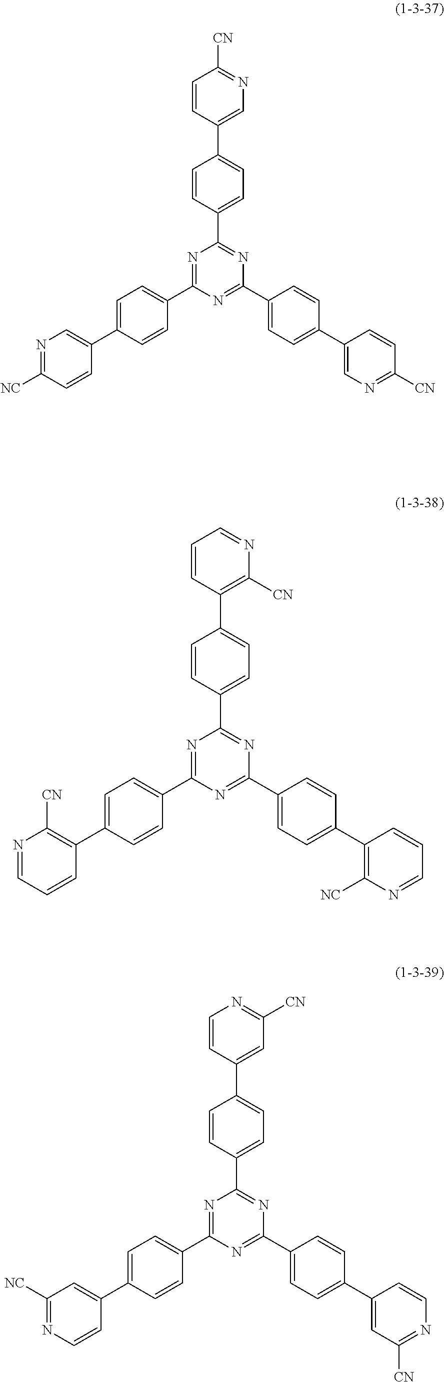 [SMILES: N#Cc1ccc(-c2ccc(-c3nc(-c4ccc(-c5ccc(C#N)nc5)cc4)nc(-c4ccc(-c5ccc(C#N)nc5)cc4)n3)cc2)cn1 ; N#Cc1ncccc1-c1ccc(-c2nc(-c3ccc(-c4cccnc4C#N)cc3)nc(-c3ccc(-c4cccnc4C#N)cc3)n2)cc1 ; N#Cc1cc(-c2ccc(-c3nc(-c4ccc(-c5ccnc(C#N)c5)cc4)nc(-c4ccc(-c5ccnc(C#N)c5)cc4)n3)cc2)ccn1]

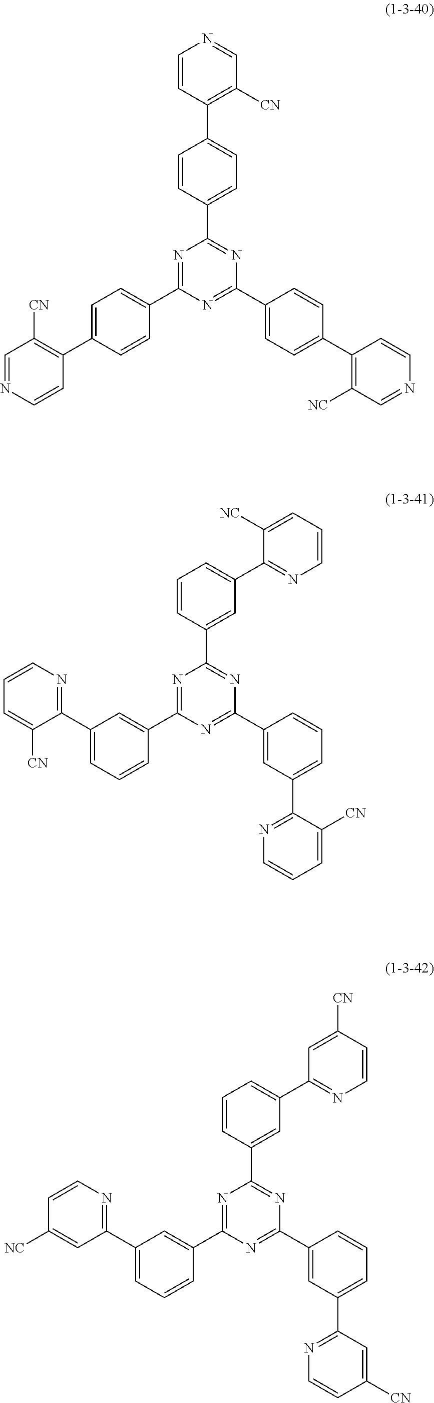 [SMILES: N#Cc1cnccc1-c1ccc(-c2nc(-c3ccc(-c4ccncc4C#N)cc3)nc(-c3ccc(-c4ccncc4C#N)cc3)n2)cc1 ; N#Cc1cccnc1-c1cccc(-c2nc(-c3cccc(-c4ncccc4C#N)c3)nc(-c3cccc(-c4ncccc4C#N)c3)n2)c1 ; N#Cc1ccnc(-c2cccc(-c3nc(-c4cccc(-c5cc(C#N)ccn5)c4)nc(-c4cccc(-c5cc(C#N)ccn5)c4)n3)c2)c1]

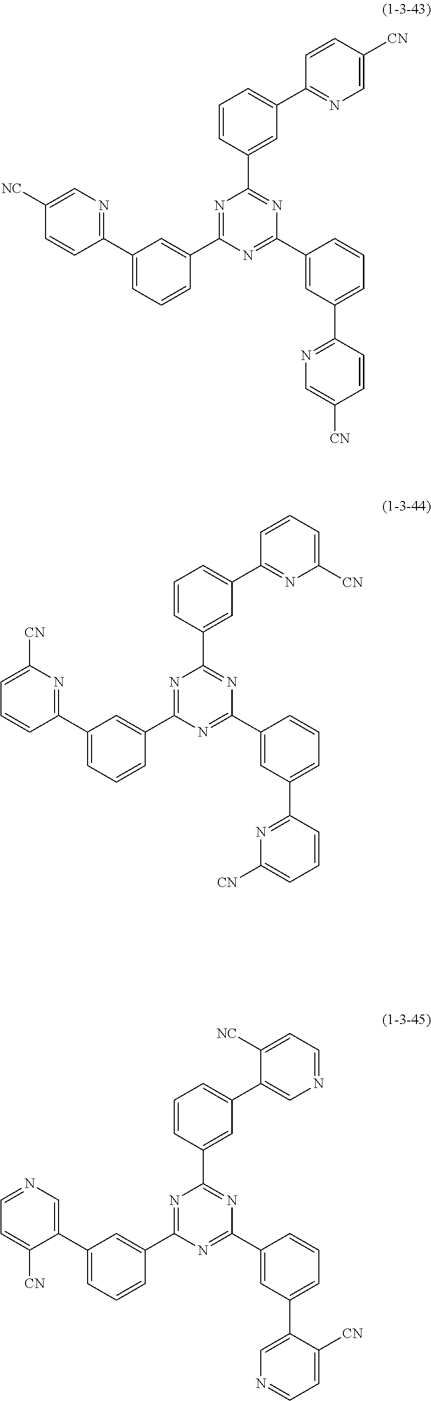

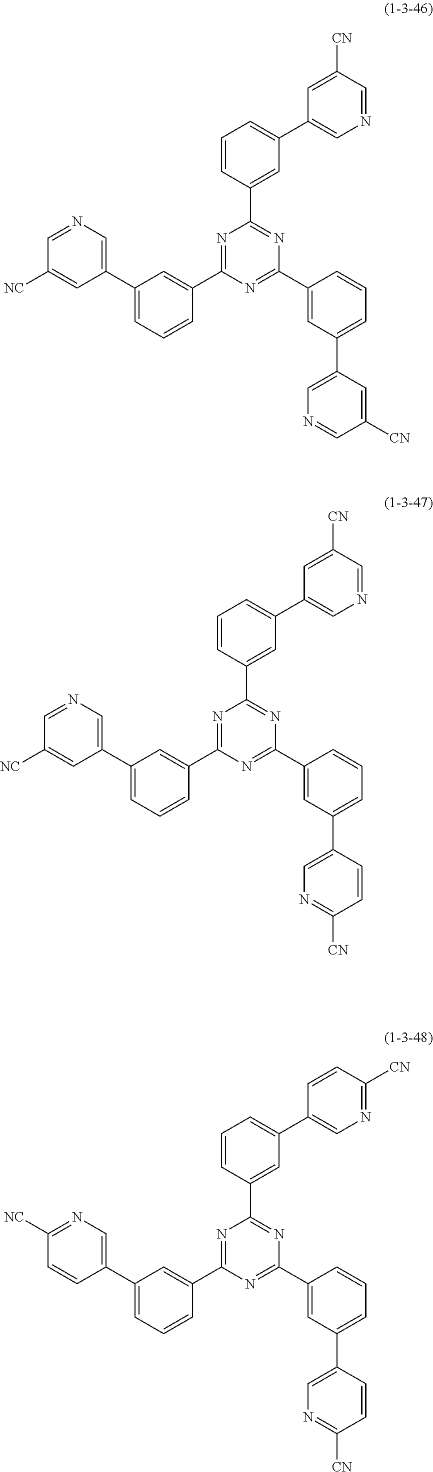 [SMILES: N#Cc1cncc(-c2cccc(-c3nc(-c4cccc(-c5cncc(C#N)c5)c4)nc(-c4cccc(-c5cncc(C#N)c5)c4)n3)c2)c1 ; N#Cc1cncc(-c2cccc(-c3nc(-c4cccc(-c5ccc(C#N)nc5)c4)nc(-c4cccc(-c5cncc(C#N)c5)c4)n3)c2)c1 ; N#Cc1ccc(-c2cccc(-c3nc(-c4cccc(-c5ccc(C#N)nc5)c4)nc(-c4cccc(-c5ccc(C#N)nc5)c4)n3)c2)cn1]

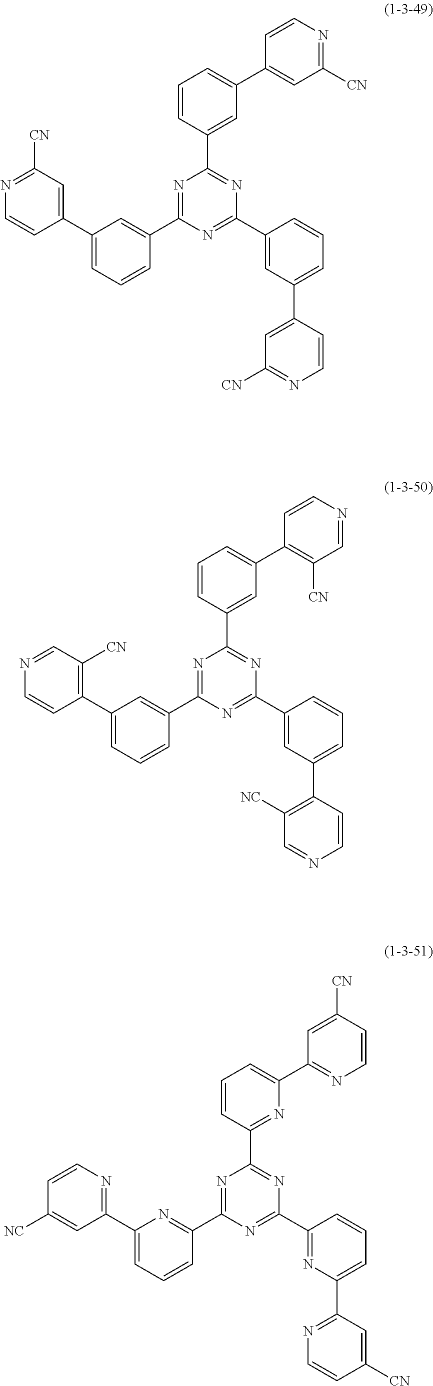

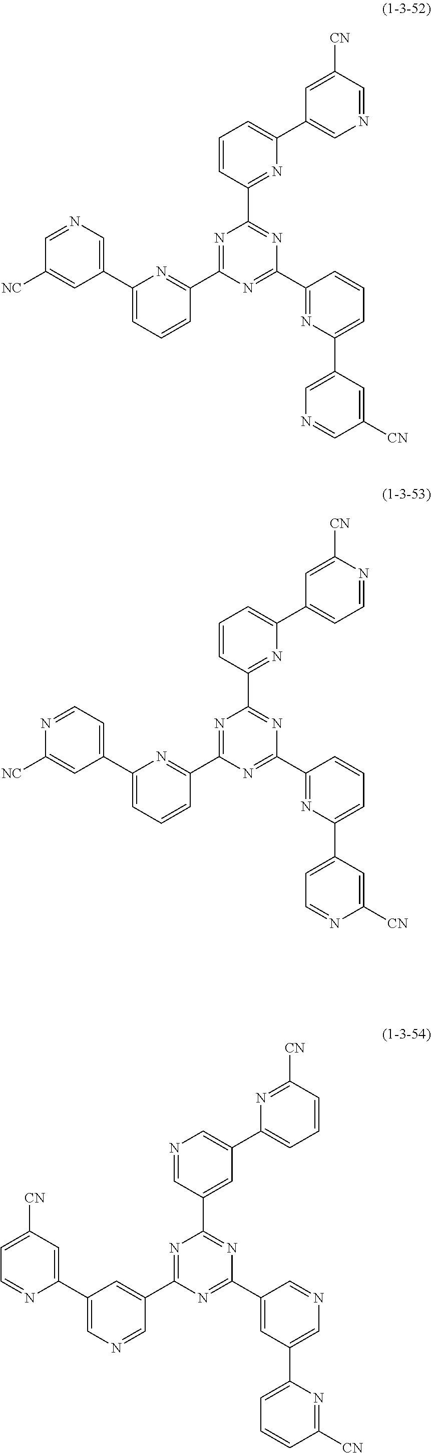 [SMILES: N#Cc1cncc(-c2cccc(-c3nc(-c4cccc(-c5cncc(C#N)c5)n4)nc(-c4cccc(-c5cncc(C#N)c5)n4)n3)n2)c1 ; N#Cc1cc(-c2cccc(-c3nc(-c4cccc(-c5ccnc(C#N)c5)n4)nc(-c4cccc(-c5ccnc(C#N)c5)n4)n3)n2)ccn1 ; N#Cc1ccnc(-c2cncc(-c3nc(-c4cncc(-c5cccc(C#N)n5)c4)nc(-c4cncc(-c5cccc(C#N)n5)c4)n3)c2)c1]

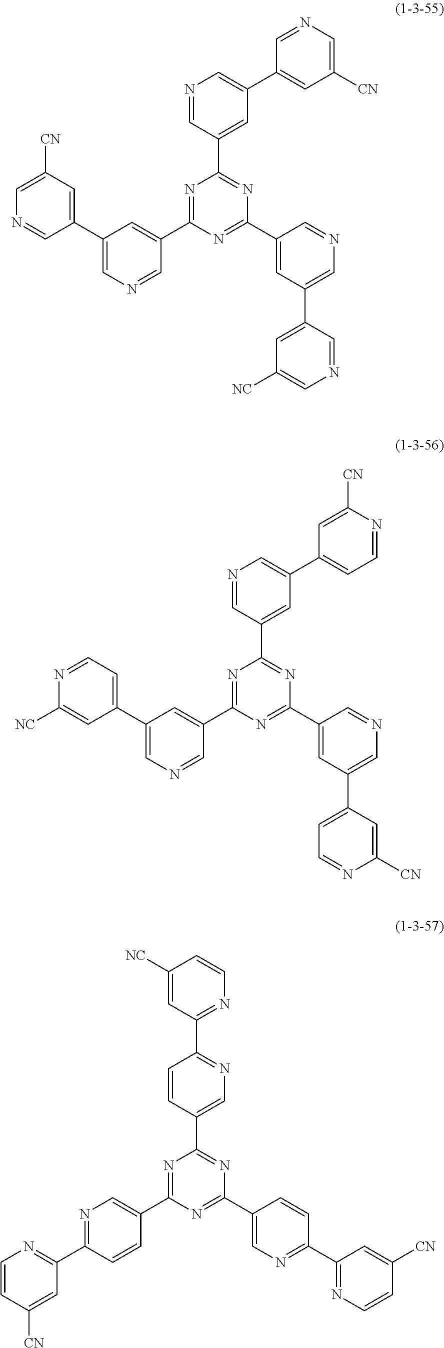 [SMILES: N#Cc1cncc(-c2cncc(-c3nc(-c4cncc(-c5cncc(C#N)c5)c4)nc(-c4cncc(-c5cncc(C#N)c5)c4)n3)c2)c1 ; N#Cc1cc(-c2cncc(-c3nc(-c4cncc(-c5ccnc(C#N)c5)c4)nc(-c4cncc(-c5ccnc(C#N)c5)c4)n3)c2)ccn1 ; N#Cc1ccnc(-c2ccc(-c3nc(-c4ccc(-c5cc(C#N)ccn5)nc4)nc(-c4ccc(-c5cc(C#N)ccn5)nc4)n3)cn2)c1]

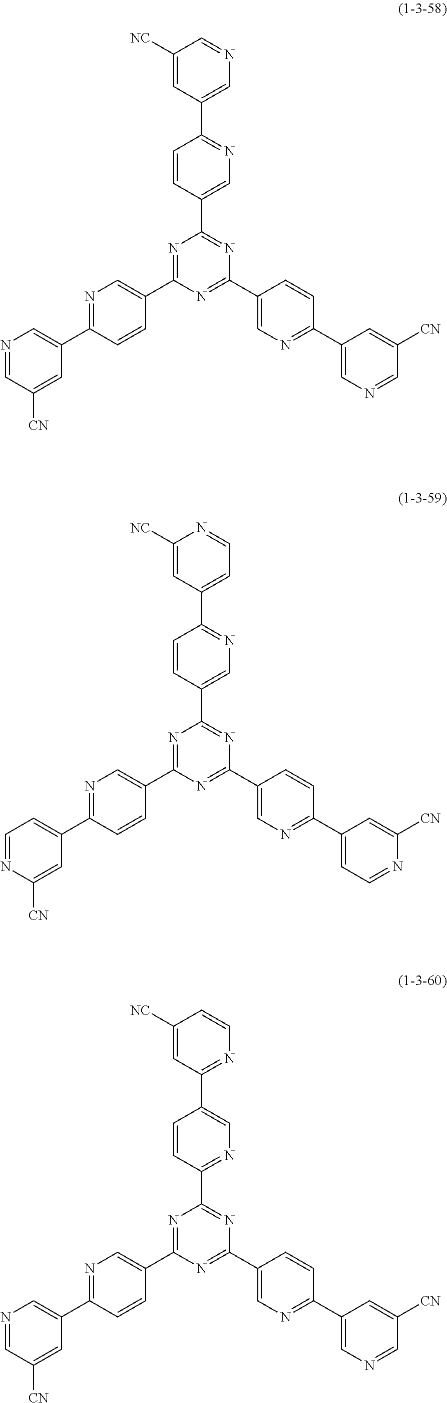 [SMILES: N#Cc1cncc(-c2ccc(-c3nc(-c4ccc(-c5cncc(C#N)c5)nc4)nc(-c4ccc(-c5cncc(C#N)c5)nc4)n3)cn2)c1 ; N#Cc1cc(-c2ccc(-c3nc(-c4ccc(-c5ccnc(C#N)c5)nc4)nc(-c4ccc(-c5ccnc(C#N)c5)nc4)n3)cn2)ccn1 ; N#Cc1cncc(-c2ccc(-c3nc(-c4ccc(-c5cncc(C#N)c5)nc4)nc(-c4ccc(-c5cc(C#N)ccn5)cn4)n3)cn2)c1]

Specific examples of the compound represented by formula (1) when m=4 are shown by formulae (1-4-1) to (1-4-13) below.

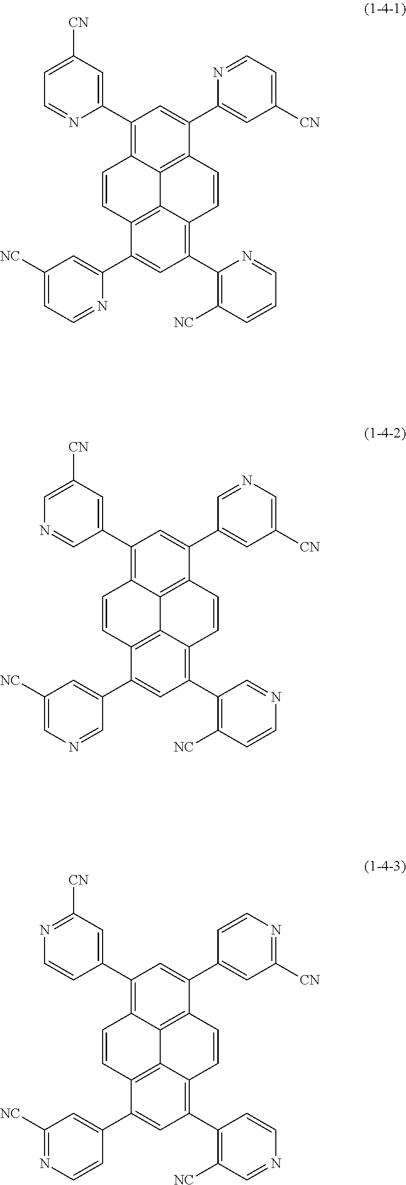 [SMILES: N#Cc1ccnc(-c2cc(-c3cc(C#N)ccn3)c3ccc4c(-c5ncccc5C#N)cc(-c5cc(C#N)ccn5)c5ccc2c3c54)c1 ; N#Cc1cncc(-c2cc(-c3cncc(C#N)c3)c3ccc4c(-c5cnccc5C#N)cc(-c5cncc(C#N)c5)c5ccc2c3c54)c1 ; N#Cc1cc(-c2cc(-c3ccnc(C#N)c3)c3ccc4c(-c5ccncc5C#N)cc(-c5ccnc(C#N)c5)c5ccc2c3c54)ccn1]

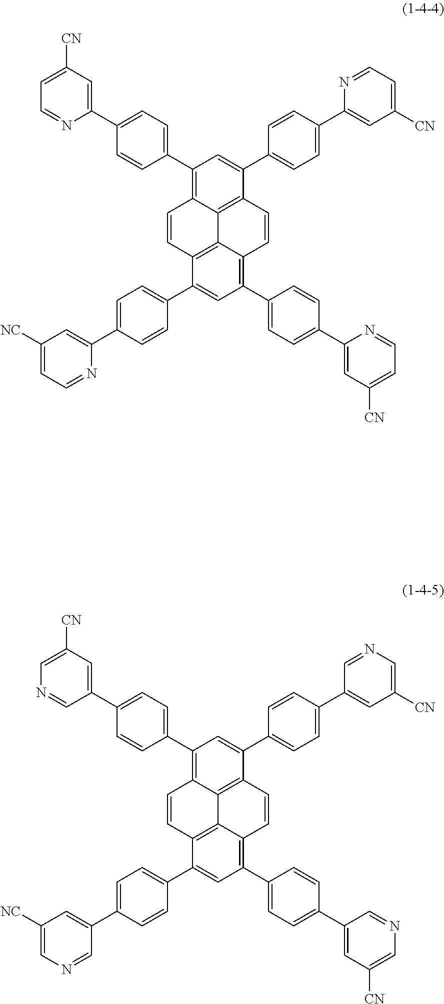 [SMILES: N#Cc1ccnc(-c2ccc(-c3cc(-c4ccc(-c5cc(C#N)ccn5)cc4)c4ccc5c(-c6ccc(-c7cc(C#N)ccn7)cc6)cc(-c6ccc(-c7cc(C#N)ccn7)cc6)c6ccc3c4c65)cc2)c1 ; N#Cc1cncc(-c2ccc(-c3cc(-c4ccc(-c5cncc(C#N)c5)cc4)c4ccc5c(-c6ccc(-c7cncc(C#N)c7)cc6)cc(-c6ccc(-c7cncc(C#N)c7)cc6)c6ccc3c4c65)cc2)c1]

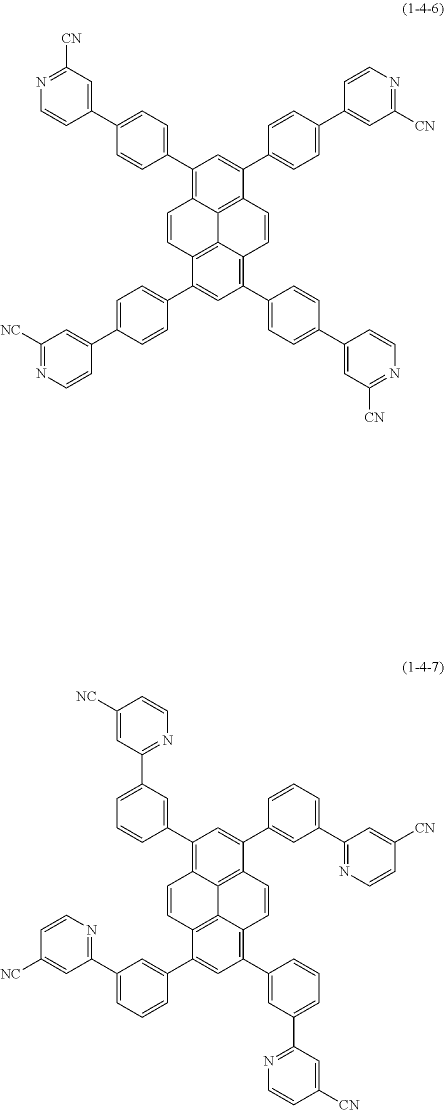 [SMILES: N#Cc1cc(-c2ccc(-c3cc(-c4ccc(-c5ccnc(C#N)c5)cc4)c4ccc5c(-c6ccc(-c7ccnc(C#N)c7)cc6)cc(-c6ccc(-c7ccnc(C#N)c7)cc6)c6ccc3c4c65)cc2)ccn1 ; N#Cc1ccnc(-c2cccc(-c3cc(-c4cccc(-c5cc(C#N)ccn5)c4)c4ccc5c(-c6cccc(-c7cc(C#N)ccn7)c6)cc(-c6cccc(-c7cc(C#N)ccn7)c6)c6ccc3c4c65)c2)c1]

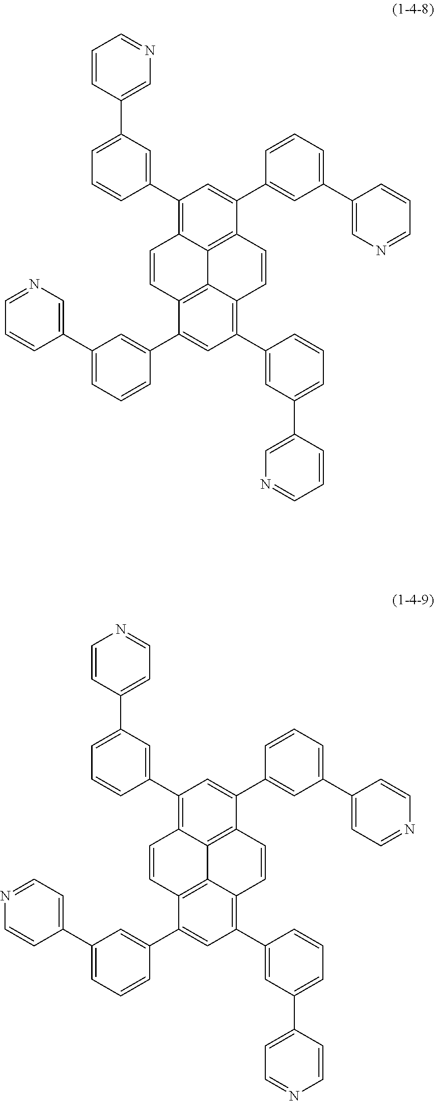 [SMILES: c1cncc(-c2cccc(-c3cc(-c4cccc(-c5cccnc5)c4)c4ccc5c(-c6cccc(-c7cccnc7)c6)cc(-c6cccc(-c7cccnc7)c6)c6ccc3c4c65)c2)c1 ; c1cc(-c2ccncc2)cc(-c2cc(-c3cccc(-c4ccncc4)c3)c3ccc4c(-c5cccc(-c6ccncc6)c5)cc(-c5cccc(-c6ccncc6)c5)c5ccc2c3c54)c1]

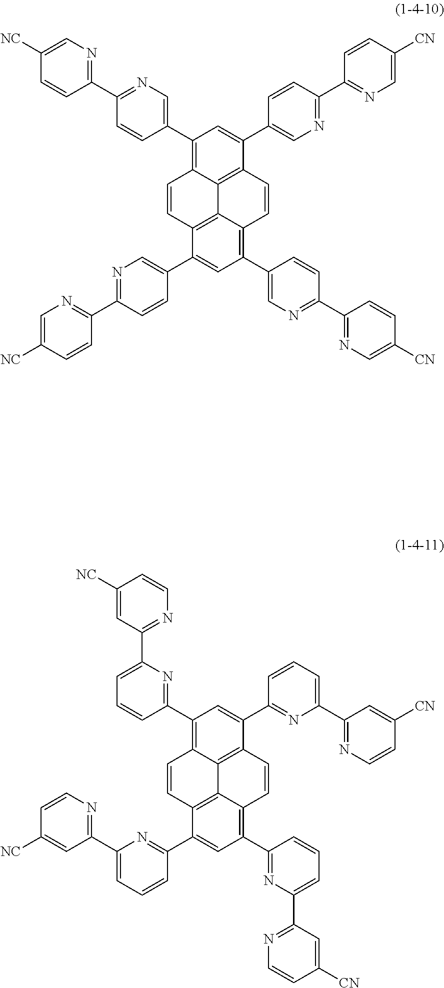 [SMILES: N#Cc1ccc(-c2ccc(-c3cc(-c4ccc(-c5ccc(C#N)cn5)nc4)c4ccc5c(-c6ccc(-c7ccc(C#N)cn7)nc6)cc(-c6ccc(-c7ccc(C#N)cn7)nc6)c6ccc3c4c65)cn2)nc1 ; N#Cc1ccnc(-c2cccc(-c3cc(-c4cccc(-c5cc(C#N)ccn5)n4)c4ccc5c(-c6cccc(-c7cc(C#N)ccn7)n6)cc(-c6cccc(-c7cc(C#N)ccn7)n6)c6ccc3c4c65)n2)c1]

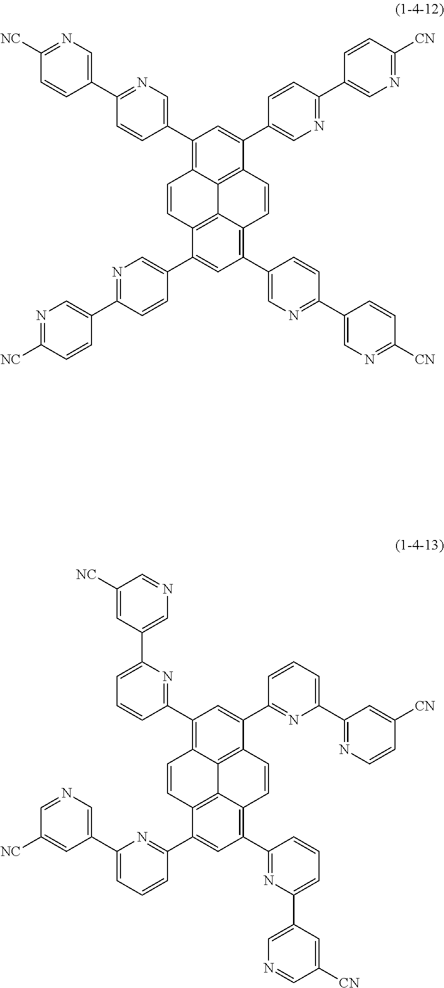 [SMILES: N#Cc1ccc(-c2ccc(-c3cc(-c4ccc(-c5ccc(C#N)nc5)nc4)c4ccc5c(-c6ccc(-c7ccc(C#N)nc7)nc6)cc(-c6ccc(-c7ccc(C#N)nc7)nc6)c6ccc3c4c65)cn2)cn1 ; N#Cc1cncc(-c2cccc(-c3cc(-c4cccc(-c5cncc(C#N)c5)n4)c4ccc5c(-c6cccc(-c7cc(C#N)ccn7)n6)cc(-c6cccc(-c7cncc(C#N)c7)n6)c6ccc3c4c65)n2)c1]

Preferable compounds among the aforementioned examples are compounds (1-1-1) to (1-1-80), (1-1-123) to (1-1-185), (1-1-228) to (1-1-290), (1-1-333) to (1-1-404), (1-1-447) to (1-1-500), (1-1-525) to (1-1-548), (1-1-609) to (1-1-620), (1-2-1) to (1-2-56), (1-2-73) to (1-2-90), (1-2-109) to (1-2-144), (1-2-169) to (1-2-204), (1-2-229) to (1-2-264), (1-2-289) to (1-2-312), (1-2-361) to (1-2-373), (1-2-506) to (1-2-515), and (1-3-1) to (1-3-50).

More preferable compounds are compounds (1-1-1) to (1-1-59), (1-1-72) to (1-1-80), (1-1-123) to (1-1-155), (1-1-174) to (1-1-185), (1-1-228) to (1-1-290), (1-1-333) to (1- 1-404), (1-1-447) to (1-1-500), (1-1-525) to (1-1-548), (1-2-1) to (1-2-50), (1-2-82) to (1-2-90), (1-2-109) to (1-2-141), (1-2-169) to (1-2-201), (1-2-229) to (1-2-264), (1-2-289) to (1-2-312), (1-2-361) to (1-2-373), (1-2-506) to (1-2-515), and (1-3-1) to (1-3-20).

Even more preferable compounds are compounds (1-1-1) to (1-1-41), (1-1-72) to (1-1-80), (1-1-123) to (1-1-155), (1-1-270) to (1-1-290), (1-1-333) to (1-1-404), (1-1-447) to (1-1-488), (1-2-1) to (1-2-50), (1-2-82) to (1-2-90), (1-2-109) to (1-2-132), (1-2-133) to (1-2-141), (1-2-169) to (1-2-192), (1-2-253) to (1-2-264), (1-2-289) to (1-2-312), (1-2-361) to (1-2-373), and (1-2-506) to (1-2-515).

### Synthesis Method of Compound

Next, a method for manufacturing the compound according to the present invention will be described. Basically, the compound of the present invention can be synthesized by using a known compound according to a known method, for example, a Suzuki coupling reaction or a Negishi coupling reaction (for example, described in “Metal-Catalyzed Cross-Coupling Reactions—Second, Completely Revised and Enlarged Edition” and the like). Also, the compound can be prepared by combining both reactions. An example of a scheme for synthesizing the compound according to the present invention by the Suzuki coupling reaction or the Negishi coupling reaction is shown below.

When manufacturing the compound of the present invention: (1) a method synthesizing a group in which cyanopyridyl (end group) and a linker L are bonded, and then bonding the L of this group to Ar; and (2) a method bonding a linker L to a desired position of Ar and then bonding the L of this group to a cyanopyridyl group can be mentioned.

Firstly, (1) the method synthesizing a group in which cyanopyridyl (end group) and a linker L are bonded, and then bonding the L of this group to Ar is described.

The synthesis example of the compound represented by formula (1) when m=2 is described below as an example.

### Synthesis Method of Compound Represented by Formula (1) (Part 1)

### Synthesis of Boronic Acid or Boronic Acid Esters of Cyanopyridine

As shown in reaction scheme (1) below, a diboronic acid ester of cyanopyridine can by synthesized by lithiating cyanobromopyridine using an organolithium reagent, or converting cyanobromopyridine into a Grignard reagent using an organic magnesium reagent, and then reacting the resulting material with trimethyl borate, triethyl borate, or triisopropyl borate. Furthermore, as shown in reaction scheme (2) below, boronic acid of cyanopyridine can be synthesized by hydrolyzing this boronic acid ester of cyanopyridine.

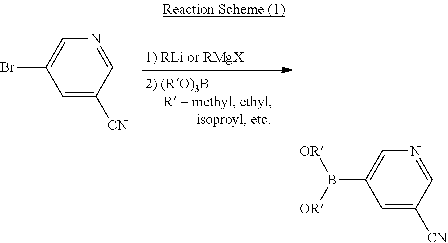

In reaction scheme (1) above, R′ represents a linear or branched alkyl group, and is preferably a linear alkyl group having 1 to 4 carbons, or a branched alkyl group having 3 or 4 carbons.

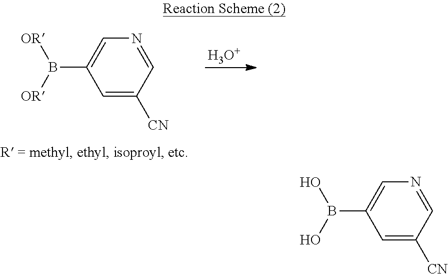

Also, as shown in reaction scheme (3) below, a boronic acid ester can be synthesized by allowing a coupling reaction between cyanobromopyridine and bis(pinacolato)diboron or 4,4,5,5-tetramethyl-1,3,2-dioxaborolane using a palladium catalyst and a base. Note that a commercially-available product can be used as the cyanobromopyridine.

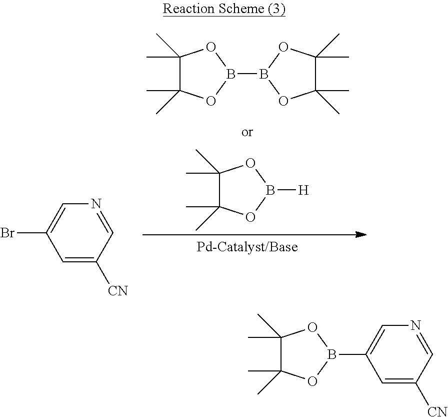

Such boronic acid or boronic acid esters may be arbitrarily subjected to the coupling reaction below. Hereinafter, not being limited to cyanopyridine, the boronic acid and boronic acid esters of a certain substrate may be collectively abbreviated as “boronic acids”. The diboronic acid and diboronic acid esters of a certain substrate may be collectively abbreviated as “diboronic acids”.

### Coupling of Cyanopyridine and Linker L by Suzuki Coupling Method

Next, in reaction scheme (4) below, a compound in which cyanopyridine, which is a precursor to be coupled to Ar, and L having an atom with high reactivity are coupled can be synthesized by reacting boronic acids of cyanopyridine and a desired compound such as 1,3-dibromobenzene, 1,4-dibromobenzene, 2,6-dibromopyridine, 3,5-dibromopyridine, or 2,5-dibromopyridine, which becomes L. Here, a bromoiodide form or a diiodide form can be used instead of the aforementioned dibromo form. Also, although a synthesis method using 3-bromo-5-cyanopyridine as a raw material is exemplified herein, a compound in which cyanopyridine and L having an atom with high reactivity are coupled can be synthesized using various cyanobromopyridines as the raw material. Furthermore, cyanoiodopyridine or cyanochloropyridine may be used instead of cyanobromopyridine. Note that although the boronic acids used here can be synthesized like reaction schemes (1) to (3) above, a commercially-available product may also be used. Ar′ in the scheme below is a divalent group equivalent to L.

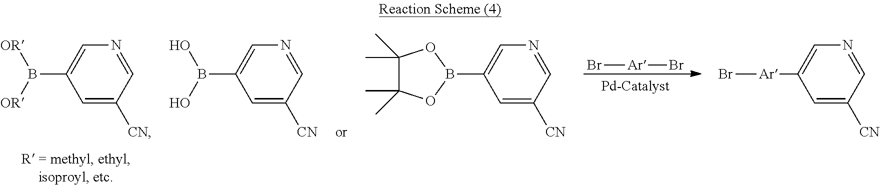

### Synthesis of Zinc Complex of Cyano-Substituted Pyridine

As shown in reaction scheme (5) below, a zinc complex of cyanopyridine can by synthesized by lithiating cyanobromopyridine using an organolithium reagent, or converting cyanobromopyridine into a Grignard reagent using magnesium or an organic magnesium reagent, and then reacting the resulting material with zinc chloride or a tetramethylethylenediamine complex of zinc chloride (ZnCl₂.TMEDA). Although a synthesis method using 3-bromo-5-cyanopyridine as a raw material is exemplified herein, a zinc complex can be similarly synthesized even if various cyanobromopyridines are used as the raw material.

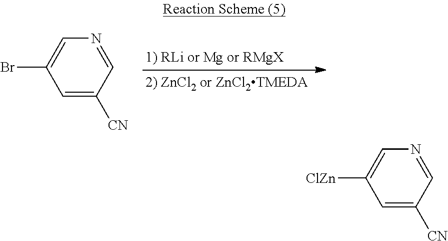

In the reaction scheme (5) above, R represents a linear or branched alkyl group, and is preferably a linear alkyl group having 1 to 4 carbons, or a branched alkyl group having 3 or 4 carbons. Note that it can be similarly synthesized even when a chloride or an iodide is used instead of a bromide.

### Coupling of Cyanopyridine and Linker L by Negishi Coupling Method

As shown in reaction scheme (6) below, a compound in which cyanopyridine, which is a precursor to be coupled to Ar, and L having an atom with high reactivity are coupled can be synthesized by reacting a zinc complex of cyanopyridine and a desired compound such as 1,3-dibromobenzene, 1,4-dibromobenzene, 2,6-dibromopyridine, 3,5-dibromopyridine, or 2,5-dibromopyridine, which becomes L. Here, similar to the Suzuki coupling method, a bromoiodide form or a diiodide form can be used instead of the aforementioned dibromo form. Furthermore, a compound in which cyanopyridine and L having an atom with high reactivity are coupled can be synthesized using a zinc complex of various cyanobromopyridines instead of 3-bromo-5-cyanopyridine as a raw material.

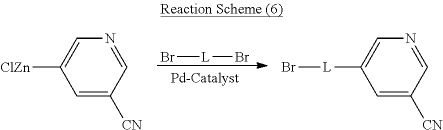

The compound of the present invention can be synthesized by coupling Ar to the thus synthesized compound in which cyanopyridine and L having an atom with high reactivity are coupled. Firstly, regarding the synthesis method using the Suzuki coupling reaction is described.

### Synthesis of Dibromo Form of Ar

Firstly, as shown in reaction scheme (7) below, a dibromo form of Ar is obtained by brominating Ar using a suitable brominating reagent. As suitable brominating reagents, bromine or N-bromosuccinimide (NBS) can be mentioned.

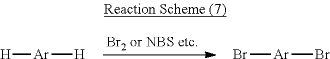

### Synthesis of Diboronic Acids of Ar

Next, as shown in reaction schemes (8) to (10) below, diboronic acids of Ar can be synthesized from dibromo forms of Ar by methods in accordance with the aforementioned schemes (1) to (3). The definition of R in the schemes below is the same as that in reaction scheme (5) above.

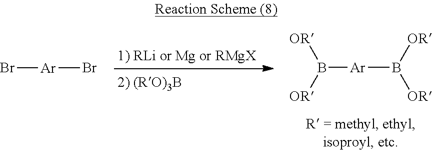

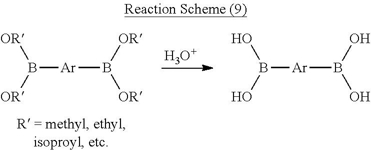

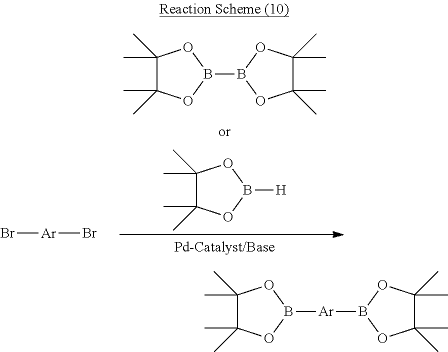

### Synthesis of Compound of Present Invention by Suzuki Coupling Reaction

Lastly, as shown in reaction scheme (11) below, the compound of the present invention can be synthesized by reacting, in the presence of a palladium catalyst and a base, 2-fold moles of a compound in which cyanopyridine and L having an atom with high reactivity are coupled with diboronic acids of Ar synthesized like mentioned above.

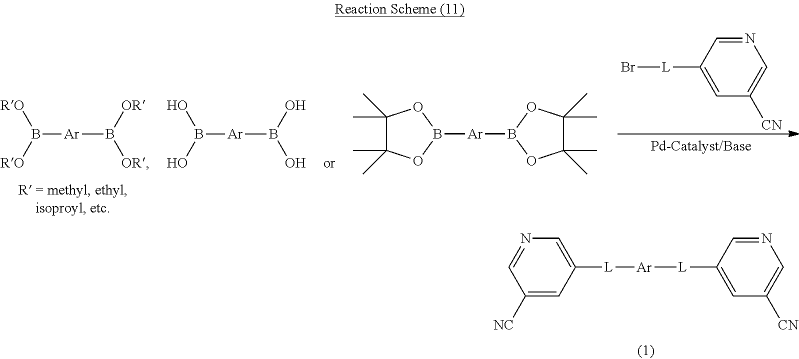

Also, as shown in reaction scheme (12) below, the compound of the present invention can be synthesized by reacting, in the presence of a palladium catalyst and a base, a dibromo form of Ar with 2-fold moles of boronic acids of a compound in which cyanopyridine and L are coupled. Note that boronic acids of compound in which cyanopyridine and L are coupled can be synthesized by methods in accordance with reaction schemes (1) to (3) above from the aforementioned compound in which cyanopyridine and L having an atom with high reactivity are coupled.

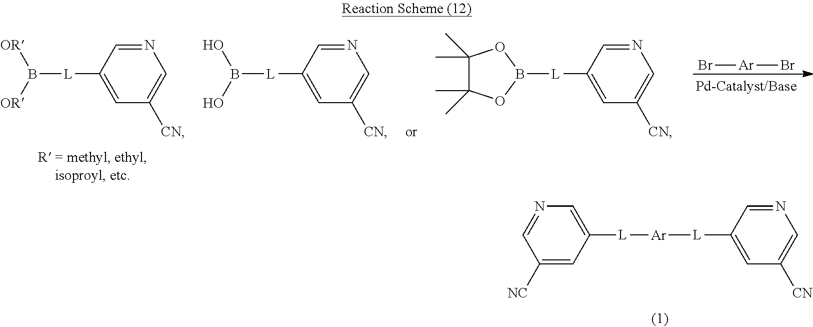

Also, the compound of the present invention can be synthesized by using the Negishi coupling reaction instead of the Suzuki coupling reaction.

### Synthesis of Dizinc Complex of Ar

As shown in reaction scheme (13) below, a dizinc complex of Ar can be synthesized in accordance with the method shown in reaction scheme (5) above.

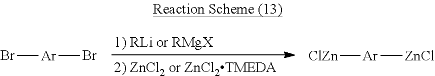

In reaction scheme (13) above, R represents a linear or branched alkyl group, and is preferably a linear alkyl group having 1 to 4 carbons, or a branched alkyl group having 3 or 4 carbons. Note that it can be similarly synthesized even when a chloride or an iodide is used instead of a bromide like a dibromo form of Ar.

### Synthesis of Compound of Present Invention by Negishi Coupling Reaction

Also, as shown in reaction formula (14) below, the compound of the present invention can be synthesized by reacting, in the presence of a palladium catalyst, 2-fold moles of a compound in which cyanopyridine and L having an atom with high reactivity are coupled with a dizinc complex of Ar synthesized like above.

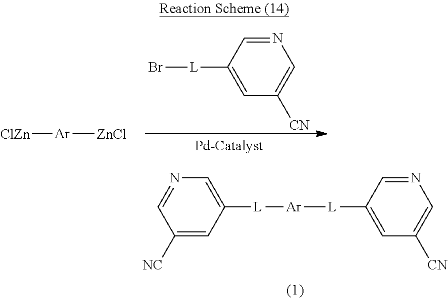

Also, as shown in reaction scheme (15) below, the compound of the present invention can also be synthesized by reacting, in the presence of a palladium catalyst, 2-fold moles of a zinc complex of cyanopyridine and L synthesized in accordance with reaction scheme (5) above from a compound in which cyanopyridine and L having an atom with a high reactivity are coupled with a dibromo form of Ar.

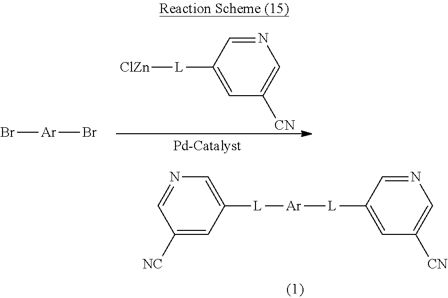

Subsequently, the compound of the present invention can also be synthesized by using a method in which L is bonded to a desired position of Ar and a cyanopyridyl group is bonded to this L. The synthesis example of the compound when Ar is anthracene is described below.

### Synthesis of Monometallized Bromo Aryl

Firstly, as shown in reaction scheme (16) below, a monometallized halogen form of L can by synthesized by lithiating a desired compound such as 1,4-dibromobenzene, 1,3-dibromobenzene, 3,5-dibromopyridine, and 2,6-dibromobenzene, which becomes L, using one equivalent of an organolithium reagent, or converting into a Grignard reagent using one equivalent of magnesium or an organic magnesium reagent. Although an example using a dibromo form is shown herein, a dichloro form, a diiodide form, or the like can be used.

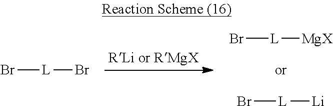

In reaction scheme (16) above, R′ represents a linear or branched alkyl group, and is preferably a linear alkyl group having 1 to 4 carbons, or a branched alkyl group having 3 or 4 carbons.

### Synthesis of Compound in which Anthracene and L Having Atom with High Reactivity are Coupled Using Anthraquinone

As shown in reaction scheme (17) below, a compound in which anthracene and L having an atom with high reactivity are coupled can be synthesized by reacting 2-fold moles of a halogen form of monometallized L and anthraquinone to give a diol form, and then reacting the diol form with sodium phosphonate monohydrate and potassium iodide in acetic acid. Also, the used anthraquinone may by an anthraquinone having a substituent like 2-phenylanthraquinone.

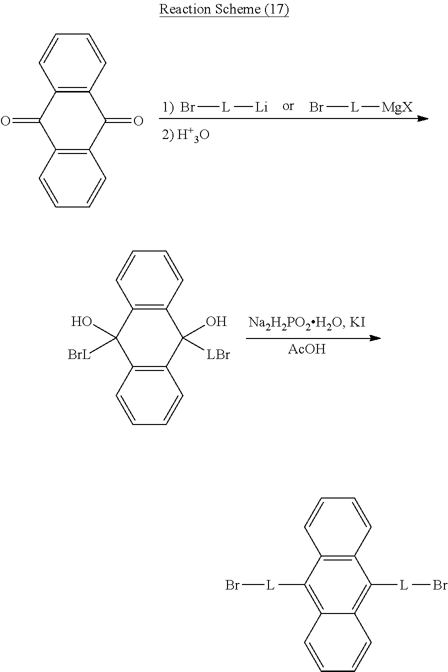

### Synthesis of Compound of Present Invention by Suzuki Coupling Reaction

Also, as shown in reaction scheme (18) below, the compound of the present invention can be synthesized by reacting, in the presence of a palladium catalyst and a base, 2-fold moles of boronic acids of cyano-substituted pyridine with a compound in which anthracene and L having an atom with high reactivity are coupled synthesized like mentioned above. The boronic acids of cyano-substituted pyridine can be synthesized by the methods in accordance with reaction schemes (1) to (3) above.

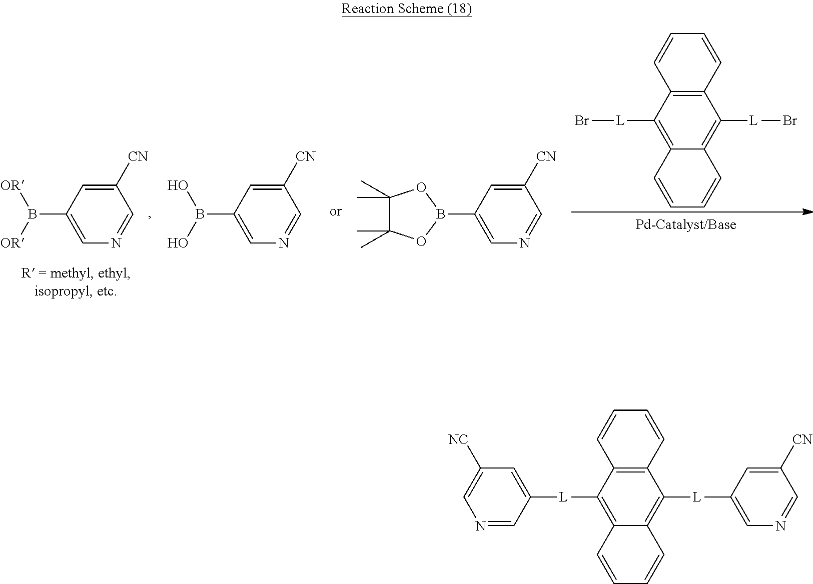

Furthermore, as shown in reaction scheme (19), the compound of the present invention can be synthesized by reacting, in the presence of a palladium catalyst and a base, 2-fold moles of cyano-substituted bromopyridine with diboronic acids which can be synthesized by the methods in accordance with reaction schemes (1) to (3) above from a compound in which anthracene and L having an atom with high reactivity are coupled. The boronic acids can be synthesized by the methods in accordance with reaction schemes (1) to (3) above.

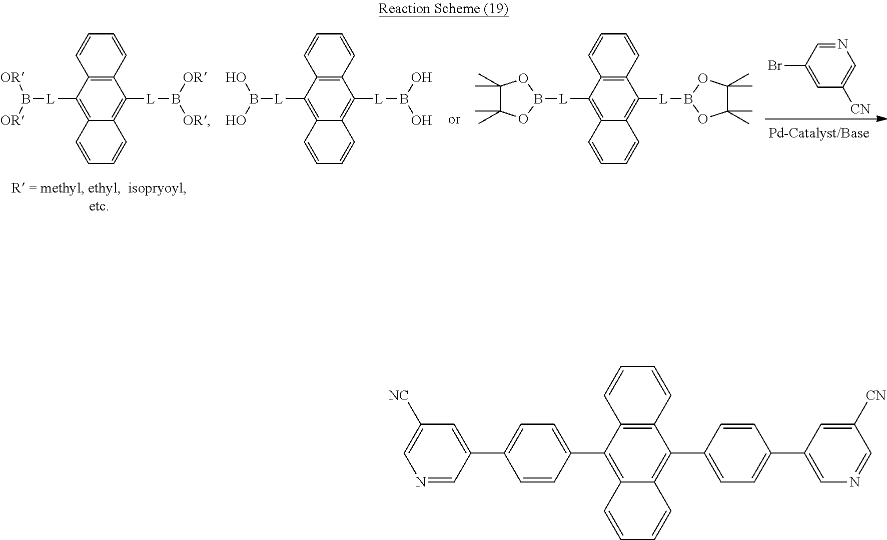

### Synthesis of Compound of Present Invention by Negishi Coupling Reaction

As shown in reaction scheme (20) below, the compound of the present invention can be synthesized by reacting, in the presence of a palladium catalyst, two-fold moles of a zinc complex of cyanopyridine with the compound in which anthracene and L having an atom with high reactivity are coupled synthesized like mentioned above.

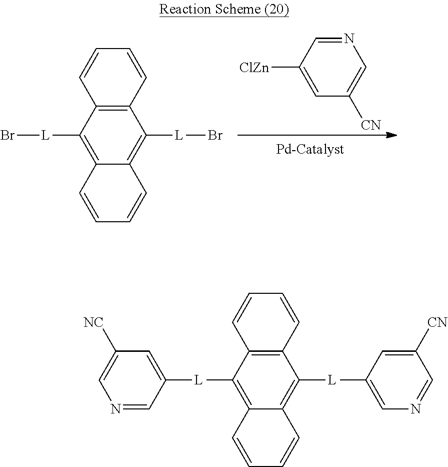

Also, as shown in reaction scheme (21) below, the compound of the present invention can be synthesized by reacting, in the presence of a palladium catalyst, 2-fold moles of cyanobromopyridine with a zinc complex that can be synthesized by the method in accordance with reaction scheme (5) above from a compound in which an anthracene ring and L having an atom with high reactivity are coupled.

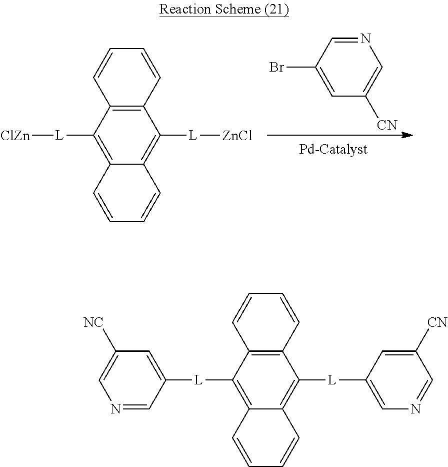

Next, the synthesis example of the compound represented by formula (1) when m=1 is described below as an example.

Firstly, as shown in reaction scheme (22) below, 9-phenylanthracene is synthesized. Bromobenzene is reacted with magnesium metal in THF to give a Grignard reagent and 9-bromoanthracene is reacted with this in the presence of a catalyst to give 9-phenylanthracene. The coupling of a benzene ring and an anthracene ring is not limited to the aforementioned method, and is also possible by the Negishi coupling reaction, the Suzuki coupling reaction, and the like. These usual methods can be appropriately used according to the situation. Also, a commercially-available product can be used as 9-phenylanthracene.

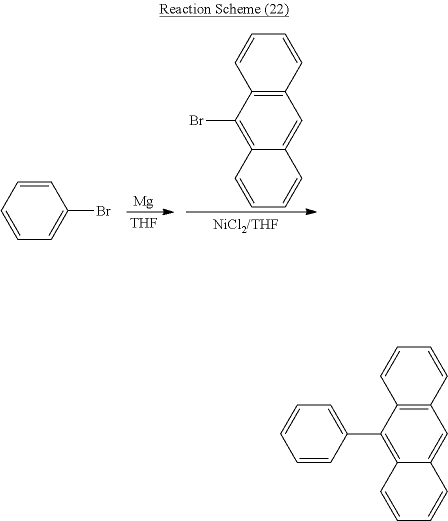

As shown in reaction scheme (23) below, 10-position of 9-phenylanthracene is brominated using N-bromosuccinimide (NBS). A regularly-used brominating reagent other than N-bromosuccinimide such as bromine can also be used herein.

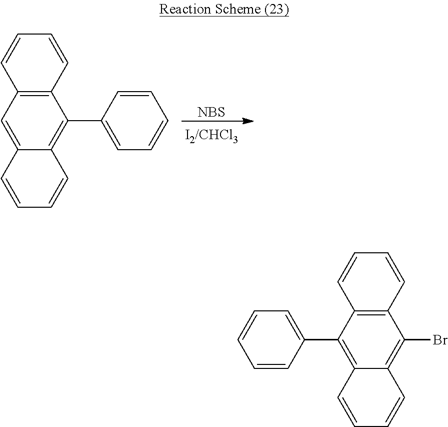

As shown in reaction scheme (24) below, an anthracene ring and a naphthalene ring are coupled. Firstly, 2-bromo-6-methoxynapthalene is converted into a Grignard reagent in accordance with a usual method, and 9-bromo-10-phenylanthrancene is reacted with this in the presence of a catalyst to synthesize 9-(6-methoxynaphtalen-2-yl)-10-phenylanthracene.

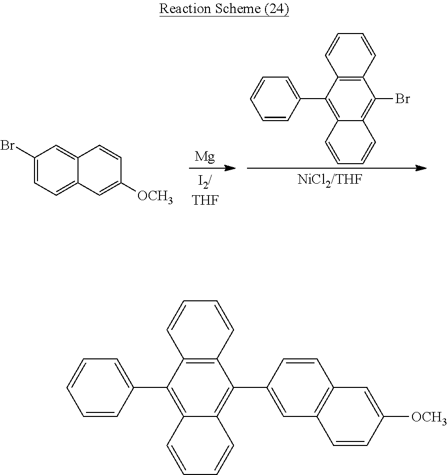

As shown in reaction scheme (25) below, the methoxy group of 9-(6-methoxynaphtalen-2-yl)-10-phenylanthracene is demethylated using boron tribromide to give naphthol. A reagent regularly used in a demethylation reaction such as pyridine hydrochloride can also be appropriately used herein.

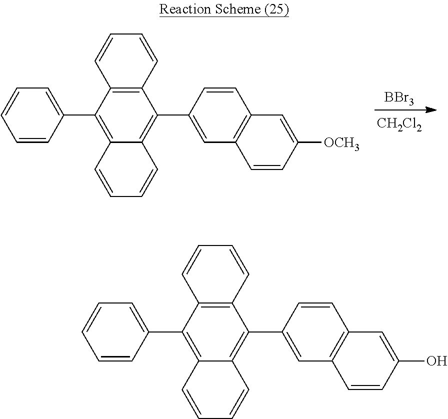

As shown in reaction scheme (26) below, the —OH of naphthol can be converted to trifluoromethylsulfonate (triflate) using anhydrous trifluoromethanesulfonic acid in the presence of a base such as pyridine. —OTf in the reaction scheme is an abbreviation for —OSO₂CF₃.

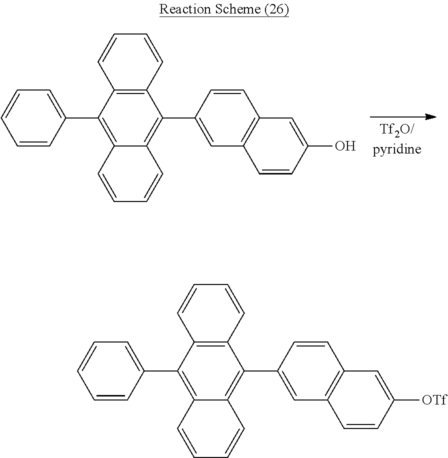 [SMILES: Oc1ccc2cc(-c3c4ccccc4c(-c4ccccc4)c4ccccc34)ccc2c1 ; O=S(=O)(Oc1ccc2cc(-c3c4ccccc4c(-c4ccccc4)c4ccccc34)ccc2c1)C(F)(F)F]

Also, as shown in reaction scheme (27) below, a boronic acid ester can be synthesized by allowing a coupling reaction between triflate and bis(pinacolato)diboron or 4,4,5,5-tetramethyl-1,3,2-dioxaborolane using a palladium catalyst and a base.

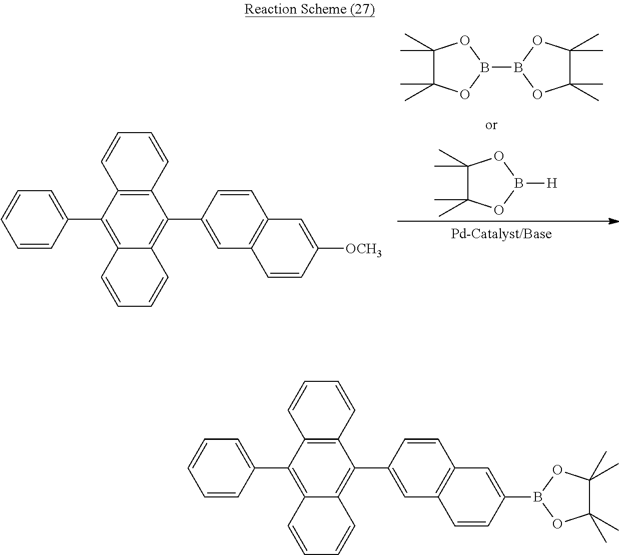

Lastly, the compound of the present invention can be synthesized by the Suzuki coupling reaction of the boronic acid ester obtained by reaction scheme (27) above and cyano-substituted bromopyridine.

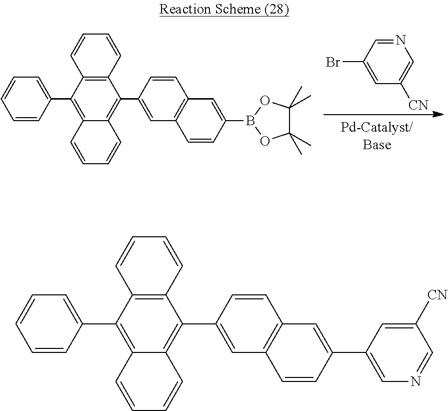 [SMILES: CC1(C)OB(c2ccc3cc(-c4c5ccccc5c(-c5ccccc5)c5ccccc45)ccc3c2)OC1(C)C ; N#Cc1cncc(Br)c1 ; N#Cc1cncc(-c2ccc3cc(-c4c5ccccc5c(-c5ccccc5)c5ccccc45)ccc3c2)c1]

Also, like in reaction scheme (29) below, the compound of the present invention can be synthesized also by the Suzuki coupling reaction of triflate with the boronic acids of cyano-substituted pyridine obtained by reaction schemes (1) to (3) above.

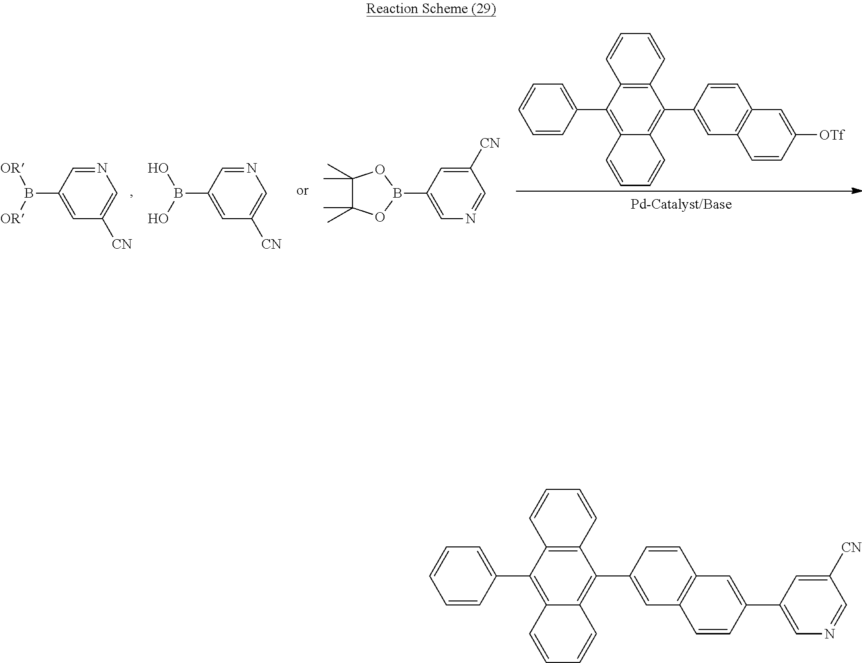

Furthermore, like in reaction scheme (30) below, the compound of the present invention can be synthesized by a Negishi coupling reaction of triflate and the zinc complex of cyano-substituted pyridine obtained by reaction scheme (5) above.

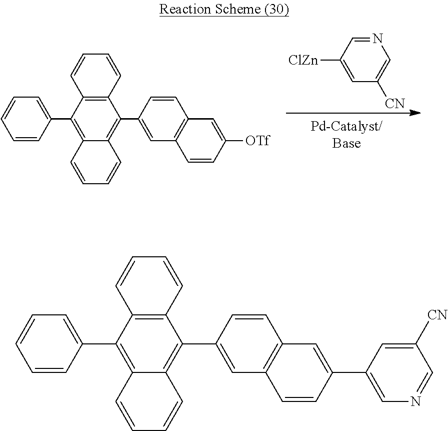

Although the case when Ar is an anthracene substituted with phenyl has been mentioned herein, the compound of the present invention can be synthesized by synthesizing in accordance with the methods mentioned in schemes (8) to (14) above by using a monobromo compound of another Ar.

Thus far, the synthesis methods for coupling the same pyridyl phenyl to Ar for compounds in which m=1 and 2 have been described. When synthesizing a compound in which m=3 or a compound in which m=4, they can be synthesized in accordance with the aforementioned method using Ar having a highly reactive atom or functional group in 3 or 4 positions respectively.

When the linker L is a single bond, the compound of present invention can be synthesized by reacting a brominated Ar and the boronic acids of cyanopyridine obtained by reaction schemes (1) to (3) above in the presence of a palladium catalyst and a base or by reacting a zinc complex of cyanopyridine obtained by reaction scheme (5) above in the presence of a palladium catalyst. Also, the compound of the present invention can be similarly synthesized by reacting boronic acids of Ar and cyanobromopyridine in the presence of a palladium catalyst and a base or by reacting a zinc complex of Ar and cyanobromopyridine in the presence of a palladium catalyst.

In the final coupling reaction, in order to make two or more “groups formed by cyanopyridine and L” in the compound represented by formula (1) have different structures, after firstly reacting Ar having a highly reactive atom or functional group (hereinafter, these are collectively referred to as “reactive site”) with one equivalent of cyanopyridine having a reactive site or a compound in which cyanopyridine and L having a reactive site are coupled, a different cyanopyridine having a reactive site or a different compound in which cyanopyridine and L having a reactive site are coupled is reacted with this intermediate. In other words, it is sufficient for the reaction to be carried out by dividing into 2 or more steps.

Furthermore, a method synthesizing by the following procedure can also be mentioned. One position of Ar is brominated. The used amount of brominating reagent when do so is about ½ of that when obtaining a dibromo form. A mono-substituted form is synthesized by reacting, in the presence of a palladium catalyst and a base, the obtained monobromo form of Ar with equal moles of boronic acids of a compound in which cyanopyridine and brominated L are bonded. Then, this mono-substituted body is further brominated. Next, boronic acids of compound in which cyanopyridine and brominated L are bonded which are different from the first reaction are similarly reacted with the obtained compound, so as to be able to synthesize a compound represented by formula (1) having two different “groups formed by cyanopyridine and L”. Also, for this case, a zinc complex instead of boronic acids can be reacted in the presence of a palladium catalyst. Furthermore, by replacing the compound in which cyanopyridine and a brominated L are bonded with cyanobromopyridine, a compound in which the linker L is a single bond can be synthesized.

Also, although compounds in which at least one hydrogen has been replaced by deuterium are also encompassed by the compound represented by formula (1), such derivatives can by synthesized the same as above by using a raw material in which the desired position has been deuterated.

As examples of the palladium catalyst used in the Suzuki coupling reaction, tetrakis(triphenylphosphine)palladium(0): Pd(PPh₃)₄, bis(triphenylphosphine)palladium(II) dichloride: PdCl₂(PPh₃)₂, palladium(II) acetate: Pd(OAc)₂, tris(dibenzylideneacetone)dipalladium(0): Pd₂(dba)₃, tris(dibenzylideneacetone)dipalladium(0) chloroform complex: Pd₂(dba)₃.CHCl₃, and bis(dibenzylideneacetone)palladium(0): Pd(dba)₂ can be mentioned.

Also, in order to promote the reaction, a phosphine compound may be optionally added to these palladium compounds. As specific examples of the phosphine compound, tri(t-butyl)phosphine, tricyclohexylphosphine, 1-(N,N-dimethylaminomethyl)-2-(di-t-butylphosphino)ferrocene, 1-(N,N-dibutylaminomethyl)-2-(di-t-butylphosphino)ferrocene, 1-(methoxymethyl)-2-(di-t-butylphosphino)ferrocene, 1,1′-bis(di-t-butylphosphino)ferrocene, 2,2′-bis(di-t-butylphosphino)-1,1′-binaphtyl, 2-methoxy-2′-(di-t-butylphosphino)-1,1′-binaphtyl, and 2-dicyclohexylphosphino-2′,6′-dimethoxybiphenyl can be mentioned.

As specific examples of the base used in the reaction, sodium carbonate, potassium carbonate, cesium carbonate, sodium hydrogen carbonate, sodium hydroxide, potassium hydroxide, barium hydroxide, sodium ethoxide, sodium t-butoxide, sodium acetate, tripotassium phosphate, and potassium fluoride can be mentioned.

Also, as specific examples of the solvent used in the reaction, benzene, toluene, xylene, 1,2,4-trimethylbenzene, N,N-dimethylformamide, tetrahydrofuran, diethyl ether, t-butyl methyl ether, 1,4-dioxane, methanol, ethanol, cyclopentyl methyl ether, and isopropyl alcohol can be mentioned. These solvents can be appropriately selected, and may be used alone or as a mixed solvent. Moreover, at least one solvent above and water can also be used in combination.

As examples of the palladium catalyst used in the Negishi coupling reaction, tetrakis(triphenylphosphine)palladium(0): Pd(PPh₃)₄, bis(triphenylphosphine)palladium(II) dichloride: PdCl₂(PPh₃)₂, palladium(II) acetate: Pd(OAc)₂, tris(dibenzylideneacetone)dipalladium(0): Pd₂(dba)₃, tris(dibenzylideneacetone)dipalladium(0) chloroform complex: Pd₂(dba)₃.CHCl₃, bis(dibenzylideneacetone)palladium(0): Pd(dba)₂, bis(tri-t-butylphosphino)palladium(0), and (1,1′-bis(diphenylphosphino)ferrocene)dichloropalladium(II) can be mentioned.

Also, as specific examples of the solvent used in the reaction, benzene, toluene, xylene, 1,2,4-trimethylbenzene, N,N-dimethylformamide, tetrahydrofuran, diethyl ether, t-butyl methyl ether, cyclopentyl methyl ether, and 1,4-dioxane can be mentioned. These solvents can be appropriately selected, and may be used alone or as a mixed solvent.

When the compound of the present invention is used in an electron injection layer or an electron transport layer in an organic EL device, the organic EL device is stable during applicant of an electric field. Such shows that the compound of the present invention is superior as an electron injection material or an electron transport material of an electroluminescent device. Herein, electron injection layer means a layer for receiving an electron from a cathode to an organic layer, and electron transport layer means a layer for transporting an injected electron to an emission layer. Also, the electron transport layer can simultaneously serve as the electron injection layer. The material used in each layer is referred to as electron injection material and the electron transport material respectively.

### Description of Organic EL Device

A second aspect of the present invention refers to an organic EL device including the compound represented by formula (1) of the present invention in an electron injection layer or an electron transport layer. In the organic EL device of the present invention, driving voltage is low, and durability during driving is high.

Although the structure of the organic EL device of the present invention includes various aspects, it is basically a multilayer structure in which at least a hole transport layer, an emission layer, and an electron transport layer are sandwiched between an anode and a cathode. Specific examples of the constitution of the device include (1) anode/hole transport layer/emission layer/electron transport layer/cathode, (2) anode/hole injection layer/hole transport layer/emission layer/electron transport layer/cathode, (3) anode/hole injection layer/hole transport layer/emission layer/electron transport layer/electron injection layer/cathode, and the like.

The compound of the present invention has high electron injection properties and high electron transport properties, and thus can be used in the electron injection layer or electron transport layer in the form of a simple substance or in combination with other materials. The organic EL device of the present invention can obtain blue, green, red, or white emission by combining a hole injection layer, a hole transport layer, an emission layer, and the like in which other materials are used in the electron transport material of the present invention.

An emission material or emissive dopant that can be used in the organic EL device of the present invention is an emission material such as a daylight fluorescent material, a fluorescent whitening reagent, a laser dye, an organic scintillator, and various kinds of fluorescence analysis reagents as described in “Optical Functional Materials,” Polymer Functional Material Series, edited by The Society of Polymer Science, Japan, Kyoritsu Shuppan Co., Ltd., (1991), p. 236; a dopant material like described in “Organic EL Material and Display”, under supervision of Junji Kido, published by CMC Co., Ltd., 2001, pp. 155-156; an emission material of a triplet light emitting material like described in “Organic EL Material and Display”, under supervision of Junji Kido, published by CMC Co., Ltd., 2001, pp. 170-172; and the like.

Compounds that can be used as the emission material or the emissive dopant are a polycyclic aromatic compound, a heteroaromatic compound, an organometallic complex, a dye, a polymer-based emission material, a styryl derivative, an aromatic amine derivative, a coumarin derivative, a borane derivative, an oxazine derivative, a compound having a spiro ring, an oxadiazole derivative, a fluorene derivative, and the like. Examples of the polycyclic aromatic compound are an anthracene derivative, a phenanthrene derivative, a naphthacene derivative, a pyrene derivative, a chrysene derivative, a perylene derivative, a coronene derivative, a rubrene derivative, and the like. Examples of the heteroaromatic compound are an oxadiazole derivative having a dialkylamino group or a diarylamino group, a pyrazoloquinoline derivative, a pyridine derivative, a pyran derivative, a phenanthroline derivative, a silole derivative, a thiophene derivative having a triphenylamino group, a quinacridone derivative, and the like. Examples of the organometallic complex are a complex between zinc, aluminum, beryllium, europium, terbium, dysprosium, iridium, platinum, osmium, gold or the like and a quinolinol derivative, a benzoxazole derivative, a benzothiazole derivative, an oxadiazole derivative, a thiadiazole derivative, a benzimidazole derivative, a pyrrole derivative, a pyridine derivative, a phenanthroline derivative, or the like. As examples of the dye, dyes such as a xanthene derivative, a polymethine derivative, a porphyrin derivative, a coumarin derivative, a dicyanomethylene pyran derivative, a dicyanomethylene thiopyran derivative, an oxobenzanthracene derivative, a carbostyryl derivative, a perylene derivative, a benzooxazole derivative, a benzothiazole derivative, and a benzimidazole derivative can be mentioned. Examples of the polymer emission material are a polyparaphenylvinylene derivative, a polythiophene derivative, a polyvinylcarbazole derivative, a polysilane derivative, a polyfluorene derivative, a polyparaphenylene derivative, and the like. Examples of the styryl derivative are an amine-containing styryl derivative, a styrylarylene derivative, and the like.

Other electron transport materials used in the organic EL device of the present invention can be arbitrarily selected from compounds that can be used as an electron transfer compound in a photoconductive material, or compounds that can be used in the electron transport layer and the electron injection layer of the organic EL device.

Specific examples of such an electron transport material are a quinolinol-based metal complex; a metal complex of a 2,2′-bipyridyl derivative, a phenanthroline derivative, a diphenylquinone derivative, a perylene derivative, an oxadiazole derivative, a thiophene derivative, a triazole derivative, a thiadiazole derivative, or an oxine derivative; a quinoxaline derivative, a polymer of a quinoxaline derivative, a benzazoles compound, a gallium complex, a pyrazol derivative, a perfluorated phenylene derivative, a triazine derivative, a pyrazine derivative, a benzoquinoline derivative, an imidazopyridine derivative, a borane derivative, and the like.

Regarding the hole injection material and the hole transport material used in the organic EL device of the present invention, an arbitrary material can be selected and used from compounds that have been conventionally commonly used as a hole charge transport material in a photoconductive material, or publicly-known compounds used in a hole injection layer and a hole transport layer of an organic EL device. Specific examples thereof are a carbazole derivative, a triarylamine derivative, a phthalocyanine derivative, and the like.

Each layer composing the organic EL device of to the present invention can be formed by making the material composing each layer into a thin film by a method such as a vapor deposition method, a spin coating method, or a casting method. The thickness of the thus formed each layer is not particularly limited, and can be appropriately set according to properties of the material. The thickness is ordinarily in the range of 2 nm to 5,000 nm. In addition, as a method for processing the emission material into a thin film, a vapor deposition method is preferably used from points such as easiness in obtaining a uniform film and difficulty in generating pin holes. When made into the thin film by using a vapor deposition method, the vapor deposition conditions thereof differ by the type of emission material of the present invention. In general, the vapor deposition conditions are preferably set appropriately in the range of a boat heating temperature of 50 to 400° C., a vacuum of 10⁻⁶ to 10⁻³ Pa, a vapor deposition rate of 0.01 to 50 nm/sec in a vacuum evaporation rate, a substrate temperature of −150 to +300° C., and a film thickness of 5 nm to 5 μm.

The organic EL device of the present invention is preferably supported on a substrate even in any structure described above. Any substrate may be used as long as the substrate has mechanical strength, thermal stability and transparency. Glass, a transparent plastic film, or the like can be used. As an anode material, a metal, an alloy, or an electric conductive compound having a work function larger than 4 eV, and a mixture thereof can be used. Specific examples thereof are a metal such as gold, CuI, indium tin oxide (hereafter, abbreviated as ITO), SnO₂, ZnO, and the like.

As a cathode material, a metal, an alloy, or an electric conductive compound having a work function smaller than 4 eV, and a mixture thereof can be used. Specific examples thereof are aluminum, calcium, magnesium, lithium, a magnesium alloy, an aluminum alloy, and the like. Specific examples of the alloy are aluminum/lithium fluoride, aluminum/lithium, magnesium/silver, magnesium/indium, and the like. In order to efficiently extract emission of the organic EL device, at least one of the electrodes is desirably set to have a light transmittance of 10% or more. Sheet resistance as an electrode is preferably set to be several hundred Ω/□ or less. In addition, film thickness, although also depending on properties of an electrode material, is set in the range of ordinarily 10 nm to 1 μm, and preferably 10 nm to 400 nm. Such an electrode can be prepared by forming a thin film using the aforementioned electrode material by a method such as vapor deposition or sputtering.

Next, as one example of a method for preparing the organic EL device by using the emission material of the present invention, a method for preparing the organic EL device consisting of the aforementioned anode/hole injection layer/hole transport layer/emission layer/electron transport material of the present invention/cathode is described. After forming a thin film of the anode material on a suitable substrate by a vapor deposition method to prepare the anode, thin films of the hole injection layer and the hole transport layer are formed on the anode. A thin film of the emission layer is formed thereon. The electron transport material of the present invention is vacuum-deposited on the emission layer to form a thin film, and the thin film serves as the electron transport layer. By further forming a thin film consisting of the material for the cathode by a vapor deposition method to serve as the cathode, the target organic EL device is obtained. In addition, in preparation of the aforementioned organic EL device, the organic EL device can also be prepared in the order of cathode, electron transport layer, emission layer, hole transport layer, hole injection layer, and anode by reversing the preparation order.

In the case where a direct current voltage is applied to the thus obtained organic EL device, the voltage may be applied by setting polarity of the anode as (+) and polarity of the cathode as (−). If a voltage of about 2 to 40 V is applied, emission can be observed from a transparent or translucent electrode side (anode or cathode, or both of the anode and the cathode). Also, the organic EL device emits light also when alternating current voltage is applied thereto. In addition, a waveform of alternating current to be applied may be arbitrary.

### EXAMPLES

The present invention will be described in more detail based on examples below. Firstly, synthesis examples of the compounds used in the examples will be described below.

### Synthesis Example 1

### Synthesis of Compound (1-1-2): 5-cyano-3-(6-(10-phenylanthracen-9-yl)naphthalen-2-yl)pyridine

2.53 g of 4,4,5,5-tetramethyl-2-(6-10-phenylanthracen-9-yl)naphthalen-2-yl)-1,3,2-dioxaborolane prepared by referring to the method described in WO 2012/060374, 1.01 g of 3-bromo-5-cyanopyridine, 0.17 g of tetrakis(triphenylphosphine)palladium(0), 2.12 g of tripotassium phosphate, 20 mL of pseudocumene, 5 mL of t-butyl alcohol, and 1 mL of water were put into a flask, and the resulting mixture was stirred at reflux temperature for 8 hours under a nitrogen atmosphere. The reaction solution was cooled to room temperature and, after extracting with toluene, the organic layer was dried with sodium sulfate. The crude product obtained by distilling off the solvent under reduced pressure was purified by silica gel column chromatography (eluent: toluene/ethyl acetate=95/5 (volume ratio)), to obtain 0.19 g of 5-cyano-3-(6-(10-phenylanthracen-9-yl)naphthalen-2-yl)pyridine.

¹H-NMR (CDCl₃): 9.2 (d, 1H), 8.9 (d, 1H), 8.3 (t, 1H), 8.2 (d, 1H), 8.2 (d, 1H), 8.1 (d, 1H), 8.1 (s, 1H), 7.8 (dd, 1H), 7.8-7.7 (m, 5H), 7.6 (m, 2H), 7.6 (m, 1H), 7.5 (m, 2H), 7.4-7.3 (m, 4H).

### Synthesis Example 2

### Synthesis of Compound (1-2-27): 9,10-(bis(4-(5-cyanopyridin-3-yl)phenyl)-2-phenylanthracene

### Synthesis of 2-phenylanthraquinone

125.0 g of 2-chloroanthraquinone, 75.4 g of phenylboronic acid, 1.79 g of tetrakis(triphenylphosphine)palladium(0), 109.3 g of tripotassium phosphate, 400 mL of pseudocumene, 100 mL of t-butyl alcohol, and 20 mL of water were put into a flask, and the resulting mixture was stirred at reflux temperature for 3.5 hours under a nitrogen atmosphere. The reaction solution was cooled to room temperature, the precipitated crystals were collected by filtration, and, after washing the collected crystals with water, they were purified by a silica gel short column (eluent: toluene), to obtain 106.0 g of 2-phenylanthraquinone.

### Synthesis of 9,10-bis(4-bromophenyl)-2-phenyl-9,10-dihydroanthracene-9,10-diol

47.2 g of 1,4-dibromobenzene and 250 mL of dehydrated cyclopentyl methyl ether were put into a flask, and the resulting mixture was cooled to −78° C. 78 mL of n-butyl lithium (2.69 M hexane solution) was added dropwise thereto while stirring and, after the dropwise addition, the resulting mixture was stirred for 0.5 hours. 22.7 g of 2-phenylanthraquinone was added thereto, and the resulting mixture was stirred as is for 5 hours. Water was added thereto to stop the reaction and, after extracting with toluene, the organic layer was dried with magnesium sulfate. The solid obtained by distilling off the solvent under reduced pressure was purified by a silica gel short column (eluent: toluene/ethyl acetate=4/1 (volume ratio)), to obtain 42.5 g of 9,10-(bis(4-bromophenyl)-2-phenyl-9,10-dihydroanthracene-9,10-diol.

### Synthesis of 9,10-(4-bromophenyl)-2-phenylanthracene

41.9 g of 9,10-bis(4-bromophenyl)-2-phenyl-9,10-dihydroanthracene-9,10-diol, 90.3 g of sodium phosphinate monohydrate, 30.2 g of potassium iodide, and 200 mL of acetic acid were put into a flask, and the resulting mixture was stirred at reflux temperature for 3 hours. The reaction solution was cooled to room temperature, water was added thereto, and the precipitated solid was collected by filtration. The solid was washed with water, methanol, and then ethyl acetate. This crude product was purified by a silica gel short column (eluent: toluene), to obtain 38.5 g of 9,10-(4-bromophenyl)-2-phenylanthracene.

### Synthesis of 2,2′-((2-phenylanthracen-9,10-diyl)bis(4,1-phenylene))bis(4,4,5,5-tetramethyl-1,3,2-dioxaborolane)

12.0 g of 9,10-(4-bromophenyl)-2-phenylanthracene, 12.2 g of bis(pinacolato)diboron, 0.49 g of (1,1′-bis(diphenylphosphino)ferrocene)palladium(II) dichloride dichloromethane complex, 7.85 g of potassium acetate, and 40 mL of cyclopentyl methyl ether were put into a flask, and the resulting mixture was stirred at reflux temperature for 6 hours. The reaction liquid was cooled to room temperature, water was added thereto, and after extracting with toluene, the organic layer was dried with magnesium sulfate. The crude product obtained by distilling off the solvent under reduced pressure was purified by a silica gel short column (eluent: toluene), to obtain 11.5 g of 2,2′-((2-phenylanthracen-9,10-diyl)bis(4,1-phenylene))bis(4,4,5,5-tetramethyl-1,3,2-dioxaborolane).

### Synthesis of Compound (1-2-27): 9,10-bis(4-(5-cyanopyridin-3-yl)phenyl)-2-phenylanthracene

2.69 g of 2,2′-((2-phenylanthracen-9,10-diyl)bis(4,1-phenylene))bis(4,4,5,5-tetramethyl-1,3,2-dioxaborolane), 1.98 g of 3-bromo-5-cyanopyridine, 0.16 g of tetrakis(triphenylphosphine)palladium(0), 2.29 g of tripotassium phosphate, 20 mL of pseudocumene, 5 mL of t-butyl alcohol, and 1 mL of water were put into a flask, and the resulting mixture was stirred at reflux temperature for 8 hours under a nitrogen atmosphere. The reaction solution was cooled and, after extracting with toluene, the organic layer was dried with sodium sulfate. The crude product obtained by distilling off under reduced pressure the solvent was purified by silica gel column chromatography (eluent: toluene/ethyl acetate=9/1 (volume ratio)), to obtain 0.1 g of 9,10-bis(4-(5-cyanopyridin-3-yl)phenyl)-2-phenylanthracene.

¹H-NMR (CDCl₃): 9.2 (d, 2H), 8.9 (dd, 2H), 8.3 (m, 2H), 7.9-7.8 (m, 6H), 7.8-7.7 (m, 7H), 7.6 (m, 2H), 7.4 (m, 4H), 7.4-7.3 (m, 1H).

### Synthesis Example 3

### Synthesis of Compound (1-2-48): 9,10-bis((5′-cyano-2,3′-bipyridin-6-yl)2-phenylanthracene

### Synthesis of 3-cyano-5-(4,4,5,5-tetramethyl-1,3,2-dioxaborolan-2-yl)pyridine

3-bromo-5-cyanopyridine (10 g), bis(pinacolato)diboron (15.3 g), potassium acetate (10.7 g), (1,1′-bis(diphenylphosphino)ferrocene)palladium(II) dichloride dichloromethane complex (1.34 g), and cyclopentyl methyl ether (100 mL) were put into a flask, and the resulting mixture was stirred at reflux temperature for 8 hours under a nitrogen atmosphere. The reaction liquid was cooled to room temperature, and two-liquid separation was carried out by added water and further toluene thereto. After separating the organic layer, the organic layer was dried and concentrated, and then passed through an activated carbon short column (eluent: toluene). The target compound (6.70 g) was obtained thereafter by concentrating and reprecipitating with heptane.

### Synthesis of Compound (1-2-48): 9,10-bis((5′-cyano-2,3′-bipyridin-6-yl)2-phenylanthracene

9,10-bis(6-bromopyridin-2-yl)-2-phenylanthracene (3.00 g) synthesized by the method disclosed in Japanese Unexamined Patent Application, First Publication No. 2014-82479, 3-cyano-5-(4,4,5,5-tetramethyl-1,3,2-dioxaborolan-2-yl)pyridine (2.92 g), potassium carbonate (2.93 g), tetra-n-butylammonium bromide (0.34 g), bis(ditertiarybutyl(4-dimethylaminophenyl)phosphine)dichloropalladium (0.11 g), 1,2,4-trimethylbenzene (20 mL), and water (2 mL) were put into a flask, and the resultant mixture was stirred at reflux temperature for 4 hours under a nitrogen atmosphere. The reaction liquid was cooled to room temperature, and two-liquid separation was carried out by adding water and further toluene thereto. After separating the organic layer, the organic layer was dried and concentrated, and then the crude product was purified by an NH silica gel column (eluent: toluene/ethyl acetate=4/1 (volume ratio)). The target compound (1.30 g) was obtained thereafter by carrying out sublimation purification.

¹H-NMR (CDCl₃): 9.5 (d, 2H), 8.9 (m, 2H), 8.7 (m, 2H), 8.1 (dt, 2H), 8.0 (dd, 2H), 7.8-7.3 (m, 14H).

### Synthesis Example 4

### Synthesis of Compound (1-2-173): 2,7-bis(3-(5-cyanopyridin-3-yl)phenyl)-5,5′-(9,9′-spirobi[fluorene])

### Synthesis of 3-(5-cyanopyridin-3-yl)phenylbromide

3-cyano-5-(4,4,5,5-tetramethyl-1,3,2-dioxaborolan-2-yl)pyridine (17.5 g), 1-bromo-3-iodobenzene (28.0 g), potassium carbonate (21.0 g), tetra-n-butylammonium bromide (4.90 g), bis(ditertiarybutyl(4-dimethylaminophenyl)phosphine)dichloropalladium (1.61 g), 1,2,4-trimethylbenzene (100 mL), and water (10 mL) were put into a flask, and the resultant mixture was stirred at reflux temperature for 8 hours under a nitrogen atmosphere. The reaction liquid was cooled to room temperature, and two-liquid separation was carried out by adding water and further toluene thereto. After separating the organic layer, the organic layer was dried and concentrated, and the crude product was purified by an NH silica gel column (eluent: toluene/ethyl acetate=9/1 (volume ratio)), to obtain the target compound (6.00 g).

### Synthesis of 2,7-bis(4,4,5,5-tetramethyl-1,3,2-dioxaborolan-2-yl)-5,5′-(9,9′-spirobi[fluorene])

2,7-dibromo-5,5′-(9,9′-spirobi[fluorene] (8.0 g)), bis(pinacolato)diboron (10.3 g), potassium acetate (6.62 g), (1,1′-bis(diphenylphosphino)ferrocene)palladium(II) dichloride dichloromethane complex (0.41 g), and cyclopentyl methyl ether (100 mL) were put into a flask, and the resulting mixture was stirred at reflux temperature for 5 hours under a nitrogen atmosphere. The reaction liquid was cooled to room temperature, and two-liquid separation was carried out by adding water and further toluene thereto. After separating the organic layer, the organic layer was dried and concentrated, and then passed through an activated carbon short column (eluent: toluene). The target compound (9.00 g) was obtained thereafter by concentrating and reprecipitating with heptane.

### Synthesis of Compound (1-2-173): 2,7-bis(3-(5-cyanopyridin-3-yl)phenyl)-5,5′-(9,9′-spirobi[fluorene])

2,7-bis(4,4,5,5-tetramethyl-1,3,2-dioxaborolan-2-yl)-5,5′-(9,9′-spirobi[fluorene]) (3.00 g), 3-bromo-5-cyanopyridine (3.28 g), potassium carbonate (2.92 g), tetra-n-butylammonium bromide (0.34 g), bis(ditertiarybutyl(4-dimethylaminophenyl)phosphine)dichloropalladium (0.037 g), 1,2,4-trimethylbenzene (20 mL), and water (2 mL) were put into a flask, and the resultant mixture was stirred at reflux temperature for 4 hours under a nitrogen atmosphere. The reaction liquid was cooled to room temperature, and two-liquid separation was carried out by adding water and further toluene thereto. After separating the organic layer, the organic layer was dried and concentrated, and then the crude product was purified by an NH silica gel column (eluent: toluene/ethyl acetate=4/1 (volume ratio)). The target compound (1.78 g) was obtained thereafter by carrying out sublimation purification.

¹H-NMR (CDCl₃): 9.0 (d, 2H), 8.8 (d, 2H), 8.1 (t, 2H), 8.0 (d, 2H), 7.9 (d, 2H), 7.7 (dd, 2H), 7.6 (t, 2H), 7.5 (m, 2H), 7.5-7.4 (m, 6H), 7.1 (dt, 2H), 7.0 (d, 2H), 6.8 (d, 2H).

### Synthesis Example 5

### Synthesis of Compound (1-2-179): 2,7-bis(5-cyanopyridin-3-yl)-5,5′-(9,9′-spirobi[fluorene])

2,7-dibromo-5,5′-(9,9′-spirobi[fluorene]) (2.73 g), 3-cyano-5-(4,4,5,5-tetramethyl-1,3,2-dioxaborolan-2-yl)pyridine (3.18 g), potassium carbonate (1.59 g), tetra-n-butylammonium bromide (0.37 g), bis(ditertiarybutyl(4-dimethylaminophenyl)phosphine)dichloropalladium (0.091 g), 1,2,4-trimethylbenzene (20 mL), and water (2 mL) were put into a flask, and the resultant mixture was stirred at reflux temperature for 9 hours under a nitrogen atmosphere. The reaction liquid was cooled to room temperature, and two-liquid separation was carried out by adding water and further toluene thereto. After separating the organic layer, the organic layer was dried and concentrated, and then the crude product was purified by an NH silica gel column (eluent: toluene/ethyl acetate=4/1 (volume ratio)). The target compound (1.59 g) was obtained thereafter by carrying out sublimation purification.

¹H-NMR (CDCl₃): 8.9 (d, 2H), 8.8 (d, 2H), 8.1 (d, 2H), 8.0 (t, 2H), 7.9 (d, 2H), 7.6 (dd, 2H), 7.4 (dt, 2H), 7.2 (dt, 2H), 6.9 (d, 2H), 6.8 (d, 2H).

### Synthesis Example 6

### Synthesis of Compound (1-2-365): 2,7-bis(3-(5-cyanopyridin-3-yl)phenyl)triphenylene

4,7-bis(4,4,5,5-tetramethyl-1,3,2-dioxaborolan-2-yl)triphenylene (1.60 g) synthesized by the method disclosed in PCT International Publication No. WO 2007/029696, 3-bromo-5-cyanopyridine (1.90 g), potassium carbonate (1.84 g), tetra-n-butylammonium bromide (0.21 g), bis(ditertiarybutyl(4-dimethylaminophenyl)phosphine)dichloropalladium (0.024 g), 1,2,4-trimethylbenzene (20 mL), and water (2 mL) were put into a flask, and the resultant mixture was stirred at reflux temperature for 6 hours under a nitrogen atmosphere. The reaction liquid was cooled to room temperature, water was added thereto, and the precipitate was collected by filtration. After dissolving the filtrate in heated chloroform, such was purified by filtering with celite and by further carrying out recrystallization with pyridine. The target compound (0.98 g) was obtained thereafter by carrying out sublimation purification.

EI-MS: m/z=584.

### Synthesis Example 7

### Synthesis of Compound (1-2-506): 2,7-bis(4-cyanopyridin-3-yl)triphenylene

2,7-bis(4,4,5,5-tetramethyl-1,3,2-dioxaborolan-2-yl)triphenylene (2.39 g), 3-bromo-4-cyanopyridine (2.00 g), potassium carbonate (2.75 g), tetra-n-butylammoniumbromide (0.32 g), bis(ditertiarybutyl(4-dimethylaminophenyl)phosphine)dichloropalladium (0.035 g), 1,2,4-trimethylbenzene (20 mL), and water (2 mL) were put into a flask, and the resultant mixture was stirred at reflux temperature for 5 hours under a nitrogen atmosphere. The reaction liquid was cooled to room temperature, water was added thereto, and the precipitate was filtered out. After dissolving the precipitate in heated chloroform, such was purified by filtering with celite and by further carrying out recrystallization with benzonitrile and pyridine. The target compound (0.54 g) was obtained by carrying out sublimation purification.

¹H-NMR (CDCl₃): 9.1 (s, 2H), 8.9 (d, 2H), 8.9-8.8 (m, 4H), 8.7 (dd, 2H), 7.9 (dd, 2H), 7.8-7.7 (m, 4H).

### Synthesis Example 8

### Synthesis of Compound (1-2-507): 3,9-bis(3-cyanopyridin-4-yl)spiro[benzo[a]fluorene-11,9′-fluorene]

### Synthesis of 3,9-bis(4,4,5,5-tetramethyl-1,3,2-dioxaborolan-2-yl)spiro[benzo[a]fluorene-11,9′-fluorene]

Spiro[benzo[a]fluorene-11,9′-fluorene]-3,9-diylbis(trifluoromethanesulfonate) (5.00 g) synthesized according to the method described in Japanese Unexamined Patent Application, First Publication No. 2009-184993, bis(pinacolato)diboron (4.60 g), potassium acetate (2.96 g), (1,1′-bis(diphenylphosphino)ferrocene)palladium(II) dichloride dichloromethane complex (0.18 g), and cyclopentyl methyl ether (50 mL) were put into a flask, and the resulting mixture was stirred at reflux temperature for 8 hours under a nitrogen atmosphere. The reaction liquid was cooled to room temperature, and two-liquid separation was carried out by adding water and further toluene thereto. After separating the organic layer, the organic layer was dried and concentrated, and then passed through an activated carbon short column (eluent: toluene). The target compound (4.00 g) was obtained thereafter by concentrating and reprecipitating with heptane.

### Synthesis of Compound (1-2-507): 3,9-bis(3-cyanopyridin-4-yl)spiro[benzo[a]fluorene-11,9′-fluorene]

3,9-bis(4,4,5,5-tetramethyl-1,3,2-dioxaborolan-2-yl)spiro[benzo[a]fluorene-11,9′-fluorene] (3.07 g), 4-bromo-3-cyanopyridine (2.00 g), potassium carbonate (2.75 g), tetra-n-butylammonium bromide (0.32 g), bis(ditertiarybutyl(4-dimethylaminophenyl)phosphine)dichloropalladium (0.11 g), 1,2,4-trimethylbenzene (20 mL), and water (2 mL) were put into a flask, and the resultant mixture was stirred at reflux temperature for 5 hours under a nitrogen atmosphere. The reaction liquid was cooled to room temperature, and two-liquid separation was carried out by adding water and further toluene thereto. After separating the organic layer, the organic layer was dried and concentrated, and the crude product was then purified by an NH silica gel column (eluent: toluene/ethyl acetate=4/1 (volume ratio)). The target compound (1.20 g) was obtained thereafter by carrying out sublimation purification.

¹H-NMR (CDCl₃): 8.7 (d, 1H), 8.6 (d, 1H), 8.2-8.0 (m, 6H), 7.9 (d, 1H), 7.7 (m, 3H), 7.5 (dd, 1H), 7.5 (dt, 2H), 7.3 (dd, 1H), 7.1 (dt, 2H), 6.9 (d, 1H), 6.9 (d, 1H), 6.7 (d, 2H).

The other derivative compounds of the present invention can by synthesized by methods according to the aforementioned synthesis examples by appropriately changing the raw material compounds.

Although examples of organic EL devices using compounds of the present invention are shown below in order to further describe the present invention, the present invention is not limited thereto.

The devices of Examples 1 to 10 and Comparative Examples 1 to 8 were prepared, and measurement of the driving voltage (V) and the external quantum efficiency (%) during emission of 1,000 cd/m², and measurement of the time (hr) for which luminance of 80% (1,200 cd/m²) or more of the initial value is maintained when a constant current drive test at the current density for which a luminance of 1,500 cd/m² is obtained were carried out. Examples are described in detail below.

The material composition of each layer in the prepared devices of Examples 1 to 6 and Comparative Examples 1 to 8 are shown in Tables 1 and 2.

TABLE 1

| | Hole | Hole | Hole | | | Electron | |
|---|---|---|---|---|---|---|---|
| | Injection | Injection | Transport | Emission Layer | | Transport | |
| | Layer 1 | Layer 2 | Layer | (20 nm) | | Layer* | Cathode |
| | (40 nm) | (5 nm) | (25 nm) | Host | Dopant | (30 nm) | (1 nm/100 nm) |
| Example 1 | HI-1 | IL | HT-1 | BH | BD | (1-1-2) | Liq/Mg:Ag |
| Example 2 | HI-1 | IL | HT-1 | BH | BD | (1-2-27) | Liq/Mg:Ag |
| Example 3 | HI-1 | IL | HT-1 | BH | BD | (1-2-48) | Liq/Mg:Ag |
| Example 4 | HI-1 | IL | HT-1 | BH | BD | (1-2-173) | Liq/Mg:Ag |
| Example 5 | HI-1 | IL | HT-1 | BH | BD | (1-2-179) | Liq/Mg:Ag |
| Example 6 | HI-1 | IL | HT-1 | BH | BD | (1-2-506) | Liq/Mg:Ag |
| Comparative | HI-1 | IL | HT-1 | BH | BD | A | Liq/Mg:Ag |
| Example 1 | | | | | | | |
| Comparative | HI-1 | IL | HT-1 | BH | BD | B | Liq/Mg:Ag |
| Example 2 | | | | | | | |
| Comparative | HI-1 | IL | HT-1 | BH | BD | C | Liq/Mg:Ag |
| Example 3 | | | | | | | |
| Comparative | HI-1 | IL | HT-1 | BH | BD | D | Liq/Mg:Ag |
| Example 4 | | | | | | | |
| Comparative | HI-1 | IL | HT-1 | BH | BD | E | Liq/Mg:Ag |
| Example 5 | | | | | | | |
| Comparative | HI-1 | IL | HT-1 | BH | BD | F | Liq/Mg:Ag |
| Example 6 | | | | | | | |

*The electron transport layer was formed so that the mixture of the compound in the table and Liq was a 1:1 weight ratio.

TABLE 2

| | Hole | Hole | Hole | | | Electron | Electron | |
|---|---|---|---|---|---|---|---|---|
| | Injection | Injection | Transport | Emission Layer | | Transport | Injection | |
| | Layer 1 | Layer 2 | Layer | (20 nm) | | Layer | Layer* | Cathode |
| | (40 nm) | (5 nm) | (25 nm) | Host | Dopant | (10 nm) | (20 nm) | (1 nm/100 nm) |
| Example 7 | HI-1 | IL | HT-1 | BH | BD | (1-2-48) | I | Liq/Mg:Ag |
| Example 8 | HI-1 | IL | HT-1 | BH | BD | (1-2-173) | I | Liq/Mg:Ag |
| Example 9 | HI-1 | IL | HT-1 | BH | BD | (1-2-179) | I | Liq/Mg:Ag |
| Example 10 | HI-1 | IL | HT-1 | BH | BD | (1-2-506) | I | Liq/Mg:Ag |
| Comparative | HI-1 | IL | HT-1 | BH | BD | G | I | Liq/Mg:Ag |
| Example 7 | | | | | | | | |
| Comparative | HI-1 | IL | HT-1 | BH | BD | H | I | Liq/Mg:Ag |
| Example 8 | | | | | | | | |

*The electron injection layer was formed so that the mixture of the compound in the table and Liq was a 1:1 weight ratio.

In Tables 1 and 2, “HI-1” represents N⁴,N⁴-diphenyl-N⁴,N⁴-bis(9-phenyl-9H-carbazol-3-yl)-[1,1′-biphenyl]-4,4′-diamine, “IL” represents 1,4,5,8,9,12-hexaazatriphenylenehexacarbonitrile, “HT-1” represents N-([1,1′-biphenyl]-4-yl)-9,9-dimethyl-N-(4-(9-phenyl-9H-carbazol-3-yl)phenyl)-9H-fluorene-2-amine, “BH” represents 9-phenyl-10-(4-phenylnaphthalen-1-yl)anthracene, “BD” represents 7,7-dimethyl-N⁵,N⁹-diphenyl-N⁵,N⁹-bis(4-trimethylsilyl)phenyl)-7H-benzo[c]fluorene-5,9-diamine, “A” represents 3-(6-(10-phenylanthracen-9-yl)naphthalen-2-yl)pyridine, “B” represents 9,10-bis(4-(3-pyridylphenyl))-2-phenylanthracene, “C” represents 9,10-bis(2,3′-bipyridin-6-yl)-2-phenylanthracene, “D” represents 2,7-bis((2,4′-bipyridin-6-yl))-5,5′-(9,9′-spirobi[fluorene]), “E” represents 2,7-bis(2,4′-bipyridin-6-yl)triphenylene, “F” represents 9,10-bis(4-cyanophenyl)-2-phenylanthracene, “G” represents 9-(4′-(dimethylboryl)-[1,1′-binaphthalen]-4-yl)-9H-carbazole, “H” represents 3-(3-(6-(9,9-dimethyl-9H-fluorene-2-yl)naphthalen-2-yl)phenyl)fluoranthene, and “I” represents 9,10-bis(2,2′-bipyridin-6-yl)-2-phenylanthracene. Chemical structures are shown below together with “Liq” used in the cathode.

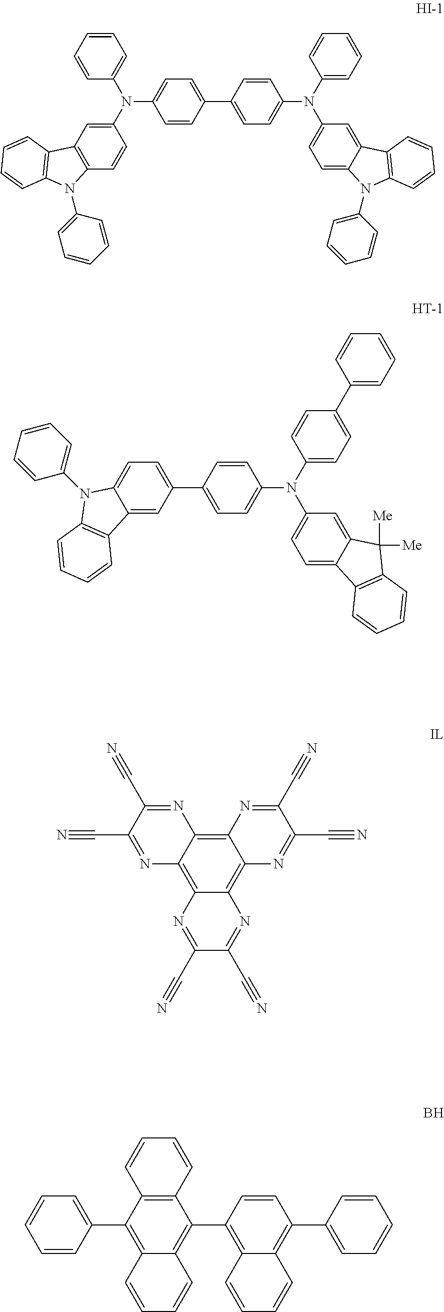

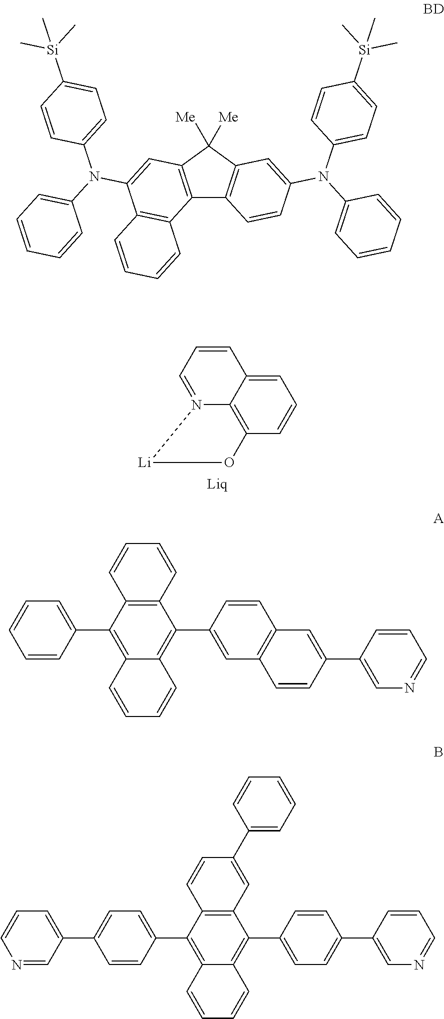

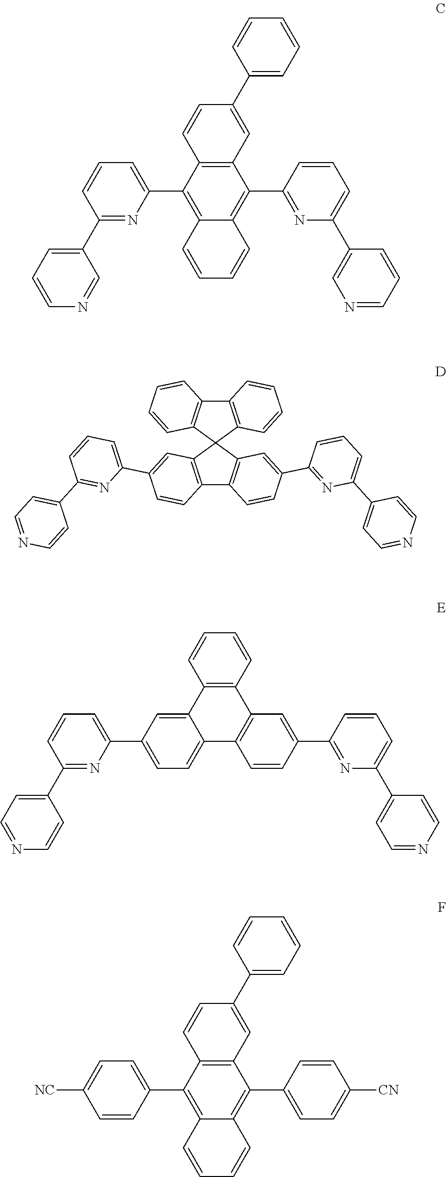 [SMILES: c1ccc(-c2ccc3c(-c4cccc(-c5cccnc5)n4)c4ccccc4c(-c4cccc(-c5cccnc5)n4)c3c2)cc1 ; c1cc(-c2ccncc2)nc(-c2ccc3c(c2)C2(c4ccccc4-c4ccccc42)c2cc(-c4cccc(-c5ccncc5)n4)ccc2-3)c1 ; c1cc(-c2ccncc2)nc(-c2ccc3c4ccc(-c5cccc(-c6ccncc6)n5)cc4c4ccccc4c3c2)c1 ; N#Cc1ccc(-c2c3ccccc3c(-c3ccc(C#N)cc3)c3cc(-c4ccccc4)ccc23)cc1]

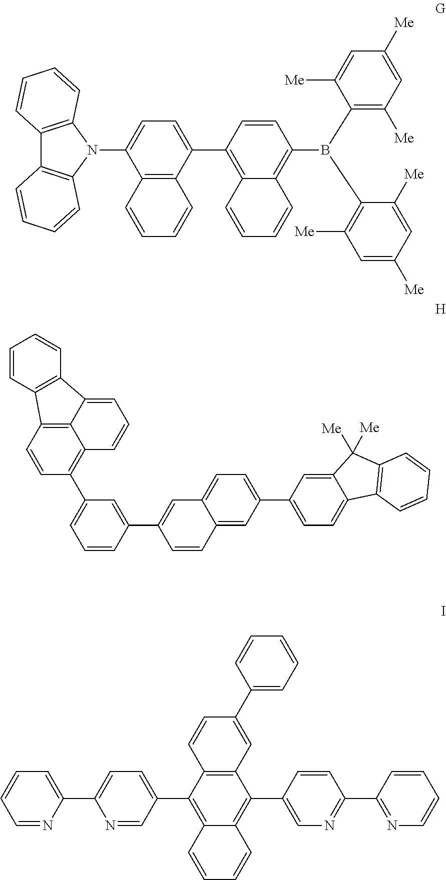

### Example 1

### Device Using Compound (1-1-2) in Electron Transport Material

A 26 mm×28 mm×0.7 mm glass substrate (manufactured by Opto Science, Inc.), on which a film of ITO has been formed at a thickness of 180 nm by sputtering and has been polished to a thickness of 150 nm, was used as a transparent support substrate. This transparent support substrate was fixed to a substrate holder in a commercially-available vapor deposition device (manufactured by Showa Shinku Co., Ltd.), and a molybdenum evaporation boat in which HI-1 was put, a molybdenum evaporation boat in which IL was put, a molybdenum evaporation boat in which HT-1 was put, a molybdenum evaporation boat in which BH was put, a molybdenum evaporation boat in which BD was put, a molybdenum evaporation boat in which compound (1-1-2) of the present invention was put, a molybdenum evaporation boat in which Liq was put, a tungsten evaporation boat in which magnesium was put, and a tungsten evaporation boat in which silver was put were mounted.

Each layer described below was sequentially formed on the ITO film of the transparent support substrate. A vacuum chamber was decompressed to 5×10⁻⁴ Pa, the evaporation boat in which HI-1 was put was first heated to vapor deposit HI-1 so as to become a film thickness of 40 nm, and further the evaporation boat in which IL was put was heated to vapor deposit IL so as to become a film thickness of 5 nm, to form a two-layered hole injection layer. Subsequently, the evaporation boat in which HT-1 was put was heated to vapor deposit HT-1 so as to become a film thickness of 25 nm, to form a hole transport layer. Next, the evaporation boat in which BH was put and the evaporation boat in which BD was put were simultaneously heated to vapor deposit BH and BD so as to become a film thickness of 20 nm, to form an emission layer. The vapor deposition rate was adjusted so as to become about a 95:5 weight ratio of BH to BD. Next, the evaporation boat in which compound (1-1-2) was put and the evaporation boat in which Liq was put were simultaneously heated to vapor deposit compound (1-1-2) and Liq so as to become a film thickness of 30 nm, to form an electron transport layer. The vapor deposition rate was adjusted so as to become about a 1:1 weight ratio of compound (1-1-2) to Liq. The vapor deposition rate of each layer was 0.01 to 1 nm/sec.

Then, the evaporation boat in which Liq was put was heated to vapor deposit Liq at a vapor deposition rate of 0.01 to 1 nm/sec so as to become a film thickness of 1 mm Subsequently, the evaporation boat in which magnesium was put and the evaporation boat in which silver was put were simultaneously heated to vapor deposit magnesium and silver so as to become a film thickness of 100 nm, to form an anode, and an organic EL device was thus obtained. When doing so, the vapor deposition rate was adjusted between 0.1 to 10 nm/sec so that the atomic ratio of magnesium to silver became 10:1.

When the characteristics during emission of 1,000 cd/m² were measured using an ITO electrode as an anode and an Mg/Ag electrode as a cathode, the driving voltage was 4.25 V and the external quantum efficiency was 4.25%. As a result of carrying out a constant current drive test at a current density for obtaining a luminance of 1,500 cd/m², the time for which a luminance of 80% (1,200 cd/m²) or more of the initial luminance was maintained was 270 hours.

### Example 2

### Device Using Compound (1-2-27) in Electron Transport Material

Other than changing compound (1-1-2) to compound (1-2-27), an organic EL device was obtained in accordance with the method of Example 1. When a direct current voltage was applied and the characteristics during emission of 1,000 cd/m² measured, the driving voltage was 4.21 V and the external quantum efficiency was 3.75%. As a result of carrying out a constant current drive test at a current density for obtaining a luminance of 1,500 cd/m², the time for which a luminance of 80% (1,200 cd/m²) or more of the initial luminance was maintained was 157 hours.

### Example 3

### Device Using Compound (1-2-48) in Electron Transport Material

Other than changing compound (1-1-2) to compound (1-2-48), an organic EL device was obtained in accordance with the method of Example 1. When a direct current voltage was applied and the characteristics during emission of 1,000 cd/m² measured, the driving voltage was 4.47 V and the external quantum efficiency was 4.21%. As a result of carrying out a constant current drive test at a current density for obtaining a luminance of 1,500 cd/m², the time for which a luminance of 80% (1,200 cd/m²) or more of the initial luminance was maintained was 298 hours.

### Example 4

### Device Using Compound (1-2-173) in Electron Transport Material

Other than changing compound (1-1-2) to compound (1-2-173), an organic EL device was obtained in accordance with the method of Example 1. When a direct current voltage was applied and the characteristics during emission of 1,000 cd/m² measured, the driving voltage was 4.30 V and the external quantum efficiency was 7.20%. As a result of carrying out a constant current drive test at a current density for obtaining a luminance of 1,500 cd/m², the time for which a luminance of 80% (1,200 cd/m²) or more of the initial luminance was maintained was 312 hours.

### Example 5

### Device Using Compound (1-2-179) in Electron Transport Material

Other than changing compound (1-1-2) to compound (1-2-179), an organic EL device was obtained in accordance with the method of Example 1. When a direct current voltage was applied and the characteristics during emission of 1,000 cd/m² measured, the driving voltage was 4.20 V and the external quantum efficiency was 4.96%. As a result of carrying out a constant current drive test at a current density for obtaining a luminance of 1,500 cd/m², the time for which a luminance of 80% (1,200 cd/m²) or more of the initial luminance was maintained was 292 hours.

### Example 6

### Device Using Compound (1-2-506) in Electron Transport Layer

Other than changing compound (1-1-2) to compound (1-2-506), an organic EL device was obtained in accordance with the method of Example 1. When a direct current voltage was applied and the characteristics during emission of 1,000 cd/m² measured, the driving voltage was 4.23 V and the external quantum efficiency was 4.75%. As a result of carrying out a constant current drive test at a current density for obtaining a luminance of 1,500 cd/m², the time for which a luminance of 80% (1,200 cd/m²) or more of the initial luminance was maintained was 303 hours.

### Comparative Example 1

Other than changing compound (1-1-2) to compound (A), an organic EL device was obtained in accordance with the method of Example 1. When a direct current voltage was applied and the characteristics during emission of 1,000 cd/m² measured, the driving voltage was 3.46 V and the external quantum efficiency was 5.55%. Also, as a result of carrying out a constant current drive test at a current density for obtaining a luminance of 1,500 cd/m², the time for which a luminance of 80% (1,200 cd/m²) or more of the initial luminance was maintained was 169 hours.

### Comparative Example 2

Other than changing compound (1-1-2) to compound (B), an organic EL device was obtained in accordance with the method of Example 1. When a direct current voltage was applied and the characteristics during emission of 1,000 cd/m² measured, the driving voltage was 3.51 V and the external quantum efficiency was 5.24%. Also, as a result of carrying out a constant current drive test at a current density for obtaining a luminance of 1,500 cd/m², the time for which a luminance of 80% (1,200 cd/m²) or more of the initial luminance was maintained was 77 hours.

### Comparative Example 3

Other than changing compound (1-1-2) to compound (C), an organic EL device was obtained in accordance with the method of Example 1. When a direct current voltage was applied and the characteristics during emission of 1,000 cd/m² measured, the driving voltage was 3.97 V and the external quantum efficiency was 5.93%. Also, as a result of carrying out a constant current drive test at a current density for obtaining a luminance of 1,500 cd/m², the time for which a luminance of 80% (1,200 cd/m²) or more of the initial luminance was maintained was 145 hours.

### Comparative Example 4

Other than changing compound (1-1-2) to compound (D), an organic EL device was obtained in accordance with the method of Example 1. When a direct current voltage was applied and the characteristics during emission of 1,000 cd/m² measured, the driving voltage was 3.75 V and the external quantum efficiency was 5.89%. Also, as a result of carrying out a constant current drive test at a current density for obtaining a luminance of 1,500 cd/m², the time for which a luminance of 80% (1,200 cd/m²) or more of the initial luminance was maintained was 116 hours.

### Comparative Example 5

Other than changing compound (1-1-2) to compound (E), an organic EL device was obtained in accordance with the method of Example 1. When a direct current voltage was applied and the characteristics during emission of 1,000 cd/m² measured, the driving voltage was 3.55 V and the external quantum efficiency was 7.45%. Also, as a result of carrying out a constant current drive test at a current density for obtaining a luminance of 1,500 cd/m², the time for which a luminance of 80% (1,200 cd/m²) or more of the initial luminance was maintained was 155 hours.

### Comparative Example 6

Other than changing compound (1-1-2) to compound (F), an organic EL device was obtained in accordance with the method of Example 1. When a direct current voltage was applied and the characteristics during emission of 1,000 cd/m² measured, the driving voltage was 4.79 V and the external quantum efficiency was 2.81%. Also, as a result of carrying out a constant current drive test at a current density for obtaining a luminance of 1,500 cd/m², the time for which a luminance of 80% (1,200 cd/m²) or more of the initial luminance was maintained was 122 hours.

The results of the aforementioned Examples 1 to 6 and Comparative Examples 1 to 6 are collectively shown in Table 3.

TABLE 3

| | | Characteristics During | | Time Luminance |
|---|---|---|---|---|
| | | Emission of 1,000 cd/m² | | 80% or More |
| | Electron | | External | of Initial |
| | Transport | Voltage | Quantum | Luminance |
| | Layer* | (V) | Efficiency (%) | Maintained (hr) |
| Example 1 | (1-1-2) | 4.25 | 4.25 | 270 |
| Example 2 | (1-2-27) | 4.21 | 3.75 | 157 |
| Example 3 | (1-2-48) | 4.47 | 4.21 | 298 |
| Example 4 | (1-2-173) | 4.30 | 7.20 | 312 |
| Example 5 | (1-2-179) | 4.20 | 4.96 | 292 |
| Example 6 | (1-2-506) | 4.23 | 4.75 | 303 |
| Comparative | A | 3.46 | 5.55 | 169 |
| Example 1 | | | | |
| Comparative | B | 3.51 | 5.24 | 77 |
| Example 2 | | | | |
| Comparative | C | 3.97 | 5.93 | 145 |
| Example 3 | | | | |
| Comparative | D | 3.75 | 5.89 | 116 |
| Example 4 | | | | |
| Comparative | E | 3.55 | 7.45 | 155 |
| Example 5 | | | | |
| Comparative | F | 4.79 | 2.81 | 122 |
| Example 6 | | | | |

*The electron transport layer was formed so that the mixture of the compound in the table and Liq was a 1:1 weight ratio.

### Example 7

### Device Using Compound (1-2-48) in Electron Transport Material of Device Having Electron Transport Layer and Electron Injection Layer

After forming until an emission layer by the same method as Example 1, an evaporation boat in which compound (1-2-48) was put was heated to vapor deposit compound (1-2-48) so as to become a film thickness of 10 nm, to form an electron transport layer. Subsequently, the evaporation boat in which compound I was put and an evaporation boat in which Liq was put were simultaneously heated to vapor deposit compound I and Liq so as to become a film thickness of 20 nm, to form an electron injection layer. The vapor deposition rate was adjusted so as to become about a 1:1 weight ratio of compound I to Liq. The vapor deposition rate of each layer was 0.01 to 1 nm/sec. Subsequently, an Liq layer and a cathode were formed by the same method as Example 1, and an organic EL device was thus obtained.

When the characteristics during emission of 1,000 cd/m² were measured using an ITO electrode as an anode and an Mg/Ag electrode as a cathode, the driving voltage was 4.63 V and the external quantum efficiency was 5.05%. Also, as a result of carrying out a constant current drive test at a current density for obtaining a luminance of 1,500 cd/m², the time for which a luminance of 80% (1,200 cd/m²) or more of the initial luminance was maintained was 420 hours.

### Example 8

### Device Using Compound (1-2-173) in Electron Transport Material of Device Having Electron Transport Layer and Electron Injection Layer

Other than changing compound (1-2-48) to compound (1-2-173), an organic EL device was obtained in accordance with the method of Example 7. When a direct current voltage was applied and the characteristics during emission of 1,000 cd/m² measured, the driving voltage was 3.49 V and the external quantum efficiency was 7.59%. As a result of carrying out a constant current drive test at a current density for obtaining a luminance of 1,500 cd/m², the time for which a luminance of 80% (1,200 cd/m²) or more of the initial luminance was maintained was 373 hours.

### Example 9

### Device Using Compound (1-2-179) in Electron Transport Material of Device Having Electron Transport Layer and Electron Injection Layer

Other than changing compound (1-2-48) to compound (1-2-179), an organic EL device was obtained in accordance with the method of Example 7. When a direct current voltage was applied and the characteristics during emission of 1,000 cd/m² measured, the driving voltage was 4.18 V and the external quantum efficiency was 5.88%. As a result of carrying out a constant current drive test at a current density for obtaining a luminance of 1,500 cd/m², the time for which a luminance of 80% (1,200 cd/m²) or more of the initial luminance was maintained was 395 hours.

### Example 10

### Device Using Compound (1-2-506) in Electron Transport Material of Device Having Electron Transport Layer and Electron Injection Layer

Other than changing compound (1-2-48) to compound (1-2-506), an organic EL device was obtained in accordance with the method of Example 7. When a direct current voltage was applied and the characteristics during emission of 1,000 cd/m² measured, the driving voltage was 4.26 V and the external quantum efficiency was 5.52%. As a result of carrying out a constant current drive test at a current density for obtaining a luminance of 1,500 cd/m², the time for which a luminance of 80% (1,200 cd/m²) or more of the initial luminance was maintained was 255 hours.

### Comparative Example 7

### Device Using Compound G in Electron Transport Material of Device Having Electron Transport Layer and Electron Injection Layer

Other than changing compound (1-2-48) to compound G, an organic EL device was obtained in accordance with the method of Example 7. When a direct current voltage was applied and the characteristics during emission of 1,000 cd/m² measured, the driving voltage was 4.02 V and the external quantum efficiency was 5.79%. As a result of carrying out a constant current drive test at a current density for obtaining a luminance of 1,500 cd/m², the time for which a luminance of 80% (1,200 cd/m²) or more of the initial luminance was maintained was 202 hours.

### Comparative Example 8

### Device Using Compound H in Electron Transport Material of Device Having Electron Transport Layer and Electron Injection Layer

Other than changing compound (1-2-48) to compound H, an organic EL device was obtained in accordance with the method of Example 7. When a direct current voltage was applied and the characteristics during emission of 1,000 cd/m² measured, the driving voltage was 3.73 V and the external quantum efficiency was 6.29%. As a result of carrying out a constant current drive test at a current density for obtaining a luminance of 1,500 cd/m², the time for which a luminance of 80% (1,200 cd/m²) or more of the initial luminance was maintained was 172 hours.

The results of the aforementioned Examples 7 to 10, and Comparative Examples 7 and 8 are collectively shown in Table 4.

TABLE 4

| | | | Characteristics During | | Time Luminance |
|---|---|---|---|---|---|
| | | | Emission of 1,000 cd/m² | | 80% or More |
| | Electron | Electron | | External | of Initial |
| | Transport | Injection | Voltage | Quantum | Luminance |
| | Layer | Layer* | (V) | Efficiency (%) | Maintained (hr) |
| Example 7 | (1-2-48) | I | 4.63 | 5.05 | 420 |
| Example 8 | (1-2-173) | I | 3.49 | 7.59 | 373 |
| Example 9 | (1-2-179) | I | 4.18 | 5.88 | 395 |
| Example 10 | (1-2-506) | I | 4.26 | 5.52 | 255 |
| Comparative | G | I | 4.02 | 5.79 | 202 |
| Example 7 | | | | | |
| Comparative | H | I | 3.73 | 6.29 | 172 |
| Example 8 | | | | | |

*The electron injection layer was formed so that the mixture of the compound in the table and Liq was a 1:1 weight ratio.

Also, in order to carry out device evaluation as a comparative example of Compound J, which is shown below and which is disclosed in the patent publication CN101412907, preparation thereof was attempted. In the patent publication CN101412907, although using 4-(2-bromoacetyl)benzonitrile as a starting material is described, other than determining that this is a typographical error for 2-bromobenzoylacetonitrile, preparation was attempted as described in the patent publication. However, only a complex mixture was obtained by the reaction of the final stage, as well as the majority of the products was a black tarry material and compound J was not able to be obtained. Also, although small amounts of multiple products were able to be confirmed by reacting a pyridine salt after separately preparing in advance an enone compound by condensing anthracene-9,10-dicarboxyaldehyde and 4-tertiary butylacetophenone under basic conditions referring to the generally-known 2,6-diphenylpyridine synthesis method, it was limited to most of the raw material enone compound being recovered as is unreacted and compound J was not able to be obtained. Also, referring to the description of Journal of Materials Chemistry, 2011, 21, 12977, although preparation of a comparative example compound was attempted by reacting benzoylacetonitrile with the enone compound prepared by condensing anthracene-9,10-dicarboxyaldehyde and 4-tert-butylacetophenone, similarly a complex mixture with the majority being a black tarry material was obtained, and the target compound J was not able to be obtained.

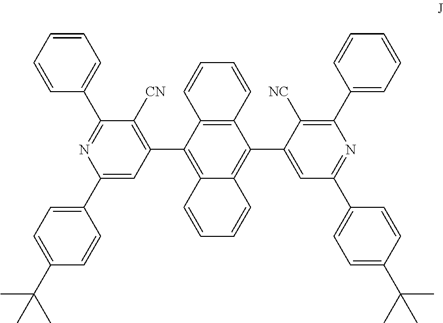 [SMILES: CC(C)(C)c1ccc(-c2cc(-c3c4ccccc4c(-c4cc(-c5ccc(C(C)(C)C)cc5)nc(-c5ccccc5)c4C#N)c4ccccc34)c(C#N)c(-c3ccccc3)n2)cc1]

### INDUSTRIAL APPLICABILITY

According to preferred aspects of the present invention, an organic EL device which can achieve a good balance in characteristics required for the organic EL device, such as driving voltage, high efficiency, and extended lifetime, and which particularly has extended lifetime as a characteristic, can be provided, and a high performance display apparatus for a full-color display and the like, can be provided.

1. A compound represented by formula (1) below: 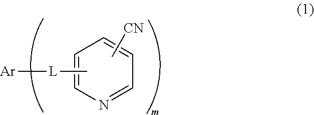
wherein, in formula (1),
Ar is a divalent group selected from the group represented by formulae (Ar2-1), (Ar2-8), (Ar2-12), and (Ar2-21) below: 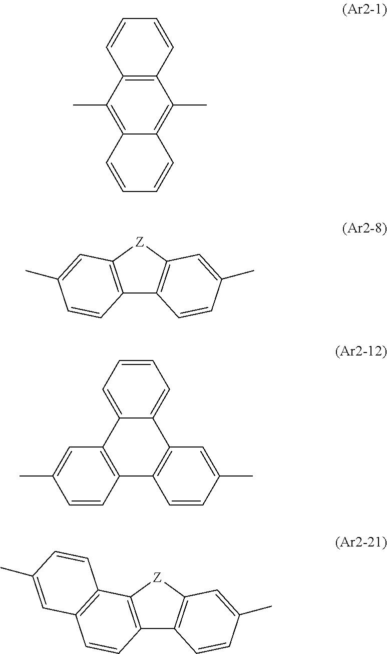
wherein
in formulae (Ar2-1), (Ar2-8), (Ar2-12), and (Ar2-21), Z is independently a divalent group represented by formula (4) below, and at least one hydrogen in each group may be replaced by an alkyl having 1 to 4 carbons: 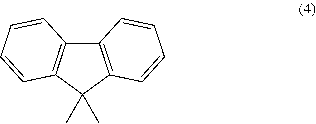
and at least one hydrogen in these groups may be replaced by an alkyl having 1 to 6 carbons;
m is 2, and a group formed by a pyridine ring and L may be identical or different;
L is a single bond or one selected from a group of divalent groups represented by formulae (L-1) and (L-2) below: 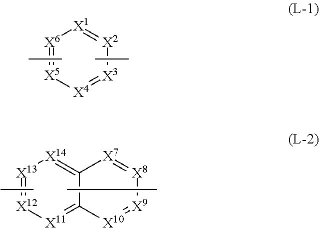
wherein,
in formula (L-1), X¹ to X⁶ are independently ═CR¹— or ═N—, at least two of X¹ to X⁶ are ═CR¹—, R¹ in two ═CR¹— of X¹ to X⁶ is a bond for bonding with Ar or the pyridine ring, and R¹ in other ═CR¹— is hydrogen,
in formula (L-2), X⁷ to X¹⁴ are independently ═CR¹— or ═N—, at least two of X⁷ to X¹⁴ are ═CR¹—, R¹ in two ═CR¹— of X⁷ to X¹⁴ is a bond for bonding with Ar or the pyridine ring, and R¹ in other ═CR¹— is hydrogen, and
at least one hydrogen of L may be replaced by an alkyl having 1 to 4 carbons or an aryl having 6 to 18 carbons;
at least one hydrogen of the pyridine ring may be replaced by an alkyl having 1 to 4 carbons, phenyl, or naphthyl; and
at least one hydrogen of each ring and alkyl in formula (1) may be replaced by deuterium.

2-6. (canceled)

7. The compound according to claim 1 represented by formula (1-2-27) below:

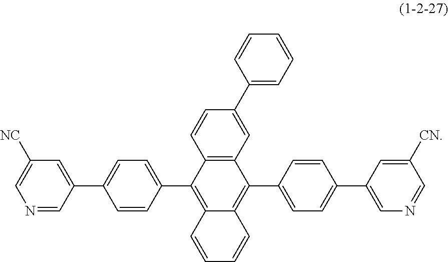 [SMILES: N#Cc1cncc(-c2ccc(-c3c4ccccc4c(-c4ccc(-c5cncc(C#N)c5)cc4)c4cc(-c5ccccc5)ccc34)cc2)c1]

8. The compound according to claim 1 represented by formula (1-2-48), (1-2-173), (1-2-179), (1-2-365), (1-2-506), or (1-2-507) below:

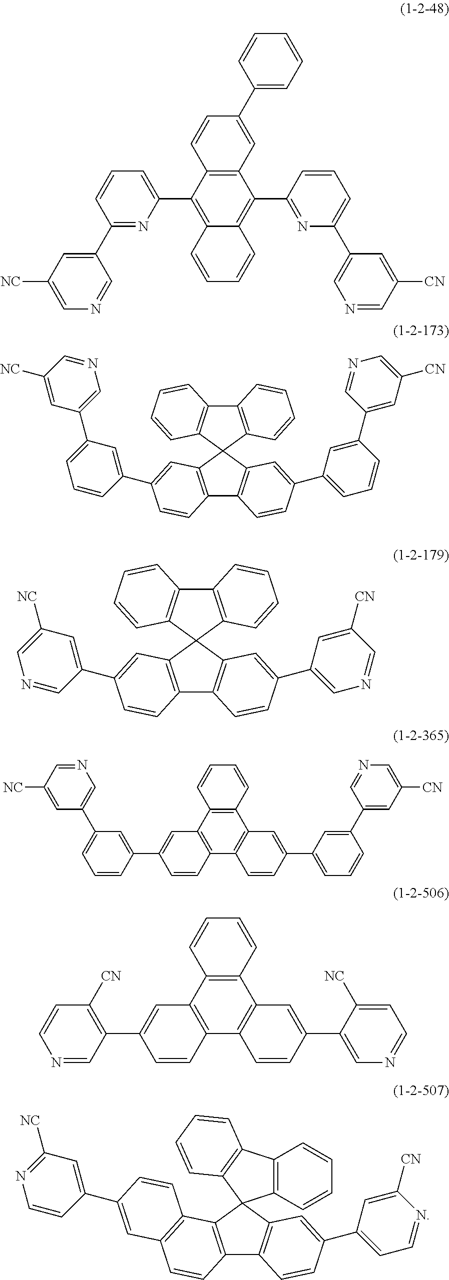

9. An electron transport material containing the compound according to claim 1.

10. An organic electroluminescent device having: a pair of electrodes formed of an anode and a cathode; an emission layer arranged between the pair of electrodes; and an electron transport layer and/or an electron injection layer which contain(s) the electron transport material according to claim 9 and which are/is arranged between the cathode and the emission layer.

11. An organic electroluminescent device having: a pair of electrodes formed of an anode and a cathode; an emission layer arranged between the pair of electrodes; and an electron transport layer and an electron injection layer which contain the electron transport material according to claim 9 and which are arranged between the cathode and the emission layer.

12. The organic electroluminescent device according to claim 10, wherein at least one of the electron transport layer and the electron injection layer further contains at least one selected from a group consisting of quinolinol metal complexes, bipyridine derivatives, phenanthroline derivatives, and borane derivatives.

13. (canceled)

14. The organic electroluminescent device according to claim 11, wherein at least one of the electron transport layer and the electron injection layer further contains at least one selected from a group consisting of quinolinol metal complexes, bipyridine derivatives, phenanthroline derivatives, and borane derivatives.

15. The organic electroluminescent device according to any one of claims 10, 11, 12, and 14, wherein at least one of the electron transport layer and the electron injection layer further contains at least one selected from a group consisting of alkali metals, alkali earth metals, rare earth metals, alkali metal oxides, alkali metal halides, alkali earth metal oxides, alkali earth metal halides, rare earth metal oxides, rare earth metal halides, organic complexes of an alkali metal, organic complexes of an alkali earth metal, and organic complexes of a rare earth metal.
